(19) United States
(12) Patent Application Publication
(10) Pub. No.: US 2016/0242682 A1
(43) Pub. Date: Aug. 25, 2016

(54) NONINVASIVE ANALYZER APPARATUS AND METHOD OF USE THEREOF FOR SEPARATING DISTRIBUTED PROBING PHOTONS EMERGING FROM A SAMPLE

(71) Applicants: Sandeep Gulati, La Canada, CA (US); Timothy Ruchti, Gurnee, IL (US); Kevin H. Hazen, Gilbert, AZ (US)

(72) Inventors: Sandeep Gulati, La Canada, CA (US); Timothy Ruchti, Gurnee, IL (US); Kevin H. Hazen, Gilbert, AZ (US)

(21) Appl. No.: 14/965,807

(22) Filed: Dec. 10, 2015

Related U.S. Application Data
(63) Continuation-in-part of application No. 14/504,065, filed on Oct. 1, 2014.
Application No. 14/504,065 is a continuation-in-part of application No. 13/963,925, filed on Aug. 9, 2013.
Application No. 13/963,925 is a continuation-in-part of application No. 13/963,933, filed on Aug. 9, 2013.
Application No. 13/963,933 is a continuation-in-part of application No. 13/941,411, filed on Jul. 12, 2013.
Application No. 13/941,411 is a continuation-in-part of application No. 13/941,389, filed on Jul. 12, 2013.
Application No. 13/941,389 is a continuation-in-part of application No. 13/941,369, filed on Jul. 12, 2013.
(60) Provisional application No. 61/672,195, filed on Jul. 16, 2012.
(60) Provisional application No. 61/700,291, filed on Sep. 12, 2012.
(60) Provisional application No. 61/700,294, filed on Sep. 12, 2012.

(51) Int. Cl. A61B 5/1455; G01N 21/41; G01N 21/359; A61B 5/145; A61B 5/00

(52) U.S. Cl. CPC A61B 5/1455; A61B 5/14546; A61B 5/7278; A61B 5/14532; G01N 21/359; G01N 21/4133; G01N 2201/061

(57) ABSTRACT

A noninvasive analyzer apparatus and method of use thereof is described for spatially separating light having noninvasively probed a tissue volume into groups, which narrows standard deviations of probed tissue pathlength for each of the groups. Reduction in tissue pathlength uncertainty subsequently enhances noninvasive analyte concentration determination accuracy. Control of individual detector distance from an illumination zone in combination with control of area of a detection zone coupled to an individual detector yields intensity control of the various groups. The intensity control is optionally aided using several intensity control elements including: control of detector response shape, hardware gain settings set as function of distance from the illumination zone, varying numerical aperture of light collection optics as a function of position from the illumination zone, multiple illumination-detector linked bundlets, micro-optics, segmented spacers, arcs of detector elements, and/or outlier analysis based on detected intensity as a function of position.

### CROSS REFERENCES TO RELATED APPLICATIONS

This application is a continuation-in-part of U.S. patent application Ser. No. 14/504,065 filed Oct. 1, 2014, which:

is a continuation-in-part of U.S. patent application Ser. No. 13/963,925 filed Aug. 9, 2013;

is a continuation-in-part of U.S. patent application Ser. No. 13/963,933 filed Aug. 9, 2013, which is a continuation-in-part of U.S. patent application Ser. No. 13/941,411 filed Jul. 12, 2013, which is a continuation-in-part of U.S. patent application Ser. No. 13/941,389 filed Jul. 12, 2013, which is a continuation-in-part of U.S. patent application Ser. No. 13/941,369 filed Jul. 12, 2013, which claims the benefit of:
U.S. provisional patent application No. 61/672,195 filed Jul. 16, 2012;
U.S. provisional patent application No. 61/700,291 filed Sep. 12, 2012; and
U.S. provisional patent application No. 61/700,294 filed Sep. 12, 2012, and

claims benefit of U.S. provisional patent application No. 62/166,063 filed May 25, 2015

all of which are incorporated herein in their entirety by this reference thereto.

### TECHNICAL FIELD OF THE INVENTION

The present invention relates to an analyzer comprising multiple source and/or detector elements and an algorithm that generates and uses information related to multiple optical pathways between the one or more sources and the one or more detectors to yield enhanced precision and/or accuracy of determination of a property of a probed non-uniform sample. The multiple pathways result from separation of photonic pathways via binning separate photonic pathways into separate spatially separated source illumination zone to detection zone distances, optionally as a function of time.

### DESCRIPTION OF THE RELATED ART

Traditional spectroscopic approaches apply to uniform samples and/or to samples that have been previously separated into components and/or reacted with another chemical/substance to form a sample that: (1) is purer allowing for a larger signal; (2) is processed to remove one or more interferences, (3) is mountable in a spectrometer in a controlled manner, and/or (4) that has a larger signal to relate to an analyte of interest, such as an introduced observable element directly or indirectly related to the analyte of interest. However, in a truly noninvasive analysis, use of: a traditional chemical/physical separation technique, a mechanical/chemical/electrical homogenization, and/or a chemical derivation in a sample preprocessing step is not applicable and the sample is necessarily treated/analyzed as a whole. For analysis of whole samples, especially in a form of reflectance analysis, contribution of signal, in terms of intensity, reflectance, absorbance, power, or the like, of probed total optical pathlength, probed optical radius, probed optical depth, and/or total probed pathlength in a layer is not adequately separated from the observed contribution or signal related to the analyte property due to sample inhomogeneities, which leads to poor accuracy and/or increased error of the analysis of the desired analyte property.

### Problem Statement

What is needed is an analyzer with enhanced bulk sampling capabilities/pathlength resolution, via hardware and/or software, yielding more accurate and/or precise analyte property information derived from spectra of the bulk sample.

### SUMMARY OF THE INVENTION

The invention comprises a spectroscopic analyzer apparatus comprising multiple and at least partially separable source illumination zone to detector detection zone pathways through a sample and means for further controlling the multiple source to detector pathways to enhance accuracy of an analyte property estimation and/or enhance precision of the analyte property estimation via pathlength control.

### BRIEF DESCRIPTION OF THE DRAWINGS

A more complete understanding of the present invention is derived by referring to the detailed description and claims when considered in connection with the Figures, wherein like reference numbers refer to similar items throughout the Figures.

FIG. 1 illustrates an analyzer system;

FIGS. 2(A-K) illustrate photonic pathways through tissue;

FIG. 3 illustrates probing tissue layers using a spatial distribution method;

FIG. 4A and FIG. 4B illustrate varying illumination zones relative to a detector;

FIGS. 5(A-O) illustrate varying detection zones relative to an illuminator;

FIG. 6A illustrates an end view of a detector array and FIG. 6B illustrates a side view of the detector array;

FIGS. 7(A-E) illustrate a coupled source detector array system, FIG. 7A; a side illuminated/detector array system, FIG. 7B; a corner illuminated/detector array system, FIG. 7C; a within array illumination system, FIG. 7D; and an illuminated array/detector array system, FIG. 7E;

FIG. 8A and FIG. 8B illustrate a first example of a multiple two-dimensional detector array system and a second example of a multiple two-dimensional detector array system, respectively;

FIG. 9A illustrates transmission spectra of longpass optical filters and FIG. 9B relates longpass filters to water absorbance;

FIG. 10 illustrates shortpass filter transmission spectra;

FIG. 11A illustrates bandpass filters relative to near-infrared spectral regions and FIG. 11B illustrates specialized bandpass filters;

FIG. 12 illustrates elements of a bimodal optical filter;

FIG. 13A and FIG. 13B illustrate a fat band filter and fat band absorbance, respectively;

FIG. 14A and FIG. 14B illustrate a glucose filter and glucose absorbance, respectively;

FIG. 15 illustrates a detector array with multiple filter array layers;

FIG. 16 illustrates a source array proximate a combined detector/filter array;

FIG. 17 illustrates a source relative to multiple two-dimensional detector arrays;

FIG. 18A and FIG. 18B illustrate an illumination array relative to multiple two-dimensional detector array types and rotated two-dimensional detector arrays, respectively;

FIG. 19A and FIG. 19B illustrate a two-dimensional detector array relative to an optic array in an expanded and assembled view, respectively;

FIG. 20A and FIG. 20B illustrate a detector array, longpass filter array, shortpass filter array, and optic array in an exploded and assembled view, respectively;

FIG. 21A and FIG. 21B illustrate a detector array, longpass filter array, shortpass filter array, and optic array in an exploded and assembled view, respectively;

FIGS. 22(A-H) illustrate light-emitting diode array-detector array combinations;

FIGS. 23(A-C) illustrate a fiber optic bundle, FIG. 23A; a first example sample interface end of the fiber optic bundle, FIG. 23B; and a second example sample interface end of the fiber optic bundle, FIG. 23C;

FIG. 24A illustrates a third example sample interface end of the fiber optic bundle and FIG. 24B illustrates a mask;

FIG. 25 illustrates a mask selection wheel;

FIG. 26A illustrates a position selection optic; FIG. 26B illustrates the position selection optic selecting position; FIG. 26C illustrates solid angle selection using the position selection optic; and FIG. 26D illustrates radial control of incident light relative to a detection zone;

FIG. 27A and FIG. 27B illustrate a pathlength resolved sample interface for a first subject and a second subject, respectively;

FIG. 28A and FIG. 28B provide a method of use of a data processing system and an analyzer control system, respectively;

FIG. 29A and FIG. 29B provide a method of using a sample mapping phase and a subsequent subject specific data collection phase, respectively

FIG. 30A and FIG. 30B illustrate a two dimensional detector array and multiple two-dimensional detector arrays, respectively;

FIG. 31A and FIG. 31B illustrate a first and second example of a multiple/parallel two-dimensional detector array readout system, respectively and

FIG. 31C illustrates an exemplary multiple LED source multiple detection optic layout;

FIG. 32 illustrates a method of linking to a secure data processing system;

FIG. 33A and FIG. 33B illustrate a first and second method of analyzing data, respectively;

FIGS. 34(A-G) illustrate interference detection;

FIG. 35 illustrates chemical absorbance band correlation; and

FIG. 36(A-C) illustrate sample, optical, and position filtered signal, respectively.

Elements and steps in the figures are illustrated for simplicity and clarity of presentation and have not necessarily been rendered according to any particular sequence. For example, steps that are performed concurrently or in a different order are illustrated in the figures to help improve understanding of embodiments of the present invention.

### DETAILED DESCRIPTION OF THE PREFERRED EMBODIMENTS

The invention comprises a spectroscopy based noninvasive analyzer using multiple illuminator element related sample illumination zone to detector array element related detection zone separation distances to narrow individual observed deviations of optical pathways through a sample.

### Intensity Control

A noninvasive analyzer apparatus and method of use thereof is described for spatially separating light having noninvasively probed a tissue volume into groups, which narrows standard deviations of probed tissue pathlength for each of the groups. Reduction in tissue pathlength uncertainty subsequently enhances noninvasive analyte concentration determination accuracy. Control of individual detector distance from an illumination zone in combination with control of area of a detection zone coupled to an individual detector yields intensity control of the various groups. The intensity control is optionally aided using a combination of several intensity control elements including: control of detector response shape, hardware gain settings set as function of distance from the illumination zone, varying numerical aperture as a function of position from the illumination zone, multiple illumination to detector linked bundlets, micro-optics, segmented spacers, arcs of detector elements, and/or outlier analysis based on detected intensity as a function of position.

### Pathlength Control

In one embodiment, traditional analyses relating an analyzer gathered spectrum to an analyte property, such as a concentration, are enhanced through use of a detector array comprising a plurality of detector elements, where the individual detector elements each yield separate yet related information on a sample state, property, or analyte concentration; localized sample structure; a localized sample property; and/or a localized sample state via enhanced accuracy and/or precision of an optically probed pathway and/or an optically probed pathlength. Generally, signals from each individual sample illumination zone to detection zone distance, as measured using individual detector elements, a subset of detector elements, and/or a detector array yields pathway and/or pathlength information comprising one or more of: a mean sample depth, a mean radial distance, a mean total pathlength, a pathlength within a sample volume of interest, a pathlength associated with a source-detector combination, and/or a probabilistically controlled optical pathway relative to integration of signals from most to all of the utilized detector elements. The controlled pathlength is used to enhance accuracy and/or precision of the analyzed sample property through reduction in error associated with pathlength. Optionally, the analyzer is configured with an algorithm to utilize, integrate, analyze, combine, and/or discard discrete signals from individual detector elements of the detector array for the analysis of a non-uniform, heterogeneous, and/or layered sample.

In another embodiment, a solid-state spectroscopic analyzer is presented configured with optical pathway and/or optical pathlength resolution capability through a combination of source wavelength emission properties, sample properties, optical filter properties, and a set of illumination zone to detection zone spacing distances. For instance, a source comprising a mid-range wavelength bandwidth or photons over a narrow range of wavelengths is provided, such as from a laser diode and/or from a set of light emitting diodes. Further, photon bunches from the sources are optically filtered by the tissue sample properties yielding a pathlength control. For instance, photons at wavelengths of high tissue sample absorbance yield shorter pathlengths and vise-versa. The pathlength control is one method of controlling optical pathway. Similarly, the tissue sample scattering coefficient and changes thereof from 900 to 2500 nanometers (nm) alter the total optical pathlength as a function of radial dispersion, which in combination with the described tissue sample absorbance further controls observable pathlengths as a function of radial distance from the illuminator. Further, large changes in tissue sample anisotropy as a function of wavelength, such as from 1100 to 1400 nm, have a large effect on depth of penetration and hence total radial dispersion of the incident photons in view of the described absorbance and the scattering of the tissue sample, which still further controls pathlength of observed photons, as described in detail infra. In addition to the source and sample pathlength control, optical filters are optionally used to further control wavelength ranges, such as by: (1) splitting of a light-emitting diode wavelength band into sections for tighter wavelength resolution; (2) by separating two or more regions of similar absorbance/scattering properties that would otherwise be detected at the same radial distance from an illumination zone; (3) by separating wavelength regions having convolved radial distances from an illumination source due to dissimilar absorbance and scattering properties yielding the same radial distance of emergence from the sample; and/or (4) by combining with the sharp cut-ons or sharp cut-offs of sample absorbance, such as water absorbance, to form a bandpass region. Thus, the source and filter combination controls wavelengths and the source and the sample properties combination controls pathlength. Still further, the filter elements are optically coupled to detector elements where the detector elements themselves are optionally used to still further control detected wavelength regions, such as via sensitivity of response, D*, as a function of wavelength, cut-off wavelength, doping, and/or temperature control of the cut-off wavelength. Combined, the source, sample, optical filter, and detector element, configured in an array of combinations as a function of radial distance from one or more illumination zones relative to one or more detection zones associated with individual detector elements and/or groups of detection elements yield a wavelength resolved, optical pathway resolved, total optical pathlength resolved, and optical depth of penetration resolved analyzer without need of a wavelength separating grating, prism, or dispersive element or a time-based wavelength resolution element, such as a time domain to frequency domain transform based spectrometer, such as a Michelson interferometer. The resulting solid state device, with optional dynamic source position, dynamic source orientation, and/or dynamic optic control, yields a highly multiplexed analyzer and the elimination of need for inventor determined sensitivity analysis of limiting analyzer components, such as fiber optic movement, and for rapid data collection and still further time reduction via detector signal binning for minimization of sample/tissue movement based spectral variations in view of the coupled algorithm, which takes advantage of the multiplexed data/data streams. The integrated wavelength and pathlength controlled analyzer is further described in the body of this application, infra.

In the embodiments described herein, the complexity of the tissue, combinations of tissue chemistry, and tissue physics allows coupling of particular optical visible and/or infrared wavelengths and corresponding particular total optical pathlengths, particular total optical radial distances, and/or a range of tissue parameters to an analyzer with source, optical transport, optical filter, and detector combinations that with current source, optic, and detector technologies allow the interactions of the tissue itself with light to aid in discriminant analysis of the tissue, such as via radial distribution of light analysis. Further, multiple wavelengths, even when overlapping in detected radial distance, are further separated using different light source, filter, and detector combinations in differing radial directions from an illumination zone. Indeed, discrete, even while overlapping, illumination zones, allow further separation of collected data related to tissue depth, tissue layer thickness, tissue inhomogeneities, total optical pathlength, mean depth of penetration, and mean radial distance. The inventor has determined and discloses herein a novel spectrometer apparatus and method of use thereof using tissue sample knowledge, such as absorbance, anisotropy, and/or scattering with optically coupled analyzer elements to discriminate optical pathways, such as total optical pathlengths, probed tissue depth, extent of probing of a tissue layer, and extent of the tissue depth probing as a function of wavelength to yield an analyzer capable of distinguishing even a low concentration analyte concentration, such as a glucose concentration, in the presence of sample inhomogeneities, sample layers of distinct chemical composition, differential scattering as a function of wavelength, and sample constituents of many magnitude larger absorbance, such as a noninvasive glucose concentration determination in skin/blood tissue of the body using light in the range of 400 to 10,000 nanometers.

The inventor notes that traditional spectrometers resolve wavelengths of light and place an emphasis on wavelength resolution elements, such as prisms, grating, and/or time-domain based spectroscopy. However, in the analyzer presented herein it is the optical pathways, such as through tissue, that are partially resolved or separated yielding a tissue pathmeter. The pathmeter is optionally coupled with a spectrometer, uses an element of a spectrometer, and/or is integrated into a spectrometer or vise-versa. Particularly, in the pathmeter an algorithm is used that benefits from multiple optical pathways through the sample, even if the multiple optical pathways are only partially spatially resolved and/or are only partially wavelength resolved. The multiple optical pathways are generated and sensed through a combination of one or more of: multiple source elements; multiple positions of incident light on the sample; varying illumination angles of the multiple source elements to the sample, the sample self-limiting light throughput as a function of wavelength, radial distance, optical depth, and/or direction; multiple wavelength resolving filters; use of micro-optics coupled to detector elements; spatially resolved light collection optics; two or more light collection optics oriented to have partially overlapping or non-overlapping detection zones on the surface of the sample; use of multiple detector types; and/or use of dynamic computer controlled optic pathways all as a function of time and/or radial distance between one or more light illumination zones on the sample and one or more light collection/detection zones, where the detection zone is optionally and preferably radially removed from the illumination zone.

The inventor further notes that in traditional spectrometers, a single reading of the state of the sample is collected and associated with a concentration of an analyte of the sample in a calibration model, where the single reading is a profile as a function of wavelength, such as intensity, reflectance, or absorbance as a function of wavelength. In stark contrast, one embodiment presented herein uses a transform of multiple signals in the calibration phase. For example, the multiple signals input to the transform are optionally multiple different mean pathways through the sample, such as in the pathmeter.

Herein, for clarity and brevity of presentation and without loss of generality, the analyzer having multiple, at least partially separable, probed optical pathways through the sample using one or more photonic sources linked to one or more detector elements is optionally referred to as: (1) a pathmeter, (2) an illumination array based analyzer, (3) a detector array based analyzer, (4) a dynamically configured analyzer, and/or a combination of the four.

Herein, for clarity of presentation and without loss of generality, the pathmeter and/or the detector array based analyzer is applied to the problem of optically noninvasively determining glucose concentration in blood/tissue of a subject. However, the detector array/multiple detector based analyzer described herein is more generally applied to an analysis of an analyte or grouping of similar sample constituents of tissue, blood, fluid, and/or a body compartment of a mammal, such as a human. Still more generally, the detector array based analyzer is applicable for analysis of a non-uniform, heterogeneous, and/or layered sample, such as a living substance and/or non-homogeneous inorganic matter. Still more generally, the detector array based analyzer is applicable to a sample having a non-flat optically probed surface. In terms of the body, the analyzer is optionally used to measure an analyte of the human body, such as in blood or tissue, with a concentration of more than 0.1 to 1.5 part-per-billion (ppb), such as insulin, or an analyte of the body with a concentration of more than 0.1, 1.0, or 10 part-per-million (ppm) and/or less than 10, 50, 100, 1000, or 10,000 ppm. However, again for clarity of presentation and without loss of generality, particular embodiments for the noninvasive analysis of glucose concentration in a patient or subject are presented, as described infra.

In a first clarifying example, the detector array based analyzer, is optionally a noninvasive, minimally invasive, or invasive analyzer apparatus and method of use thereof comprising a photon source, a detector array, and a photon transport system configured to direct photons from the source to the detector array via an analyzer-sample optical interface with or without an element separating light into narrowband regions. The analyzer provides a multitude of distinguishable optical pathlengths, yielding pathlength/probed tissue knowledge for: (1) appropriate data binning/operator combination and/or (2) internal data consistency for outlier determination.

Again, for clarity of presentation and without loss of generality, multiple examples are provided herein in terms of pathlength; however, more generally, appropriate data binning and/or appropriate data consistency checks described herein apply to derived knowledge on mean and standard deviation of a photonically probed: depth of penetration, radial dispersion, total optical pathlength, and/or probed sample layer relevance of a group of photons delivered to the detector via the sample from a light source of the photon source, such as an infrared source, where position of detection zones of individual detector elements of the detector array relative to one or more illumination zones of active source elements are known a priori and/or are derived from observed signals.

The detector array and/or individual detection elements thereof optionally optically couple to a plurality of optical transmission filters, optically couple to a plurality of light directing micro-optics, and/or optically couple to an array of light-emitting elements, such as laser diodes and/or light-emitting diodes.

The invention of a synergistic combination of light illuminators, optical filters, detector arrays, and/or extraction algorithm lies in the surprising results of the particular combination of analyzer elements described herein. The solutions provided herein are deemed non-obvious in view of a continuous stream of failed attempts at solving the noninvasive glucose concentration problem over the last 30+ years at an estimated research cost exceeding one billion dollars and in further view of the non-obvious details in the specific combinations of analyzer elements described herein. For clarity of presentation, individual elements of the noninvasive analyzer are described and combinations of the elements successively integrated into an analyzer are presented. However, separation of the synergistic elements of the analyzer for clarity of presentation should not be confused with the novel and unobvious combination of elements used to solve the noninvasive glucose concentration problem or more generally to the non-obvious and novel determination with enhanced accuracy and/or precision of an analyte property of a probed sample through enhanced resolution of the photonic pathway of a group of photons in the sample, such as via the algorithm coupled to the analyzer optical elements.

Further, again for clarity of presentation and without loss of generality, examples provided herein concentrate on breaking incident signal into sections using multiple detector elements. However, the techniques for pathlength/total optically probed pathlength also apply to variations in source position, source angle, and/or variations in the sample as a function of time, such as sample position, sample angle, and/or an induced force on the sample as well as to inherent/natural changes in a living physiological sample.

Further, for clarity of presentation, many example herein focus on specific noninvasive glucose determinations made in the spectral range of 400 to 10,000 nm, such as any of 400, 700, 900, 1000, 1100, 1400, 1900, or 2500 nm to one of 700, 1000, 1100, 1400, 1700, 1900, 2200, 2500, 2600, 4000, 5000, 7000 or 10,000 nm. However, apparatus, algorithms, and/or techniques described herein are usable in a wider range of frequency ranges within the electromagnetic spectrum, with a variety of samples, comprising a variety of sample constituents/properties.

Still further, the analyzer apparatus and/or methods of use described herein are used to control/compensate for pathlength and/or are used to compensate for optical pathlength variation, which improves accuracy and precision of analyte concentration determination through separation of variables related to (1) pathlength and (2) an observed and/or derived signal, such as power, intensity, reflectance, or absorbance. The method and apparatus comprise a combination of a source, a detector array, optical filters, focusing optics, geometry of alignment of analyzer components, and/or incorporation of algorithm routines to extract an analyte concentration with internal selection, binning, and/or consistency checks. The various interrelated elements of the analyzer are further described infra.

### Dynamic Analyzer

In another embodiment, the photon transport system includes a dynamic adjustable element, such as a dynamically positioned/dynamically focused/dynamically powered light directing unit optionally used to, within a measurement time period for a single analyte concentration determination, change any of: energy, intensity, radius, aperture, incident angle, solid angle, and/or focal depth, which each alter observed optically probed pathway for a static distance between a given source illumination and a given detector detection zone of photons entering the sample, such as skin of a subject.

### Spectrometer

The analyzer described herein optionally uses and/or is integrated with a wavelength separation device of a spectrometer, such as a grating, a prism, or an interferometer. However, in an optional preferred embodiment, the wavelength separation device for resolving illumination bands of greater than 50, 75, 100, or 125 nm in width are not necessary.

### Optically Stacked Filter Arrays

In still another embodiment, two optically stacked arrays of optical filters as a portion of a noninvasive analyzer apparatus are used. The stacked arrays of optical filters are optionally configured to pass multiple distinct and/or overlapping wavelength ranges to an array of detectors, where the filter combination in combination with illumination zone to detection zone distances of elements of the detector array resolves diffusely reflected/partially absorbed optical pathlengths and/or optical pathways through skin. Several examples are provided here for clarity of presentation. However, it is the inventor determined details of the optical stack-detector-algorithm combinations that truly enhance the analyzer performance, such as for noninvasive glucose concentration determination, as detailed in the detector, filter, sample, and algorithm sections, infra. Several early examples are provided herein to aid in understanding the use of filters for pathlength resolution in the analyzer and the benefits thereof.

### Example I

In a first example, a longpass filter and/or a shortpass filter used to pass a range of wavelengths is described in terms of pathlength and/or pathway control. Particularly, filter passed wavelengths are selected to function with and complement the sample absorbance, anisotropy, and/or scattering. Particularly, the sample absorbance and/or scattering greatly impacts radial transfer and a corresponding pathlength of the incident photons to a detector element or detector zone of a detector array, where the sample controlled radial transfer/pathlength control is further limited by the filter combination associated with a given detector element or a given detector zone associated with the optical filter(s). For example, the sample properties narrow photons to a wavelength zone, the filters further narrow the wavelength zone of photons as a function of radial distance, and elements of the detector array thus observe various depths and/or pathlengths of the initial photons as a function of distance.

### Example II

In a second example, the optical filters, skin absorbance, tissue anisotropy, and sample site scattering characteristics are used to select for a range of probed optical depths, radial travel, and/or optical pathway. Combining the optical filters with matched detector elements yields considerable multiplexed information about the probed sample, which is coupled with algorithms used to extract the continually varying tissue information to yield a map of the tissue, optionally complemented by use of the redundant information of nearby detector elements, overlapped information of slightly removed detector elements, and/or slow analog variation in tissue variation as a function of radial distance and/or as a function of detector element spacing. Combined a more accurate analyte property estimation, such as a glucose concentration, is derived with optional certainty measures and/or outlier information.

### Example III

In a third example, the combination of multiple filters with knowledge of how absorbance of the sample varies with wavelength, such as water absorbance patterns, allows selection of detector element-optical filter element combinations having proper radial distances for glucose determination for a given subject or a given subject type. Details of filter selection as a function of radial position, anisotropy as a function of wavelength, and tissue type in terms of skin layer thicknesses are further developed and detailed in the body of this application.

### Data Redundancy/Overlap

In yet another embodiment, redundant information, spatial variance information, radial variance information, and/or overlapping information is derived using data from adjacent and/or nearby elements of a two-dimensional detector array. Details of use of the internal data quality assessment approach is further described, infra.

### Data Selection/Classification

In still yet another embodiment, subsets of signals from one or more two-dimensional detector arrays are used to determine at least one of: sampled pathlengths, internal data consistency for outlier analysis, precision enhancement, skin type, photon path information, and state of the subject tested. Several examples are provided here for clarity of presentation. However, again, the details of the system, not the generalities, allow proper function of the system. Thus, the details are provided, infra, in the illuminator, detector, filter, sample, and algorithm sections.

### Example I

In a first example, the detector array has adjoining detector elements that should yield similar information. Thus, comparing a result from a single detector element with a result from an adjacent detector element yields an internal consistency check. The consistency check is optionally used for selection of data to bin, for outlier detection, and/or for mapping the sample.

### Example II

In a second example, the detector array has a set of detector elements positioned as a function of radial distance from a given illumination site or illumination zone. As the probed sample as a function of radial distance changes as a result of tissue anisotropy, scattering, absorbance, and/or temperature, then determined results from the radial vector of detector elements should vary slowly with radial distance as the photons as a function of radial distance have differing depths of penetration into the sample and the sample layers as a function of depth vary. Thus, determined sample concentration and/or composition, such as water, protein, fat, and glucose, should vary with radial distance. The internal smoothness of the data is optionally used to again determine what data to bin, such as data rich in glucose concentration information or similar optical pathways in tissue, and/or what data represents outlier data, such as photons passing through an interrupting hair follicle or pore.

### Example III

In a third example, a radial vector of detector elements of the detector array will observe different tissue samples as a function of radial distance due to the varying depth of penetration of the, subsequently detected, photons as a function of radial distance and the presence of varying tissue constituents as a function of depth, as described in the preceding example. The inventor has determined that selection of data from a subset of the radial vector of detector elements yields data representative of layers having glucose concentrations representative of blood glucose concentration. Further, the selected elements of the radial vector of detector elements is expected to be a continuous subset of elements for a given tissue type. Hence, selection of data from radially appropriate distances from an illumination zone for a given illuminator and/or wavelength range for an in-line determined tissue type yield higher signal-to-noise ratios and associated accuracy and precision of a glucose concentration determination.

### Example IV

In a fourth example, wavelengths of light reaching a given detector element of the detector array is controlled as a function of time and/or position. As the mean depths of penetration and the mean radial dispersion, described in the last two examples, is wavelength dependent, control of the wavelengths of light reaching a given detector element is optionally and preferably used to yield additional information about the sample. Control of the wavelengths of light is performed through control of light delivered to the sample, such as via use of selected light emitting diodes with narrow emission wavelengths, as described infra, and/or via use of optical filters in the light path, as described supra. Control of wavelength in this manner allows a multiplexed solid state spectral analyzer in the absence of a dispersive element, such as a prism or grating, and further in the absence of a time domain movable element, such as a movable mirror in a Fourier transform based spectrometer. Generally, the presented analyzer allows for wavelength resolution through a combination of wavelength of illumination, such as from a light-emitting diode, through use of knowledge of absorbance of the tissue sample, through use of selected narrow band filters associated with individual detector elements and/or detector groups of the detector array, and through relative position of illumination zones and detection zones on the tissue sample, which is fully discussed, infra.

### Example V

In a fifth example, the inventor has determined that a relatively small number of optical filters positioned proximate the detector elements of the two-dimensional detector are optionally used to great effect due to a change as a function of wavelength and radial position the impact of anisotropy, scattering, water absorbance, and temperature effects on observed absorbance. Thus, one filter is not used with the entire detector array as information is lost and individual filters are not necessary for each element of the detector array, which results in light loss and/or increased cost. Rather, a small group of inexpensive and standardizable filters are preferably used where the filters are specified using information developed by the inventor on the combination of water absorbance, temperature, anisotropy, and scattering of light allowing a small/inexpensive and optionally solid-state analyzer. Again, this example is provided for clarity of presentation and the details are provided infra.

### Example VI

In the previous set of examples, all of the varied and/or controlled properties additionally benefit by controlled changes of the properties as a function of time and/or position, where position is the relative position of the incident light and light emerging from the sample for detection. For instance, varying which light strikes which position of the skin as a function of time allows additional spatial mapping of the sample, pathlength control of the photons reaching a given detector element of the detector array, and/or control of probed depth of penetration for subsequent analyte property determination. Thus, in stark contrast to the time variability and tissue variability described in the prior art as complicating the measurement, the same tissue variability is used herein to yield still additional information about the sample and/or analyte of interest. For example, compression of a tissue layer results in spectroscopically observed changes from pathlength and/or density changes of the tissue layer. Again, this example is provided for clarity of presentation and the details are provided infra.

### Example VII

Any of the elements described herein are optionally combined with wavelength separation elements, such as a spatial or time based spectrometer.

### Detector Array

In yet another embodiment, a noninvasive analyzer apparatus and method of use thereof using a plurality of two-dimensional near-infrared detector arrays is described. Using multiple redundant and/or overlapping illumination zone to detection zone distances has a number of uses, such as outlier determination, determination of effectiveness of optical coupling, and/or enhancing performance of the analyzer through data combination/selection. Again, several examples are initially provided to aid in description of the invention while details are provided, infra, for clarity of presentation.

### Example I

In a first example, one or more two-dimensional detector arrays allow a plurality of closely spaced and/or matching radial distances between a mean position of an illumination zone and a mean position of a detection zone. Thus, signals from a plurality of matched and/or closely matched optical pathlengths are optionally binned for enhancing signal-to-noise ratios while additionally allowing internal outlier error detection by examining a metric of uniformity and/or deviations within the binned signals and removing outliers beyond a threshold.

### Example II

In a second example, analyzer performance is enhanced by selecting a narrow range of pathlengths, where a narrower range of optical pathlengths allows a more precise analyte concentration determination according to Beer's Law or an equivalent scattering based absorbance/scattering equation or calibration model relating signal to analyte concentration through separation of the linked variables of observed intensity and probed pathlength, where each contributes to the determined concentration. Particularly, by separating intensity or absorbance from pathlength in calibration through use of a hardware control, a subsequent calibration/prediction model yields a more robust, accurate, and precise analyte concentration determination through use of the hardware based variable separation.

### Example III

In a third example, having a range of similar observed optical pathlengths at different radial angles from the illumination zone to the detector element allows for detection of sample inhomogeneity within one or more layers. For instance, photons traveling in one direction may encounter glucose containing dermal tissue while photons traveling in another direction may additionally encounter a hair follicle and/or photon scattering from a papillary ridge or other tissue layer interface with very low and/or misleading glucose related signals. By comparing signals in multiple directions, signals in one direction crossing an inhomogeneity may be identified and removed from subsequent analyses yielding a more robust and/or accurate calibration model and/or a more accurate and precise glucose concentration determination in a prediction phase.

### Example IV

In a fourth example, having a range of closely spaced detector elements used in analysis of a matrix with relatively slowly changing properties as a function of radial distance from an illumination zone allows for algorithm enhanced resolution, such as via dithering a light source or drizzling the data.

### Example V

In a fifth example, use of signals from different positions of the two-dimensional detector array allow for determination of optical contact between a sample probe and tissue, which is useful for selection of zones of the two-dimensional detector array sensing adequate and not excessive sample probe contact with the tissue.

Again, examples provided here are non-limiting, are intended to clarify presentation of the invention, and are further described in detail, infra.

### Sample Mapping/Hardware Control

In a further embodiment, an apparatus and method of use thereof is described using acquisition of noninvasive mapping spectra of skin and subsequent optical/optical path reconfiguration for subsequent subject specific data collection. For instance, a data processing system uses a first mapping phase to set instrument control parameters for a particular subject, set of subjects, and/or class of subjects. Subsequently, the control parameters are used in a second data collection phase to collect spectra of the particular subject or class of subjects.

For example, a near-infrared noninvasive analyzer is configured with a first optical configuration used to map an individual and/or group of individuals through use of mapping spectra. The mapping spectra are analyzed and used to reconfigure the optical setup of the analyzer to a second optical configuration suited to the individual and/or group of individuals. Subsequently, collection of noninvasive spectra of the individual and/or group of individuals is performed using the second optical configuration, which is preferably optimized to yield additional information based on the skin of the individual and/or group of individuals. For example, initial measurements are used to determine a thickness of a glucose containing layer, such as the epidermis and/or dermis layers of skin. Similarly, initial measurements are used to determine a depth of an underlying glucose scarce layer, such as a subcutaneous fat layer. Results are used to: (1) reprocess previous data selecting incident light, filter, radial distance, detector combinations that have enhanced signal-to-noise levels for the analyte, such as glucose in the dermis and/or (2) establish instrument parameters for subsequent data collections, such as for a given dermis thickness of a subject or group of subjects. Again, for clarity of presentation, the details are provided infra.

Similar to the sample mapping/hardware control process, an alternative process is to collect the data, map the data, classify the data, and then use a subset of the data for subsequent analyte property determination. In this case, only a subset of the collected data is used in the final analyte property determination.

In yet another embodiment, a data processing system analyzes data from an analyzer to estimate and/or determine an analyte property, such as concentration using multiple types of data, such as from an external sensor. For instance, temperature, humidity, hydration, tissue hydration, and/or pressure sensor data complements spectroscopic information.

In yet another embodiment, a data processing system analyzes data from an analyzer to estimate and/or determine an analyte property, such as concentration using multiple types of data with two or more focusing depths. For example, information on one analyte is obtained by using an optical configuration yielding information on a first analyte at a first optical depth with a first set of detectors while simultaneously collecting data for determination of a second analyte property at a second optical depth using the illumination, filter, and detector combinations described herein.

In still another embodiment, an analyzer using light interrogates the sample using one or more of:
a spatially resolved system;
   an incident light radial distance resolved system;
   a controllable and variable incident light solid angle system;
   a controllable and variable incident light angle system; and
   a controllable and variable light throughput/resolution control aperture;
a time resolved system, where the times are greater than about 1, 10, 100, or 1000 microseconds;
collection of spectra with varying radial distances between incident light entering skin and detected light exiting the skin;
an incident angle resolved system; and
a collection angle resolved system.

Data from the analyzer is analyzed using a data processing system capable of using the at least partially spatially resolved information inherent in the multiplexed data.

In yet another embodiment, a data processing system uses interrelationships of chemistry based on a priori spectral information related to absorbance of a sample constituent and/or the effect of the environment, such as temperature, on the spectral information.

In still yet another embodiment, a data processing system uses information related to contact pressure on a tissue sample site.

In another embodiment, a data processing system uses a combination of any of:
spatially resolved information;
temporally resolved information on a time scale of longer than about one microsecond;
temporally resolved information on a sub one hundred picosecond timeframe;
incident photon angle information;
photon collection angle information;
interrelationships of spectral absorbance and/or intensity information;
environmental information;
temperature information; and
information related to contact pressure on a tissue sample site.

In another embodiment, subject specific data is analyzed using a hybrid sample-reference based calibration model and tissue physiology model.

For clarity, the interrelationships of spectral absorbance and/or intensity information is further briefly further described here and detailed, infra. A given chemical bond or directly linked chemical bonds interact with different wavelengths of light differently, yet the given molecule is the same. Thus, redundant information is obtainable at at least two different wavelengths. For example, a given carbon-hydrogen bond of glucose absorbs light at two or more distinct wavelengths, which yields multiple readings on the single carbon-hydrogen bond of glucose. In the near-infrared, the inventor has determined that two carbon-hydrogen bonds and one oxygen-hydrogen bond of glucose form a first triplet of absorbance bands in the combination band spectral region and a second triplet of absorbance bands in the first overtone spectral region. However, the combination band region is only optically probed at small depths of penetration while the first overtone region is additionally probed at a mid-level depth of penetration yielding information on where the glucose molecules reside as a function of depth of penetration. Similar information is derived in different spectral regions and/or for different molecular bonds, as described infra.

In yet still another embodiment, an apparatus and method of use thereof is described using a plurality of time resolved sample illumination zones coupled to at least one two-dimensional detector array monitoring a plurality of detection zones linked to the sample illumination zones.

In still another embodiment, the analyzer uses any combination and/or permutation of elements described herein.

### Axes

Herein, axes systems are separately defined for an analyzer and for an interface of the analyzer to a patient, where the patient is alternatively referred to as a subject, an individual, and/or a person.

Herein, when referring to the analyzer, an x, y, z-axes analyzer coordinate system is defined relative to the analyzer. The x-axis is in the direction of the mean optical path. The y-axis crosses the mean optical path perpendicular to the x-axis. When the optical path is horizontal, the x-axis and y-axis define a x/y horizontal plane. The z-axis is normal to the x/y plane. When the optical path is moving horizontally, the z-axis is aligned with gravity, which is normal to the x/y horizontal plane. Hence, the x, y, z-analyzer coordinate system is defined separately for each optical path element or section. If necessary, where the mean optical path is not horizontal, the optical system is further defined to remove ambiguity.

Herein, when referring to the patient, an x, y, z-axes patient coordinate system is defined relative to a body part interfaced to the analyzer. Hence, the x, y, z-axes body coordinate system moves with movement of the body part. The x-axis is defined along the length of the body part, the y-axis is defined across the body part. As an illustrative example, if the analyzer interfaces to the forearm of the patient, then the x-axis runs longitudinally between the elbow and the wrist of the forearm and the y-axis runs across the forearm. Together, the x,y plane tangentially touches the skin surface at a central point of the interface of the analyzer to the body part, which is referred to as the center of the sample site, sample region, illumination zone, or sample site. The z-axis is defined as orthogonal to the x,y plane. Rotation of an object is further used to define the orientation of the object, such as an analyzer probe tip, to the sample site. For example, in some cases a sample probe of the analyzer is rotatable relative to the sample site. Tilt refers to an off z-axis alignment, such as an off z-axis alignment of a probe of the analyzer relative to the sample site.

### Analyzer

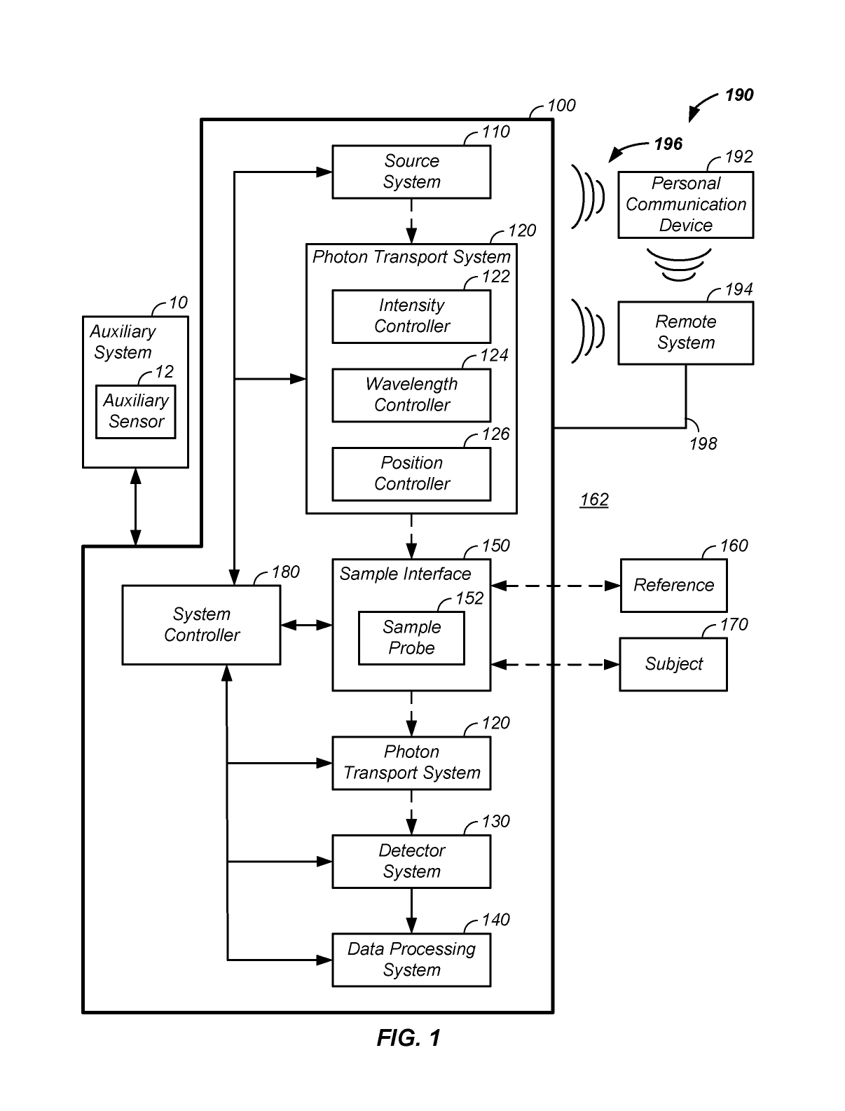

Referring now and throughout to FIG. 1, an analyzer 100 is illustrated. The analyzer comprises at least: a light source system 110, a photon transport system 120, a detector system 130, and a data processing system 140, where the data processing system is optionally remotely located from the source/sample/detector system. In use the analyzer 100 estimates and/or determines a physical property, a sample state, a sample constituent property, and/or a concentration of an analyte. Components/systems of the analyzer 100 are introduced in the following subsections.

### Source

Still referring to FIG. 1, the source system 110 generates photons in any of the visible, infrared, near-infrared, mid-infrared, and/or far-infrared spectral regions. In one case, the source system generates photons in the near-infrared region from 1100 to 2500 nm or any range therein, such as within the range of about 1200 to 1800 nm; at wavelength longer than any of 400, 700, 800, 900, 1000, and 1100 nm; and/or at wavelengths shorter than any of 2600, 2500, 2000, or 1900 nm. The source system 110 generates/provides photons, such as via use of one or more of: a set of light-emitting diodes, a laser diode, a tunable laser, a laser, a blackbody source, an incandescent lamp, and/or a halogen lamp.

### Photon Transport System

Still referring to FIG. 1, the photon transport system 120 is further described. Generally, the photon transport system 120 comprises any set of hardware, software, and/or light guiding optics used to guide light from the source to the reference 160 and/or subject 170 via the sample interface 150 and/or from the reference 160 and/or subject 170 via the sample interface 150 to the detector system 130. Examples of light guiding optics include: fiber optics, micro-optics, and/or dynamic optics. The photon transport system optionally refers to an optical filter or set of optical filters.

The photon transport system 120 optionally: (1) couples with and/or is integrated into a sample interface 150 and/or (2) couples with and/or is integrated into the detector system 130. For instance, the photon transport system optionally uses a fiber optic that passes into and through the sample interface to transport light to the sample and/or uses a fiber optic to collect light from the skin and direct the light to one or more detector elements of the detector system. Hence, for clarity of presentation, herein when the photon transport system 120 is referred to contacting or proximately contacting the subject 170, the photon transport system optionally additionally or separately refers to the sample interface 150 and/or the detector system 130.

### Sample Interface

Still referring to FIG. 1, the sample interface 150 contacts the subject 170, proximately contacts the subject 170, or does not contact the subject 170. However, in an optional and preferred embodiment, a first patient interface surface section of the sample interface 150 transmitting photons from the subject 170 contacts the subject 170 while, at the same time, a second section of the sample interface 150 transmitting photons from the subject 170 does not contact the subject 170 and the data processing system 140 determines one or more regions of the sample interface 150, and detector elements of the detector system 130 linked thereto, properly contacting the subject 170, such as with a specified distance between the sample interface 150 and a sampled volume of the subject 170 and/or with a specified force/pressure delivered to the sampled volume of the subject 170.

### Patient/Reference

Still referring to FIG. 1, an example of the analyzer 100 is presented. In this example, the analyzer 100 includes a sample interface 150, which interfaces to a reference material 160 and/or to a subject 170. Herein, for clarity of presentation a subject 170 in the examples is representative of a person, animal, a prepared sample, and/or a patient. In practice, the analyzer 100 is used by a user to analyze the user, referred to as the subject 170, and/or is used by a medical professional to analyze a patient.

### Detector System

Still referring to FIG. 1, the detector system 130 is further described. Generally, the detector system 130 comprises one or more detector elements of one or more detector types, such as 1, 2, 3, 4, 5, or more detector types, used to detect photons from the source system 110 after having probed a sample volume of the subject 170. Optionally, the detector system 130 additionally detects photons from the source system 110 directly or semi-directly to monitor source luminance and/or after encountering and interacting with the reference 160. Examples of detector types include: a visible wavelength detector, a silicon (Si) based detector, a complementary metal-oxide semiconductor (CMOS) detector, a doped silicon detector, an infrared wavelength detector, an indium gallium arsenide (InGaAs) based detector, a germanium (Ge) comprising detector, a lead-salt detector, a lead-sulfide (PbS) comprising detector, a mid-infrared detector, and/or a mercury cadmium telluride (MCT) comprising detector. A detector of the detector system 130 is optionally a single element detector, a multi-element detector, a two-column detector, a multi-column detector, and/or a detector array. Optional and preferred detector array configurations and orientations are further described, infra.

### Data Processing System

Still referring to FIG. 1, the data processing system 140 collects, combines, and/or analyzes data from the auxiliary system 10, an auxiliary sensor 12 thereof, and/or from the detector system 130, as further described infra.

### Controller

Still referring to FIG. 1, the analyzer 100 optionally includes a system controller 180 or controller. The system controller 180 is used to control one or more of: the light source system 110 or a light source 112 thereof, the photon transport system 120, the detector system 130 or a detector 132 thereof, the sample interface 150, position of the reference 160 relative to the sample interface 150, position of the subject 170 relative to the sample interface 150, and/or communication to an outside system 190, such as a personal communication device 192, a smart phone, and/or a remote system 194 using a wireless communication system 196 and/or a hard wired communication system 198. For example, the remote system includes a secure algorithm system, a data processing system, a data storage system, a data transfer system, and/or a data organization system.

The system controller 180 optionally comprises one or more subsystems stored on a client. The client is a computing platform configured to act as a client device or other computing device, such as a computer, personal computer, a digital media device, and/or a personal digital assistant. The client comprises a processor that is optionally coupled to one or more internal or external input device, such as a mouse, a keyboard, a display device, a voice recognition system, a motion recognition system, or the like. The processor is also communicatively coupled to an output device, such as a display screen or data link to display or send data and/or processed information, respectively. In one embodiment, the system controller 180 is the processor. In another embodiment, the system controller 180 is a set of instructions stored in memory that is carried out by the processor. In still another embodiment, the remote system 194 is the processor.

The client includes a computer-readable storage medium, such as memory. The memory includes, but is not limited to, an electronic, optical, magnetic, or another storage or transmission data storage medium capable of coupling to a processor, such as a processor in communication with a touch-sensitive input device linked to computer-readable instructions. Other examples of suitable media include, for example, a flash drive, a CD-ROM, read only memory (ROM), random access memory (RAM), an application-specific integrated circuit (ASIC), a DVD, magnetic disk, an optical disk, and/or a memory chip. The processor executes a set of computer-executable program code instructions stored in the memory. The instructions may comprise code from any computer-programming language, including, for example, C originally of Bell Laboratories, C++, C#, Visual Basic® (Microsoft, Redmond, Wash.), Matlab® (MathWorks, Natick, Mass.), Java® (Oracle Corporation, Redwood City, Calif.), and JavaScript® (Oracle Corporation, Redwood City, Calif.).

An exemplary method of use of the analyzer 100 is provided. The system controller 180 controls one or more of the subsystems to accurately and precisely deliver photons to a sample site of the subject 170. For example, the system controller 180 directs the analyzer 100 to collect a spectrum and/or a set of optical pathways of a portion of a body part of the subject 170. The system controller 180 also obtains position information, timing information, predetermined settings, and/or dynamically controlled hardware settings from the memory and/or the data processing system 140. The system controller 180 then optionally controls the source system 110 to inject a stream, set, group, and/or bunch of photons into the photon transport system 120. The system controller 180 optionally controls one or more elements of the photon transport system, such as a photon directing unit, a photon limiting unit, and/or a dynamically controlled micro-optic. For example, the system controller 180 optionally and preferably controls targeting of the photon beam through a scanning/targeting/delivery sub-system of the photon transport system 120 and or of the sample interface 150. The system controller acquires data from the detector system 130 optionally via the data processing system 140. Further, display elements of the display system are preferably controlled via the system controller 180. Displays, such as display screens, which are typically provided to one or more operators and/or to one or more patients. The system controller 180 optionally links to the outside system 190, remote system 194, and/or the personal communication device 192.

Herein, the system controller 180 refers to a single system controlling the analyzer 100, to a single controller controlling a plurality of subsystems controlling the analyzer 100, or to a plurality of individual controllers controlling one or more sub-systems of the analyzer 100.

Still referring to FIG. 1, the optional system controller 180 operates in any of a predetermined manner or in communication with the data processing system 140. In the case of operation in communication with the data processing system 140, the controller generates control statements using data and/or information about the current state of the analyzer 100, current state of a surrounding environment 162 outside of the analyzer 100, information generated by the data processing system 140, and/or input from a sensor, such as a sample interface sensor, a sample probe 152, an auxiliary system 10, and/or an auxiliary sensor 12 thereof. Herein, the auxiliary system 10 is any system providing input to the analyzer 100.

Still referring to FIG. 1, the optional system controller 180 is used to control: photon intensity of photons from the source using an intensity controller 122, wavelength distribution of photons from the source system 110 using a wavelength controller 124, physical routing of photons from the source system 110 using a position controller 126, and/or timing of photon delivery.

Still referring to FIG. 1, for clarity of presentation the optional outside system 190 is illustrated as using a personal communication device 192, such as a smart phone. However, the personal communication device 192 is optionally a cell phone, a tablet computer, a phablet, a computer network, a personal computer, cloud computing, and/or a remote data processing center. Similarly, the smart phone also refers to a feature phone, a mobile phone, a portable phone, and/or a cell phone. Generally, the personal communication device 192 includes hardware, software, and/or communication features carried by an individual that is optionally used to offload requirements of the analyzer 100. For example, the personal communication device 192 includes a user interface system, a memory system, a communication system, and/or a global positioning system. Further, the personal communication device 192 is optionally used to link to the remote system 194, such as a data processing system, a medical system, and/or an emergency system. In another example at least one calculation of the analyzer 100 in noninvasively determining a glucose concentration of the subject 170 is performed using the personal communication device 192. In yet another example, the analyzer gathers information from at least one auxiliary sensor 12 and relays that information and/or a processed form of that information to the personal communication device 192, where the auxiliary sensor is not integrated into the analyzer 100. Optionally data from the analyzer 100 is processed in the cloud or a remote computing facility. Optionally, the personal communication device 192 is used as a portal between the analyzer 100 and the cloud.

The remote system 194 is optionally a data processing center and/or a cloud processor configured to receive signal from more than one analyzer and to return a calculated analyte concentration and/or an analyte property to the corresponding analyzer and/or to a communication device of the user of the corresponding analyzer. For instance, a given analyzer is optionally configured to collect data on demand and/or at a programmed interval and to relay the data to the remote system 194 where it is processed. Communication is optionally encoded/encrypted to secure personal medical information of the user and/or to secure data generated using the analyzer 100. The remote system 194 optionally contains extensive computing power for performing analysis difficult to implement on a small, low power, portable device. Further, the remote system 194 optionally performs time-series analysis on the incoming data to establish trends in an analyte property, such as an glucose concentration increasing into a hyperglycemic concentration, passing a threshold concentration, and/or falling toward a hypoglycemic glucose concentration. The remote system 194 optionally and preferably relays the analyte and/or glucose concentration back to the user where it is displayed, to a hospital rack system, monitoring system, legal system, and/or the like. Further, the remote system optionally prognosticates a glucose concentration going hypoglycemic through the time-series analysis and/or through combined use of the user's glucose history, food intake, and/or exercise log relayed to the remote system. Still further, data uploaded from the analyzer 100 to the remote system 194 is optionally configured to enhance a global model, subject specific model, and/or real-time parameter selected model through combined data collection and reference glucose concentration determinations obtained through hundreds of uses by a user and/or via data from hundreds, thousands, or millions of users, where the larger data set is used to develop a more robust model and/or to develop a plurality of more narrowly specified models, where the more narrowly specified models allow an enhanced prediction performance. To facilitate the updated model in terms of ease of implementation and in view of regulatory requirements, the analyzer is optionally modularly constructed where all subsystem data is sent to the system controller 180 and/or all subsystems controls are received directly from the system controller 180, with no direct system to subsystem communication of data and/or controls, which allows individual subsystems to be updated/improved with validation of code only for the upgraded subsystem in the absence of re-validation of the entire analyzer.

Optionally, the personal computing device 192 is used: (1) in communication between the analyzer 100 and the remote system 194 and/or (2) in display of results from the remote system 194 to the subject 170. Use of the personal communication device 192 allows many elements of the analyzer 100 to be removed from the analyzer 100, such as a display screen, internet access, Wi-Fi linking elements, computing requirements, battery related computing requirements, security, a user input interface, and/or a sound system.

### Photon-Skin Interaction

Various physical properties and chemical traits of tissue control how light propagates through tissue. A detailed examination of spectra of tissue and/or optical pathways through tissue in the near-infrared reveals some general trends, such as for some wavelengths and for some optical configurations that a mean photon depth of penetration into the tissue, with subsequent detection at the incident surface of the subject, increases with mean radial distance between a photon illumination zone and a photon detection zone. For example, for photons transmitting from a sample illumination zone, through the subject, and through a photon detection zone, such as at a subject/analyzer interface:
at a first radial distance, photons penetrate with a mean maximum depth of penetration into an epidermal layer of a subject;
at a second larger radial distance, photons penetrate with a mean maximum depth of penetration into a dermal layer of the subject; and
at a third still larger radial distance, photons penetrate with a mean maximum depth of penetration into a subcutaneous fat layer of the subject.

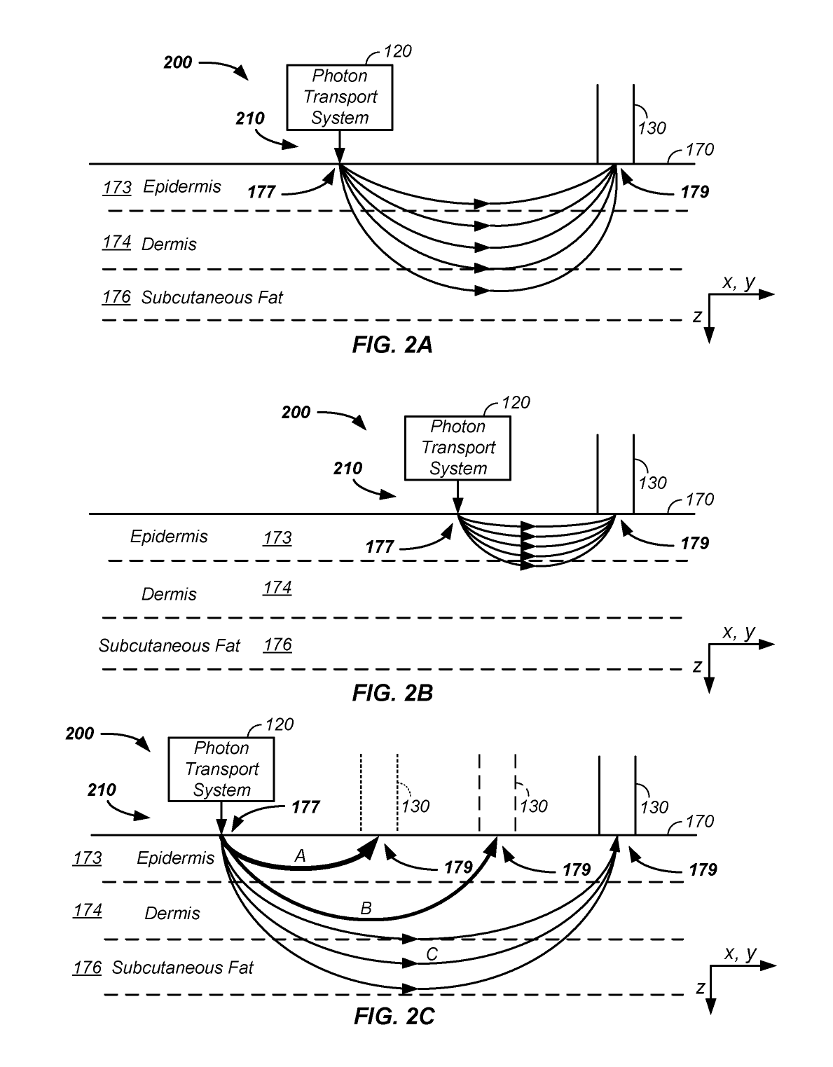

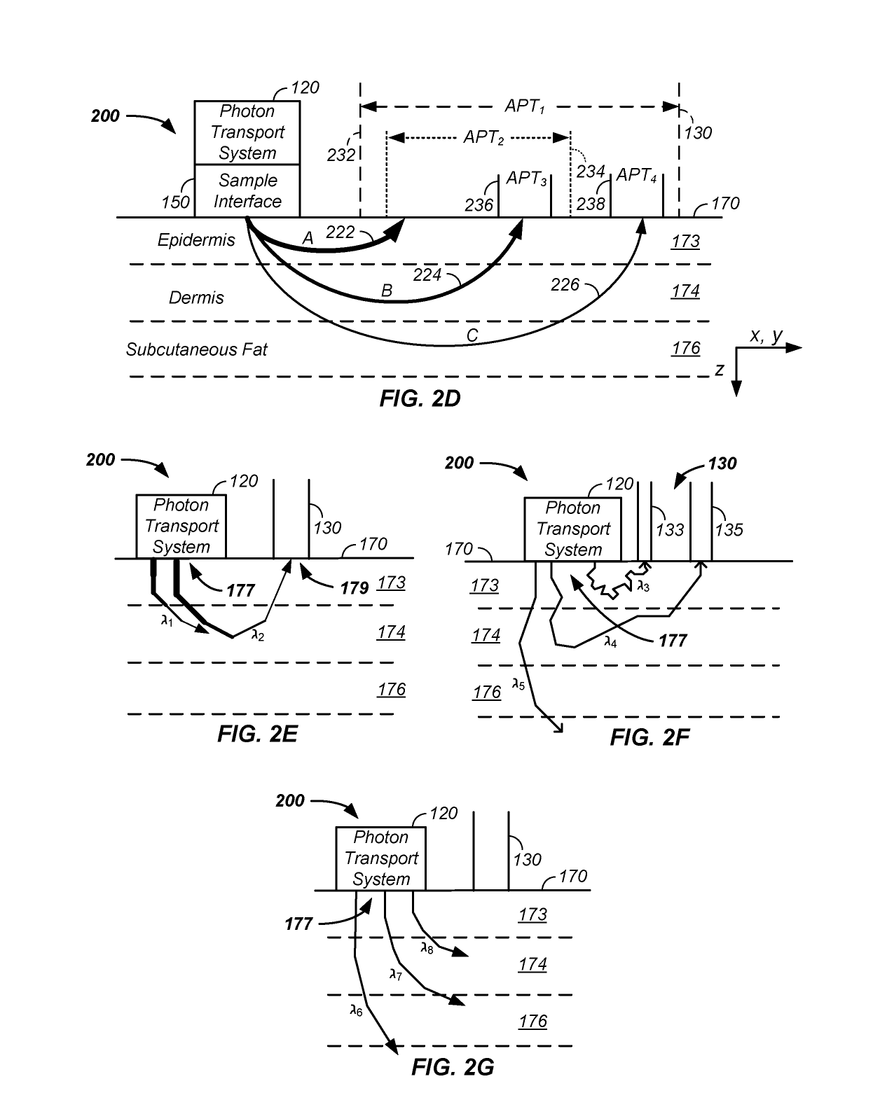

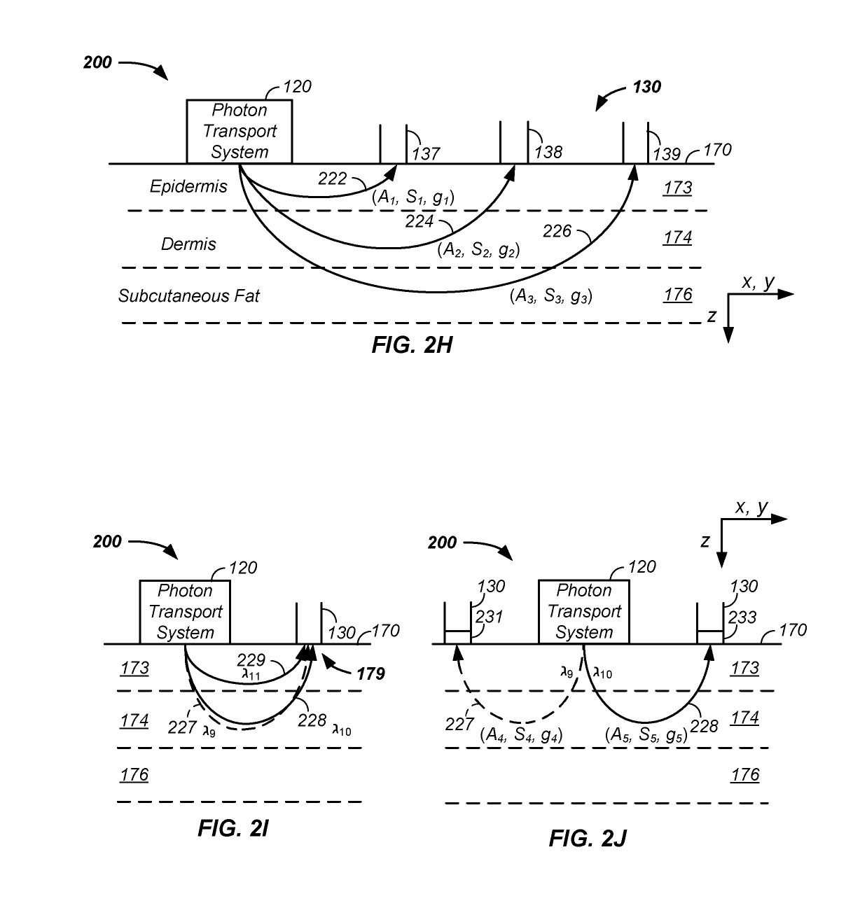

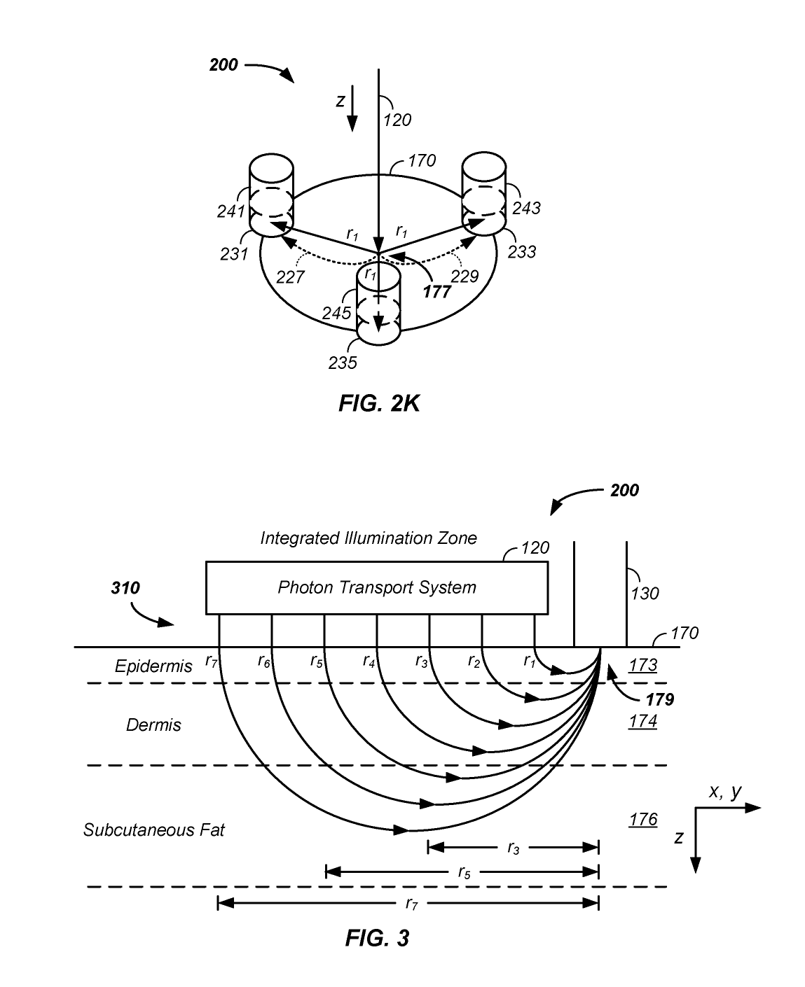

In fact, the actual situation is far more complex as factors like the absorbance of each tissue constituent as a function of wavelength greatly complicate the analysis. Consideration of scattering as a function of wavelength further complicates the analysis as does consideration of temperature, anisotropy, sample layer thicknesses, sample layer interfaces, changes in hydration, and tissue inhomogeneity, even within a layer. Thus, the prior art has taken the approach of collecting large amounts of data and throwing the bulk data into various algorithmic approaches. However, the inventor has determined that within the complexity of the tissue, combinations of tissue chemistry and tissue physics allows coupling of particular wavelengths, particular total pathlengths, particular total radial distances, probed tissue pathways, and many more tissue parameters to an analyzer with source, optical transport, optical filter, and detector combinations that with current source, optics, and detector technologies allow the interactions of the tissue itself with light to aid in analysis of the tissue. For example, the tissue itself limits some optical pathways of some frequencies and allows transmittance of photons at other wavelengths. By coupling first analyzer elements sensitive to wavelengths of light diffusely reflected from a first source zone to a first detection zone and coupling second, third, fourth, and n^{th} to analyzer elements sensitive to wavelengths of light diffusely reflected from a second, a third, a fourth, and an n^{th} source zone to detection zone distance, the n wavelengths are at least partially resolved as a function of distance, where n is a positive integer of at least 5, 10, 15, 20, 25, 30, 35, 40, or 45. Further, n wavelengths, even when overlapping in radial distance are optionally further separated using different light source, filter, and detector combinations, such as using a first optical filter-detector combination in a first direction and a second optical filter-detector combination in a second radial direction. Indeed, discrete, even while overlapping, illumination zones, allow further separation of collected data related to tissue depth, tissue layer thickness, tissue inhomogeneities, total optical pathlength, mean depth of penetration, mean optical pathway, and mean radial distance. Indeed, the inventor has determined an apparatus and method of use thereof capable of distinguishing even a low concentration analyte concentration, such as a glucose concentration, in the presence of sample inhomogeneities, sample layers of distinct chemical composition, differential scattering as a function of wavelength, and sample constituents of many magnitude larger absorbance. Due to the complexity of the interacting variables, the inventor presents herein a breakdown of the sample complexity and then builds up analyzer elements-algorithm combinations that use the sample complexity in the successful analysis of analyte property estimation, such as a noninvasive glucose concentration. The inventor notes that while many individual tissue property and/or analyzer elements are known, it is the novel synergistic combination of the tissue properties with analyzer elements that allows the analysis of the, heretofore considered too complex, skin tissue-blood sample for noninvasive glucose concentration determination using near-infrared light. An initial breakdown of the sample complexity is addressed in reference to FIGS. 2(A-J), infra.

### Photon Transport in Tissue

Photonic pathways in tissue affect construction and use of calibration models. As the analyzer 100 described herein is based upon tissue knowledge and patterns of photonic pathways therein, parameters affecting a measured signal, such as an intensity, absorbance, reflectance, and/or a measured power spectrum, such as pathlength, scattering, anisotropy, tissue layers, and aperture are addressed herein.

### Pathlength

Herein, for clarity, without loss of generality, and without limitation, Beer's Law is used to describe photon interaction with skin, though those skilled in the art understand deviation from Beer's Law result from sample scattering, index of refraction variation, sample inhomogeneity, turbidity, anisotropy, sample layer states of: thickness, geometry, structure, and/or composition, changes in temperature, changes in sample density, changes in pressure or force applied to the sample, and/or absorbance out of a linear range of the analyzer 100. Beer's Law, equation 1, states that:

AαbC (eq. 1)

where A is absorbance, b is pathlength, and C is concentration.

Typically, spectral absorbance is used to determine concentration, such as via transmission through a fixed pathlength optical cell in a sample holder. However, as the absorbance is additionally related to pathlength and the optically probed pathlength in tissue varies under a number of conditions, changes in pathlength result in changes in an estimated analyte concentration if not compensated for. Two cases illustrate:

In a first case of a reduced pathlength in the sample versus a pathlength used in a calibration data set, a simple calibration may relate an absorbance of 2 (A=2) with a concentration of 100 (C=100) at a fixed pathlength of 1 (b=1). However, if the pathlength is not controlled in the tested sample and reduces to a pathlength of ½ (b=½), then the calibration will still relate an observed absorbance of 2 with a concentration of 100, while the actual concentration of the tested sample is 200, which is double the calibrated value and an error of one hundred percent. In Table 1, this case is illustrated by the first and second row of values, where in the equation A=bC, b*C must equal 100 according to the non-pathlength controlled/adjusted calibration model.

In a second case of an uncontrolled longer pathlength in the sample compared to a pathlength used in standards/samples used in preparation of the calibration, the same simple calibration still relates an absorbance of 2 with a concentration of 100 at a fixed pathlength of 1. However, if the pathlength is not controlled in the prediction samples and doubles to a pathlength of 2 (b=2), then the simple calibration will still relate an observed absorbance of 2 with a concentration of 100, while the actual concentration in the sample is 50, which is one-half of the calibrated value. In Table 1, this second case is illustrated by the second and third rows of values, where in the equation A=bC, b*C must still equal 100, using the same non-pathlength controlled calibration model.

TABLE 1
Univariate Calibration Using Fixed Pathlength

| Phase | Absorbance | Concentration | Pathlength |
|---|---|---|---|
| Prediction | 2 | 200 | 0.5 |
| Calibration | 2 | 100 | 1.0 |
| Prediction | 2 | 50 | 2.0 |

Hence, an uncontrolled change in pathlength and use of a model that does not compensate for pathlength potentially results in large errors. In the case of optically probing skin using light in the wavelength range of 1000 to 2500 nm, variations in pathlength result for a number of sources, such as: physiological variations between people, physiological inhomogeneities across a sample site, changes in water concentration, changes in scattering component density, changes in relative thickness of probed layers, changes in shape of an interface between two tissue layers, and/or changes in temperature.

Therefore, apparatus and/or methods used to control/compensate for pathlength changes drastically improve accuracy and precision of analyte concentration determination. Herein and throughout, a connected set of detectors, optical filters, light directing optics, optical train geometry, sample knowledge, and algorithms yield a calibration model and an analyzer that are surprising. Particularly, those skilled in the art know that a noninvasive glucose concentration analyzer has not been previously developed with in home/unmonitored use requirements of an organization like the Food and Drug Administration despite approximately one billion dollars of research and development effort. The presented analyzer/algorithm combination is beyond the sum of its parts and is not obvious, even with extensive research, from previous publications.

### Scattering

Changes in scattering manifest as changes that affect accuracy and/or precision of a calibration model. For example, a change in scattering in tissue, in the spectral region of 1000 to 2500 nm, will result in changes or variation in one or more of: total optical pathlength, standard deviation of total pathlength, optical pathlength in a given tissue layer, mean optical depth of penetration, mean optical pathway, and standard deviation in optical depth of penetration.

Since scattering changes typically manifest as a change in the optical pathlength traveled by detected photons, changes in scattering of the sample yield errors in an unmanaged calibration, while hardware/software controls overcome the changed optical pathlength problem, as described infra.

### Photonic Pathways in Tissue

Referring now to FIG. 2A, a photon-skin transport system 200 through skin layers of the subject 170 is illustrated. The photon transport system 120 and/or sample interface 150 optionally uses one or more fiber optics, optical guides, mirrors, and/or lenses to direct light from the source system 110 to the detector system 130 via skin of a subject 170. In this example, the photon transport system 120 guides light from a source 112 of the source system 110 to the subject 170. Further, in this example, the photon transport system 120 irradiates skin of the subject 170 over a narrow illumination zone 177, such as having an area of less than about 16, 9, 4, 1, 0.25, 0.1, and/or 0.01 mm², where photons enter the skin of the subject 170. For clarity of presentation, the photons are depicted as entering the skin at a single point. A portion of the photons traverse, or more particularly traverse through and/or diffusely travel through, the skin to a detection zone 179. The detection zone 179 is a region of the skin surface where the detector system 130 gathers and subsequently detects the traversing or diffusely reflected photons exiting the skin of the subject 170. Optionally, the illumination zone 177 comprises one or more irradiation zones associated with one or more sources and/or a changing illumination area as a function of time as incident photons enter the tissue at different locations as a function of time. Similarly, the detection zone 179 optionally comprises one or more light collection areas and/or a changing detection area as a function of time as detected photons detected with one or more detectors emerge from a corresponding one or more areas of the skin and/or change position with time, such as dependent upon a changing illumination zone location as a function of time and/or a subject tissue change as a function of time.

Still referring to FIG. 2A, the source system 110 is illustrated with an optional air gap 210 between a last optic of an illumination system of the source system 110 and skin of the subject 170. Optionally, the photons are delivered to the skin of the subject 170 through an optic or set of optics proximately contacting, but not actually contacting, the skin, such as less than about 0.5, 1.0, or 2.0 millimeters of the skin.

Still referring to FIG. 2A, photons from the source 112 travel on a complex path into and through tissue of the subject 170. Herein, all pathways of photons through skin are illustrative in nature as actual pathways of the more than tens, hundreds, or thousands of photons are extremely diverse. The illustrated photonic pathways represent statistical averages and/or representative optical pathways for given sets of photons at discrete wavelengths and/or over a range of wavelengths. Further, for clarity of presentation, photons reaching a detector element are generally presented. The various photons traversing or diffusely scattering through the skin sequentially encounter as a function of depth a stratum corneum, an epidermis 173 or an epidermis layer, a dermis 174 or dermis layer, and subcutaneous fat 176 or a subcutaneous fat layer. As depicted in FIG. 2A, the diffuse reflectance of the various photons through the skin detected by the detection system 130 follow a variety of optical paths through the tissue, such as shallow paths through the epidermis 173, deeper paths through the epidermis 173 and dermis 174, and still deeper paths through the epidermis 173, dermis 174, and subcutaneous fat 176. However, for a large number of photons, there exists a mean photon path for photons from a point entering the skin at the illumination zone 177 until exiting the skin at the detection zone 179 and being detected by the detection system 130. In the illustrations, optical pathlengths are illustrated as straight lines and/or curved lines for clarity of presentation; in practice light travels in straight lines between individual scattering events of multiple scattering events and light interacts with skin constituents and/or groupings of constituents through laws of physics to scatter and transmit through skin voxels also referred to as volume pixels or skin volumes.

### Dermis Probe

Still referring to FIG. 2A, it is illustrated that at an intermediate distance between the illumination zone 177 and the detection zone 179, a relatively higher percentage of photons probe the dermis 174 layer of tissue. More particularly, at the intermediate distance, all of the detected photons probe at least the epidermis layer, though the intermediate distance allows an increasing percentage of the incident photons reaching the dermis 174 scattering back to the detection zone 179 and ultimately to the one or more detectors 132 while a still relatively small percentage of photons probing the subcutaneous fat 176 layer reach the intermediate distance detection zone 179. The relative percentage of photons probing the epidermis 173, dermis 174, and subcutaneous fat 176 is further described in relation to at least FIG. 2B and FIG. 2C.

### Epidermis Probe

Referring now to FIG. 2B and still referring to FIG. 2A, the photon transport in tissue is further described in terms of photons from the source system 110 probing the epidermis 173 of the subject 170. Generally, small radial distances between the illumination zone 177 and detection zone 179 result in a larger percentage of detected photons having penetrated only the epidermis 176 without penetration to deeper tissue depths. However, many conditions affect the actual percentage of detected photons probing dominantly the epidermal layer. For instance, in the near-infrared, high water absorbance regions at some frequencies extremely limit detection of photons having penetrated to deeper tissue layers due a sensitivity limit of a near-infrared detector being rapidly reached with a high absorbance multiplied by a large pathlength. Similarly, photons probing tissue with a very high anisotropy, such as greater than 0.91 or 0.93, readily penetrate to deeper depths limiting detection of photons having an intermediate absorbance, again due to the product of pathlength times absorbance exceeding readily available near-infrared detector sensitivity limits. Hence, for tissue, due to high absorbance of sample constituents like water, and further in view of scattering and anisotropy, the majority of photons observed at small radial distances between the illumination zone 177 and the detection zone 179 have larger spectral features related to the epidermis 173, as opposed to the deeper tissues layers, such as the dermis 174 tissue layer and/or the tissue layer of subcutaneous fat 176.

### Subcutaneous Fat Probe

Referring now to FIG. 2C and still referring to FIG. 2A and FIG. 2B, near-infrared photons probing the layer of subcutaneous fat 176 are further described. Generally, at still larger radial distances between the illumination zone 177 and the detection zone 179, compared to the short radial distance described above for probing the epidermis 173 and the medium radial distance described above for probing the dermis 174, an increasing percentage of the photons collected have penetrated into and thus probed the layer of subcutaneous fat 176. Again, as described supra, and as illustrated, multiple photonic pathways probe the layer of subcutaneous fat 176. Naturally, as photons reaching the subcutaneous fat 176 have necessarily traversed the epidermis 173 and dermis 174, detected photons have additional information related to those layers. However, the relative percentage of detected photons having probed the subcutaneous fat increases with radial distance to a distance of 2, 3, or 4 millimeters, depending on a wavelength of probing light. Additionally, wavelength discrimination additionally uses spectral absorbance features that vary as a function of tissue depth and skin layer thicknesses, as described infra.

Still referring to FIG. 2C, transport of near-infrared photons in tissue is further described, as a function of depth and radial distance. In FIG. 2C, three representative mean optical pathways are illustrated: pathway A has a first radial distance dominantly associated with the epidermis 173, as described supra; pathway B has a second, longer, radial distance with an enhanced percentage of photons probing the dermis 174 layer relative to shorter and longer radial distances, as described supra; and pathway C has a third, still longer, radial distance with a relatively larger signal related to the layer of subcutaneous fat 176. Generally, as the radial distance increases between the illumination zone 177 and the detection zone 179, the mean depth of penetration also increases, as described infra. However, for optimal results, wavelength dependent absorbance, wavelength dependent scattering, and/or wavelength dependent anisotropy is also considered and is discussed relative to at least FIG. 2E, FIG. 2F, and FIG. 2G for the absorbance, scattering, and anisotropy parameters, respectively.

### Sample Interface Contact

Referring still to FIG. 2C and referring now to FIG. 2D, proximate contact of the photon transport system 120 and/or the sample interface 150 to the subject 170 is described. In FIG. 2C, the photon transport system 120 is illustrated with the above described optional gap 210. In FIG. 2D, the photon transport system 120 is illustrated as proximately contacting or contacting the skin of the subject 170, which is done to minimize specularly reflected light off of the sample. Herein, proximately contacting the skin means that the distance between a proximately contacting surface of the photon transport system is less than 1.0, 0.5, 0.25, or 0.1 mm from the surface of the skin.

As the subject 170 moves with time, maintaining contact or proximate contact of a portion of the photon transport system 120, the sample interface 150, and/or a sample probe tip of the analyzer 100 with the skin optionally uses dynamic control to maintain contact of any of the photon transport system 120, sample interface 150, and/or sample probe 152 contact with the skin of the subject 170. For example, a distance between the analyzer 100 and the skin of the subject 170 is maintained with a vibration and/or shake reduction system. For instance, a section of the analyzer 100 optically contacting the skin of the subject 170 is optionally dynamically controlled. As movement of skin tissue is slow relative to a controlled electromechanical positioner, the contacting optics optionally react to shake or movement of the subject. For example, a dynamic vibration reduction system, such as used on a single lens reflux (SLR) camera, is optionally used to dynamically adjust for movement of the sample site. For instance, shake of the sample site is monitored and an element of the optical system and/or a portion of the analyzer 100 is dynamically adjusted to compensate for movement of the sample site. The shake is optionally monitored with probing photons from the noninvasive glucose analyzer source at time of measurement and/or at intervals between measurements, such as at time intervals of less than about 0.01, 0.05, 1/10, ⅕, ½, or 1 second. Optionally, shake of the skin of the subject 170 is monitored with photons from a second optical source, such as a source with shorter wavelengths at less than 400 or 700 nm for enhanced motion sensitivity.

### Aperture

Referring again to FIG. 2C and still referring to FIG. 2D, aperture of the detection system 130 is described. For clarity of presentation, the three mean optical pathways (A-C) illustrated in FIG. 2C probing the epidermis 173, dermis 174, and layer of subcutaneous fat 176 are illustrated in FIG. 2D relative to four different apertures (APT₍₁₋₄₎), where an aperture controls light throughput. Particularly, the illustrated apertures additionally limit: (1) mean depth of penetration of the collected probing photons as further described herein, (2) standard deviation of the collected probing photons as further described herein, (3) a mean optical pathway of the collected probing photons, and (4) spread of total pathlength, as described infra. Generally, the aperture controls light throughput from the skin of the subject 170 to photon transport system 120 and/or to the detector system 130, such as by providing a photon blocker outside the aperture and/or providing a light directing optic inside the aperture. For clarity of presentation, the four apertures are further described by way of the six subsequent examples. The four described apertures are illustrative in nature for clarity of presentation and are representative of any aperture shape and/or diameter.

### Example I

Still referring to FIG. 2D, in a first example, the relatively large first aperture 232, APT₁, is described, where the first aperture 232 couples light from a large surface area of skin of the subject 170. As illustrated, the first aperture 232 captures light from all of: (1) pathway A, dominantly associated with the epidermis 173; (2) pathway B, with an enhanced percentage of photons probing the dermis 174 layer, and (3) pathway C, additionally probing the layer of subcutaneous fat 176. As such, at least for a single wavelength, the relatively large first aperture 232 has limited ability to resolve information related to tissue layer as all layers are sampled in the mixed and combined signal. Further, the relatively large first aperture 232 collects photons with: relatively short optical pathlengths, pathway A; intermediate pathlengths, pathway B; and relatively long optical pathlengths, pathway C. As such, at least for a single wavelength, ability to determine an analyte concentration is hindered due to the link of concentration to both the observed signal and pathlength. More generally, an uncontrolled change in pathlength and use of a model that does not compensate for pathlength results in large errors relating an observed signal to a concentration, such as a link of absorbance, A, without control of pathlength, b, to a concentration, C, in Beer's Law as described supra in the pathlength section. Examples of the large first aperture 232 are: (1) collection of light in multiple fiber optics where the collected light is sent to a single detector element, (2) where the total light collection area exceeds two square millimeters and/or (3) collection of light over a spread of radial distances from the illumination zone of greater than 1500 μm to a given detector element of the detector system 130.

### Example II

Still referring to FIG. 2D, in a second example, the intermediate second aperture 234, APT₂, is described, where the second aperture 234 couples light from an intermediate surface area of skin of the subject 170, such as over a spread of radial distances from the illumination zone 177 of 3, 4, or 5 fiber diameters and/or over a radial distance of greater than 500, 750, or 1000 μm and less than a radial distance of 1250 or 1500 μm. Generally, the second aperture 234 restricts light to a narrower radial distance than the first aperture 232. As illustrated, the second aperture 234 restricts collected light to include pathway A 222 and pathway B 224, while excluding light from pathway C 226, though the second aperture 234 could, if positioned further radially outward from the illumination zone 177, similarly collect light from pathways B and C, while excluding light from pathway A. The second aperture 234, being narrower, especially in terms of total range of radial distance from the illumination zone 177 restricts: (1) the range of total optical pathlengths collected and (2) the standard deviation of the mean depth of penetration of each of the collected photons. Reduction in the range of total optical pathlengths collected enhances certainty in concentration, C, through enhanced certainty of/reduction in deviation of pathlength, b, such as in Beer's Law. Further, reduction in the standard deviation of the depth of penetration of the collected photons reduces interferences or signals related to sample layers not containing the analyte of interest, such as the layer of subcutaneous fat 176. Thus, the intermediate radial distance spread of the second aperture 234 yields a higher percentage of photons probing a desired tissue layer and a reduction and/or near elimination of photons penetrating into a deeper tissue layer than desired. Combined, the total pathlength spread reduction, the reduction of spread of photons in terms of depth in the sample, and the enhanced targeting of photons to desired depths enhances accuracy of a resulting determination of an analyte concentration and/or reduces uncertainty of the determined analyte concentration, albeit with a decrease in the total number of photons collected, which is addressed infra, in terms of multiple detection sites and/or integration time.

### Example III

Still referring to FIG. 2D, in a third example, the relatively small third aperture 236, APT₃, is described, where the third aperture 236 couples light from a relatively small surface area of skin of the subject 170, such as over a spread of radial distances from the illumination zone 177 of a radial diameter of 1-2 fibers and/or less than 100, 200, 300, 400, or 500 um. As illustrated, the third aperture 236 collects light along pathway B while excluding light along both pathway A and pathway C. The narrower third aperture 236: (1) reduces the standard deviation of the mean depth of penetration of each of the detected photons and (2) reduces the standard deviation of the total optical pathlength of the detected photons. As to the first point, the reduced standard deviation of the mean depth of penetration of each of the detected photons means that the certainty of the amount of pathlength probed by a given photon or group of photons in a narrow range of depths of tissue is enhanced. Thus, the certainty of the photons, in aggregate, probing the desired tissue layer, such as the dermis 174, is enhanced. As to the second point, the correlated reduction in standard deviation of the total optical pathlength further: (1) enhances the certainty of the photons traversing the desired depth for a longer time period and (2) enhances the certainty an analyte property determination, such as glucose concentration, being accurate as the relationship with the detected signal, such as the absorbance, is enhanced through reduction of the confounding pathlength, such as in the relation of concentration, C, to both absorbance, A, and pathlength, b, in Beer's law, as detailed supra.

### Example IV

Still referring to FIG. 2D, in a fourth example, more than one aperture is optionally used over the same time period, such as the third aperture 236 figuratively linked to pathway B and a first detector element and a fourth aperture 238, APT₄, figuratively linked to pathway C and a second detector element. The use of 2, 3, 4, 5, or more apertures coupled to a corresponding 2, 3, 4, 5, or more detectors of the detector system 130 allows a collection of a spatially resolved set of signals, each of the set of signals coupled to a distinct optical pathway in the tissue with known and usable sampling constraints, as further described infra. The use of multiple apertures with associated detectors increases the number of photons collected per unit time with an associated increase in an overall signal-to-noise ratio, but with discretization of total optical pathlength, depth of penetration, and pathlength in a targeted range of tissue depth.

### Example V

Still referring to FIG. 2D, in a fifth example, multiple apertures are used. For example, each of n apertures are linked to a corresponding detector element of n detector elements, where n is a positive integer of 2, 3, 4, 5, 10, 20, 50, or more. Individual apertures are optionally and preferably proximately contacting or contacting skin of the subject 170 between the subject and the corresponding detector element. Individual apertures are optionally an opening through an optical blocking material, such as a series of holes in a plastic sheet or a set of fiber optic ends. Optionally, the size of the aperture increases with increased radial distance from the illumination zone 177 and/or the aperture size is inversely related to the number of photons collected as a function of time.

### Example VI

Still referring to FIG. 2D, in a sixth example, an individual aperture varies in cross-sectional area of the opening as a function of time and/or is dynamically set on the analyzer 100 based upon one or more dominant factors defining a tissue type of the subject, such as a thickness of the subject's dermis or the mean depth within the tissue of an interface between the dermis 174 and the subcutaneous fat 176.

### Tissue Properties

The following three sections further address: (1) tissue sample absorbance, (2) scattering of light in tissue, and (3) anisotropy of photon travel in skin tissue, all in terms of the analyzer 100 and using the sample to aid in noninvasive glucose concentration determination in tissue using the properties of the tissue to aid in wavelength separation and/or pathway/pathlength separation, optionally with and optionally without traditional use of traditional wavelength separation elements.

### Sample

In the case of noninvasive glucose determination in human skin in the wavelength ranges of 1000 to 2500 nm, the sample affects the resultant spectra in a number of ways that yield information on the sample. Three non-limiting examples are described. In a first example, water absorbance is a very large contributor. In a second example, scattering as a function of wavelength generally decreases from 1000 to 2500 nm. A combination of just water absorbance and scattering as a function of wavelength yields a first approximation of both optically probed depth of penetration and total optical pathlength of detected probing photons. In a third example, anisotropy further alters early and/or primary parameters of a model modeling the largest variation of detected photons, especially at wavelength ranges of relatively rapid change in anisotropy as a function of wavelength, such as from 900 or 1000 nm to 1300 or 1400 nm.

### Absorbance

Referring now to FIG. 2E, the photon-skin transport system 200 is further described in terms of absorbance. Particularly, the absorbance of skin as a function of wavelength is addressed. In FIG. 2E, two illustrative photonic pathways are presented for a first wavelength, λ₁, of high absorbance and a second wavelength, λ₂, of lower absorbance, where the percentage of photons reaching a given distance along the illustrative path is represented by the width of the illustrated pathway. In this absorbance dominated case, the first wavelength represents a wavelength or wavelength region of high absorbance, such as a region of high water absorbance from 1350 to 1450 nm or 1850 to 2050 nm. In the first wavelength region of high absorbance, it is observed that few photons penetrate to greater tissue depths and return to the detection zone 179. Conversely, at the second wavelength, such as from 1500 to 1700 nm, photons penetrating into the deeper tissue layers, such as the dermis 174 are detected at a radial distance, where the first radial distance and aperture is limited by: (1) the detection system 130, (2) a detector optic, and/or (3) a first aperture, as described in relation to FIG. 2D above. Thus, using the absorbance of the sample, the analyzer 100 functions as a spectrometer separating two wavelengths of light. The separation of two wavelengths of light is: (1) absolute where the absorbance differ greatly or (2) is partial at similar absorbances of light. In the case of partial separation, (1) radial distance further discriminates the two wavelengths and (2) the number of photons from each wavelength range is weighted by absorbance and radial distance factors. It is noted that variations in absorbance are optionally used to partially separate greater than 2, 3, 4, 5, 10, 20, 50, or 100 wavelengths as a function of radial distance. Notably, traditional absolute separation of wavelengths of light as in a traditional spectrometer is not needed as the resolution of pathlength is key, as described above, and the partial separation of wavelengths further aids analysis in a multiplexed analysis, described infra.

### Scattering

Referring now to FIG. 2F, the photon-skin transport system 200 is further described in terms of scattering. Particularly, the scattering of skin as a function of wavelength is addressed as a means for separating wavelengths of light. In FIG. 2F, three illustrative photonic pathways are presented for a third wavelength, λ₃, with a high scattering coefficient, a fourth wavelength, λ₄, with a medium scattering coefficient, and a fifth wavelength, λ₅, with a low scattering coefficient. In this case representing scattering of skin tissue, the third wavelength represents a wavelength or wavelength region of high scattering, such as from 800 to 1300 nm, the fourth wavelength of intermediate scattering, such as from 1300 to 1900 nm, and the fifth wavelength of low scattering, such as from 1900 to 3000 nm. In the third wavelength region of high scattering, it is observed that photons penetrate to a shallow tissue depth, such as into the epidermis 173, return to the incident light surface, and are detected by the detector system at a small radial distance, such as less than 0.5, 0.75, or 1.0 mm from the illumination zone. Here, the small radial distance collected photons are illustrated as passing through a first light collecting element 133 of the detector system 130, such as a first aperture and/or a first optic. In the fourth wavelength region of medium scattering, it is observed that photons penetrate to a deeper tissue depth, such as into the dermis 174, and return to the incident light surface where they are detected by the detector system at an intermediate radial distance, such as from about 1.0 to about 2.5 mm from the illumination zone 177. Here, the intermediate radial distance collected photons are illustrated as passing through a second light collecting element 135 of the detection system 130, such as a second aperture and/or a second optic. The second aperture of the second light collection element 135 is optionally larger than a corresponding aperture of the first light collection element 133, as further described infra. In the fifth wavelength region of low scattering, it is observed that photons penetrate to a deeper tissue depth, such as into the layer of subcutaneous fat 176 for subsequent detection at the incident surface or do not return to the incident illumination surface, as illustrated. Thus, using the scattering of the sample, the analyzer 100 functions as a spectrometer radially separating two wavelengths of light, such as the third and fourth wavelengths. It is noted that variations in scattering of skin are optionally used to partially separate greater than 2, 3, 4, 5, 10, 20, 50, or 100 wavelengths as a function of radial distance using a corresponding greater than 2, 3, 4, 5, 10, 20, 50, or 100 apertures and/or light collection optics. Again, complete, absolute, and/or high resolution, such as better than 0.1, 1, 2, or 5 nm resolution, separation of the wavelengths using scattering is not essential as a primary factor in collection of a narrow range of pathlengths for a photonically sampled tissue volume associated with individual detector elements, as described above and the partial separation of wavelengths adds to discrimination of the tissue optically sampled, in terms of probability, which is used by the multiplexed analysis, as described infra.

### Anisotropy

Referring now to FIG. 2G, the photon-skin transport system 200 is further described in terms of anisotropy or forward light scattering. Particularly, the anisotropy of skin as a function of wavelength is addressed. The inventor has discovered that anisotropy is not constant across the wavelength range of 1100 to 2500 nm as reported in the literature. Instead, the anisotropy falls rapidly from about 0.97 to about 0.93 from 1100 to 1400 nm and then falls slowly to about 0.90 to 2500 nm. In FIG. 2G, the effect of anisotropy is illustratively presented for a sixth wavelength, λ₆, of high anisotropy, such as greater than about 0.95; at a seventh wavelength, λ₇, of intermediate anisotropy, such as about 0.91 to 9.95, and at an eighth wavelength of, λ₈, of still lower anisotropy, such as 0.8 to 0.91. As illustrated, if considering only anisotropy, deep tissue layers are probed and few photons return to the incident light surface. However, photons penetrating into the skin of the subject 170 at the illumination zone 177 interact with the tissue and scatter centers therein in view of the absorbance coefficient and the scattering coefficient described above in addition to the anisotropy of the various tissue layers. Thus, anisotropy as a function of wavelength is optionally used to separate two or more wavelengths. The combined effect of absorbance, scattering, and anisotropy is further described in the next section.

### Absorbance, Scattering, and Anisotropy

Referring now to FIG. 2H, the photon-skin transport system 200 is further described in terms of a combination of absorbance, scattering, and anisotropy of skin/tissue/blood layers of the subject 170 in terms of wavelength, pathlength, and/or pathway separation. The photon-skin transport is optionally further considered in view of additional skin and/or optic parameters, such as: temperature, wavelength, and protein absorbance as described infra. In FIG. 2H, a first mean photonic path 222 is illustrated for a first combination of absorbance, scattering, and anisotropy light transport properties as well as for a second mean photonic path 224 and third mean photonic path 226 for a second and third combination of absorbance, scattering, and anisotropy light transport properties, respectively. As illustrated, the first combination of the three light transport properties yields photons at a first radial distance being collected and detected by a third light collecting element 137 of the detection system 130. Similarly, the second and third combination of the three light transport properties yields photons at a second and third radial distance being collected and detected by a fourth light collecting element 138 and a fifth light collecting element 139 of the detection system 130, respectively. Hence, radial discrimination of the mean tissue pathways 222, 224, 226 is performed using relatively small discrimination apertures at three distinct radial distances. The three illustrated small apertures corresponding to the three combinations of light transport properties yields a relatively small spread of total optical pathlengths, depths of penetration, and mean time/mean pathway/mean pathlength in a tissue layer as described above in terms of: (1) the mathematical product of pathlength times concentration and (2) the discrimination of pathlengths with relatively small apertures. It is noted that the illustrated discrimination of three optical pathways in terms of absorbance, scattering, and anisotropy is optionally applied to greater than 2, 3, 4, 5, 10, 20, 50, 100, 500, 1000 optical pathways through use of associated light collection zones and/or discriminated detection zones, which are physically separated examples of the detection zone 179. The larger number of discriminated optical pathways are further described below.

### Multi-Radial Direction Optical Pathway Discrimination

Referring now to FIG. 2I and FIG. 2J, a case of multiple distinct combinations of tissue optical pathways or a set of multiple wavelengths of light overlapping at a given radial distance is addressed. As illustrated in FIG. 2I, a first combination of the tissue properties of absorbance, scattering, and anisotropy yield a first optical pathway 227, a second combination of the tissue properties of absorbance, scattering, and anisotropy yield a second optical pathway 228, and a third combination of the tissue properties of absorbance, scattering, and anisotropy yield a third optical pathway 229, where all three optical pathways have a common or approximately common mean radial distance from the illumination zone to the detection zone 179. As illustrated, the first, second, and third optical pathways have distinct wavelengths, λ₉, λ₁₀, λ₁₁, and/or have a distinct combination of absorbance, scattering, and anisotropy, (Aₙ, Sₙ, gₙ), where n comprises a positive integer. Separation of the three wavelengths and separation of the three combinations of absorbance, scattering, and anisotropy having the same mean radial distance between the illumination zone 177 and detection zone 179 is described in the following two examples.

### Example I

Still referring to FIG. 2I and FIG. 2J, a first case of separation of two photonic pathways having both: (1) a common radial distance and (2) highly overlapped in tissue pathways between the illumination zone and the detection zone is described. As described above, radial distance from an illumination zone is optionally used to separate photonic pathways. In this example, the radial distance is further considered in two directions from the illumination zone 177 in combination with a first optical filter 231 optically linked to a first detector of the detector system 130 in a first direction and a second optical filter 233 optically linked to a second detector of the detector system in a second direction, where: (1) the first optical filter 231 passes a wavelength of light associated with the first optical pathway 227 while blocking a wavelength of light associated with the second optical pathway 228 and (2) optionally, the second optical filter 233 passes the wavelength of light associated with the second optical pathway 228 while blocking the wavelength of light associated with the first optical pathway 227, thereby separating the sampling of the two highly overlapped photonic pathways having a common radial distance of detection from a common illumination zone, optionally without use of a grating element or a time-domain moveable element of a spectrometer.

### Example II

Still referring to FIG. 2I and FIG. 2J and now referring to FIG. 2K, a second case of separation of two photonic pathways having both: (1) a common radial distance and (2) substantially non-overlapped tissue mean optical pathways between the illumination zone 177 and the detection zone 179 is described. As described above, radial distance from an illumination zone is optionally used to separate photonic pathways. In this example, the radial distance is further considered in three directions from the illumination zone 177 in combination with: a first optical filter 231 optically linked to a first detector 241 of the detector system 130 in a first direction; a second optical filter 233 optically linked to a second detector 243 of the detector system 130; and a third optical filter 235 optically linked to a third detector 245 of the detector system 130, where: (1) the first optical filter 231 passes a wavelength of light associated with the first optical pathway 227 while blocking wavelengths of light associated with both the second optical pathway 228 and third optical pathway 229; (2) optionally, the second optical filter 233 passes the wavelength of light associated with the second optical pathway 228 while blocking wavelengths of light associated with both the first optical pathway 227 and the third optical pathway 229; and (3) optionally, the third optical filter 235 passes the wavelength of light associated with the third optical pathway 229 while blocking wavelengths of light associated with both the first optical pathway 227 and the second optical pathway 228, thereby separating the sampling of the three photonic pathways having a common radial distance of detection from a common illumination zone.

From the previously described first and second examples in this section, it is clear that n filters are optionally associated with n wavelength regions that are distinct or partially overlap in transmitted wavelengths to separate as a function of wavelength photonic pathways having similar mean radial distributions even if the depth of penetration of the pathways differ, where n is a positive integer of more than 3, 4, 5, 10, 15, or 25.

Further, from the previously described first and second examples in this section, it is clear that providing 2, 3, 4, or more illumination zones, where the 2, 3, 4, or more illumination zones use distinct wavelength, such as from LED sources, that the common radial distance problem described in this section is overcome as the distance from a first, second, or third illumination source at different physical locations on the skin of the subject 170 results in previously overlapped radial distances becoming at least partially separated. The variation in illumination zone position 177 is further described in the next section.

The inventor notes that discriminations of optical pathways as a function of the tissue properties of absorbance, scattering, and anisotropy are further aided by wavelength discrimination of the sources, knowledge of sample properties in terms of absorbance, scattering, and anisotropy as a function of radial position, apertures as a function of radial distance, optical filter properties as a function of radial distance, and detector type, as described below. Still further discrimination is provided by focal length, incident angle of illumination, numerical aperture of light providing elements, numerical aperture of light collection elements, and detection angle of collection optics all as a function of wavelength, as further described below.

Combined, the above described parameters of pathlength, scattering, sample, and wavelength range in terms of absorbance, scattering, anisotropy, wavelength of light, and radial separation of illumination zones and detection zones yield considerable information on the sample being analyzed. Inclusion of this information in: (1) a design of the analyzer, (2) configuration of the analyzer for a given subject or subject type, and/or (3) a data processing system or algorithm used to extract information results in decreased error in analyte concentration determination and enhanced precision of the analyte concentration. Particulars of the combination of the design of the analyzer, the configuration of the analyzer for a given subject, and the data processing system are further described, infra. Non-limiting examples of a detector array, algorithm, and spatial analyzer are presented in the following sections with additional detail provided infra.

While absorbance, scattering, and anisotropy are optionally used to separate wavelengths of light and pathways traversed by the light in a first system, so also an illumination array-detector array system, with or without use of optical filters, is optionally used to separate wavelengths of light and pathways traversed by the light in a second system. The inventor notes that the first system and the second system are optionally integrated in the analyzer 100. In the next section, the source array-detector array system is described before describing the integration of the two systems, infra.

### Illumination Array/Detector Array

The illumination array and detection array are described in detail in the following sections. In this section, the general effect of use a illumination array and/or detector array on pathlength control is presented. Generally, if a set of photons penetrate into skin in an incident light zone, then after traversing a section of the skin, a portion of the photons exit the skin. Generally, position of the photons exiting the skin relative to the incident light zone yields an approximation of the total optical pathlength of the photons and/or the mean depth of penetration of the photons into the skin. Thus, combining all of the photons into one bin for subsequent detection results in a range of optical pathlengths, which is not optimal. Conversely, detecting the returning photons with an array of detectors allows simultaneous collection of photons with reduced variation in optical pathlengths and/or reduced variation in mean depths of penetration at each detector element. The analog change in signal as a function of radial position is therefore useful for: (1) mapping the sample; (2) analyzing a narrower spread of depth of penetration of photons into the sample; (3) selecting detector elements receiving a higher percentage of photons probing a tissue layer of interest, such as the glucose containing dermis; and (4) a large range of algorithmic calibration, prediction, quality control, outlier detection, and consistency approaches, described infra.

### Spatial Resolution

Spatial resolution was described, supra, in relation to aperture and FIG. 2D. Herein, spatial resolution and aperture is further addressed. For clarity of presentation and without limitation in the examples provided herein, the photons in most examples are depicted as radially traversing from a range of input zones to a range of detection zones. Similarly, photons are optionally delivered, simultaneously and/or as a function of time, from an input zone to a range of detection zones. Still further, photons are optionally directed to a series of input zones, as a function of time, and one or more detection zones are used to detect the photons directed to the series of input zones, simultaneously and/or as a function of time. Yet still further, sets of photons of controlled wavelengths are delivered to corresponding incident positions on the skin and filters and/or detectors are configured at additional locations on the skin. Still yet further, time of emission of light emitting diodes at selected wavelengths complemented by optical filter selection is optionally used to still further enhance resolution. For example an LED emitting light from 1300 to 1350 nm in combination with filters having longpass cut-on wavelengths of 1280, 1290, 1300, 1310, 1320, 1330, 1340, 1350, and 1360 nm yields corresponding narrower wavelength pass-bands and thus enhanced resolution.

The first method of spatial resolution contains two general cases of (1) photons entering a range of illumination zones and (2) photons being detected from a range of detection zones. Herein, in the first case photons are depicted traversing from a range of input points on the skin to a radially located detector to derive photon interrogated sample path and/or depth information. However, in a second case, similar systems optionally use a single input zone of the photons to the skin and a plurality of radially located detector zones to determine optically sampled photon paths and/or depth information. Still further, a combination of the first two cases, such as multiple sources or multiple illumination zones, and/or multiple detectors or multiple detection zones, is optionally used to derive photon path information in the skin. Combining the two cases with wavelength resolution via use of (1) light sources providing particular wavelengths of light, (2) optical filters limiting provided wavelengths of light to narrower wavelength ranges or spectral regions, and (3) using the sample to still further narrow wavelengths reaching a given radial distance, such as via absorbance, scattering, and anisotropy, from a source illumination zone to a detector detection zone 179 is further described after describing the two general cases.

### Integrated Illumination Zone

In the first system, referring now to FIG. 3, the photon-skin transport system 200 of FIG. 2 is illustrated where the photon transport system 120 and/or the sample interface 150 is used to irradiate the skin of the subject 170 over an integrated together wide illumination area 310 with a corresponding wide range of distances to the detection zone 179, such as where the wide illumination area 310 is less than about 0.1, 0.2, 0.3, 0.4, or 0.5 millimeters from a center or edge of the detection zone 179 to more than about 1.0, 1.2, 1.4, 1.6, 1.8, or 2.0 millimeters from a center or edge of the detection zone 179 and/or has a radial range of 3, 4, 5, or more fiber optics for bringing photons to the skin.

Still referring to FIG. 3, in the illustrated example, a mean photon path is provided as a function of radial distance from the illumination zone to the detection zone, where seven radial distances are illustrated, r₁-r₇. Generally, over a range of about zero to less than about two millimeters from the detection zone and in the range of 1100 to 2500 nm, the mean optical path of the detected diffusely scattered photons increases in depth as a function of radial distance. Using an integrated illumination zone, some photons, such as at the first radial distance, r₁, penetrate and sample just the epidermis layer 173, while other photons, such as at radial distances, r₂-r₄, additionally penetrate and probe the dermis layer 174, while still other photons, such as at radial distances, r₅-r₇, penetrate still further into the skin and sample the layer of subcutaneous fat 176. With the wide illumination area 310 and use of an integrated illumination zone, the photons probing the wide range of layers are mixed together, are integrated at a given detector, have a mix of optical pathlengths, and hence convolve pathlength with concentration. In stark contrast, use of an array of illuminators allows separation of the convolved variables, as described throughout and in the next section.

### Array of Illuminators

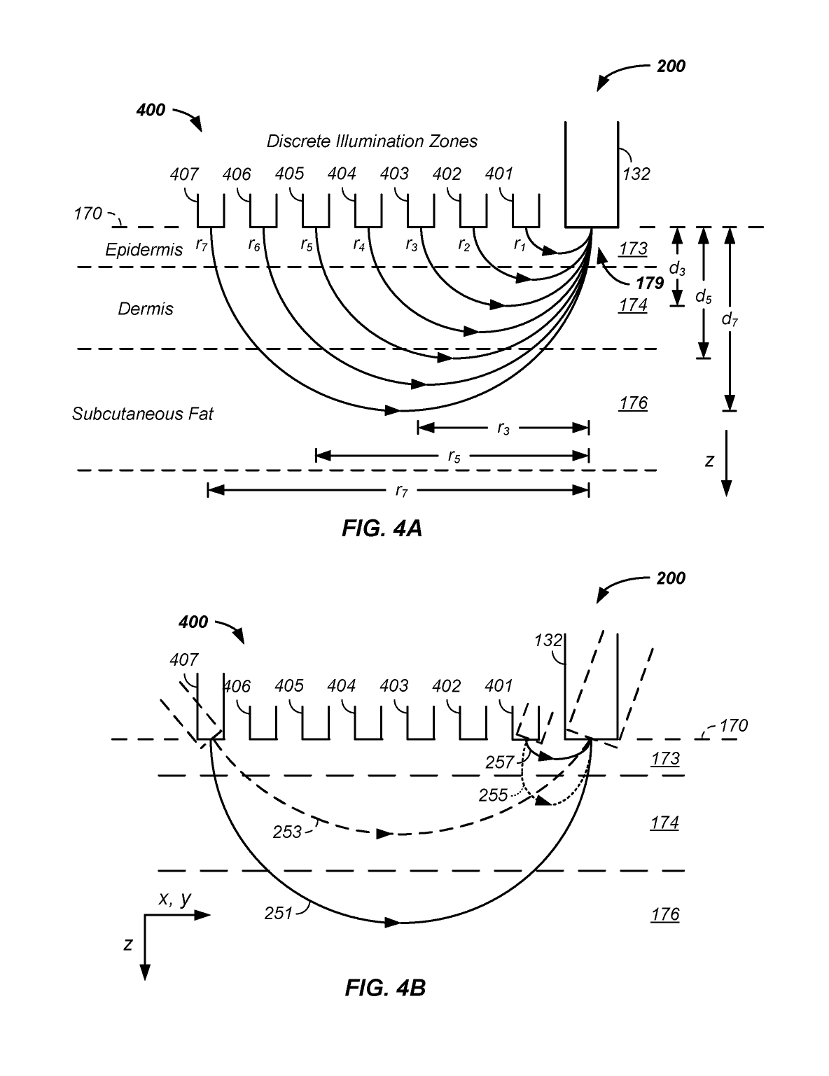

Referring now to FIG. 4A and FIG. 4B, use of an array of illuminator sources 400, when properly configured as described herein, yields smaller standard deviations in probed optical pathways of detected photons, yielding a smaller error in analyte concentration estimation, as described supra.

In a first case of the spatial resolution method, referring now to FIG. 4A, the photon-skin transport system 200 uses a vector or array of illumination sources 400, of the source system 110, in a spatially resolved pathlength/pathway determination system. For example, the illumination sources are an array of fiber optic cables, an array of light emitting diodes, light passing through an array of optical filters, and/or an array of illumination zones. Generally, the illumination zone 177 is optionally a series of local illumination zones. In this example, a set of seven fiber optics 401, 402, 403, 404, 405, 406, 407 are positioned, radially from a detection zone along the x,y-plane of the subject 170 to provide a set of illumination zones, relative to a detection fiber at a detection zone 179. As illustrated the third illumination fiber optic 403-detector 132 combination yields a mean photon path having a third mean depth of penetration, d₃, for a third fiber optic-to-detector radial distance, r₃; the fifth illumination fiber optic 405-detector 132 combination yields a mean photon path having a fifth mean depth of penetration, d₅, for a fifth fiber optic-to-detector radial distance, r₅; and the seventh illumination fiber optic 407-detector 132 combination yields a mean photon path having a seventh mean depth of penetration, d₇, for a seventh fiber optic-to-detector radial distance, r₇. Generally, for photons in the near-infrared region from 1100 to 2500 nanometers, both a mean depth of penetration of the photons and a total optical pathlength increases with increasing illumination zone-to-detection zone distance, where the illumination zone-to-detection zone distance is less than about three millimeters. More particularly, total optical pathlength, radial illumination zone-to-detection zone distance, and mean depth of penetration are dominated by water absorbance, tissue scattering, and tissue anisotropy as a function of wavelength, as described infra.

Referring again to FIG. 3 and still referring to FIG. 4A, the seven radial distances, r₁-r₇, are compared for the integrated illumination zone system and the discrete illumination zones system. As described, supra, using the integrated illumination zone system illustrated in FIG. 3, the detected photons have probed a wide range of optical pathways, radial distances, and depths. In stark contrast, each of the radial distances, r₁-r₇, in the discrete illumination zones system illustrated in FIG. 4A are optionally used independently allowing substantial separation of optical pathways, radial distances, and depths of detected photons. For example, detected photons passing at a first time through the first illumination fiber optic 401 at the first radius, r₁, dominantly sample shallow tissue depths, such as the epidermis 173, without detection of photons from the second to seventh illumination fiber optics 402-407 at the second to seventh radial distances, r₂-r₇, if illuminated at different times and/or use a wavelength-filter-sample property-detector combination to separate wavelengths, as detailed above. Again, as described above the reduced standard deviation in the optical pathway traveled allows increased accuracy in determination of the analyte property concentration, such as a noninvasive glucose concentration determination. Similarly, at the first time and/or at a second time, detected photons passing through the second, third, and/or fourth illumination fiber optics 402-404 at the second to fourth radii, r₂-r₄, target an intermediate sample shallow tissue depth, such as the dermis 174, without detection of photons from the first and fifth to seventh illumination fiber optics 401, 405-407 at the first and fifth to seventh radial distances, r₁, r₅-r₇, if illuminated at different times and/or use a wavelength-filter-sample property-detector combination to separate wavelengths, as detailed above. Generally, use of a narrow range of illuminators in terms of radial distance, such as via the discrete illumination zones, from an optically linked detector, such as a radial spread of illumination zone distances of one or two fiber optics and/or a radial spread of distances of less than 250, 500, or 750 μm results in a narrower spread or standard deviation of optical pathways of detected probing photons with a corresponding increased measure of accuracy of the targeted analyte compared to use of photons from a wider radial spread of illumination zone distances, such as from three or more adjacent fiber optics or greater than 0.75, 1, 1.5, or 2 millimeters, in the wide illumination area 310 integrated illumination zone system.

Referring again to FIG. 4A and referring now to FIG. 4B, targeting a tissue depth through control of incident angle of illumination and/or collection angle of detection is described, in terms of absorbance, scattering, and anisotropy of tissue in the near-infrared wavelength region. Three non-limiting examples are used to describe cases of angle control of the incident optics and/or of orientation of a solid angle of collection.

### Example I

Still referring to FIG. 4A and FIG. 4B, a first case of increasing the mean depth into tissue of detected photons by initially directing incident photons away from a detector element is described. In the near-infrared, when the illumination zone 177 is near the detection zone 179, such as at distances less than 400, 300, 200, or 100 μm, a shallow photon penetration mean optical path 257 of detected photons results in photons penetrating to shallow tissue depths, such as to the epidermis layer 173 without significant sampling of deeper tissue layers, such as the dermis layer 174 of skin. The inventor has determined that initially directing the incident photons on a radially outward path relative to the detector zone 179, the anisotropy, scattering, and absorbance yield a first medium photon penetration mean optical path 255 that penetrates into the dermis layer 174 of skin at radial distances, where an otherwise mean perpendicular angle of incident photons yields the shallow photon penetration mean optical path 257. Thus, useful signals sampling the glucose rich dermis layer 174 are achieved through control of a mean angle of the incident photons, such as greater than 10, 20, 30, 40, or 50 degrees off of perpendicular and away from the detection zone 179.

### Example II

Still referring to FIG. 4A and FIG. 4B, a second case of decreasing the mean depth into tissue of detected photons by initially directing incident photons at an illumination zone toward a detector element or detection zone 179 is described. In the near-infrared, when the illumination zone 177 is distant from the detection zone 179, such as at distances more than 1.5, 2, 2.5, or 3 mm, a deep photon penetration mean optical path 251 of detected photons results in photons penetrating to deep tissue depths, such as to the layer of subcutaneous fat 176, which is a glucose concentration poor layer. The inventor has determined that initially directing the incident photons on a radially inward path relative to the detector zone 179, the anisotropy, scattering, and absorbance yield a second medium photon penetration mean optical path 253 that penetrates into the dermis layer 174 of skin at radial distances without substantial optical probing of the layer of subcutaneous fat 176 at radial distances, where an otherwise mean perpendicular angle of incident photons yields the deeper photon penetration mean optical path 251. Thus, useful signals sampling the glucose rich dermis layer 174 are achieved through control of a mean angle of the incident photons, such as greater than 10, 20, 30, 40, or 50 degrees off of perpendicular and toward the detection zone 179 at larger distances between a local illumination zone and the detection zone 179, such as at the distances of more than 1.5, 2, 2.5, or 3 mm.

### Example III

Still referring to FIG. 4A and FIG. 4B, a third case of altering the mean depth into tissue of detected photons by tilting the solid angle collection cone of a collection optic at the detector is illustrated. Generally, by tilting the solid angle of the detection optic toward the illumination zone 177 or local illumination zone, the mean optical depth of the detected photons is reduced, such as from the layer of subcutaneous fat 176 to the higher glucose concentration containing dermis layer 174. The solid angle of the detection optic is optionally tilted by more than 10, 20, 30, or 40 degrees toward an associated illumination zone.

Additional examples of controlling a mean depth of penetration of, subsequently detected, photons probing into the dermis layer 174 of skin include: (1) tilting a detection optic toward the illumination zone; (2) tilting the solid angle of a detection optic optically linked with a detection element away from an illumination zone; (3) reducing or increasing the solid angle of illumination; (4) reducing or increasing the solid angle observed by a detection element; and/or (5) dynamically changing any of: the mean solid angle illumination direction, the mean solid angle of illumination, the mean solid angle detection direction, and/or the mean solid angle of detection. In addition, any combinations of the pathway controlling optics described herein are optionally used in the analyzer 100.

### Integrated Detection Zone

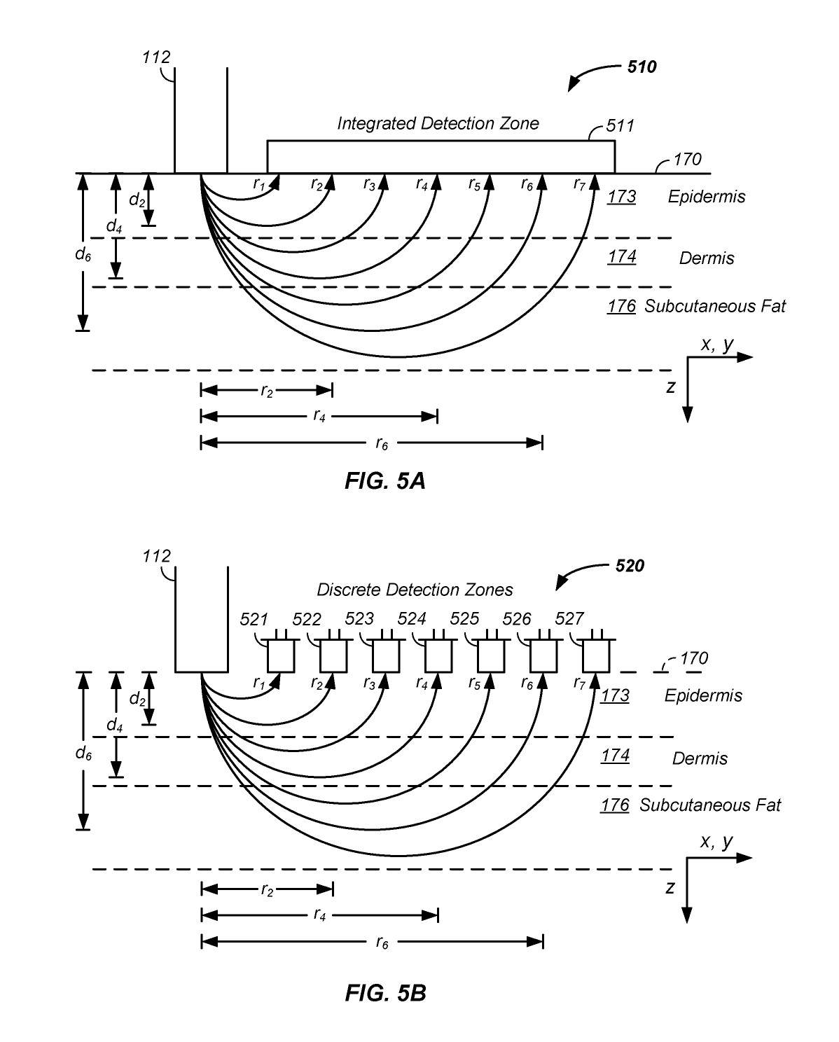

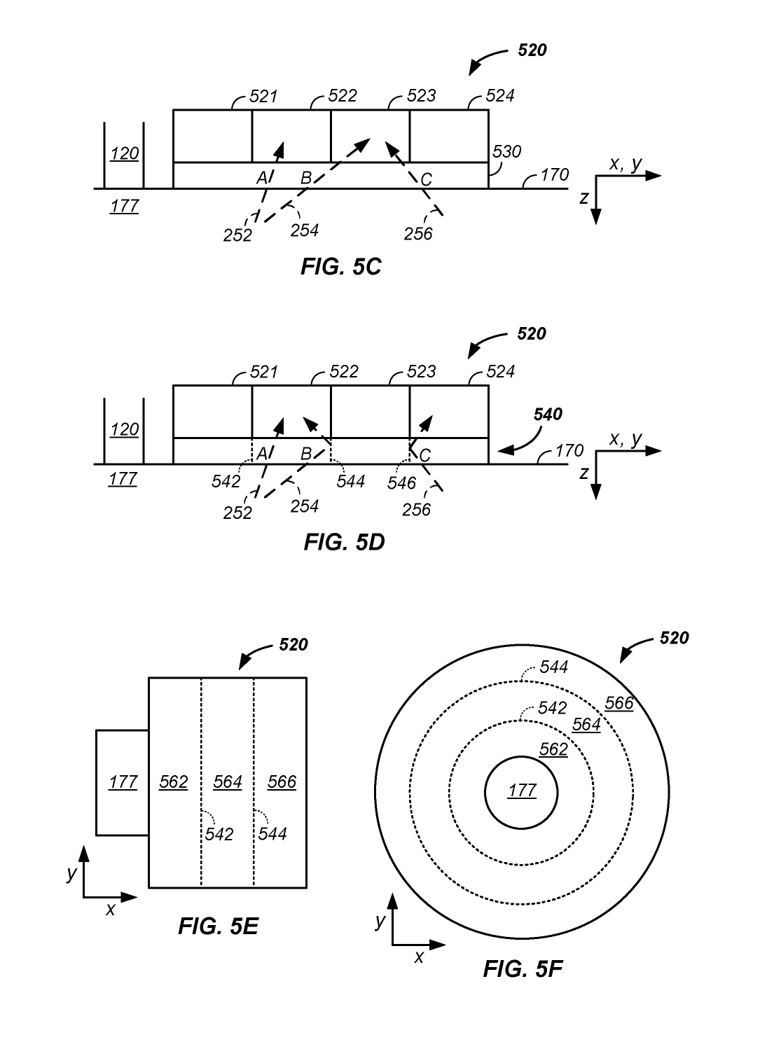

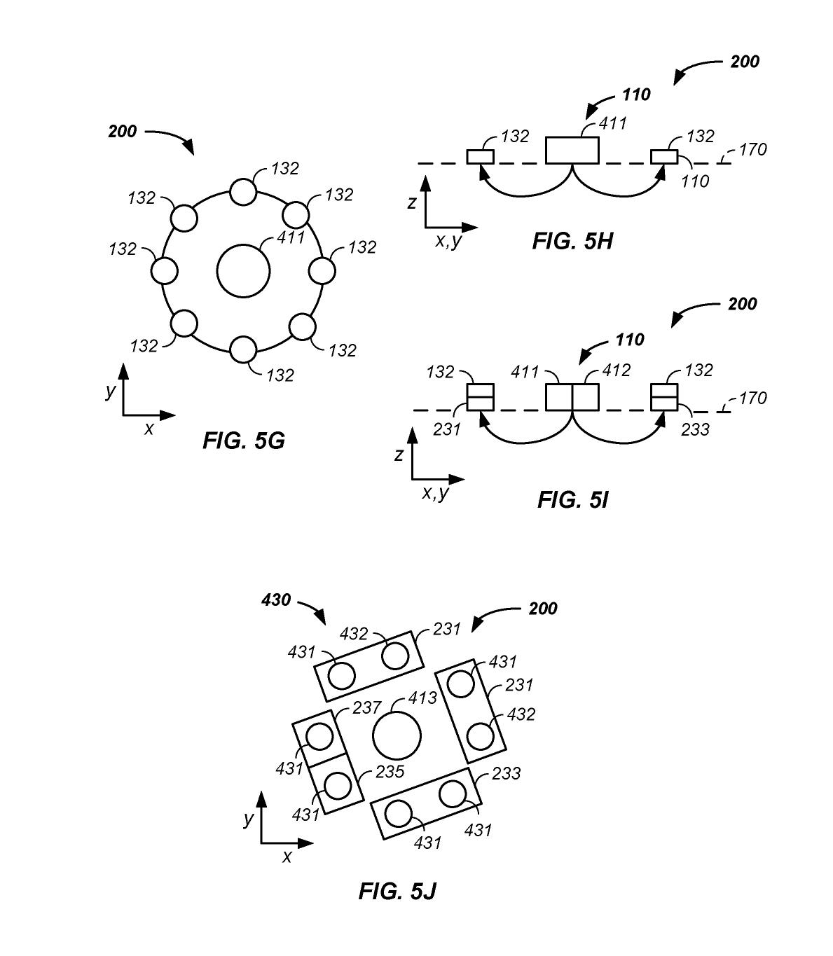

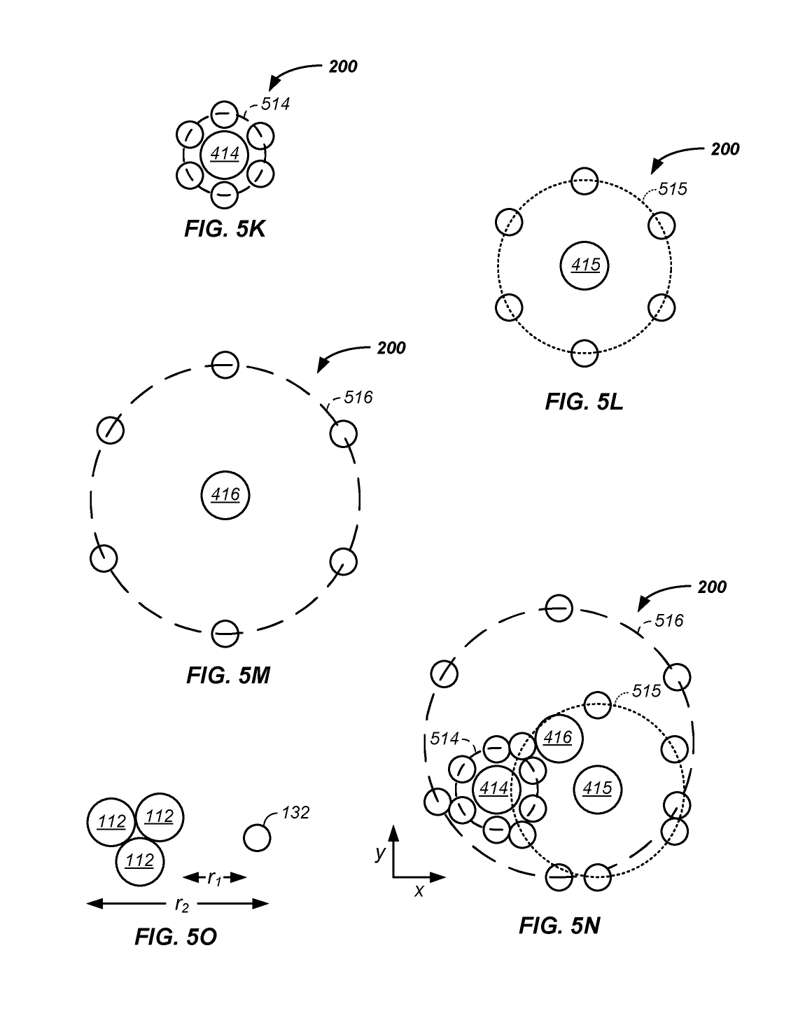

In the second system, referring now to FIG. 5A, the photon-skin transport system 200 of FIG. 2 is illustrated where the photon transport system 120, source 112, and/or the sample interface 150 is used to irradiate the skin of the subject 170 and an integrated together wide detection area 510 is used to gather photons and/or detect photons, similar to the integrated together wide illumination area 310 described above. The wide detection area 510 corresponds to a wide range of distances from the illumination zone, such as where the wide detection area 310 is at less than about 0.1, 0.2, 0.3, 0.4, or 0.5 millimeters from a center or edge of the illumination zone to more than about 1.0, 1.2, 1.4, 1.6, 1.8, or 2.0 millimeters from a center or edge of the illumination zone and/or has a radial range of 3, 4, 5, or more detection fiber optics for bringing photons from the skin to the detector 132.

Still referring to FIG. 5A, in the illustrated example, a mean photon path is provided as a function of radial distance from the illumination zone to the integrated detection zone 511, where seven radial distances are illustrated, r₁-r₇. The integrated detection zone 511 represents a light collection area where photons from the area are mixed and detected with a single detector. Generally, over a range of about zero to less than about two millimeters from the illumination zone and in the range of 1100 to 2500 nm, the mean optical path of the detected diffusely scattered photons increases in depth as a function of radial distance. Using an integrated illumination zone, some photons, such as at the first and second radial distances, r₁-r₂, penetrate and sample just the epidermis layer 173, while other photons, such as at radial distances, r₃-r₄, additionally penetrate and probe the dermis layer 174, while still other photons, such as at radial distances, r₅-r₇, penetrate still further into the skin and sample the layer of subcutaneous fat 176. With the wide detection area 510 coupled to an integrating detector, the photons probing the wide range of layers are mixed together, are integrated at a given detector, and have a mix of optical pathlengths; hence the detected photons represent photons having a pathlength variable that are convolved, mixed, and or a product with concentration. Stated again, the integrated detection area observes a large standard deviation of optical pathways. In stark contrast, use of an array of detector allows separation of the convolved variables, as described throughout and in the next section.

### Array of Detectors

As in the use of an array of illuminator sources 400, described above, the use of an array of detectors, also referred to as a detector array, when properly optically configured yields smaller standard deviations in probed optical pathways of a set or bunch of detected photons, yielding a smaller error in analyte concentration estimation.

In a second case of the spatial resolution method, referring now to FIGS. 5(B-N), the photon-skin transport system 200 uses a vector or array of detectors 520 in the detection system 130. For example, an illumination zone source, such as a single fiber optic source, sends radially distributed light to an array of staring detectors or collection optics coupled to a set of detectors. Generally, the detection zone 177 is optionally a series of local detection zones.

Referring now to FIG. 5B, an illustrative and non-limiting example is provided to clarify the invention where an array of detectors 520, such as an illustrative set of seven detectors 521, 522, 523, 524, 525, 526, 527, are positioned radially outward from the illumination zone along the x,y plane to provide a set of detection zones relative to the illumination zone. As illustrated the source 112-second detector 522 combination yields a mean photon path having a second mean depth of penetration, d₂, for a second illumination zone-to-detection zone radial distance, r₂; the source 112-fourth detector 524 combination yields a mean photon path having a fourth mean depth of penetration, d₄, for a fourth illumination zone-to-detection zone radial distance, r₄; and the source 112-sixth detector 526 combination yields a mean photon path having a sixth mean depth of penetration, d₆, for a sixth illumination-to-detection zone radial distance, r₆. Generally for photons in the near-infrared region from 1400 to 2500 nanometers both the mean depth of penetration of the photons into skin and the total optical pathlength in skin increases with increasing illumination zone-to-detection zone distance, where the illumination zone-to-detection zone distance, such as a fiber optic-to-detector optic distance, is less than about three millimeters. Further, the use of multiple detection zones yields a smaller standard deviation in: total optical pathlength, mean depth of penetration, mean radial distance traveled, and mean pathway sampled compared to use of the single integrated detection zone 510. Still further, data collected with an analyzer configured, as in the second case, with a multiple detector design generally corresponds to the first case of a multiple source design, albeit with different sampled tissue volumes due to tissue layers, tissue inhomogeneity, and tissue scattering properties.

### Segmented Spacers

Referring now to FIG. 5C and FIG. 5D, an example of an optional segmented spacer 540 is illustrated, which further enhances resolution of radial distance between an illumination zone 177 and a local detection zone. In both FIG. 5C and FIG. 5D three photon paths are illustrated, a first photon path 252, a second photon path 254, and a third photon path 256. The three photon paths terminate at an element of the array of detectors 520, such as a first detector element 521, a second detector element 522, a third detector element 523, and a fourth detector element 524. Referring now to FIG. 5C, an intermediate optic layer 530 is illustrated between the skin of the subject 170 and the array of detectors 520, where the intermediate optic layer 530 is of any thickness, such as less than 2, 1, 0.5, 0.25, and 0.1 mm thick. The intermediate optic layer 530 is optionally a protective layer between the bulk of the analyzer 100 and the subject 170, such as a protective layer to keep dirt and/or grease away from the array of detectors 520 and/or an electrical isolation layer between the powered electronics of the analyzer 100 and the subject 170. The intermediate optic layer 530 is optionally an array of micro-optics and/or a protective layer isolating the micro-optics from the subject 170.

Referring now to FIG. 5C, the first photon path 252 is illustrated as striking the second detector element 252 that covers a region directly along the z-axis from the exit point, A, of the photon from the subject 170. Thus, the second detector element 252 correctly detects a photon exiting the skin of the subject 170 in front of the detector. However, photons along the second photon path 254 and third photon path 256, respectively exiting the skin at points B and C in front of the second detector element 522 and fourth detector element 524 are each detected by the third detector element 523 due to the exit angle of the photons from the subject 170 and the thickness of the intermediate optic layer 530. As a result, resolution of radial distance from the illumination zone 177 is relatively degraded as the third detector element 523 is actually observing a radial distance along the x and/or y-axes that exceeds the diameter of a housing of third detection element 523. The radial resolution spread is addressed with use of a segmented spacer 540, infra.

Referring now to FIG. 5D, a segmented spacer 540, which is a species of the intermediate optic layer 530 resolves the radial distances from the illumination zone 177 to the respective detector elements through the addition of a barrier penetrating at least partway through the segmented spacer with a dominant z-axis direction of individual spacers. In the non-limiting example, three segmenting spacers are illustrated: a first segmenting spacer 542 positioned radially from the illumination zone 177 between the first detector element 521 and the second detector element 522, a second segmenting spacer 544 positioned radially from the illumination zone 177 between the second detector element 522 and the third detector element 523, and a third segmenting spacer 546 positioned radially from the illumination zone 177 between the third detector element 523 and the fourth detector element 524. The segmenting spacer is optionally any material, gap, or structure that redirects a photon along the x/y-plane toward a line perpendicular to the x/y-plane passing through the exit point of the photon from the subject 170, such as a mirror coating; an air gap; and/or a change in refractive index or index of refractions, as measured by Snell's Law, sufficient to redirect photons striking the segmented spacer back toward an axis running perpendicular to the face and through a point of exit of the photons from the subject, such as an index of refraction change between two surfaces of greater than 5, 10, 20, 50, or 100 percent. As illustrated, the second photon path 254 exiting the subject 170 at point B in front of the second detector element 522 now strikes the second detector element 522 and the third photon path 256 exiting the subject 170 at point C in front of the fourth detector element 524 now strikes the fourth detector element 524, thereby enhancing the radial resolution of the analyzer 100 by redirecting photons to a detector element radially associated with a radial exit point of a photon and/or blocking transmission of a photon exiting the subject 170 at a position in front of a detector element from being detected by another detector element.

Still referring to FIG. 5C and FIG. 5D and now referring to FIG. 5E and FIG. 5F, x/y-plane geometry of the segmenting spacers is further described in two non-limiting examples provided to further clarify the invention. Referring now to FIG. 5E, segmenting spacers are illustrated as parallel spacers radially positioned between sets of detection elements and/or between individual detection elements. Referring now to FIG. 5F, segmenting spacers are illustrated between concentric rings of detection elements. Vertical, horizontal, arced, and circular arrays of detection elements are further described, infra.

### Radial Resolution/Filters

Referring now to FIG. 5G and FIG. 5H, the use of light collection optics linked to multiple detector elements and/or detectors 132 circumferentially positioned about and/or radially positioned in at least two directions from an illumination optic is described. In this non-limiting example provided for clarity of presentation, eight optics linking light from a detection zone to the detectors is illustrated. However, more than 1, 2, 3, 4, 5, 10, 15, or 20 detectors 132 and/or optics linking light from respective detection zones to the detectors are optionally and preferably used in the analyzer 100. The multiple detectors function to increase the number of photons collected from the common illuminator 411. As illustrated, the common illuminator 411 is centrally located within a ring of detector elements; however, the common illuminator 411 is optionally positioned off center relative to the detection elements or outside of the detection elements as described, infra. Key here is that more than one detector element is used to detect a larger number of photons from a common illuminator compared to the use of one detection element, yet each detection element has a smaller standard deviation of an optical pathway, a smaller range of depths of penetration, and/or a smaller range of total pathlengths compared to the use of a single wide area detection element. As illustrated in FIG. 5H, the two or more detection elements 132 optionally detect an essentially similar optical pathway, though in different sections of tissue of the subject 170, allowing a post-integrated signal in software while maintaining the smaller standard deviation of sampled pathlength and/or an outlier analysis.

Referring now to FIG. 5I, an example of multiple illuminators types with a common radial distance of detection is described. In this non-limiting example provided for clarity of presentation, the light source system 110 is illustrated providing a first wavelength of light 411 and a second wavelength of light 412, though, the source system 112 optionally provides more than 2, 3, 5, 10, 15, or 20 bands of wavelengths of light with mean wavelengths separated by at least 10 nm. As illustrated, the first wavelength 411 and the second wavelength 412 interact with the tissue of the subject 170, such as in terms of absorbance, scattering, and anisotropy, to yield a common mean radial distance from the illumination zone 177 to a detection sub-zone position. Hence, without more, the detector 132 associated with each sub-detection zone position would detect both the first and second wavelengths 411, 412 of light. As illustrated, photons in a first detection sub-zone encounter a first optical filter 231 that passes the first wavelength 411 and rejects or absorbs the second wavelength 412 allowing the associated detector element to detect only the first wavelength 411. Similarly, photons in a second detection sub-zone encounter a second optical filter 233 that passes the second wavelength 412 and rejects or absorbs the first wavelength 411 allowing the associated detector element to detect only the second wavelength 412. Combined, the first filter-detector combination and the second filter-detector combination resolve the first and second wavelengths 411, 412 of light. More generally, referring still to FIG. 5I and referring again to FIG. 5G, more than 2, 3, 4, 5, 10, or 15 wavelengths of light are optionally resolved, even at a common radial distance from the source, using a corresponding set of 2, 3, 4, 5, 10, or 15 optical filters discriminating the wavelengths, without use of a grating element or time-domain resolution element in the analyzer 100. Combinations of filter types and detector types are described in the next paragraph. The combined use of wavelength discrimination at a common radial distance from a source in combination with wavelength discrimination as a function of radial distance resultant from the tissue properties, such as absorbance, scattering, and anisotropy, as well as in combination with an array or light sources, light source positions, an array of detectors, detector types, and/or detector positions is further described, infra.

Referring now to FIG. 5J, a basic case of multiple optical filter types and multiple detector types is introduced. Expansion of the optical filter types/detector combinations is greatly expanded infra. While the common illuminator 411 delivers any number of wavelengths of light, in this example the common illuminator delivers two bands of light, such as from two LEDs with mean wavelengths differing by at least 10 nm, which are separated at common radial distances from an illumination zone of the common illuminator using combinations of optical filters and detector types as described into five distinct mean pathways, where data related to each pathway is transformed using the algorithm as described, infra.

### Example I

Still referring to FIG. 5J, in a first example of the exemplary photon-tissue transport system 200, more than one detector type 430 is illustrated, a first detector type 431 and a second detector type 432. Detector types 430 are differentiated by type of material, such as a mercury-cadmium-telluride material versus and indium gallium arsenide material or are differentiated by doping, such as a 1.7 μm cutoff InGaAs detector versus a 1.9 μm cutoff InGaAs detector. As illustrated, the first detector type 431 is a 1.7 μm InGaAs detector illustrated at the 10 o'clock position, the second detector type 432 is a 1.9 μm InGaAs detector illustrated at the 12 o'clock position, and a first optical filter 231 is a 1650 nm longpass filter. Optionally, different InGaAs detectors have different thicknesses, such as a difference of more than 10, 20, 50, or 100 percent, where the change in thickness is used to alter respective wavelength responses of the different InGaAs detectors. Optionally, the InGaAs detectors directly contact the sample during use and do not have an intervening optical surface. If the common source 411 provides light from 1500 to 1600 nm and from 1680 to 1720 nm, then the first detector only observes the 1680 to 1700 nm light reaching a detection zone whereas the second detector observes a wider spectral region of 1680 to 1720 nm light. While the two detectors observe overlapping information, the two detectors observe different mean optical pathways associated with two optically probed volumes of tissue, such as in terms of depth of penetration, and receive two distinct pieces of information, handed to the transform system as described in the algorithm section, infra.

### Example II

Still referring to FIG. 5J, in a second example of the exemplary photon-tissue transport system 200, the first detector type 431, the second detector type 432, and the first optical filter 231 used at the 10-12 o'clock position is repeated at the 1-3 o'clock position at a common radial distance. Hence, identical signals would be observed in the ideal situation and differences between the common signals, such as at the first detector type 431 at the 10 o'clock and 1 o'clock positions, yield information on tissue structure differences. The differences are useful for outlier detection, signal averaging, and/or sample mapping as described, infra.

### Example III

Still referring to FIG. 5J, in a third example of the exemplary photon-tissue transport system 200, the common illuminator providing light from 1500 to 1600 nm and from 1680 to 1720 nm, the first detector type 431, and the radial distance from the illumination zone to respective detection zones used in the first example is again used, but in combination with a second optical filter 233, a 1650 nm shortpass filter replacing the first optical filter 231, the 1650 nm longpass filter. The new filter allows the first detector type 431 to see only the 1500 to 1600 nm band of light, which was not observed in the first example by the first detector type 431. Thus, a third pathway and third tissue volume is observed in the third example that is distinct from the two pathways/tissue volumes of the first example, where the distinct pathways are defined in terms of the wavelength of probing light. Notably, in the third example, replicate information is obtained at the 5 and 6 o'clock positions allowing outlier detection, signal averaging, and/or sample mapping as was obtained in the second example.

### Example IV

Still referring to FIG. 5J, in a fourth example of the exemplary photon-tissue transport system 200, the common illuminator providing light from 1500 to 1600 nm and from 1680 to 1720 nm, the first detector type 431, and the radial distance from the illumination zone to respective detection zones used in the first and third examples is again used, but in combination with a third optical filter 235, a 1550 to 1600 nm bandpass filter, and a fourth optical filter 237, a 1600 to 1650 nm bandpass filter, replacing the first optical filter 231, the 1650 nm longpass filter, and the second optical filter 233, the 1650 nm shortpass filter, respectively. The new filters 235, 237 allows the first detector type 431 to detect data representing only the 1500 to 1550 nm band of light at the 8 o'clock position and only the 1550 to 1600 nm light at the 9 o'clock position, which represent a fourth and fifth distinct mean pathway associated with a fourth and fifth probed tissue volume, where the corresponding fourth and fifth set of data is also sent to the transform algorithm.

Generally, the four examples illustrate that even at a common radial distance, the combination of detector type and filter type allows separation of mean optical pathways, optionally with replicates at each pathway, where data collected for the different optical pathways yields distinct information on the sample that is transformed using the algorithm. Combined with the above description of selection of radial distances-wavelength combinations for probing a given tissue depth yields a multivariate data collection set of the sample in terms of radial distance, wavelength of detected light, total pathlength, and/or total pathlength in a tissue layer that is transformed to an analyte concentration via use of calibration and prediction data sets with or without use of a grating or time-domain to frequency domain transform. The concepts presented here are optionally further multiplexed using positionally separated light sources, as described in the next section.

### Positionally Separated Light Sources

Referring now to FIGS. 5(K-N) positionally separated light sources are described, by way of example, where the sample itself is used to further discriminate mean pathways of probed light, which is subsequently transformed by the algorithm.

### Example I

Referring now to FIG. 5K, in a first example, a first illuminator 414, a species of the common illuminator 411, provides wavelengths of light that optimally probe the dermis layer and arrive at a detection zone with a short radial distance to a first ring of detectors 514, such as in a region of high sample absorbance, at about 1500 nm, and/or with a radial distance between the illumination zone and detection zone of less than 0.75 mm.

### Example II

Referring now to FIG. 5L, in a second example, a second illuminator 415, a species of the common illuminator 411, provides wavelengths of light that optimally probe the dermis layer and arrive at a detection zone with an intermediate radial distance to a second ring of detectors 515, such as in a region of high sample absorbance, at about 1550 nm, and/or with a radial distance between the illumination zone and detection zone of less than 0.75 to 1.25 mm.

### Example III

Referring now to FIG. 5M, in a third example, a third illuminator 416, a species of the common illuminator 411, provides wavelengths of light that optimally probe the dermis layer and arrive at a detection zone with a long radial distance to a third ring of detectors 516, such as in a region of high sample absorbance, at about 1500 nm, and/or with a radial distance between the illumination zone and detection zone of greater than 1.25 mm.

In each of examples I-III, the illuminator optionally comprises LEDs of multiple wavelengths, where the radial distance between the illumination zone and detection zone are similar. For instance, in the first example, the first illuminator 414 optionally provides light with wavelengths of about 1430, 1500, 1850, and/or 2150 nm and uses separate optical filters associated with individual wavelength bands on respective optically coupled detector elements of the first ring of detectors 514. Similarly, in the second example, the second illuminator 415 optionally provides wavelengths of light at about common sample absorbance-scattering-anisotropy combined levels, such as at about 1550 and 1820 nm again using optical filters to differentiate the mixed light to different detector elements of the second ring of detectors 515.

### Example IV

Referring again to FIG. 5(K-M) and now referring to FIG. 5N, in a fourth example, the first, second, and third illuminators 414, 415, 416, are spatially separated such that any of the corresponding first, second, and third ring of detectors 514, 515, 516 are eccentrically positioned relative to one another or intersect in two locations. As illustrated the first and third rings of detectors 515, 516 are eccentrically positioned, while the second ring of detectors 515 intersects both the first ring of detectors 514 and third ring of detectors 516. Generally, providing different wavelengths of light at y/z-plane separated positions, separated by at least one detection zone, allows narrow ranges of radial distance between a give illuminating LED zone-detection zone pair. In contrast, referring now to FIG. 5O, the three closely spaced illumination areas couple light to a given detection zone with a range of radial distances, such as illustrated by radius 1, r₁, and radius 2, r₂, where the larger spread of radial distance increases the standard deviation of total optical pathlengths, which leads to increased uncertainty in a corresponding concentration as described above.

Referring again to FIGS. 5(B-N), a noninvasive near-infrared analyzer is configured to control variation of detected optical pathlength of the sample and/or spatially separate collected light having probed different sample volumes using a combination of:
discrete detection zones as illustrated in FIG. 5B;
segmented spacers as illustrated in FIG. 5D;
rings and/or arcs of detector elements as illustrated in FIG. 5G;
optical filters as illustrated in FIG. 5J;
illuminator to detector distance control as illustrated in FIG. 5O;
distributed illumination areas and/or interconnected detection zones as illustrated in FIG. 5N;
use of micro-optics, as described infra;
controlled or varying detector types having different response shapes, as described infra;
a sub-set of detector elements optically coupled to a smaller detection zone versus combined signals from a larger number of detector elements detecting photons from a larger range of illumination zone to detection zone distances; and/or
detecting and removing outlier signals resultant from probing photons interacting with tissue inhomogeneities, such as a hair follicle, a localized refractive index change, a localized scattering change, and/or a geometric variation at an interface of two or more tissue layers.

Generally, control of optical depth of penetration, total pathlength in a desired tissue zone, and/or total tissue pathlength is achieved with any one of the described controls. However, the inventor has determined that combinations of three or more, such as 4, 5, 6, 7, 8 or more, of the described controls interact to still further control the optical depth of penetration, total pathlength in a desired tissue zone, and/or total tissue pathlength observed with one or more detector elements of a sub-group of the overall detector array.

Expansion of the low number of illumination zones, such as from a few LEDs; expansion of the number of optical filters; and expansion of the low number of detection zone, such as from 1 to 6 detectors per LED to arrays of sources, filters, and/or detection zones is described in later sections. However, before expanding the number of illumination elements, filters, and/or detectors, the general condition of the mean depth of penetration and total optical pathlength increasing with radial distance from the illumination zone is further refined in terms of water absorbance and scattering in the next section.

### Water Absorbance and Scattering

Water and scattering effects are two large contributors to observed absorbance in diffuse reflectance near-infrared spectroscopy of tissue.

### Water Absorbance

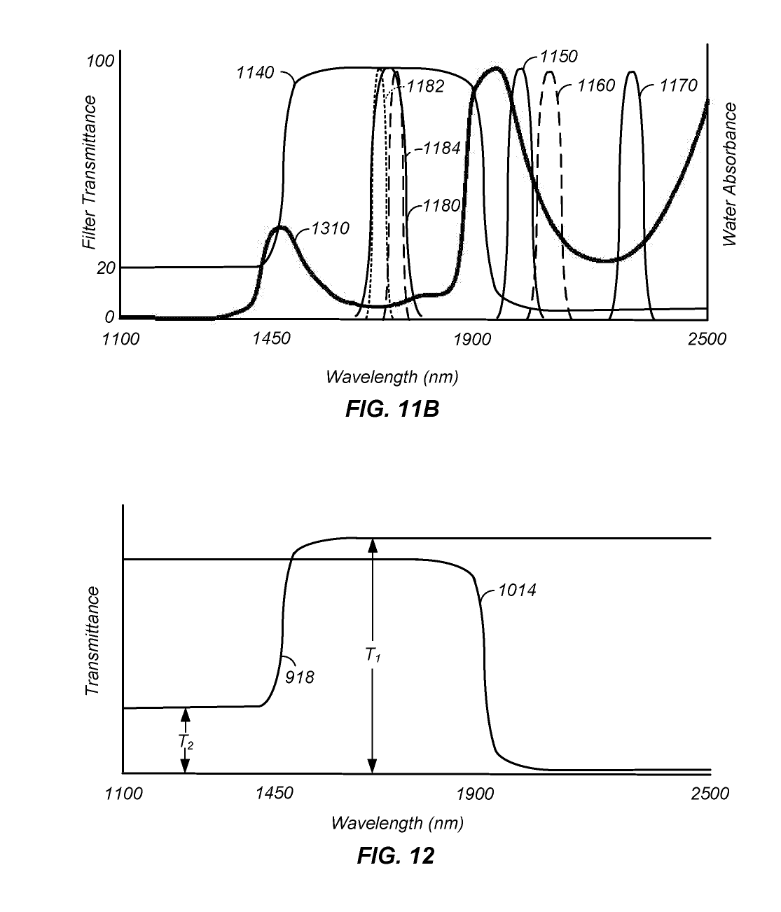

Referring now to FIG. 11A, relative water absorbance 1310 is illustrated in the near-infrared region from 1100 to 2500 nm. Erroneously, but commonly used denotations of the near-infrared regions of a second absorbance band spectral region 970, a first absorbance band spectral region 960, and a combination band spectral region 950 are divided by water absorbance maxima at about 1450 and 1950 nm, with another large water absorbance band at 2600 nm. Within the three described near-infrared spectral regions, water absorbance is relatively largest in the combination band spectral region 950, intermediate in the first overtone spectral region 960, and smallest in the second overtone spectral region 970.

### Scattering

Referring again to FIG. 11A, a scattering coefficient 1140, scattering, and/or an effect of scattering is illustrated. In the near-infrared region from 1100 to 2500 nm, scattering is strongest at 1100 nm and is observed to drop off at longer wavelengths. In tissue, scattering generally dominates in the second overtone spectral region 970, has a significant contribution in the first overtone spectral region 960, and is still smaller, but necessary for diffuse reflectance, in the combination band spectral region 950. Generally, more scattering results in a smaller mean radial pathlength between an illumination zone, where photons enter tissue, and a detection zone, where photons exit the tissue. Additionally, more scattering generally results in a higher percentage of incident photons reemerging from the tissue sample before absorbance of the tissue results in insignificant quantities of returning photons, in terms of a signal-to-noise ratio.

### Interplay of Water and Scattering

The combination of water absorbance and scattering as a function of wavelength yields considerable information for analyzer design and algorithm usage. As described, supra, the details of the interaction of the physics and chemistry of the sample, in this case in terms of water absorbance and scattering, allows for the detailed and/or specific instrument designs described herein, in terms of optical filters, illumination zones, color of incident light as a function of time, and detection zones, all as a function of time. Several examples are provided in this section to further clarify the invention.

### Example I

The interaction of water absorbance and scattering of light is described in the combination band spectral region 950 in a first example in terms of glucose containing layers of skin and optical filters.

In the combination band spectral region 950, water absorbance is generally high. For example, absorbance of water has a local minimum at about 2272 nm of one absorbance unit per millimeter with even higher water absorbance at both shorter wavelengths, toward 1900 nm, and longer wavelengths, toward 2500 nm. The high absorbance of water requires small total optical pathlengths, such as 2.5, 2.0, 1.5 mm or less, with greatly increased observed signal and intensity-to-noise ratios at still short total optical pathlengths, such as less than 1.2, 1.0, 0.8, or 0.6 mm.

Scattering 1140 is relatively low in the combination band spectral region 950, meaning that photons must travel on average further to strike a scattering center or scattering element. Photons typically require striking multiple scattering centers to return to the incident surface, such as at the detection zone. Hence, longer optical pathlengths result. Anisotropy as a function of wavelength is highly correlated with scattering as a function of wavelength with both curves dropping off rapidly from 1100 to 1300 nm and progressively slower from 1300 to 2500 nm.

The large absorbance of water requiring short pathlengths and the low scattering coefficient requiring long pathlengths results in a narrow region of radial distances yielding significant percentages of photons returning to the surface in the detection zone, where the narrow region of radial distances between the illumination zone 177 and the detection zone 179 is small, such as less than 1, 0.8, 0.6, or 0.4 mm.

The analyzer 100 optionally and preferably still further restricts photons returning to the surface at still shorter pathlengths from reaching detector elements, such as via use of a mechanical-optical filter blocker or mechano-optical filter, which results in: (1) a still narrower range of radial distances between the illumination zone and the detection zone and (2) a greater relative percentage of photonic pathways penetrating into deeper glucose containing tissue layers with subsequent detection at the detection zone.

The inventor has determined that optical designs of the analyzer 100 are optionally and preferably used to still further narrow mean radial distances observed at individual detector elements of an array detector, where the narrower mean radial distances results in smaller deviations in pathlength, b, and enhanced accuracy and precision of the calculated concentration, C, such as the glucose concentration, as described supra, in relation to equation 1.

Several sub-examples are used to further describe enhancements provided by the use of narrow band optical filters and/or longpass or shortpass filters in combination with sample absorbance and scattering to yield narrow ranges of pathlengths, spectral resolution, and relatively high signal-to noise ratios for detected glucose absorbance in skin. Notably, the sub-examples applied to narrow spectral regions generally apply to other spectra regions of similar absorbance/scattering ratios.

### Example I_{A}

Still referring to FIG. 11A, a region of high absorbance and low scattering 951, such as region A, has requirements of a small radial distance between an illumination zone and a detection zone due to the high water absorbance and a larger radial distance between the illumination zone and the detection zone due to the low scattering coefficient, as described supra. Thus, a first narrowband optical filter, such as an about 2125 to 2175 nm passband filter associated with a detector element where the associated detector element is at a radial distance of the small intersection of the competing radial distance requirements of water absorbance and scattering, allows the detector element to observe:
a narrow spectral wavelength region, for enhanced resolution; and
an enhanced percentage of photons probing deeper tissue layers containing tissue layers at concentrations physiologically relatable to blood glucose via use of mechanical optical restriction of photons dominantly not reaching glucose.

The first narrowband optical filter optionally and preferably transmits wavelengths related to a sample constituent absorbance band, such as the glucose absorbance band at 2150 nm.

The inventor notes that the first narrowband optical filter is optionally a shortpass filter, such as with a cut-off of 2175 nm as the rapidly increasing water absorbance at shorter wavelengths will act as a natural longpass filter, especially when used in combination with a filter layer blocking the first and second overtone wavelengths. The use of layered optics to replicate this effect in multiple wavelength regions is further described, infra.

### Example I_{B}

Still referring to FIG. 11A, a region of medium absorbance and low scattering 952, such as region B, has requirements of a radial distance between an illumination zone and a detection zone that is slightly larger than the small radial distance described in Example 1_{A} due to the slightly lower water absorbance and similar scattering. More particularly, the lower water absorbance allows for slightly longer total optical pathlengths and thus an optimal zone for detection of probing photons having passed through the glucose containing dermis layer of skin having a greater radial distance from the illumination zone. Similar to the first example about region A, region B benefits from a second optical filter passing the narrow region for resolution and a narrow pathlength region for enhanced accuracy of the glucose concentration determination by limiting variance in pathlength, b, thereby enhancing confidence in the relationship between absorbance, A, and concentration, C, as described above in relation to equation 1.

Combining factors presented in Example I_{A} and Example I_{B}, the inventor notes that the second optical filter-second detector element combination for the lower absorbing water spectral region with similar scattering is preferentially positioned further away from the illumination zone that the first optical filter-first detector element combination for the higher absorbing water spectra region with similar scattering.

### Example I_{C}

Still referring to FIG. 11A, a region of still lower absorbance and low scattering 952, such as region C, relative to region B has requirements of a radial distance between an illumination zone and a detection zone that is still larger than the small radial distance described in Example 1_{B} due to the still lower water absorbance and similar scattering. Thus, comparing Examples I_{(A-C)}, for similar scattering, as the absorbance decreases, the associated optical filter and detector elements are preferably located at increasing radial distances from the illumination zone.

### Example I_{D}

Still referring to FIG. 11A, region B and region D have essentially equivalent absorbance and scattering. Thus, optical filters and detector elements associated with regions B and D are preferably located at about equivalent distances from an illumination zone. Secondary parameters are then used to determine relative placements, such as ease of placement of overlapping optical filters, temperature effects, ability to collect redundant data for internal consistency checks, data binning, surface contact, and many others, as described throughout.

### Example I_{E}

Still referring to FIG. 11A, a region of relative low absorbance and medium to high scatter 955, such as region E, requires radial distance based upon not only absorbance and scatter, but also glucose absorbance along with interference considerations. More particularly, as the glucose absorbance in the first overtone region 960 is substantially less than glucose absorbance in the combination band spectral region 950, longer total optical pathlengths are required. Unlike the combination band spectral region, there are several possibilities for obtaining the longer total optical pathlength. First, a longer radial distance between the illumination zone and detection zone is possible as the water absorbance allows the longer pathlength without resulting in absolute absorbance levels that are so high as to seriously degrade signal-to-noise ratios. In this first option, depth of penetration of the photons statistically probe the glucose containing dermis layer of skin. Second, a smaller radial distance between the illumination zone and the detection zone is possible as the higher scattering results in a substantial number of photons returning to the skin surface as small radial distances. Again, in the second option, depth of penetration of the photons statistically probe the glucose containing dermis layer of skin. In a third option, an intermediate distance between the illumination zone and detection zone is possible as both scattering and water absorbance allow adequate depth of penetration of photons to glucose containing layers of skin and adequate signal-to-noise ratios in detected signals at the skin surface. However, the inventor has determined that enhanced performance of the analyzer 100 is obtained by separating the conditions, as described in the following paragraph.

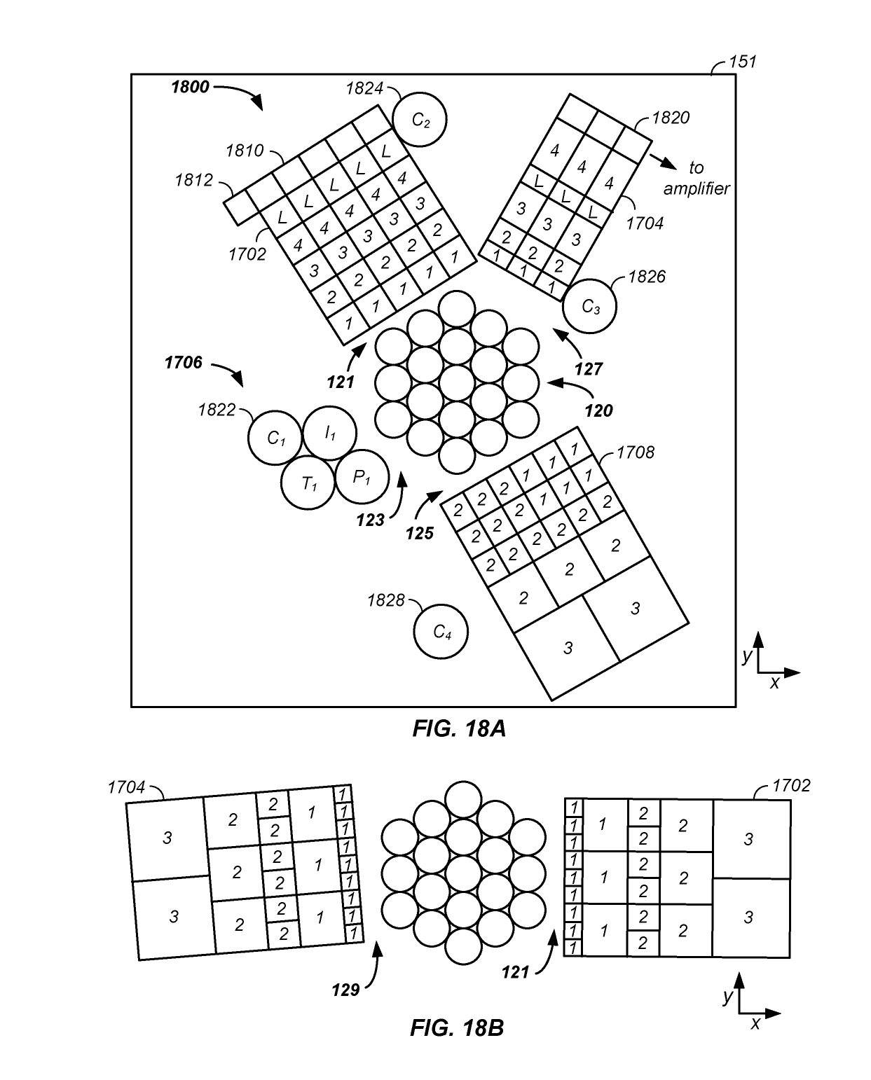

Still referring to FIG. 11A, as described in the preceding paragraph, short, medium, and long radial distances between the illumination zone and detection zone are all possible in the condition of relatively low water absorbance and medium to high scattering, such as region E. However, each of the three conditions optically samples overlapping yet largely distinct tissue volumes. For instance, in the short radial distance condition, more photons return having sampled exclusively the epidermis. Similarly, the intermediate radial distance condition has have a higher percentage of detected photons with a deepest depth of penetration into the dermal layer of skin. Still further, in the long radiation distance condition, a considerably higher percentage of the photons probe the subcutaneous fat layer. Thus, the glucose containing tissue pathlength will differ for the three conditions. Blending the three conditions as is traditionally done with a single detector detecting a wide range of radial distance, results in an increasing uncertainty in the pathlength parameter, b, which leads to an increased uncertainty in the concentration of glucose, C, as explained above in relation to equation 1. However, the inventor has determined that with an array detector and especially with a two-dimensional array detector, the uncertainly in the pathlength parameter, b, is reduced yielding a more accurate and certain concentration of glucose, C, with detector elements radially distributed from the illumination zone 177 as explained above. The hardware used to achieve the separation is a detector array with individual detection elements detecting each of the three conditions. Notably, a physically single optical filter, such as an optical filter isolating region E, is optionally coupled to detector elements for all three conditions, see for instance filter 2 in the fourth detector array 1708 in FIG. 18A, where filter 2 is placed at at least four radial distance from the illumination zone. The single physical filter greatly enhances manufacturability. The inventor notes that the single physical filter is readily partially overlapped with other filters for still further enhanced wavelength resolution and less obviously still further control of pathlength, b. For instance, a filter blocking the higher or lower wavelengths of region E in combination with the second filter alters the scattering, as the scattering has a non-zero slope of the region E and the change in scattering will alter optical pathlengths of detected photons penetrating into the glucose containing dermal layer of skin.

Still further, the internal quality checks are still further enhanced using a common optical filter coupled to a radially distributed range of detector elements, as described infra, in the tissue mapping section.

Comparing region E, as described in Example I_{E}, with higher scattering and lower water absorbance with regions A-D, as described in Examples I_{(A-D)}, it is observed that generally the filter-detector combinations associated with the first overtone spectral region 960 have the same or larger preferred radial distances from the illumination zone compared to filter-detector combinations associated with the combination band spectral region 950. For instance, the first overtone 960 filter/detector combinations are preferably associated with radial distances between the illumination zone and detection zone that are at least 1.2, 1.5, 2, or 3 times the radial distance between the illumination zone and detection zone for the combination band 950 filter/detector combinations.

### Example I_{F}

Still referring to FIG. 11A, a region of low absorbance and high scatter 956, such as region F, optionally uses still larger radial distance to yield a significant percentage of detected photons having sampled the deeper glucose containing layers of skin. Thus, filter-detector element combinations associated with the second overtone region 970 are preferably associated with radial distances between the illumination zone and detection zone that are at least 2, 3, or 4 times the radial distance between the illumination zone and detection zone for the combination band 950 filter-detector combinations and at least 1.5 or 2 times the radial distance between the illumination zone and detection zone for the first overtone region 960 filter-detector combinations.

Examining Examples I_{(A-F)}, the inventor has determined that there exists a benefit of using a two-dimensional detector array in a noninvasive glucose analyzer 100 in terms of: (1) breaking the illumination zone-detector zone distance into subset distances, (2) associating optical filters with subsets of radial distances for enhanced wavelength resolution, (3) associating filters with subsets of radial distances from an illuminator for enhancing accuracy and certainty, and (4) coupling exceedingly well with use of a small number of light emitting diodes having distinct and/or overlapped spectral bandwidths of emission. Accordingly, two-dimensional detector array systems are extensively described in the subsequent section.

### Two Dimensional Detector Array System

Referring again to FIGS. 4(A-B) and FIGS. 5(A-O), the number of illumination zones, where light enters skin of the subject 170, from one or more source elements, is optionally more than 1, 2, 3, 4, 5, 10, 20, 50, or 100 and the number of detection zones, where light exiting the skin of the subject 170 is detected by one or more detection elements and/or systems, such as more than 1, 2, 3, 4, 5, 10, 20, 50, 100, 500, 1000, 5000, 10,000, or 50,000 detection elements.

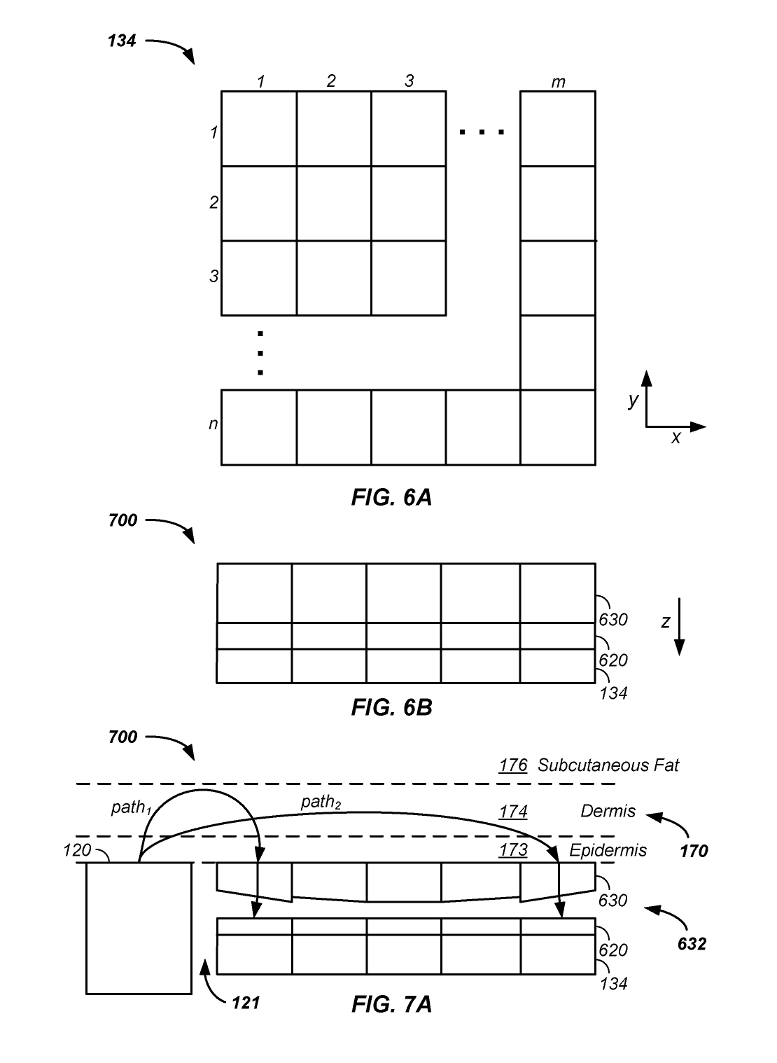

Referring now to FIG. 6A, a m×n two-dimensional detector array 134 is illustrated, which is an example of the detector 132 in the detector system 130. Herein, the m×n two-dimensional detector array 134 is illustrated as a matrix of m columns by n rows, where m and n are each, not necessarily equal, positive integers, such as greater than 1, 2, 3, 4, 5, 10, 20, 50, 100. Optionally, the two-dimensional detector array 134 is of any geometric configuration, shape, or pattern.

Referring now to FIG. 6B, an optional configuration of the two-dimensional detector array 134 is further described. Optionally, one or more elements of the two-dimensional detector array 134 are coated or coupled with an optical detector filter 620. In a first case, the optical detector filter 620 is uniform across the two-dimensional detector array 134. In a second case, the optical detector filter 620 comprises an array of filters, where individual elements, grids, or zones of the optical filter correspond to individual elements of the two-dimensional detector array 134. For example, a group of at least 1, 2, 4, 9, 16, or 25 elements of the two-dimensional detector array 134 are optically coupled with a first optical filter and a group of at least 1, 2, 4, 9, 16, or 25 elements of the two-dimensional detector array 134 are optically coupled to a second filter. Optionally, any number of filter types are used with a single detector array, such as 1, 2, 3, 4, 5, 10, 20 or more filter types. In a preferred embodiment, a first, second, third, fourth, and fifth filter type correspond with peak transmittance in ranges in the 1100 to 1450 nm range, 1450 to 1900 nm range, 1100 to 1900 nm range, 1900 to 2500 nm range, or 1100 to 2500 nm range, respectively, with lower transmittances, such as less than 50, 25, or 10 percent at higher and/or lower frequencies. In a third case, the optical filter 120 comprises a repeating pattern of transmittances and/or absorbances as a function of y, z-position.

Still referring to FIG. 6B, the two-dimensional detector array 134 is optionally coupled to a detector optic/micro-optic layer 630. In a first case, individual optical elements of the micro-optic layer 630 optionally:
alter a focal depth of incident light onto the two-dimensional detector array 134;
alter an incident angle of incident light onto the two-dimensional detector array 134;
focus on an individual element of the two-dimensional detector array 134; and/or
focus on groups of detection elements of the two-dimensional detector array 134.

In a second case, individual lines, circles, geometric shapes covering multiple detector elements, and/or regions of the micro-optic layer optionally:
alter a focal depth of incident light onto a line, circle, geometric shape, and/or region of the two-dimensional detector array 134;
alter an incident angle of incident light onto a line, circle, geometric shape, and/or region of the two-dimensional detector array 134; and/or
focus onto a line, circle, geometric shape, and/or region of a group of elements of two-dimensional detector array 134.

Further the individual optical elements of the micro-optic layer 630 and/or the individual lines, circles, geometric shapes, or regions of the micro-optic layer 630 are optionally controlled by the system controller 180 to change any of the focal depth and/or an incident angle of incident light as a function of time within a single data collection period for a particular subject and/or between subjects, which is optionally previously determined using an analysis of tissue type of the subject 170.

Still referring to FIG. 6B, the optical detector filter 620 is:
optionally used with or without the detector optic/micro-optic layer 630;
optionally contacts, proximately contacts, or is separated by a detector filter/detector gap distance from the two-dimensional detector array 134; and/or
is positioned between the optical detector filter 620 and the two-dimensional detector array 134.

Similarly, the detector optic/micro-optic layer 630 is:
optionally used with or without the optical detector filter 620; and/or
optionally contacts, proximately contacts, or is separated by a micro-optic/detector gap distance 632 from the two-dimensional detector array 134.

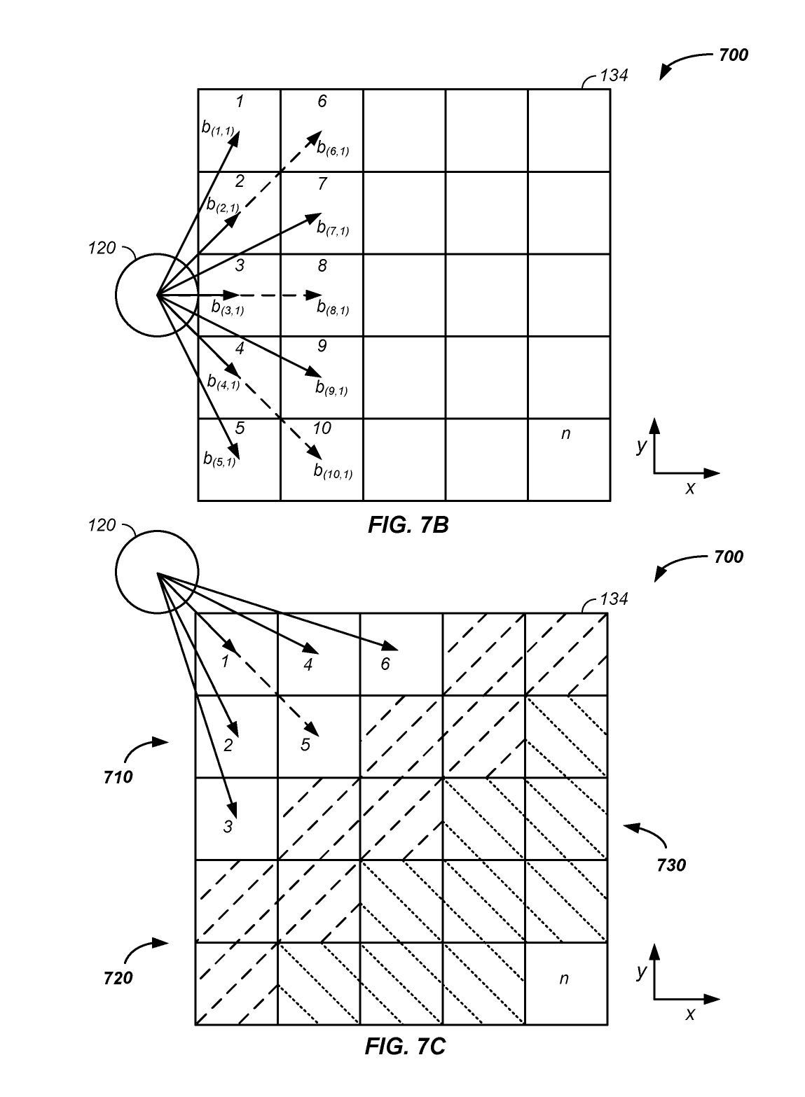

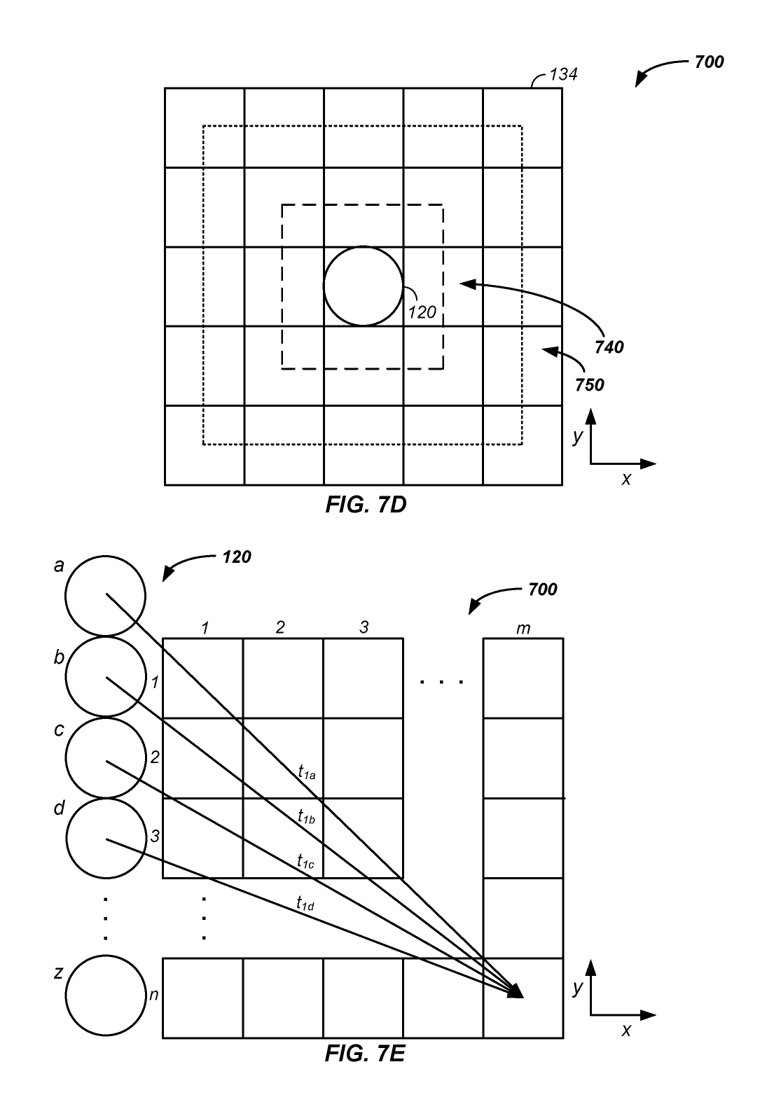

Referring now to FIGS. 7(A-E), optionally and preferably an incident optic/two-dimensional detector array system 700 is enclosed in a housing. For example, optionally and preferably, the detector array, first optical filter array, second optical filter array, and/or focusing optic array are sandwiched together, where two or more of the stacked layers are substantially contacting along an interfacing plane. The first and/or second optical filter arrays are optionally placed along the optical axis on either side of the focusing optic/light gathering array. The housing serves a number of purposes, such as the ability to prevent dust/particulate infiltration; is to function as an enclosure sealed against moisture, allowing the detectors to be operated below a dew point, such as via use of 2, 3, or 4 layers of Peltier coolers; allows use of a partial vacuum within the enclosure; and/or allows a substantially non-water containing gas to be placed in the housing to minimize condensation.

Referring still to FIGS. 7(A-E), for clarity of presentation, the incident optic/two-dimensional detector array system 700 is illustrated in multiple representative configurations, without loss of generality or limitation.

Referring now to FIG. 7A, a first example of the incident optic/two-dimensional array system 700 is illustrated with the photon transport system 120 used to deliver photons to the subject 170 proximate the two-dimensional detector array 134. In a first example, a portion of photons from the photon transport system diffusely scatter through skin of the subject 170 and after radial movement emerge from the skin of the subject 170 where a portion of the incident photons are detected by elements of the two-dimensional detector array 134. In a first example, photons are illustrated travelling along: (1) a first mean path, path₁, and are detected by a first detector element of the two-dimensional detector array 134 at a first, smaller, mean radial distance from a tissue illumination zone of the photon transport system and (2) a second mean path, path₂, are detected by a second detector element of the two-dimensional detector array 134 at a second, longer, mean radial distance from a tissue illumination zone of the photon transport system relative to path₁. In this first example, optionally:
a first element of the optical detector filter 620 is preferably a filter designed for a shorter optically probed mean tissue pathlength, such as about 0 to 1.5 millimeters, such as a combination band optical filter with a peak transmittance in a range of 2000 to 2500 nm;
a second element of the optical detector filter is preferably a filter designed for a longer optically probed mean tissue pathlength, such as about 5.0 to 10 millimeters, such as a second overtone optical filter with a peak transmittance in a range of 1100 to 1450 nm; and
a third element of the optical detector filter is preferably a filter designed for an intermediate optically probed mean tissue pathlength, such as about 1.5 to 5.0 millimeters, such as a first overtone optical filter with a peak transmittance in a range of 1450 to 1900 nm.

In the first example,
a first element of the detector optic/micro-optic layer 630 is optionally configured to preferably collect incident skin interface light having an angle aimed back toward the photon transport system, which yields a slightly shorter mean tissue pathlength, such as about 0.2 to 1.7 millimeters compared to an optic that is flat/parallel relative to the skin of the subject 170;
a first element of the detector optic/micro-optic layer 630 is optionally configured to redirect collected incident skin interface light back away from the photon transport system 120 as illustrated, such as onto a center of a detector or detector array element closer to the illumination zone;
a second element of the detector optic/micro-optic layer 630 is optionally configured to preferably collect incident skin interface light having an angle aimed away from the incident illumination zone of the skin, which yields a slightly shorter mean tissue pathlength compared to an optic that is flat/parallel relative to the skin of the subject 170;
a second element of the detector optic/micro-optic layer 630 is optionally configured to redirect collected incident skin interface light back toward the incident skin illumination zone, such as onto a center of a detector or detector array element further from the illumination zone;
a third element of the detector optic/micro-optic layer 630 is optionally flat/parallel relative to a mean plane between the skin of the subject 170 and the two-dimensional detector array 134.

As described, supra, the individual optical elements of the micro-optic layer 630 and/or the individual lines, circles, geometric shapes, or regions of the micro-optic layer 630 are optionally dynamically controlled by the system controller 180 to change any of a detector layer incidence acceptance angle, the focal depth, an incident angle, and/or an emittance angle or exit angle of photons as a function of time within a single data collection period for a particular subject and/or between subjects.

Still referring to FIG. 7A, an optional micro-optic layer/detector array gap 632 is illustrated between the detector optic/micro-optic layer 630 and elements of the two-dimensional detector array 134, such as a gap less than 0.2, 0.5, 1, 2, 5, or 10 millimeters. Further, an optional spacer gap 121 is illustrated between a final incident optic of the photon transport system 120 and any of the two-dimensional detector array 134, the optical detector filter 620, and/or the detector optic/micro-optic layer 630, such as a gap of less than about 0.1, 0.2, 0.3, 0.4, 0.5, 0.75, and/or 1.0 millimeter.

Referring now to FIG. 7B, a second non-limiting example of the incident optic/two-dimensional detector array system 700 is illustrated with the photon transport system 120 used to deliver photons to the subject 170 proximate a first side of the two-dimensional detector array 134, where the array has n detector elements, where n is a positive integer greater than three. In this second example, ten radial distances to ten detector elements are illustrated. In this example, some radial distances are equal, such as a first radial distance to detector elements 1 and 5 and a second radial distance to detector elements 2 and 4. Generally, detector elements are optionally grouped or clustered into radial distances relative to an illumination zone of 1, 2, 3, or more incident light directing elements where each group or cluster is individually associated with an average mean optical probed tissue pathlength, subsequently used in pathlength resolution, and/or analyte concentration estimation.

Still referring to FIG. 7B, optionally, different clusters of radial distances are treated optically differently, such as with a different optical detector filter 620. Representative and non-limiting examples include:
a combination band filter for filtering photons having mean radial distances of 0 to 1 millimeter, the combination band filter comprising:
   a transmittance greater than seventy percent at 2150 nm, 2243, and/or 2350 nm, and/or
   an average transmittance of greater than seventy percent from 2100 to 2400 nm and an average transmittance of less than twenty percent from 1100 to 1900 nm and/or from 2400 to 2600 nm;
a first overtone band filter for filtering photons having mean radial distances of 0.3 to 1.5 millimeters, the first overtone filter comprising:
   a transmittance greater than seventy percent at 1550 nm, 1600, and/or 1700 nm, and/or
   an average transmittance of greater than seventy percent from 1500 to 1800 nm and an average transmittance of less than twenty percent from 1100 to 1400 nm and/or from 2000 to 2600 nm;
a combination band/first overtone band filter for filtering photons having mean radial distances of 0 to 1.5 millimeters, the combination/first overtone filter comprising:
   a transmittance greater than seventy percent at 1600 and 2100 nm, and/or
   an average transmittance of greater than seventy percent from 1500 to 2300 nm and an average transmittance of less than twenty percent from 700 to 1400 nm and/or from 2500 to 2800 nm;
a second overtone band filter for filtering photons having mean radial distances of 0.5 to 3.0 millimeters, the second overtone filter comprising:
   a transmittance greater than seventy percent at 1200 nm, 1300, and/or 1400 nm, and/or
   an average transmittance of greater than seventy percent from 1100 to 1400 nm and an average transmittance of less than twenty percent from 700 to 1000 nm and/or from 1500 to 2000 nm;
a first overtone band/second overtone band filter for filtering photons having mean radial distances of 0.5 to 3.0 millimeters, the first overtone band/second overtone band filter comprising:
   a transmittance greater than seventy percent at 1300 and 1600 nm, and/or
   an average transmittance of greater than seventy percent from 1200 to 1700 nm and an average transmittance of less than twenty percent from 700 to 1000 nm and/or from 2000 to 3000 nm;
a sloping overtone band filter or step function overtone band filter for filtering photons having mean radial distances of 0.5 to 3.0 millimeters, the sloping overtone band filter comprising:
   a mean transmittance greater than ten percent at 1300 nm, less than fifty percent at 1300 nm, and greater than seventy percent at 1600 nm, and/or
   an average transmittance between 1100 and 1300 nm in the range of ten to fifty percent and an average transmittance between 1500 and 1700 nm of greater than seventy percent with optional out of band blocking from 700 to 1000 nm and/or from 2500 to 3000 nm of greater than ninety percent; and/or
a luminance filter for filtering photons having mean radial distances of 0 to 5 millimeters, the luminance filter comprising:
   an optical spacing element designed to maintain focal length;
   a mean transmittance greater than seventy percent from 1100 to 1800 nm, and/or
   a mean transmittance greater than seventy percent from 1100 to 2400 nm and an average transmittance of less than twenty percent from 700 to 1100 nm and/or from 2000 to 2600 nm.

Referring now to FIGS. 7B, 7C, and 7D, the photon transport system 120 is illustrated as delivering light to an edge, corner, and interior region of the two-dimensional detector array 134, respectively. Descriptions, herein, to the edge, corner, or interior illumination options optionally apply to the other cases.

Referring again to FIG. 7B, the photon transport system 120 is illustrated delivering photons using at least one fiber optic and/or through one or more optics to a point or illumination zone along an edge of the two-dimensional detector array 134. For clarity of presentation, in a first case, the photon transport system 120 is illustrated delivering photons to a center of an edge of the two-dimensional detector array 134; however, the photon transport system 120 optionally delivers photons to any point along the edge of the two-dimensional detector array 134 and/or at any distance from an edge or corner of the two-dimensional detector array.

Still referring to FIG. 7B, as illustrated the photon transport system delivers photons that are detected with an array of mean pathlengths and associated mean depths of penetration into the tissue of the subject 170, at each detector element. For example, the first detector element, 1, detects photons having a first mean pathlength for a first illumination point, herein denoted b_{(pathlength, illuminator)}. In the first case, using a simplifying assumption of tissue homogeneity for clarity of presentation, the mean probed pathlength is the same at the first and fifth detector elements. Similarly, the mean probed pathlength is similar and/or tightly grouped at the second and fourth detector element. In addition, groups of detector elements observe photons traversing similar or grouped pathlengths. For example, a first sub-group of the first, sixth, and seventh detector elements observe similar probed tissue pathlengths and depths of penetration. Similarly, a second sub-group of the fifth, ninth, and tenth detector elements observe similar probed tissue pathlengths and depths of penetration. In this case, the first sub-group and second sub-group are optionally placed into a single group as the first sub-group and second sub-group observe similar, exact if the tissue is homogenous, probed tissue pathlengths. Similarly, a first sub-group is optionally one, two, three, or more elements of a first column of detector elements and a second sub-group is optionally one, two, three, or more elements of a second column of the detector elements. Generally, the detector elements are optionally treated individually or in sub-groups, such as by distance from a mean sample illumination point, sub-groups of one or more rows of detector element, sub-groups of one or more columns of detector elements, and/or groups of sub-groups.

Still referring to FIG. 7B, any two-dimensional detector array 134 element, sub-group, column, row, region, and/or group is optionally individually coated or coupled to any filter, such as the filters described supra, and/or is optionally individually coupled with a focusing optic and/or a dynamic focusing optic, as further described, infra.

Referring now to FIG. 7C, a second case of an illumination optic and/or a group of illumination optics of the photon transport system 120 used to illuminate an illumination zone relative to a corner of the two-dimensional detector array 134 is illustrated. As with the first side illumination case, individual elements, sub-groups, and/or groups of detector elements observe at differing radial distances from the illumination zone where the differing radial distances have corresponding average observed tissue pathlengths, depths of penetration, and/or sampled regions of skin of the subject 170. Here, three groups or detection zones are illustrated. The first group 710 is illustrated as detection elements 1, 2, 3, 4, 5, and 6, where the commonality is a short radial distance between the illumination zone and the detection zone, such as used for the combination band spectral region and/or for small mean depths of penetration of the photons into the tissue of the subject 170. The second group 720 is illustrated with long rising dashes, where the commonality is a medium radial distance between the illumination zone and the detection zone, such as used for the first overtone spectral region. The third group 730 is illustrated with short falling dashes, where the commonality is a long radial distance between the illumination zone and the detection zone, such as used for the second overtone spectral region. As described, supra, any detector element, sub-group, and/or group is optionally associated with an individual filter, an individual optic, an individual dynamic optic, and/or a group of optics. Further, any detector element, sub-group, and/or group is optionally associated with any position and/or wavelength of illuminators, such as with a light-illuminating diode illumination array.

Still referring to FIG. 7C, detector elements are optionally associated with one optical filter for ease of production, yet detector elements 1-6 still measure separate sample volumes/tissue pathways, yielding a preferable larger number of sample measurements for the transform function of the algorithm. Further, detector elements 1-6 additionally provide internal data consistency information, such as detector elements 2 and 4 should yield roughly equal signals as should detector elements 3 and 6, while detector element 5 should yield a larger absorbance than detector element 1 just as detector elements 3 and 6 should yield a larger absorbance signal than detector element 5; all derived from absorbance increasing as a function of radial distance at increasing distances from the illumination zone.

Referring now to FIG. 7D, a third case of an illumination optic and/or a group of illumination optics of the photon transport system 120 used to illuminate an illumination zone within a section within the two-dimensional detector array 134 is illustrated. As with the first side illumination case and the second corner illumination case, individual elements, sub-groups, and/or groups of detector elements observe at differing radial distances from the illumination zone where the differing radial distances have corresponding average observed tissue pathlengths, depths of penetration, and/or sampled regions of skin of the subject 170. Here, two groups or detection zones are illustrated. The third group 740 is a first section, arc, quadrant, zone, ring, square, rectangle, and/or polygon of detection elements at a first range of distances from the illumination zone, illustrated here with detector elements intersecting with a long-dashed/square shape. The fourth group 750 is a second section, arc, quadrant, zone, ring, square, rectangle, and/or polygon of detection elements at a second range of distances from the illumination zone, shown here with detector elements intersecting with a short-dashed/square shape. The fourth group 740 and fifth group 750 are illustrative of n groups where n is a positive integer of 2, 3, 4, 5, 10 or more where individual groups differ by 1, 2, 3, 4 or more cross-sectional distances of a detector element. As described, supra, any detector element, group, sub-group, and/or group is optionally associated with an individual filter, an individual optic, and/or an individual dynamic optic.

Still referring to FIG. 7D, in one optional filter arrangement, optical filters are stacked. For example, a first optical filter is a first long pass or a first short pass filter covering a wide range of first detector elements; a second optical filter is stacked relative to the first optical filter along the z-axis, which is the optical axis. The second optical filter is a second long pass, a second short pass, or a band pass filter covering a subset of the first detector elements. For example, the first optical filter is a long pass filter passing wavelengths longer than 1100 nm covering all of the fourth group 740 and fifth group 750, and the second optical filter is a short pass filter passing wavelength shorter than 1450 nm covering all of the fifth group, which yields a first and second overtone filter for the fourth group 740 and a second overtone filter for the fifth group 750. Combinations of stacked filters for various groups include any of 2, 3, 4, or more filters described herein, such as the combination band filter, the first overtone band filter, the combination band/first overtone band filter, the second overtone band filter, the first overtone band/second overtone band filter, the sloping overtone bands filter, and the luminance filter described, supra, in the description of FIG. 7B. The inventor notes that cutting larger stackable filters reduces costs and more importantly light loss associated with placing individual filters over individual detector elements of the two-dimensional detector array 134.

Referring now to FIG. 7E and FIG. 5N, a fourth example of multiple illumination zones from the photon transport system 120 positioned about and within the two-dimensional detector array 134 are, respectively, illustrated. In this fourth example, a matrix of illuminators, herein represented by a single column for clarity of presentation, are denoted as illuminators a-z. At a given point in time, any set or subset of the matrix of illuminators are used to deliver photons to the tissue of the subject 170. For example, at a first point in time, illuminators a-b are used; at a second point in time illuminators a-d are used; at a third point time illuminators d-g are used, and so on. As illustrated, illuminators a-d are used and a detection element m,n is used. Generally, sets of illuminators are optionally used as a function of time where the illuminators define the number of photons delivered and provide a first part of a illumination zone-to-detection zone distance and selected detector elements as the same function of time define the second part of the illumination zone-to-detection zone distance. Optionally, the illumination array a light-emitting diode (LED) array used in combination with a filter array allowing an analyzer without use of a time-domain interferometer and/or a grating described above.

Referring again to FIGS. 7(B-E), notably, detector elements associated with a first sub-group or first group at a first point in time are optionally associated with an n^{th} sub-group or n^{th} group at a n^{th} point in time when the same and/or a different set of illuminators are used, where n is a positive integer of 2, 3, 4, 5, 10 or more.

Generally, use of a detector array allows multiple pathlengths to be resolved. Additionally, as described below, the multiple pathlengths are optionally resolved as a function of wavelength and/or time. Additionally, as described below, the use of two-dimensional detector arrays allows control over temperature, pressure, and spatial resolution of the probed tissue sample.

### Multiple Two-Dimensional Detector Arrays

For clarity of presentation and efficiency of description, any of the elements in the following description of multiple two-dimensional detector arrays optionally apply to system comprising a single two-dimensional detector array.

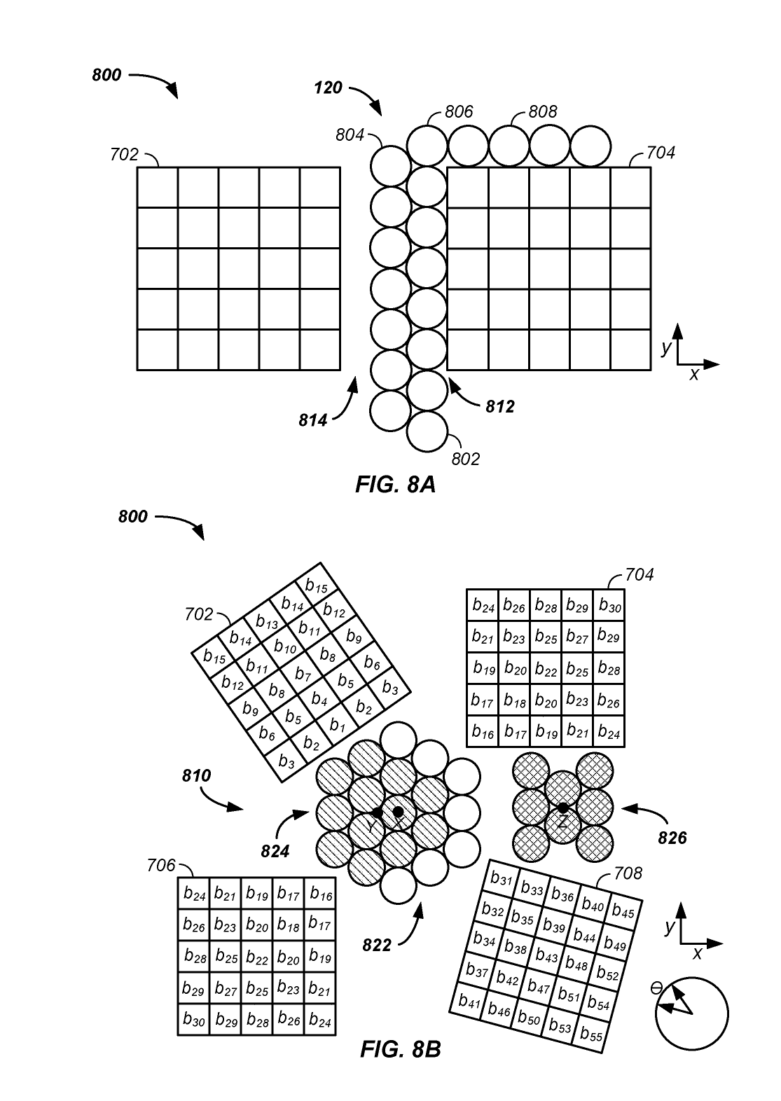

Referring now to FIG. 8A and FIG. 8B, a multiple luminance/multiple detector array system 800 is described. Generally, one and preferably two or more illumination zones are provided by the photon transport system within and/or about two or more detector arrays, such as two or more of the two-dimensional detector arrays 134. For clarity of presentation and without loss of generality, several examples are provided, infra, of the multiple luminance/multiple detector array system 800.

### Distributed/Targeted Illumination

Referring now to FIG. 8A, a first example of the photon transport system 120 delivering light to the skin of the subject 170 at multiple illumination positions relative to two or more detector arrays, such as a first detector array 702 and a second detector array 704, is provided. In this first example, the photon transport system delivers light: (1) by the side 802, (2) removed from the side 804, (3) at the corner 806, and/or (4) around the corner 808 of a detector array, such as the second detector array 704. As illustrated, illumination zones are provided in a first column and in a second column relative to the side of the second detector. The first column 802 and the second column 804 of illuminators are illustrated proximately touching, with a first illuminator/detector gap 812, an edge of the second detector array 704 and with a second illuminator/detector gap 814 an edge of the first detector array 702, where the first illuminator/detector gap 812 and the second illuminator/detector gap 814 are optionally different by greater than ten percent and are, respectively, less than and greater than, about 1, ½, ¼, ⅛, 1/16, or 1/32 of a millimeter. The illuminator/detector gap distances are further described below in terms of water absorbance and scattering of light. Generally, illumination and detection of photons in areas of high absorbance of water have smaller illuminator/detector gap distances for a given scattering range.

For noninvasive glucose concentration determination in the near-infrared, the high absorbance of water limits optical pathlengths to less than 0.5, 0.75, 1.0, or 1.5 mm at wavelengths where water absorbance is high, such as an absorbance of greater than one for a 1 mm pathlength. Slightly longer optical pathlengths of about 1, 2, or 3 mm yield adequate signal-to-noise ratios at wavelengths with an intermediate absorbance of water, such as an absorbance of 0.5 to 1.0 for a pathlength of 1 mm. Still longer optical pathlengths of more than 3, 4, 5, 6, 7, 8, 9, or 10 mm yield adequate signal-to-noise ratios at wavelengths where water has lower absorbance, such as less than 1, 0.5, or 0.25 absorbance units/mm.

For noninvasive glucose concentration determination, in addition to water absorbance, scattering of light has a strong influence of radial distances of light travel in tissue. The more scattering of the light, the smaller the distance of radial travel. Generally, scattering decreases as function of wavelength from 1100 to 2500 nm. Hence, considering scatter only, radial distance of the probing photons in skin tends to increase with wavelength.

Combined, water absorbance and scattering of light in tissue strongly affects the radial distance and intensity of observed photons. From the combination of water absorbance and scatter of light, the inventor has determined that close proximity of illuminator to the detector is required and that the closest proximity is necessary for wavelengths of highest water absorbance. The inventor has also determined that the natural absorbance/scattering/anisotropy properties of tissue act as a natural optical filter of light at selected wavelengths so physical optical filters are unnecessary for some combinations of light illumination sources and detector radial separation distances. Using the water absorbance, tissue scattering properties, and natural optical filtering along with position of glucose absorbance bands and interfering sample constituent absorbance bands, the inventor has devised a set of optical filter combinations, described infra, that allow a successful noninvasive glucose concentration determination with accuracy and precision levels needed for in-home glucose concentration determination.

### Differential Pressure/Force

Referring again to FIGS. 7(A-E) and 8A, any detector array is optionally tilted along the y- and/or z-axes to yield varying degrees of force applied to a sampled tissue sample as a function of detector position when directly contacting the tissue or indirectly contacting the tissue via a fronting detector layer during sampling. For example, the two-dimensional detector array 134 is optionally positioned perpendicular or at an angle to a x/y-plane of the sample at the contact point, where each has benefits. Preferably, but optionally, the two-dimensional detector array 134 is positioned perpendicular and axial to the optical light path at the detector and/or parallel to the skin, which yields a large number of contact points between an associated optical probe tip and the sample site with a minimal applied force/pressure. Optionally, the two-dimensional detector array 134, a probe tip configured to hold the two-dimensional detector array 134, or a portion of the detector array is tilted off of the perpendicular axis, such as less than 1, 2, 3, 5, 10, or 15 degrees toward the skin of the subject 170, which yields a range of applied pressures between the two-dimensional detector array and the skin when the two-dimensional detector array 134 or a layer thereon contacts the skin. The off-axis configuration provides a range of contact forces on the skin ensuring contact in some algorithmically detected areas.

In noninvasive glucose concentration determination, sufficient yet not too much applied force and resulting pressure on the tissue sample yields adequate sample probe/tissue contact for optical coupling while avoiding altering the local concentration of sample constituents, such as via water containing glucose being pushed away from the sample probe. To that end, the inventor has determined that applying a sample probe tilted along the x/y-plane when approaching the tissue sample along an z-axis allows detection of a region of the two-dimensional detector array making first optical contact and a region of the two-dimensional detector array not yet making contact due to the pedestal effect of a large amount of light being reflected off of the surface of skin rapidly changing to a low observed intensity when contact is made due to water absorbance and scattering. Accordingly, there are intermediate positions of the two-dimensional detector array making the light contact with the sample. A two-step algorithm first determines regions of first and hence excessive contact and a region of non-contact and subsequently selects a range of intermediate detector elements for subsequent spectral analysis. Similarly, the first step may select detector elements just making contact and as the sample probe continues to move relative to the sample, either intentionally or via movement of the subject, then selecting a new group of detector elements just making contact with the tissue. More generally, the use of contact and non-contact areas allows selection of individual or groups of detector elements having collected data for subsequent data processing. The selected group of detector elements optionally changes with time.

In noninvasive glucose concentration determination, varying force applied to the tissue sample via a sample probe results in a sample site with changing properties, where the algorithm optionally uses a differential measurement approach and/or knowledge of rates of movement of sample constituents and resultant effect on spectra to determine more accurately an analyte concentration, such as a glucose concentration. For example, the varying pressure determined as described above across a two-dimensional array and/or the varying pressure resultant from a controlled movement of the sample probe relative to the sample site results in data comprising varying and/or controllable pressure, where data from detector elements observing the varying pressure, as a function of time and/or position, is selected for subsequent data processing, such as via binning, grouping, correlations, and/or differential measures.

### Temperature

Still referring to FIGS. 7(A-E) and 8A, any detector array is optionally differentially cooled along the x- and/or y-axes, such as with a Peltier cooler on one side of the detector array, to yield varying degrees of temperature as a function of detector position when directly contacting the tissue or indirectly contacting the tissue via a fronting detector layer during sampling. Similarly, if two or more two-dimensional detector arrays are used, separate detector arrays are optionally controlled at different temperatures. The controlled variance of a portion of a sample probe will heat or cool local skin temperature upon contact. The inventor has determined that absorbance as a function of wavelength of certain constituents of skin, such as water, are temperature sensitive while the absorbance of other skin constituents, such as glucose, are insensitive to temperature. More particularly, with increased temperature, the water bands in the near-infrared region from 1000 to 2500 nm blue shift while the glucose absorbance bands are stationary. Thus, spectra collected at different temperature yield information on what portion/percentage of the absorbance is due to water. The effects are related to depth of penetration of the photons into the skin as a function of probe contact time for changing the temperature and of body circulation for regulating the temperature. Thus, in addition to the analytes having a separable element in terms of wavelength, the analytes are separable as a function of depth. As the detector array is two-dimensional, the analyte are additionally spectroscopically separable as a function of position. One or more of these effects are optionally used to create a map of the sample site. The map is useful for selection of premium spectra for subsequent analysis and/or for determination of outliers. Generally, the varying temperature results in data comprising varying and/or controllable temperature for ease in subsequent data processing, such as via binning, grouping, correlations, and/or differential measures, such as for analysis of temperature sensitive absorbance bands and/or water absorbance bands.

The effects of illumination/detector proximity, applied force and resultant pressure applied to a tissue sample, and localized temperature changes on absorbance positions and magnitude of tissue constituents are preferably combined with pathlength, time, optical filter, and spatial resolution benefits of using two-dimensional detector arrays, which are described in the next section.

### Multiple Pathlengths

Referring now to FIG. 8B, a second example of the photon transport system 120 delivering light to the skin of the subject 170 at multiple illumination positions relative to two or more detector arrays is provided.

The inventor notes that the illumination array and detector array combination is both non-additive and synergistic based upon the particular nuances of a particular sample, such as a noninvasive glucose concentration analysis. For example, merely putting an illumination array next to a detector array does not solve the noninvasive glucose concentration determination problem pursued for the last thirty years at the cost of an estimated one billion dollars. However, proper placement of an illuminator array relative to a two-dimensional detector array along with one of more of: (1) careful consideration of spatial separation of a given illumination element and a given detector element; (2) consideration of scattering and absorbance of the sample in terms of total optical pathlength; (3) precise combinations of optical filters as a function of spatial location across a photon spread of an incident light beam in tissue; (4) timing of illumination with sets of photons at wavelengths having different mean radial travel before exiting the sample; (5) careful binning of pathlength; and (6) proper algorithms applied to the resulting overlapped spectral data sets in terms of time, position, resolution, wavelength range, pressure, and/or temperature yields a successful noninvasive glucose concentration analyzer that is much more than the sum of its parts. As such, while individual elements of the analyzer are described herein in different sections, it is the interplay of these elements that leads to a successful implementation of the analyzer. The interplay of the elements are detailed below at a level understood by an expert in the field and/or one skilled in the art.

### Illuminator Arrays

Referring now to FIG. 8B, a non-limiting example of an illuminator array 810 is illustrated. Generally, the illuminator array 810 is a set of illumination points and/or an illumination area of any geometric cross-sectional shape along the y/z-plane. The illuminator illuminates the sample tissue, where illumination is also referred to as irradiation and/or incident light. The illuminator elements described herein are in the 700 to 2600 nm spectral range and more preferably in the 1000 to 1900 nm spectral range.

Referring again to FIG. 8B, three non-limiting examples of illuminator arrays 810 are illustrated, each representing different illumination cases where many additional cases are possible. Notably, deviation from the principles or designs described herein are at high risk of degraded signal-to-noise ratios making noninvasive glucose concentration difficult.

### First Illuminator Array

In a first case, a first illuminator array 822 is illustrated comprising an about circular illumination pattern, here represented as nineteen illumination areas and/or a rough circle of illumination. The illuminated circle represents many cross-sectional shapes of the incident photon beam. However, in the first illuminator array 822, uniform light optionally travels through each illuminator element or light of different wavelengths travels to the sample through grouped sections of the illumination circle.

For instance, at the perimeter of the first illumination array, 1, 2, 3, or more fiber optics optionally carry wavelengths of light that are strongly absorbed and/or strongly scattered, such as at the strongly water absorbed wavelength of 1550 nm, as the photons are: (1) limited in radial travel through tissue before being too highly absorbed, (2) probe adequate sample for a preferred signal-to-noise ratio, and (3) are emitted for detection due to scattering properties at the selected wavelength.

Similarly, in the center of the illumination circle that is further from surrounding detector elements, light having wavelengths that have enhanced mean radial travel distances is optionally delivered to the sample, such as wavelengths of lower water absorbance and/or less scattering at about 1600 nm. For the central region of illumination: (1) the lower absorbance yields sufficient depth of penetration for probing glucose containing tissue layers while and (2) the intermediate scattering properties of the tissue yield photons exiting the tissue at illumination-to-detector distances appropriate for the incident photons in the center of the illuminator having to pass under the outer regions of the incident illumination zone before reaching an associated detector element.

Indeed, for wavelengths of even lower absorbance and still lower scatting, such as at about 1650 nm, the associated detector element is preferably still further from the illumination zone, thereby yielding a proper illumination-to-detector distance to achieve long enough optical paths for a sufficient signal-to-noise ratio. Thus, as shown, results vary significantly across a narrow wavelength region. Particularly one design is illustrated at 1500 nm, a second at 1550 nm, and a third at 1600 nm. Whereas, reversing the order and/or blending the photon wavelength range may seriously degrade signal-to-noise ratios of resulting data sets. For instance, placing the 1500 nm illuminators in the center of the illumination bundle and using optical filters passing the 1500 nm radially well into the detector array yields high absorbance levels having a seriously degraded signal-to-noise ratio.

Still referring to FIG. 8B, additional illumination arrays are described. A second illuminator array 824, here represented as twelve illumination regions and/or a subset of the first illuminator array 822; and a third illuminator array 826, which represents an about square and/or rectangular illumination array, which does not overlap any of the first illuminator array 822 are illustrated. Additionally, a fourth illuminator array optionally overlaps a portion of any other illuminator array as a function of time, not illustrated. Generally, the illuminator array is of any geometrical shape; is optionally operated separated illumination elements, such as illustrated in FIG. 5N; and is positioned anywhere relative to a detector. However, distance between a given illuminator and a given detector are preferably selected based on a priori knowledge and/or knowledge measured for a given sample, such as absorbance as a function of wavelength, a scattering coefficient as a function of wavelength, a measure of anisotropy as a function of wavelength, a tissue layer depth, and/or a tissue layer thickness.

### Detector Arrays

Still referring to FIG. 8B, an illustrative and non-limiting example of a three illuminator area system coupled to a four area detection system is described, where the four area detection system comprises: a first detector array 702, a second detector array 704, a third detector array 706, and a fourth detector array 708. In this second example, four detector arrays are illustrated about the three illumination arrays 822, 824, 826, which are representative of any number of illumination elements and/or any number of illumination arrays. For ease of presentation, this section refers to a center mean illumination point for each of the three illumination arrays 822, 824, 826, which in the present case is the center of the symmetrically illustrated light illumination arrays labeled X, Y, and Z, respectively.

Still referring to FIG. 8B and now referring to the first detector array 702 and the first illumination array 822 having center X, the inventor notes that the first row of the detector array contains detector elements at three optical pathlengths from the center of the illumination array. A first pathlength, b₁, is observed at the center element of the first row of detector elements. A second pathlength, b₂, is observed with each of the detector elements, in the first row of the detector array, adjacent the center detector element in the first row of the first detector array 702. Data collected at the redundant pathlengths comprise multiple uses, such as additional pathway data for the transformation and/or a narrowed standard deviation of pathlength linked to a corresponding reduction in determined concentration, as described supra, and precision determination, outlier detection, tissue variation estimation, and/or tissue mapping, as described infra. A third pathlength, b₃, is observed with each of the detector elements at the outer ends of the first row of the first detector array 702. Similarly, the second row of the detector array observes three additional pathlengths, described here as the fourth, fifth, and sixth pathlengths, b₄, b₅, b₆. Similarly, the third, fourth, and fifth rows of the detector array contains fifteen additional detector elements observing an additional three pathlengths per row or nine additional pathlengths, b₇-b₁₅. The fifteen observed radial distance also yield fifteen mean pathways where each pathway has a narrower standard deviation versus the combined twenty-five detection zones with a corresponding enhancement of certainty of pathlength and, intensity allowing, a corresponding noninvasive glucose concentration determination. The inventor notes that the first detector array 702, represented as a 5×5 matrix of detector elements, is optionally an m×n array of detector elements, as described in relation to FIG. 6A, with a corresponding number of observed mean optical pathlengths and mean optical depths.

Still referring to FIG. 8B, as described, supra, in relation to FIG. 5 and further described, infra, as the median pathlength of the probing photons increases, the depth of penetration of the mean photon increases for each wavelength in the range of 1100 to 2500 nm until an absorbance limit of detection is reached. Thus, as illustrated, the first detector array 702 is configured to observe fifteen pathlengths, three per row, where ten of the pathlengths are observed twice with intentionally separated sample tissue volumes.

Still referring to FIG. 8B and referring now to the second detector array 704 and still referring to the first illuminator array 822, the second detector array 704 is rotated about the x-axis relative to the first detector array 702 placing a corner of the second detector array closet to the mean illumination point of the first illumination array, X, as opposed to the first detector array 702 having a side closest to the mean illumination point, X. Rotation of the second detector array 704 allows another set of observed pathlengths, even when a duplicate detector array design is used. For example, as illustrated the corner of the second detector array 704 represents a sixteenth pathlength, b₁₆. Similarly, the second diagonal of the second detector array 704 contains two additional detector elements observing a seventeenth pathlength, b₁₇, in duplicate due to symmetry about a line through the center of the first illuminator array 822 and nearest corner of the second detector array 704. Similarly, the third to ninth diagonal of the second detector array 704 contain twenty-two additional detector elements observing thirteen additional pathlengths, b₁₈-b₃₀.

Still referring to FIG. 8B and referring now to the third detector array 706 and still referring to the first illuminator array 822, the third detector array 706 is positioned opposite the second detector array 704. The symmetrical positioning of the third detector array 704 relative to the second detector array 704 and the first illuminator array 822 yields pathlengths mirroring those observed using the second detector array; particularly, pathlengths sixteen to thirty, b₁₆-b₃₀. The mirrored pathlengths allows repetitive data for an internal check of results, validation of results, outlier detection, concentration estimation bounding, and/or additional algorithmic uses. Notably, by merely shifting a detector array and/or a source array along the y-z-axes, instead of repeated pathlengths, the new illuminator/detector combination will observe new pathlengths; twenty-five new pathlengths for the illustrated 5×5 detector element array.

Still referring to FIG. 8B and referring now to the fourth detector array 708 and still referring to the first illuminator array 822, the fourth detector array 708 is rotated an angle theta relative to the first detector array 702. The rotation of the fourth detector array 708 breaks symmetry along a line from the center of the first illuminator array 822, X, and a center of the fourth detector array 708. Now, intentionally, lacking rotational symmetry the fourth 5×5 detector array observes twenty-five additional pathlengths, b₃₁-b₅₅, compared with the fifteen pathlengths observed by the first detector array 702 and fifteen distinct pathlengths observed using the second detector array 704.

Still referring to FIG. 8B and now referring to the second illuminator array 824, the center of the second illuminator array 824, Y, is offset along the x- and y-axes relative to the center of the first illuminator array, X, which breaks symmetry relative to each of the four detector arrays 702, 704, 706, 708. The intentional breaking of the symmetry allows the four detector arrays 702, 704, 706, 708 to observe one hundred (25×4) new pathlengths by merely changing the optical illumination configuration. Similarly, now referring to the third illuminator array 826, moving the center of illumination to a third point, Z, yields an additional one hundred new observed pathlengths (25×4). To illustrate the number of observed pathlengths still further, use of four 50×50 detector arrays without symmetry relative to five illumination patterns yields 50,000 (2500 detectors/array×4 arrays×5 illumination zones) observed pathlengths. Detector arrays of m×n dimension where m and/or n are independently any positive integer of 1, 2, 3, 5, 10, 100, 500, 100 or more thus yields tens, hundreds, thousands, and/or millions of detector elements. Hence, with a two-dimensional detector array, even using one detector design, and an illumination source, even statically positioned, may readily yield hundreds of thousands or millions of observed pathlengths in a period of less than 1, 2, 3, 4, 5, 10, 20, or 30 seconds as the detectors are optionally used in parallel. The tens, hundreds, thousands, or millions of pathways and the associated algorithm transform combine to form a new spectrometer type, referred to herein as a pathmeter or sample induced/spatially enhanced general transform function, where the pathmeter functions through discrimination of the sample as a function of pathway with or without use of traditional wavelength separation devices, such as a grating or a time domain to frequency domain element or algorithm.

Still referring to FIG. 8B, detector elements of the sample interface 150 optionally have i-symmetry, identity symmetry, S₂-symmetry, rotational C₂-symmetry, and/or Cₙ-symmetry, where n is a positive integer of at 3, 4, 5, or more.

Still referring to FIG. 8B, replicates of a single detector array are illustrated to reduce manufacturing costs. More generally, the n detector arrays are optionally of different shapes and/or have different numbers of detection elements, where n is a positive integer of more than 1, 2, 3, 4, 5, or 10.

### Filters

Herein, optical filters optically coupled with elements of the detector arrays are described.

### Longpass Filters

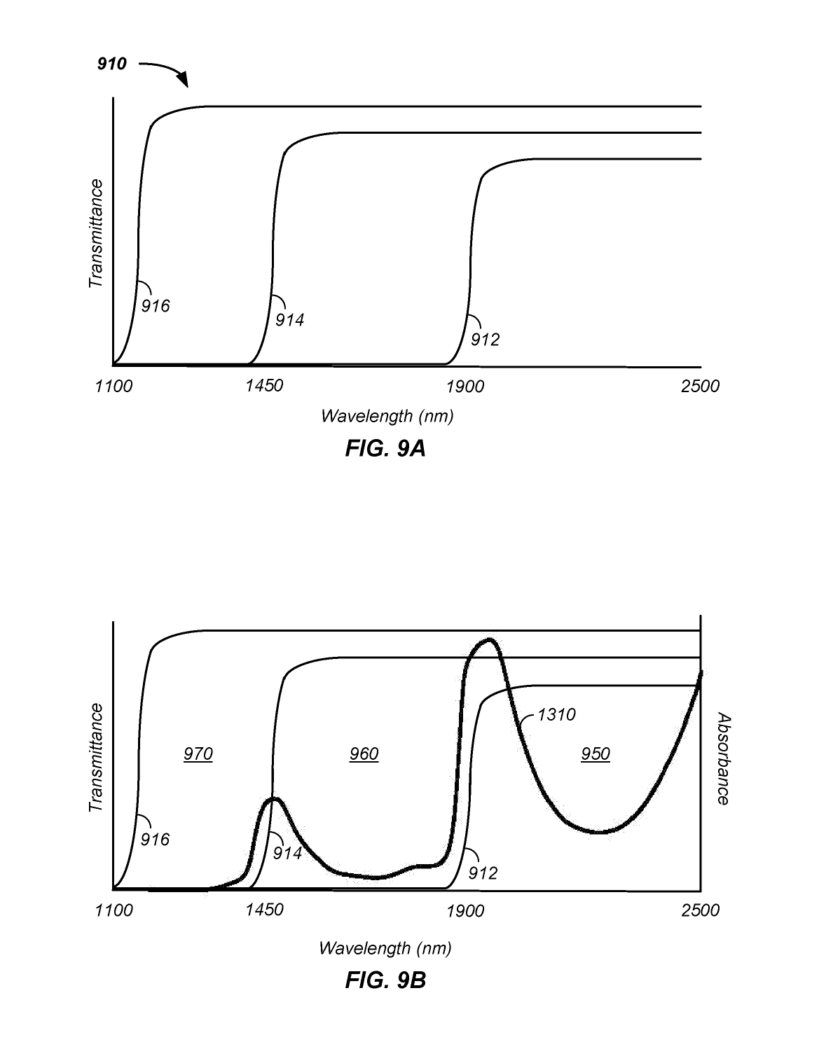

Referring now to FIG. 9A, a series of longpass filters are described. A longpass filter is an optical interference and/or coloured glass filter that attenuates and/or blocks shorter wavelengths and transmits and/or passes longer wavelengths over a range of wavelengths. Longpass filters optionally have a high slope described by a cut-on wavelength at a wavelength passing fifty percent of peak transmission. Herein, longpass filters refer to filters comprising a fifty percent cut-on wavelength in the range of 900 to 2300 nm. More preferably, for analysis of tissue spectra, the inventor has determined that longpass filters complementing water absorbance bands offer multiple advantages relating to detector dynamic range. Optionally, the longpass filters are used to divide light from a light-emitting diode or group of light emitting diodes into two or more intersecting wavelength bands.

Referring still to FIG. 9A and referring now to FIG. 9B, a first longpass filter 912 is illustrated comprising a fifty percent cut-on wavelength in the range of 1850 to 2050 nm, such as at about 1900, 1950, or 2000 nm. The first longpass filter 912 is designed to transmit photons in a region referred to herein as a ‘combination band region’ 950, which comprises a first region of low water absorbance and three glucose absorbance bands. The inventor has determined that by having the sharp, often temperature sensitive and/or difficult to analyze region due to rapid changes in transmittance as a function of wavelength, cut-on wavelength in the wavelength range of the large water absorbance band spectral feature, that the filter weaknesses are masked by the water absorbance band while the filter strengths are optimized, as described herein. First, the first longpass filter 912 transmits a high percentage of light, such as greater than 70, 80, or 90 percent, in the desirable range of 2100 to 2350 nm where water absorbance 1310 and scattering combine to yield detected photons in the glucose rich dermis layer of skin and where glucose has three prominent absorbance bands at 2150, 2272, and 2350 nm. Second, the first longpass filter 912 has a transition cut-on range, that hinders analysis due to the rapid change in transmittance as a function of wavelength and is susceptible to temperature induced spectral shifts, that is placed in a region where water absorbance prevents detection of photons penetrating into the dermis, thereby eliminating the problem. Third, the first longpass filter 912 has a blocking range from a detector cut-on of about 700 nm to about 1900 nm, which blocks photons otherwise filling a dynamic range of an element of the detector array, such as a 2.6 μm InGaAs detector sensitive to light from 700 to 2600 nm, which allows an enhanced signal-to-noise ratio, using proper detector gain electronics and/or integration time, in the desirable range of 2100 to 2350 nm. The inventor notes that the water absorbance band at circa 2500 nm functions as a natural shortpass filter, which combines with the first longpass filter 912 to form a combination band bandpass filter.

Referring still to FIG. 9A and FIG. 9B, a second longpass filter 914 is illustrated comprising a fifty percent cut-on wavelength in the range of 1350 to 1490 nm, such as at about 1375, 1400, 1425, 1450, or 1475 nm. The second longpass filter 914 is designed to transmit photons in a region referred to herein as a ‘first overtone region’ 960, which comprises a second region of low water absorbance and three glucose absorbance bands. As with the first longpass filter 912, the second longpass filter 914 is designed to function in a complementary manner with water absorbance of tissue. Particularly, the second longpass filter 914 transmits three prominent glucose bands in the first overtone region centered at circa 1640, 1692, and 1730 nm, which are in a region where the dominant absorber water and tissue scattering combine to yield detectable photons having sampled the glucose rich dermal layer of tissue in diffuse reflectance mode. Further, the second longpass filter blocks/substantially blocks light from about 700, 800, 900, 1000, and/or 1100 to 1450 nm, which would otherwise contribute to filling a dynamic range of a detector array element, such as a 1.7 or 1.9 μm InGaAs detector sensitive to wavelengths as short as 700 nm. Blocking the second overtone light, described infra, thus allows full use of a dynamic range of a detector in the first overtone region and a correlated enhancement in a signal-to-noise ratio of the three first overtone glucose absorbance bands. Still further, the second longpass filter 914 benefits from the water absorbance band at 1950 nm, which functions as a natural shortpass filter to the second longpass filter 914 forming a first overtone bandpass filter from about 1450 to 2000 nm or a spectral region therein. Thus, the sample is used as a filter in the pathmeter.

The inventor notes that traditional spectroscopic analysis of tissue using near-infrared light does not: (1) combine light from the combination band region 950 with light from the first overtone region 960 using separate detectors, which are optionally individually optimized for a spectral region, or (2) use separate longpass filters, bandpass filters, or optics coupled to the multiple detectors to simultaneously enhance spectral quality of the first overtone region and combination band region.

The inventor has determined that the three glucose absorbance bands in the combination band region are linked at an atomic/chemical energy level to the three glucose absorbance bands in the first overtone region. Hence, detection of signals from corresponding bands of the combination band region and first overtone region are optionally compared to enhance glucose concentration estimations.

Referring still to FIG. 9A and FIG. 9B, a third longpass filter 916 is illustrated comprising a fifty percent cut-on wavelength in the range of 700 to 1200 nm, such as at about 800, 900, 1000, or 1100 nm. The third longpass filter 916 is designed to transmit photons in a region referred to herein as a ‘second overtone region’ 970 from about 1000 or 1100 to 1400 nm. Similar to the first and second longpass filters 912, 914, the third longpass filter 916 is designed to optimize signal-to-noise ratios in the second overtone region, function with the use of water absorbance bands at 1450 and 1900 nm as natural shortpass filters, and to be used with detector array elements observing photons having sampled at least the dermis skin layer. It is noted that the first, second, and third longpass filters 912, 914, 916 are illustrated with differing maximum light throughput for clarity of presentation, but each optionally function as a longpass filter as described supra.

### Shortpass Filters

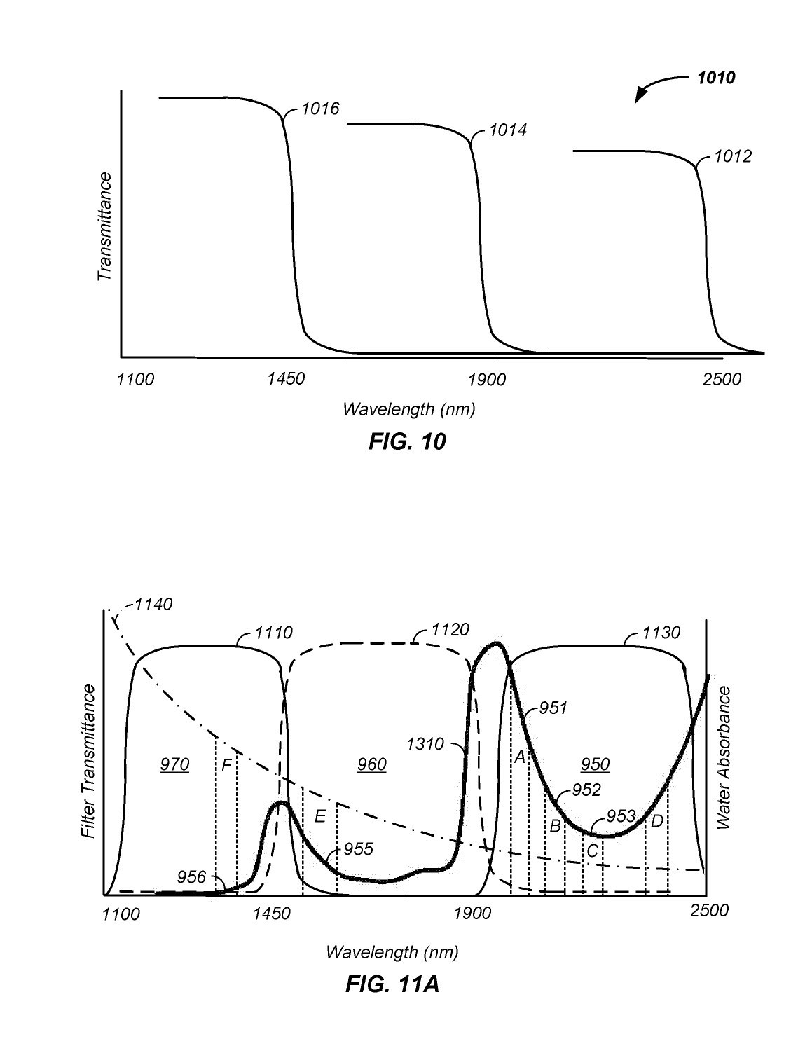

Referring now to FIG. 10, a series of shortpass filters are illustrated. A shortpass filter is an optical interference and/or coloured glass filter that attenuates and/or blocks longer wavelengths of light and transmits and/or passes shorter wavelengths of light over a spectral range. Herein, shortpass filters refer to filters comprising a fifty percent cut-off wavelength in the range of 1400 to 3000 nm. More preferably, for analysis of tissue spectra, the inventor has determined that shortpass filters complementing water absorbance bands offer multiple advantages relating to detector dynamic range. Optionally, a shortpass filter is used to enhance wavelength or spectral resolution by dividing a light emitting diode wavelength band into two or more sections. A shortpass filter preferable passes greater than 60, 70, 80, or 90 percent of light in the passed wavelength or spectral region and transmits less than 1, 5, 10, 20, 30, or 40 percent of the light in the attenuated spectral region.

Referring again to FIG. 10 and referring now to FIG. 11A, a first shortpass filter 1012 is illustrated comprising a fifty percent cut-off wavelength in the range of 2350 to 3000 or more nanometers. The first shortpass filter 1012 is designed to transmit photons in the second overtone 970, first overtone 960, and/or combination band region 950. The first shortpass filter 1012 is designed to block infrared heat at wavelengths greater than about 2350 nm, where otherwise transmitted heat would alter temperature of parts of the tissue and result in shifting of oxygen-hydrogen water band positions. Preferably, the first shortpass filter 1012 is combined with a longpass filter, such as with the first longpass filter 912 to form a combination band bandpass filter 1130 for the combination band region, with the second longpass filter 914 to form a bandpass filter for the first overtone/combination band spectral region, with the third longpass filter 916 to form a second overtone/first overtone/combination band bandpass filter, or with a longpass filter to form a bandpass filter.

Referring again to FIG. 10 and FIG. 11A, a second shortpass filter 1014 is illustrated comprising a fifty percent cut-off wavelength in the range of 1800 to 2100 nm, such as at about 1900, 1950, or 2000 nanometers. The second shortpass filter 1014 is designed to transmit photons in the second overtone 970 and first overtone 960 regions. The second shortpass filter 1014 is designed to block infrared heat at wavelengths greater than about 2000 nm, where otherwise transmitted heat would alter temperature of parts of the tissue and result in shifting of oxygen-hydrogen water band absorbance positions of molecules in the skin. Preferably, the second shortpass filter 1014 is combined with a longpass filter, such as with the second longpass filter 914 to form a first overtone bandpass filter 1120 for the first overtone region or with the third longpass filter 916 to form a second overtone/first overtone bandpass filter.

Referring again to FIG. 10 and FIG. 11A, a third shortpass filter 1016 is illustrated comprising a fifty percent cut-off wavelength in the range of 1300 to 1600 nm, such as at about 1400, 1450, or 1500 nanometers. The third shortpass filter 1016 is designed to transmit photons in the second overtone 970 and optionally part of the first overtone 960 spectral regions. The third shortpass filter 1130 is designed to block photons from about 1600 to 2500 nm that would otherwise contribute to filling a detector well depth and/or dynamic range of a near-infrared detector, such as an indium gallium arsenide detector. Preferably, the third shortpass filter 1016 is combined with a longpass filter, such as with the third longpass filter 916, to form a second overtone bandpass filter 1110 for the second overtone region and/or a portion of the first overtone region, such as about 1500 to 1580 nm.

As illustrated in FIG. 11A, the fifty percent cut-off of the shortpass filter is preferably in a region of strong water absorbance to maximize transmitted photons while minimizing detection of out of band photons by using the water absorbance properties of skin.

Referring now to FIG. 11B, narrowband bandpass filters or bandpass filters are optionally used to enhance the signal-to-noise ratio in a narrow spectral region, such as about 25, 50, 100, 150, or 200 nm wide. Optionally, the bandpass filters are associated with a light intensity limiting sample constituent, such as water. For example, in the combination band spectral region 950, a minimum water absorbance is observed at about 2270 with higher water absorbances observed at both longer and shorter wavelengths, such as at about 2150 or 2350 nm. Without an optical filter or an independent wavelength selection device, a multiplexed signal, such as obtained using a Fourier transform near-infrared spectrometer, will dominantly fill a well of a detector with photons from the spectral region transmitting more light, such as at about the water absorbance minimum, while obtaining fewer photons from spectral regions of higher absorbance. Thus, the signal-to-noise ratio in regions of higher water absorbance is degraded compared to use of the narrowband bandpass filter in a region of higher water absorbance and/or total observed absorbance. A narrowband bandpass filter or a set of narrowband bandpass filters in combination with multiple detectors, such as a two-dimensional detector array allows for each region to fully use a dynamic range of the detector elements, if properly matched with amplifier circuitry and integration time. For example, a first narrowband bandpass filter 1150 is optionally used at a first set of wavelengths correlated with a larger water absorbance. Similarly, a second narrowband bandpass filter 1160 and/or a third narrowband bandpass filter 1170 are optionally used at spectral regions of intermediate and low water absorbance, respectively. Generally, n narrowband bandpass filters are optionally used, where n is a positive integer of 2, 3, 4, 5, 6, 7, 8, 9, 10, 15, 20, 30, 50 or more. Combined, signals collected, preferably simultaneously, with the set of narrowband bandpass filters allows coverage of large regions of the near-infrared region where signal-to-noise ratios are significantly enhanced for given subsets of the near-infrared spectral region associated with each narrowband bandpass filter.

Still referring to FIG. 11B, an example of enhancing spectral or wavelength resolution using a narrowband light source and one or more bandpass filters is illustrated. For instance, a narrowband band of light 1180 is illustrated, such as from an LED is illustrated, where the x-axis of the LED band of emitted light is expanded for clarity of presentation. An optional first narrowband bandpass filter 1182 resolves the high energy side of the narrowband band of light 1180. Similarly, an optional second narrowband bandpass filter 1184 resolves the low energy side of the narrowband band of light 1180. By extension, the inventor notes that one or more narrowband filters are optionally placed over a corresponding set of one or more detector elements, such as in the two-dimensional detector array 134 to separate wavelengths of light from any of: the source system 110, light source 112, photon transport system 120, sample interface 150, array of illumination sources 400, an individual LED, and/or from discrete illumination zones to yield enhanced wavelength band resolution, which is also referred to herein as spectral resolution.

The narrowband filters are optionally used in combination with an array of LEDs, where LED wavelength regions are optionally radially configured relative to the associated filter in the filter arrays as a function of water absorbance. For instance, a first LED illuminating at a wavelength where water absorbance in skin is high is positioned close to one or more filters passing light emitted by the first LED, such as within about 0.2 to 0.75 millimeters. Similarly, a second LED illuminating at a wavelength where water in skin has medium absorbance is positioned at an intermediate distance from the one or more filters passing light emitted by the second LED, such as within about 0.5 and 1.5 millimeters. Similarly, a third LED illuminating at a wavelength where water in skin has low absorbance is positioned at a still further distance from the one or more filters passing light emitted by the third LED, such as within about 1.0 and 2.5 millimeters. Generally, the distance between an LED and a filter configuration passing light of the LED is a function of absorbance and/or scattering, such as according to equation 2,

$$distance ∼ \frac{1}{abs} * \frac{1}{scattering} \qquad ( eq . 2 )$$

where a correlation with the function is at least 0.4, 0.5, 0.6, 0.7, 0.8, or 0.9, where abs is absorbance of the sample at the given wavelength, such as approximated by water absorbance at the given wavelength, and scattering is the scattering coefficient and/or relative scattering coefficient at the given wavelength relative to neighboring wavelengths.

Referring now to FIG. 12 and referring again to FIG. 11B, a step-function bandpass filters is described. In this example, a first step-function bandpass filter 1140 is illustrated with a large percent transmittance in the first overtone spectral region 960 and a lower transmittance, such as about 10, 20, 30, 40, or 50 percent transmittance, in the second overtone spectral regions 970. A major benefit of the first step-function bandpass filter 1140 is simultaneous collection of light from the first and second overtone spectral regions 960, 970, where the lower sample transmittance of the first overtone region 960, relative to the second overtone region 970, is compensated for by the greater transmittance in the first overtone region 960, relative to the second overtone region 970, of the first step-function bandpass filter and the difference in detectivity, D*, between the two regions. The two-level bandpass filter allows the dynamic range of the detector, such as a 1.7 or 1.9 μm InGaAs detector, to be roughly or completely filled across the response range instead of being dominated by second overtone light, which enhances the signal-to-noise ratio in the first overtone region while maintaining the signal-to-noise ratio in the second overtone region.

Still referring to FIG. 12, the first step-function bandpass filter is optionally a combination of a shortpass and longpass filter, such as the second shortpass filter 1014 and a fourth longpass filter 918, where the fourth longpass filter 918 intentionally leaks light, such as less than 30, 20, 10, or 5 percent, at wavelengths shorter than about 1450 nm. Generally, the step-function bandpass filter has any transmittance profile. However, preferably, sections of 25, 50, 100, 200, or more nanometers of the step-function bandpass filter are anti-correlated with water absorbance, scattering, or a combination thereof, with a correlation coefficient of less than about −0.9, −0.8, −0.7, or −0.6.

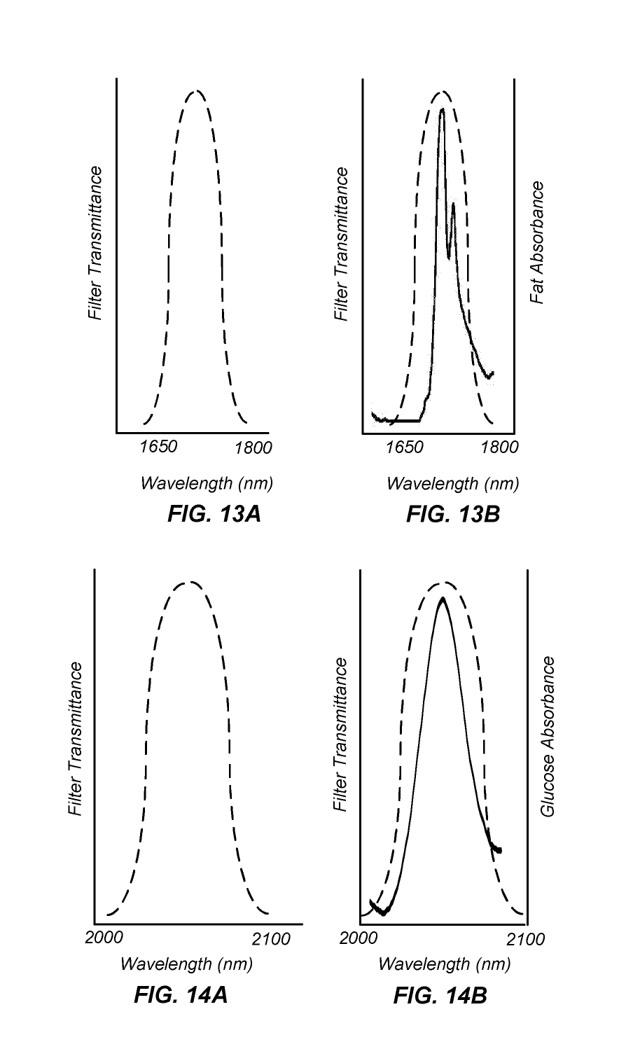

Referring now to FIGS. 13A, 13B, 14A, and 14B, narrowband filters are illustrated relative to absorbance features of blood and/or skin constituents. In FIG. 13A, a first analyte narrowband bandpass filter is illustrated; overlaid with fat absorbance bands in FIG. 13B. In FIG. 14A, a second analyte narrowband bandpass filter is illustrated; overlaid with a glucose absorbance bands in FIG. 14B. Generally, a set of n individual filters, where each filter passes wavelengths dominated by a limited number of sample constituents, are optionally used. Optionally and preferably the n individual filters are associated with individual detectors or groups of detectors of the two-dimensional detector array 134, as described infra. Notably, since the analyte narrowband filters, such as in FIGS. 13A, 13B, 14A, and 14B, occur at different wavelengths where the total absorbance, dominated by water absorbance and/or scattering, varies, preferably the detector array uses different gain settings and/or integration times for different detector elements, within the two-dimensional detector array 134, associated with different optical filters. For example, a hardware gain electrical circuit using at least an operational amplified and a resistor is used to convert detected current into voltage and to amplify the resultant signal. Larger gain settings are optionally used for detectors comprising a further radial distance from a sample illumination source, such as an increase in gain setting of at least 10, 20, 50, or 100 percent. Resultant signals of the pathmeter represent separate probed sample paths with wavelength resolution, again with or without need of a traditional wavelength separation element outside of the sample itself.

### Detector Array/Filter Array Combinations

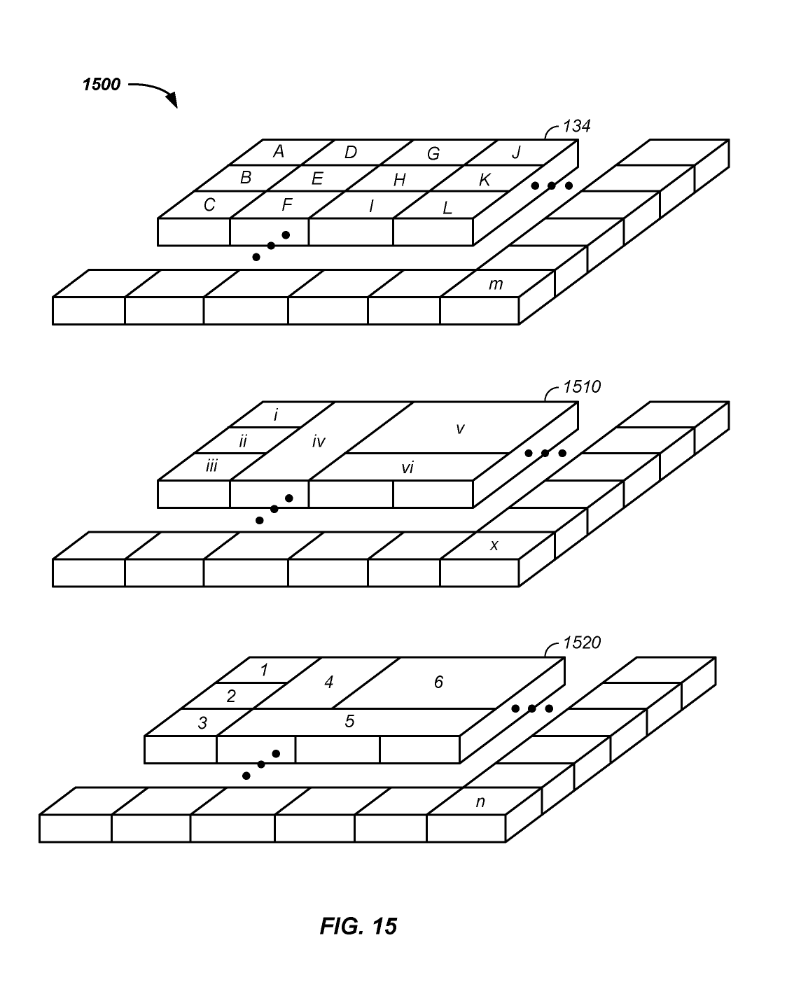

Referring now to FIG. 15, a detector array-filter array assembly 1500 is illustrated. For clarity of presentation and without limitation, the detector array-filter array assembly 1500 is illustrated and described as a single unit. However, optionally, the one or more two-dimensional filter arrays are optionally proximate the two-dimensional detector array 134, such as within less than 5, 2, 1, or 0.5 millimeters or are well removed from the two-dimensional detector array 134, such as at any position in the optical train between the source system 110 or the subject 170 and the detector system 130. Further, for clarity of presentation and without limitation, two two-dimensional filter arrays are described, which are representative of 1, 2, 3, 4, 5, or more filter arrays. Still further, the two-dimensional filter arrays presented are optionally presented in reverse or any order in the optical train.

Still referring to FIG. 15, a first example of the detector array-filter array assembly 1500 is described, which is an optical element of the incident optic-two-dimensional array system 700. In this example, the two-dimensional detector array 134 is combined with 1, 2, 3, 4 or more two-dimensional optical filter arrays, such as a first optical filter array 1510 and a second optical filter array 1520. Several features of the detector array-filter array assembly 1500 are noted. First, optionally individual detector elements, A, B, C, optically align with individual filters of the first optical filter array 1510, i, ii, iii, and/or optically align with individual filters of the second optical filter array 1520, 1, 2, 3. Second, optionally two or more detector elements, D, E, F, optically align with a single filter element of the first optical filter array 1510, iv, which aligns with two or more elements of the second optical filter array 1520, 4, 5. Third, optionally, a single optical filter element of the first optical filter array 1510, iv, optically aligns with two or more elements of the second optical filter array 1520, 4, 5. Fourth, optionally, a single optical filter element of the second optical filter array 1520, 5, optically aligns with two or more elements of the first optical filter array 1520, iv, vi. Fifth, optionally columns and/or rows of detector elements, (D, E, F), (F, I, L) align with a column optic, iv, or row optic, 5, respectively. Fifth, a single two-dimensional filter array, such as the first two-dimensional optical filter array 1510, optionally contains more than 2, 3, 4, 5, 10, 20, or 50 filter types. Sixth, a single two-dimensional filter array, such as the first two-dimensional optical filter array 1510, optionally contains more than 2, 3, 4, 5, 10, 20, or 50 filter shapes.

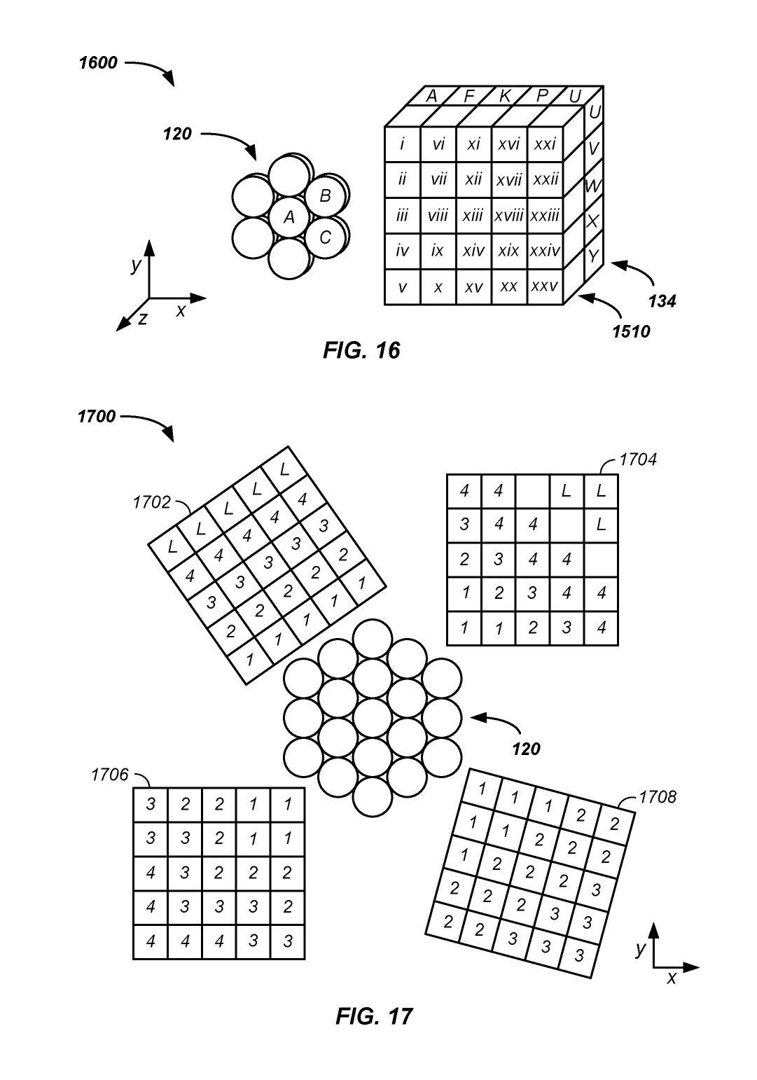

Referring now to FIG. 16, a multiple illumination zone-multiple detection zone system 1600 is illustrated. For example, an array of illumination points delivered from the photon transport system 120 is illustrated launching photons out of the page along the z-axis into the skin of the subject 170, not illustrated. An array of detection zones are achieved, monitoring photons moving into the page along the z-axis, using the two-dimensional detector array 134 and as illustrated the optional first optical filter array 1510. Similar to the systems described supra when referring to FIG. 8B, the illustrated illumination array optionally illuminates all illuminators; a single illuminator, such as element A, B, or C; and/or subsets of illuminators, such as elements A and B or A, B, and C. As described, supra, the optionally varying position of illumination coupled with the two-dimensional detector array 134 yields discrete pathlength and depth of penetration information about the optically sampled skin tissue of the subject 170 for each detector element of the two-dimensional detector array 134 and/or separate pathways. By varying wavelength of light as differing illumination zones, such as A, B, or C, and by coupling appropriate filters, such as i-xxv, based on sample parameters as described above, a wavelength zone-sample zone resolved pathmeter is generated. As illustrated, the first two-dimensional optical filter array 1510 provides additional insight as to the sampled skin by selectively filtering: (1) regions, such as the combination band region 950, the first overtone region 960, and/or the second overtone region 970; (2) analytes, such as through use of the narrowband analyte filters; and/or (3) based on intensity of the observed signal, such as through narrowband filters designed for a narrow range of absorbances of the sample tissue, where detector gain elements and/or integration times are optionally individually configured for each element or group of elements, such as along a column or row, of the two-dimensional detector array 134 to optimize even filling of dynamic wells of the detector element and enhance signal-to-noise ratios, as further described infra.

### Detector

Physical sample size, limited radial optical pathways, and tissue constraints limit a sample interface size between the analyzer 100 and subject 170. For example, there is limited room about an incident light source where photons diffusely reflect back to the surface. Particularly, a noninvasive glucose concentration analyzer is optionally and preferable a compact package limited to a small area on a curved arm, where radial arm curvature limits an analyzer-sample contact area to less than 1, 5, 10, 25, or 50 square millimeters. As such, minimizing use of non-optical parameters in the sample interface is beneficial. In an alternative configuration, since the pathmeter optionally uses widely spaced illumination zones, the analyzer-sample contact area optionally covers a long radial distance along the sample site, such as along the arm, where the long radial distance is more than 5, 10, 25, 50, 75, 100, or 125 mm.

In one embodiment, a readout element of a CCD array is placed on an outer perimeter of the sample interface area or outside of the perimeter. If two or more detector arrays are used, the readout elements of the detector arrays are optionally on opposite sides of the sample interface to stay out of the sampling area or are on adjacent sides of the sample interface, such as at about ninety degrees from each other along the outside of the sample area to stay out of the sampling area. Similarly, if three of more detector arrays are utilized, the readout positions of the multiple detector arrays optionally circumferentially surround the sample interface area. Further, having multiple detector arrays allows a more rapid readout of the data as the readouts are optionally at least partially in parallel. Parallel readout of the gathered signal allows: (1) faster readout, (2) timing of readout corresponding to an expected signal-to-noise ratio, and/or (3) an ability to initiate calculations before all data is collected by the analyzer, such as initiation of a tissue-specific tissue map, initiation of data transfer to a remote computing facility, and/or part of a rolling glucose concentration estimation. Still further, columns/rows of a traditional CCD array are optionally configured along arcs, chords, circles, and/or along a detector segment, allowing detector elements to be positioned in concentric rings or other non-rectangular patterns. Still further, optionally the individual rows, columns, and/or curved sets of detector elements are optionally read out individually allowing an inner set, relative to an illuminator, where absorbance is smallest to be read out first and/or more often than outer detector sets, where larger absorbance of tissue leads to longer sample integration times to fill dynamic wells of the detector area to the same degree as radially inner detector elements and/or detector elements positioned to receive a larger number of photons per unit time.

### Multiple Two-Dimensional Detector Arrays

Referring now to FIG. 17, a multiple two-dimensional detector array system 1700 is illustrated. As illustrated, the photon transport system 120 delivers photons proximate a plurality of two-dimensional detector arrays denoted here as a first detector array 1702, a second detector array 1704, a third detector array 1706, and a fourth detector array 1708. Generally, any number of two-dimensional detector arrays are optionally used, such as 2, 3, 4, 5, 10, 20, or more detector arrays. Configurations of the detector arrays 1702, 1704, 1706, 1708 are described, infra.

Referring still to FIG. 17 and referring now to the first detector array 1702, the first detector array is optionally positioned with an edge of the first detector array 1702 proximate an outer border or edge of an illumination point, zone, or array of illuminators of the photon transport system 120. In this example, the first detector array 1702 is optically and/or physically coupled to a series of filters, such as: a first filter, 1, coupled to a first detector element row; a second filter, 2, coupled to a second detector row; a third filter, 3, coupled to a third detector row; a fourth filter, 4, coupled to a fourth detector row; and a fifth filter, L, coupled to a fifth detector row. Here the first, second, third, fourth, and fifth filter, 1, 2, 3, 4, L, are optionally a combination band filter, a first overtone filter, a first and second overtone filter, a second overtone filter, and luminance filter, respectively. Similarly, the first, second, third, fourth, and fifth filter, 1, 2, 3, 4, L, are optionally an analyte narrowband bandpass filter, a spectral region filter, a first overtone filter, a second narrowband analyte filter, and luminance filter, respectively. Generally, the individual filters are any optical filter. The individual filters optionally cover a column, row, geometric sector, and/or two-dimensional region of the two-dimensional detector array 134. Optionally, physical edges of the optical filters fall onto unused detector elements, such as a column, row, or line of filter elements. Optionally, the center of the array of illuminators provide wavelengths benefitting from a long radial distance to a detection zone, such as at low absorbance and low scattering wavelengths and/or edges of the illuminator array provide wavelengths of light benefitting from short radial distances, such as at high water absorbance wavelengths.

Referring still to FIG. 17 and referring now to the second detector array 1704, additional detector-filter configurations are described. First, the second detector array 1704 is optionally positioned with a corner proximate the outer border or edge of an illumination point, zone, or array of the photon transport system 120. Rotation of the second detector array 1704 relative to the first detector array 1702 yields a second set of distinct pathlengths, correlated depths of penetration, to probed optical pathways compared to those observed using the first detector array, as described supra. Second, the same filter elements, as used on the first detector array 1702, are optionally used on the second detector array 1704, which reduces manufacturing costs and research and development time understanding finer points, such as temperature stability of the filters. However, the physical mounting configuration of the filters are optionally different, which yields an additional set of measures of state of the subject 170. For example, the first filter, 1, as illustrated is positioned along two diagonals of the second detector array 1704, which yields three optical filter-detector combinations not observed with the first detector array 1702, where the three new combinations relate to three additional sampled pathlengths and depths of penetration of the subject 170. Similarly, the second, third, fourth, and fifth filters, 2, 3, 4, L, are positioned along diagonals across the second detector array 1704 yielding, as illustrated, eighteen additional measurements of the state of the subject 170. Further, in this example, one set of detector elements are not associated with a filter, which yields yet another set of measurements of the state of the subject 170. Further, if multiple illuminators are used providing different wavelengths, such as illuminators A, B, C, then the total number of probed pathways goes up as the product of the number of illuminators if the illuminators are sequentially used.

Referring still to FIG. 17 and referring now to the third detector array 1706, additional detector-filter configurations are described. First, the first, second, third, and fourth filters, 1, 2, 3, 4, are orientated in yet another set of configurations relative to the photon transport system 120. Notably, in this example, some of the source/detector element distances are intentionally redundant yielding internal precision, outlier, sample inhomogeneity checks, and/or sample interface 150 contact checks. For example, three elements of the second and third detector array 1704, 1706 have redundant positions of the first filter, 1. In addition, one detector element of the third detector array 1706 uses the first filter, 1, in a position not used with the first or second detector arrays 1702, 1704 yielding yet another measure of the state of the subject 170 via yet another probed optical pathway with independent detectors. Similarly, the second filter, 2, is configured with both redundant positions, relative to those used with the second detector array 1704, and with new positions, relative to those used with the second detector array 1704. In this example, the third filter and fourth filter, 3, 4, are configured at larger distances from a mean point of the illumination zone relative to the filter position configurations of the second detector array 1704. In practice, some of the third and fourth filter/detector positions are optimally probing the glucose containing dermal region 174, while others will yield information on the intervening and underlying epidermis and subcutaneous fat regions, respectively. Generally, the range of information gathered is used in post-processing to generate more accurate and precise analyte concentration information, such as through development and use of the same data used to form a person specific tissue map and/or via selective use of selected data where the selected data correlates with relatively longer optical pathlengths in glucose concentration rich layers and/or signal-to-noise ratios, further described infra.

Referring still to FIG. 17 and referring now to the fourth detector array 1708, still additional detector-filter configurations are described. In this example, still further illumination zone-to-detection zone distances are illustrated for the first, second, and third filters, 1, 2, 3. As illustrated, the second and third filters, 2, 3, such as a first overtone and a second overtone filter, extend to still greater radial distances from the illumination zone yielding still yet another set of measures of the state of the subject 170. Further, in this example, the fourth detector array 1708 is rotated to a non-symmetric orientation relative to the illumination zone, which yields an entirely new set of pathlengths and depths of penetration, as described supra.

Referring still to FIG. 17, any of the first, second, third, fourth, and luminance filters, 1, 2, 3, 4, L, are optionally longpass filters, shortpass filters, or bandpass filters. Optionally, the bandpass filters are associated with n ranges of interest at configured radial distances from the sources, where individual and/or groups of sources provide light in a linked source-filter-detector sub-group.

Referring still to FIG. 17, for clarity of presentation and without limitation a particular filter-detector combination of the first filter, 1, is described. Here the first filter, 1, is a combination band filter used for short distances between the illumination zone and detection zone. As described supra in the description of the first, second, third, and fourth detector arrays 1702, 1704, 1706, 1708, multiple short distances between the illumination zone and detection zone are probed, some of which optimally probe the glucose containing dermal layer 174 of the subject 170, some of which primarily probe the epidermis 173 of the subject 170, and some of which probe into the subcutaneous fat layer 176 of the subject 170. The availability of multiple measures of the state of the subject allows post-processing to derive information about the tissue layer thicknesses, tissue homogeneity, probed pathlengths, probed tissue depth, and/or analyte concentration of the subject 170 with optional use of redundant information, exclusion of outlier information, exclusion of non-optimally sampled tissue, and/or inclusion of optimally measured tissue. Similarly, use of the second, third, fourth, and fifth filter, 2, 3, 4, L, along with use of no filter at a variety of intelligently selected radial distances from the illumination zone based on scattering, anisotropy, and absorbance properties of the tissue of the subject 170 yield additional complementary and optionally simultaneous information on the state of the subject 170. Generally, the higher number of tissue measures benefits the transform algorithm, described infra, and reduces error in pathlength leading to more accurate glucose concentration determinations.

Referring still to FIG. 17, for clarity and without loss of generalization another example of the photon transport system 120 delivering light to the skin of the subject 170 at multiple illumination positions relative to two or more detector arrays is provided. In this example, the first detector array 1702 is illustrated with a plurality of filters along rows of detector elements. For example, a first filter, illustrated as filter 1, is optionally a combination band filter; a second filter, illustrated as filter 2, is optionally a first overtone filter; a third filter, illustrated as filter 3, is optionally a first and second overtone filter; a fourth filter, illustrated as filter 4, is optionally a second overtone filter; and a fifth filter, illustrated as filter L, is optionally a luminance filter/intensity filter. The inventor notes that the filters are arranged in readily manufactured rows, provide a spread of radial distances within a row, and fall in an order of wavelength inversely correlating with mean optical pathlength as a function of radial distance from the illuminator. Referring now to the second detector array 1704, the third detector array 1706, and the fourth detector array 1708 positioned about the illumination zone from the photon transport system 120, the inventor notes that the same five filters positioned in different configurations and/or orders as a function of radial distance from the illumination zone and/or as a function of rotation angle of the detector array yield a plurality of additional beneficially narrow range of pathlengths. For brevity and clarity of presentation, only the first filter, filter 1, is addressed. In the first detector array 1702, the first filter represents three distinct mean pathlengths from a mean illumination zone using the 1^{st} and 5^{th} detector elements, the 2^{nd} and 4^{th} detector elements, and the 3^{rd} detector element. Similarly, the second detector array filter 1704 monitors two additional mean pathlengths from the mean illumination zone using the first filter and individual detector elements. The third detector array 1706 could measure the same mean pathlengths as the second detector array 1704; however, preferably the third detector array 1706 measures still two more mean pathlengths using two pairs of detector elements with differing distances from the mean illumination zone. Similarly, the fourth detector array 1708 optionally measures a number of yet still further distinct mean pathlengths, such as by binning all six detector elements, or by binning rows of detector elements. Thus, at a first point in time, the four detector arrays 1702, 1704, 1706, 1708 optionally monitor at least eight mean pathlengths using only the first filter. At a second point in time, an additional distinct eight pathlengths are optionally monitored by illuminating a second pattern of the illustrated illumination points. The inventor notes that even illuminating all of the illumination points or only the first and second rings of illumination points, despite having the same mean point of illumination, will yield eight additional mean pathlengths in the tissue due to tissue inhomogeneity. Clearly, simultaneous use of the other four filters allows for simultaneous collections of spectra having at least forty pathlengths (8×5). Further, filter 1, is optionally different, in terms of a filter parameter such as a cut-on wavelength or a cut-off wavelength, for each detector array 1702, 1704, 1706, 1708 without complicating manufacturing, which yields still additional simultaneously probed optical tissue pathlengths. Still further, bandpass filters are optionally used to replace any of the described filters, where the bandpass filters have wavelength ranges of a passband of less than 5, 10, 20, or 30 nm. Generally, any number or detector elements, any number of detector arrays, any number of filters, and/or any geometry of filter layout are optionally used to obtain a desired number of simultaneously probed sample pathlengths in the pathmeter or wavelength resolved spectrometer. Optionally, signal from groups of common detector elements are binned to enhance a given signal-to-noise ratio.

Further, the two-dimensional detector arrays described herein optionally contain 1, 2, 3, or more detector materials 134 and/or types. For example, a single two-dimensional detector array 134 optionally contains 1.7, 1.9, 2.2, and/or 2.6 μm indium gallium arsenide detectors. For example, the 2.6 micrometer indium gallium arsenide detector is optionally optically coupled with longpass, shortpass and/or bandpass filters for the combination band 950 spectral region and/or the 1.7, 1.9, 2.2 μm indium gallium arsenide detectors are optionally coupled with longpass, shortpass, and/or bandpass filters for the first overtone region 960. Optionally and preferably, different detector types are joined along a joint and/or a seal, where the seal optionally corresponds with a joint or seal between two filter types or simply a set, such as a column, of unused detector elements.

Referring now to FIG. 18A, a multiple detector array system 1800 is described, which is a further example of a multiple two-dimensional detector array system. In this example, multiple detector types are optionally used, as described infra. Further, in this example, multiple detector sizes are optionally used, as described infra.

Referring still to FIG. 18A, additional examples of two-dimensional detector/filter arrays are provided. Referring now to the first detector array 1702 and the second detector array 1704, the second detector array 1704 relative to the first detector array illustrates:
that two detector arrays optionally vary in length and/or width by more than 5, 10, or 20 percent, which results in an ability to miniaturize a sample probe head and/or to enhance collection efficiency of delivered photons by increasing overall skin surface coverage by the detectors; and
that the row and/or columns of detector elements optionally have different single element sizes, which allows control over a range of pathlengths monitored and/or a standard deviation of pathlengths monitored with a given detector element.

Referring now to the third detector array 1706, the two-dimensional detector array 134 optionally contains sensors and/or optics to measure a range of parameters, such as a local tissue temperature, T₁, a local tissue pressure, P₁, a local tissue contact sensor, C₁, and/or a local illumination, I₁.

Still referring to FIG. 18A, a set of optional contact elements or contact sensors, such as an optical contact sensor or an electrical circuit sensing contact sensor, are used to provide multiple sample interface 150-subject 170 contact determination points, such as the illustrated four contact sensors C₁, C₂, C₃, and C₄. The contact sensors are optionally spaced in widely spaced areas of a sample probe tip 151. The widely spaced contact sensors allow determination of contact of various sections of the sample probe tip 151 with the subject 170 and the ability to determine contact between the various contact sensors and the subject 170. In a first example, as illustrated, if the first contact sensor, C₁, and second contact, C₂, sensor indicate contact while the third contact sensor, C₃, and fourth contact sensor, C₄, do not indicate contact between the sample probe tip 151 and the subject 170 then it is readily inferred that: (1) the sample probe tip 151 is tilted relative to the subject 170 or that the subject's skin has a localized curvature; (2) the first and third detector arrays 1702, 1706 contact the subject 170; and (3) that the second and fourth detector arrays 1704, 1709 do not contact the subject 170. In a second example, the contact sensors are placed near a detector array and are used to infer contact of the corresponding detector array with the subject 170. For instance: (1) the second contact sensor, C₂, placed near an outer perimeter of the sample probe tip 151 and in proximate contact with the first detector array 1702 is used to infer contact of the first detector array 1702 with the subject 170; (2) the third contact sensor, C₃, placed near a radially inner area of the sample probe tip 151 and in proximate contact with the second detector array 1704 is used to infer contact of any of the photon transport system 120, an illumination zone, and/or the second detector array 1704 with the subject 170; and (3) the third contact sensor, C₃, more than 0.1, 0.3, 0.5, or 1 mm and less than 2, or 3 mm from the fourth detector array 1708 is used to infer contact of the fourth detector array 1708 with the subject 170. Generally, multiple contacts signals from multiple contact sensors placed near a center and/or near a perimeter of the sample probe tip 151, in proximate contact with a detector array, and/or near a detector array are used to determine and/or infer contact with various surface areas of the sample probe tip 151 and the subject 170.

Referring now to the fourth detector array 1708, the two-dimensional detector array 134 is optionally designed to be read out in columns or sideways as rows, which allows each row to have a different detector element size. Increasing the detector element size as a function of radial distance away from an illuminator allows an enhanced/tuned signal-to-noise ratio as the detector aperture is larger as the number of photons exiting the skin with increased radial distance decreases. The larger aperture sizes of the detectors enhances signal-to-noise ratios as baseline noise remains constant and thermal noise increases at a smaller, less than linear, rate compared to the linear increase in signal with increased integration time. Referring now to the first through fourth detector arrays 1702, 1704, 1706, 1708, an optional range of illuminator/detector gaps are illustrated 121, 123, 125, 127 for the first through fourth detector arrays 1702, 1704, 1706, 1708, respectively. The range of illumination gaps further beneficially increases the number of probed pathlengths/pathways of the tissue by changing the distance between a mid-point of an illumination zone and a detection zone of an individual detector element.

Referring still to FIG. 18A, in a noninvasive analyzer, space is limited at the interface of the sample interface 150 and subject 170. Generally, photons enter the sample, travel a radial distance, and exit the sample. As the area of the photons entering the skin sample and/or the area of the photons being collected after exiting the sample is preferably as large as possible, any area used for other purposes hinders the measurement. Accordingly, a first set of detector readout/registration pixels 1810 and associated amplifier 1812 is optionally and preferably at an outer perimeter of the sample interface. Similarly, a second set of detector readout/registration pixels 1820 is optionally positioned at a separate perimeter location. Generally, parallel readouts of 2, 3, 4, 5, 6, 7, 8, 9, 10 or more detector array sections along the perimeter of the sample interface allows for more rapid parallel readouts of the multiple detector elements without obstructing the area of the photons entering and/or leaving the skin of the subject 170.

Referring now to FIG. 18B, yet another example of a multiple two-dimensional detector-filter array system is provided. In this example, a first detector array 1702 is configured with zones of regularly shaped filters over multiple individual detector element sizes. For example, the first filter, 1, such as a first overtone filter, covers two rows of detector elements, which aids in filter costs, alignment, masks, and/or installation. The first row of detector elements comprises smaller dimensions than the second row of detector elements, which enhances signal-to-noise ratios in each row as the time to fill detector wells in the first row of detector elements is less than the time to fill detector wells in the second row of detector elements due to the light transport/scattering properties in the 1450 to 1900 nm spectral region. The larger aperture of the second row detector elements gathers more light as a function of time compared to the first row detector elements as an area of a detector element in the second row is at least 2, 3, 4, 5, 6, 7, 8, 9, or 10 times larger than an area of a detector element in the first row. Similarly, the third and fourth rows of detector elements are optionally associated with the second optic, 2, such as the first overtone/second overtone band filter. The third row of detector elements are larger than the first row of detector elements due to fewer photons from an illumination zone exiting the skin at greater distances from the illumination zone and smaller than the second row of detector elements due to the enlarged spectral bandwidth of the first overtone/second overtone band filter. The fifth row of detector elements optionally uses a third, 3, filter, such as a second overtone filter. Generally, the area of detector elements is preferably manufactured to inversely match light density exiting the skin of the subject 170 in each optically filtered wavelength range. Here, the first detector array 1702 in this example is designed to optionally readout in rows, which allows different rows to comprise different sizes of detector elements. Optionally, filters at one or more detector elements positions are matched to wavelengths of an LED of a set of LEDs.

Referring still to FIG. 18B, a second detector array 1704 is presented in a rotated configuration about the x-axis relative to the first detector array 1702. The rotation of the second detector array 1704 yields a continuum of pathlength ranges for a row of detectors. For example, in the first detector array 1702, the first row of detectors monitor four average pathlengths of illuminated tissue due to C₂ symmetry of the detector elements in the first row, where for example the inner two detector elements observe a single first mean pathlength and the outer two detector elements observe a single second mean pathlength. However, in stark contrast, the first row of detector elements in the second detector/filter array 1704 monitor eight different mean optical pathlengths of light delivered by the photon transport system 120. Similarly, each row of detector elements in the second detector array 1704 observe, simultaneously, more mean pathlengths of photons from the photon transport system 120 compared to a corresponding row of detector elements in the first detector array 1702 due to the rotation of the second detector array in the y,z-plane relative to a line from a center of the second detector array to a center of the illumination zone. Detection of a range of pathlengths allows: (1) post data collection selection of data from only detector elements observing a given tissue depth of a given subject and/or (2) a beneficial larger number of probed tissue pathways for the transform algorithm.

### Detector Array-Guiding Optical Array Combinations

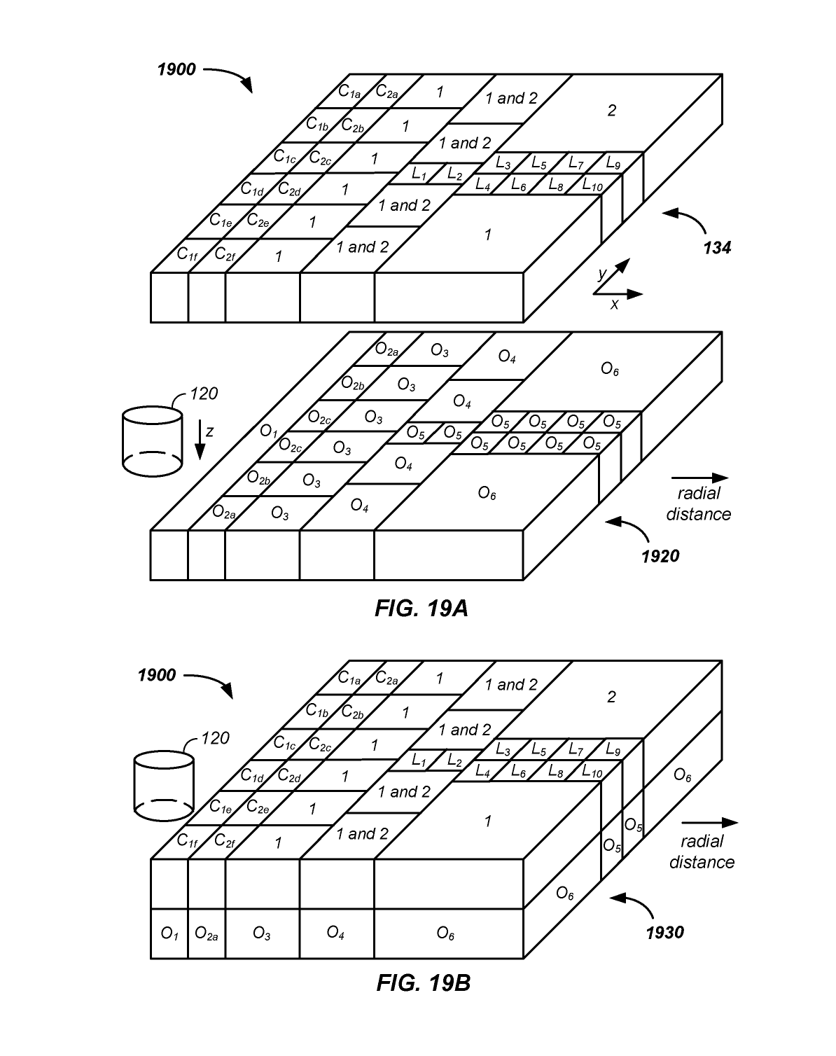

Referring now to FIG. 19A and FIG. 19B, a two-dimensional detector array-guiding optic array assembly 1900 is illustrated proximate output of the photon transport system 120, illustrated as an array of incident light optics proximate skin tissue, where the skin is not illustrated. For clarity of presentation and without limitation, the detector array-guiding optic array assembly 1900 is illustrated and described as a single assembled detector-optic unit 1930. However, optionally, the two-dimensional guiding optic array 1920 is optionally proximate the two-dimensional detector array 134, such as within less than 20, 10, 5, 2, 1, or 0.5 millimeters or is well removed from the two-dimensional detector array 134, such as at any position in the optical train between the skin of the subject 170 and the detector system 130. Further, the two-dimensional guiding optic arrays is optionally on either optical train side of one or more of the optional two-dimensional filter arrays.

Still referring to FIG. 19A and FIG. 19B, varying optional detector shape-optical filter combinations are described.

In a first case, two or more detector elements of the two-dimensional detector array 134 are optically coupled with a single optic. For example, the first column of detectors in the two-dimensional detector array 134 are coupled with a single optic, O₁. The single optic, O₁, is optionally a pathlength extending optic, which redirects light to include a vector component back toward the illumination zone resulting in a longer mean pathlength and/or a larger mean depth of penetration. The pathlength extending optic is optionally and preferably used close to the illumination zone, in this case to the left of the two-dimensional-guiding optic array assembly 1900, to yield additional photons in sampling the dermis region and fewer photons sampling solely the epidermis region, as described supra in relation to FIG. 7A. The extending optic is particularly useful with a combination band source-combination band filter-detector combination and/or with wavelengths of high absorbance, such as along shoulders of the large water absorbance band centered at 1450 and 1950 nm.

In a second case, 1, 2, 3, 4, or more individual detector elements of the two-dimensional detector array 134 are optionally each optically coupled with discrete individual optics of the two-dimensional optic array 1920. For example, as illustrated, the second column of detectors comprise combination band detectors, C_{2(a-f)}, each coupled with standard focusing optics and/or optics redirecting light to comprise a vector back toward the mean radial axis of detected incident photons, C_{2b,2e}. Optionally, the further from the mean radial axis of the detected incident photons, the greater the magnitude of the induced vector component redirecting photons back toward the mean radial axis, C_{2a,2f}.

In a third case, light gathering areas of individual optics in the two-dimensional optic array 1920 are optionally larger with increasing distance from an illumination zone proximate incident light entering skin of the subject 170 from the photon transport system 120. For example a sixth optic, O₆, optionally has a larger surface area along the x/y-plane compared to a fourth optic, O₄, which has a larger area than a third optic, O₃, which has a larger area than a second optic, O₂ₐ. For a noninvasive near-infrared spectral measurement, the generally, but not absolutely, larger collection areas as a function of radial distance from the illumination zone aid signal-to-noise ratios due to fewer photons reaching the larger radial distances. Matching of the optic size to spectral region is further described, infra.

In a fourth case, light gathering areas along the x/y-plane are chosen to enhance signal-to-noise ratios for varying spectral regions, such as the combination band region 950, the first overtone region 960, the second overtone region 970, and/or one or more narrowband analyte specific regions. For example, observed light intensity generally decreases with increased radial distance from an illumination zone for a given wavelength in the spectral region of 1100 to 2500 nm. Further, the radial distance needed to obtain quality/high signal-to-noise ratio spectra using dermal layer probing photons generally varies with radial distance from the illumination zone. The inventor has determined that a series of detector types, optical filters, and/or light gathering areas are preferentially used, such as: a combination band region detector, C₂ₐ, at close radial distance to the source coupled with a first optic collection area, O₂ₐ; a first overtone detector, 1, at an intermediate radial distance to the source coupled with a second optic collection area, O₃; a first and second overtone detector, 1 and 2, at a still further radial distance from the illumination zone coupled with a third collection optic area, O₄; and/or a second overtone detector, 2, at a yet still further radial distance from the illumination zone coupled with a fourth optic collection area, O₆.

In a fifth case, the two-dimensional detector area 134 contains a greater or first number of detector elements in a given area at a first radial distance from the illumination zone and a lesser or second number of detector elements in a second equally sized area at a second greater radial distance from the illumination zone. For example, the first number of detector elements is optionally 10, 20, 30, 40, 50, 100, 150, 200, or more percent larger than the second number of detector elements.

In a sixth case, more than one optic size, in the x/y-plane is used for a single column or row of detector elements of the two-dimensional detector array 134, such as the fourth and fifth optic, O₄ and O₅, associated with the fourth detector column in the illustrated example.

In a seventh case, one or more optical filters are optically coupled to one or more corresponding elements of the two-dimensional detector array 134 and/or to one or more corresponding elements of the two-dimensional optic array 1920.

In an eighth case, one or more detector elements of the two-dimensional detector array are optically coupled to one or more luminance filters.

In a ninth case, the optical filters or micro-optics comprise different numerical apertures at difference spatial positions in the array. For example, for a set of micro-optics, such as 2, 3, 4 or more optics positioned at increasing radial distance from an illumination zone have a corresponding 2, 3, 4 or more numerical apertures that decrease as a function of distance from an illumination zone, where differences in numerical aperture are greater than 0.5, 0.75, 1, 2, 3, 5, 10, or 20 percent. The different numerical apertures of different elements in a set of coupling optics allows coupling to a range of observed surface areas of the subject to corresponding detectors.

### Two-Dimensional Detector-Optical Filter-Guiding Optic Combinations

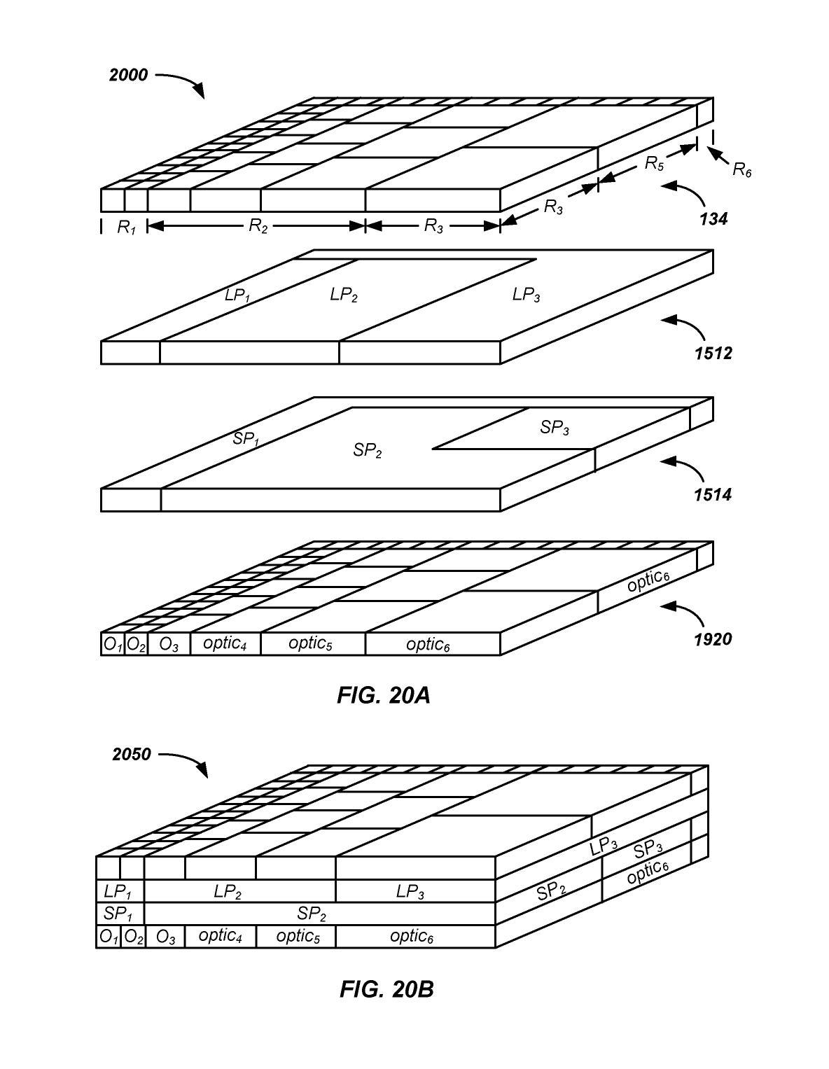

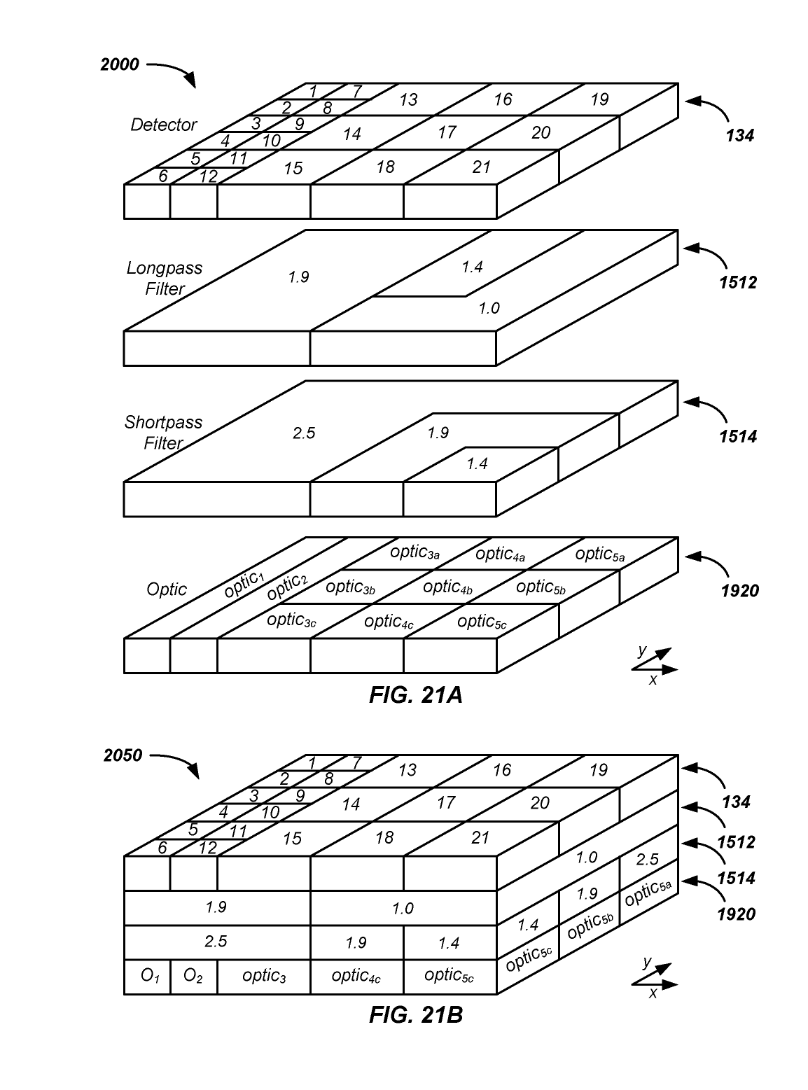

Referring now to FIGS. 20(A&B) and FIGS. 21(A&B), various exemplary combinations of the two-dimensional detector array 134-the two-dimensional filter array 1510-two-dimensional optic array 1920 are provided. Herein, examples are provided for clarity of presentation and without limitation. Generally, the examples represent any combination and/or permutation of the two-dimensional detector array 134, the two-dimensional filter array 1510, and/or the two-dimensional optic array 1920. Further, for clarity of presentation and without limitation, the two-dimensional filter array 1510 is depicted as two optional arrays, a two-dimensional longpass filter array 1512 and a two-dimensional shortpass filter array 1514. Still further, the two-dimensional detector array 134, the two-dimensional longpass filter array 1512, the two-dimensional shortpass filter array 1514, and the two-dimensional optic array 1920 are optionally individually spaced from one another, are optionally contacting each other as in a detector-filter-optic assembly 2050, and/or have gaps between one or more of the individual two-dimensional arrays.

Referring now to FIG. 20A and FIG. 20B, a first example of a two-dimensional detector-filter-optic system 2000 is described. In this first example, the two-dimensional detector array 134 contains a plurality of detectors in any geometric pattern, of one or more sizes. Further, the optional two-dimensional filter array 1510 is depicted as layers of longpass filter elements and/or shortpass filter elements, such as the two-dimensional longpass filter array 1512 and/or the two-dimensional shortpass filter array 1514. The two-dimensional longpass filter array 1512 is optionally 1, 2, 3, or more filter types, LP₁, LP₂, LP₃. Similarly, the two-dimensional shortpass filter array 1514 is optionally 1, 2, 3, or more filter types, SP₁, SP₂, SP₃. Optionally, elements of the two-dimensional shortpass filter array 1514 are present in the two-dimensional longpass filter array 1512 and vise-versa. Still further, the optional two-dimensional optic layer 1920 contains 1, 2, 3, or more optic sizes and/or types, O₁, O₂, O₃. Optionally, one or more of the longpass and/or shortpass filters overlap one or more detectors or optics of the two-dimensional detector array 134 and two-dimensional optic array 1920, respectively. Optionally, one or more edges of a longpass filter element of the two-dimensional longpass filter array 1512 do not align with one or more edges of a shortpass filter of the two-dimensional shortpass filter array 1514 or vise-versa. Generally, multiple configurations of the two-dimensional detector-filter-optic system 2000 are useful in a noninvasive analyte concentration determination, such as a noninvasive spectral determination of glucose concentration. One exemplary configuration is provided, infra.

Referring now to FIG. 21A and FIG. 21B, a second example of the two-dimensional detector/filter/optic system 2000 is described. In this second example, for clarity of presentation particular detector types, filter parameters, spectral regions, and/or optics are described that are representative of many possible detector, filter, and/or optical configurations. In this second example, filters and optics for varying spectral regions are provided in Table 2.

TABLE 2
Simultaneous Multiple Region Analysis

| | Detec- | Longpass | Shortpass | | |
|---|---|---|---|---|---|
| Detec- | tor | Filter | Filter | | |
| tor(s) | Type* | (μm) | (μm) | Optic | Region |
| 1-6 | 2.5 | 1.9 | 2.5 | Pathlength | Combination |
| | | | | Extending | Band |
| 7-12 | 2.5 | 1.9 | 2.5 | Standard | Combination |
| | | | | | Band |
| 13-15 | 2.5 | 1.9 | 2.5 | Focusing | Combination |
| | | | | | Band |
| 16 | 2.5 | 1.4 | 2.5 | Focusing | Combination |
| | | | | | Band |
| | | | | | and 1^{st} Overtone |
| 17 | 1.9 | 1.4 | 1.9 | Focusing | 1^{st} Overtone |
| 18 | 1.9 | 1.0 | 1.9 | Focusing | 1^{st} and 2^{nd} |
| | | | | | Overtone |
| 19 | 2.5 | 1.0 | 2.5 | Pathlength | Broadband |
| | | | | Reducing | |
| 20 | 1.9 | 1.0 | 1.9 | Pathlength | 1^{st} and 2^{nd} |
| | | | | Reducing | Overtone |
| 21 | 1.7 | 1.0 | 1.4 | Pathlength | 2^{nd} Overtone |
| | | | | Reducing | |

*non-limiting examples of InGaAs detector cut-off wavelengths

From Table 2, it is observed that optionally multiple spectral regions are simultaneously observed with a single two-dimensional detector array. It is further noted that observed mean sampled pathlengths and observed mean sampled depths of penetration correspond with filter types changing as a function of relative radial distance from an illumination zone; the illumination zone to the left of the illustrated two-dimensional detector and associated optics. Still further, if additional emphasis is desired for a particular spectral region, more detectors are simply used with the appropriate filter combination. For example, if more first overtone spectra are desired, the area of optics associated with detector 17 is optionally expanded along the y-axis for similar pathlengths and/or along the z-axis for longer and/or shorter mean pathlengths.

Multiple combinations of filter types and/or optic types are optionally used in the noninvasive analyte spectral determination process. Table 3 shows an exemplary configuration for a noninvasive analysis performed using the first overtone 960 and second overtone 970 spectral regions. From Table 3, it is again observed that, optionally, multiple spectral regions are simultaneously observed with a single two-dimensional detector array optically coupled to an array of filter types and/or an array of light directing optics.

TABLE 3
Simultaneous Multiple Region Analysis

| | Longpass | Shortpass | | |
|---|---|---|---|---|
| Detector | Filter | Filter | | |
| Column | (μm) | (μm) | Optic | Region |
| 1 | 1.4 | 1.9 | Pathlength | First Overtone |
| | | | Extending | |
| 2 | 1.6 | 1.4 | Standard | Analyte Band |
| 3 | 1.4 | 1.9 | Focusing | First Overtone |
| 4 | 1.6 | 1.4 | Focusing | Analyte Band |
| 5 | 1.1 | 1.9 | Standard | 1^{st} and 2^{nd} Overtone |
| 6 | 1.1 | 1.9 | Focusing | 1^{st} and 2^{nd} Overtone |
| 7 | 1.0 | 1.7 | Focusing | Extended 2^{nd} Overtone |
| 8 | 1.0 | 1.4 | Focusing | 2^{nd} Overtone |
| 9 | 1.0 | 1.4 | Pathlength | 2^{nd} Overtone |
| | | | Reducing | |

### Illumination Array

As described throughout, a spread in pathlengths of a set of detected photons within a sampling time period introduces uncertainty in an analyte concentration, such as via Beer's Law described infra. Distances between photons entering tissue and those photons exiting tissue are related to pathlength. Hence, a spread in area of illumination and/or a spread in an area of detection leads to a spread in pathlength. As described, supra, an array of small detectors is optionally substituted for a large area detector, which minimizes a spread of pathlengths entering a given detector element. Similarly, as described herein, a large illumination area on skin introduces a spread in pathlengths due to uncertainty of where a given detected photon entered the skin. Hence, another complimentary method of reducing uncertainty in pathlength is to reduce a single large illumination area into an array of small illumination areas, where individual illumination areas and/or groups of illumination areas are linked to an individual detector element and/or a group of detector elements. Generally, reducing an illumination area of the sample achieves the desired reduction in pathlength uncertainty. Multiple methods are available for irradiating a small area of skin. Herein, without loss of generality and for clarity of presentation, examples of use of a light-emitting diode (LED) array are used to illustrate near-infrared noninvasive analyte property determination, optionally using one or more detector arrays, one or more optical filter arrays, and/or one of more optic arrays.

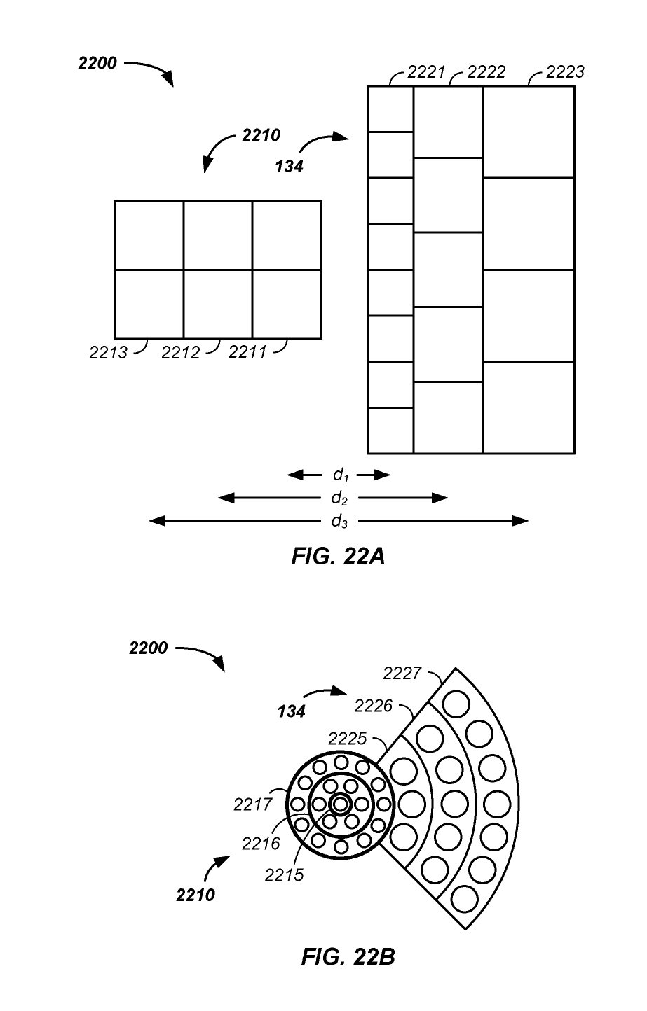

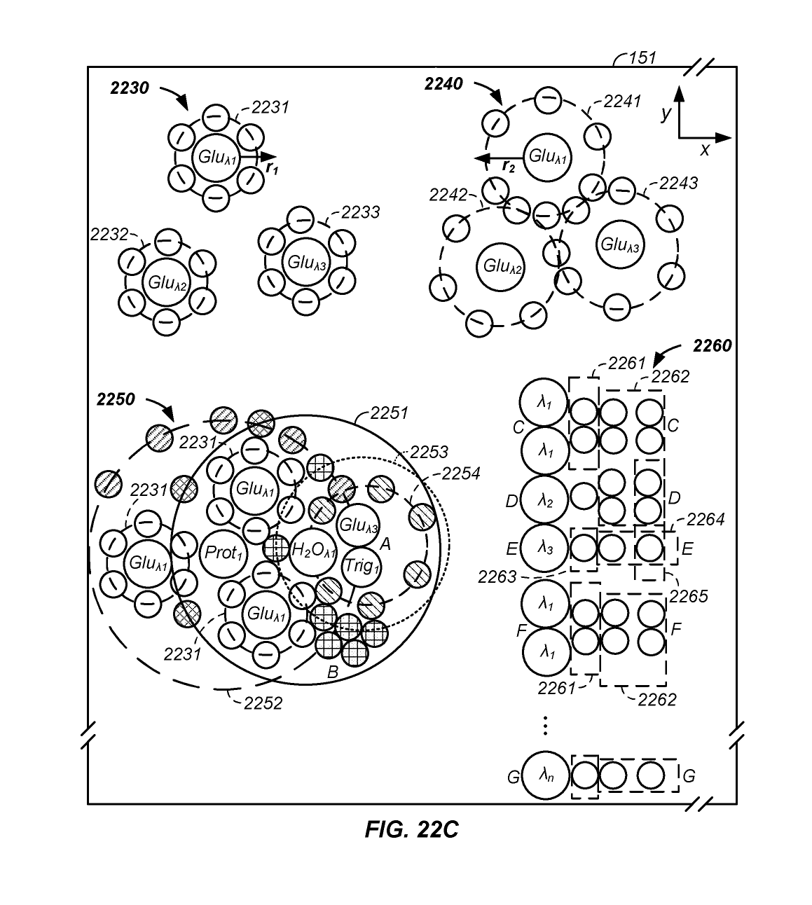

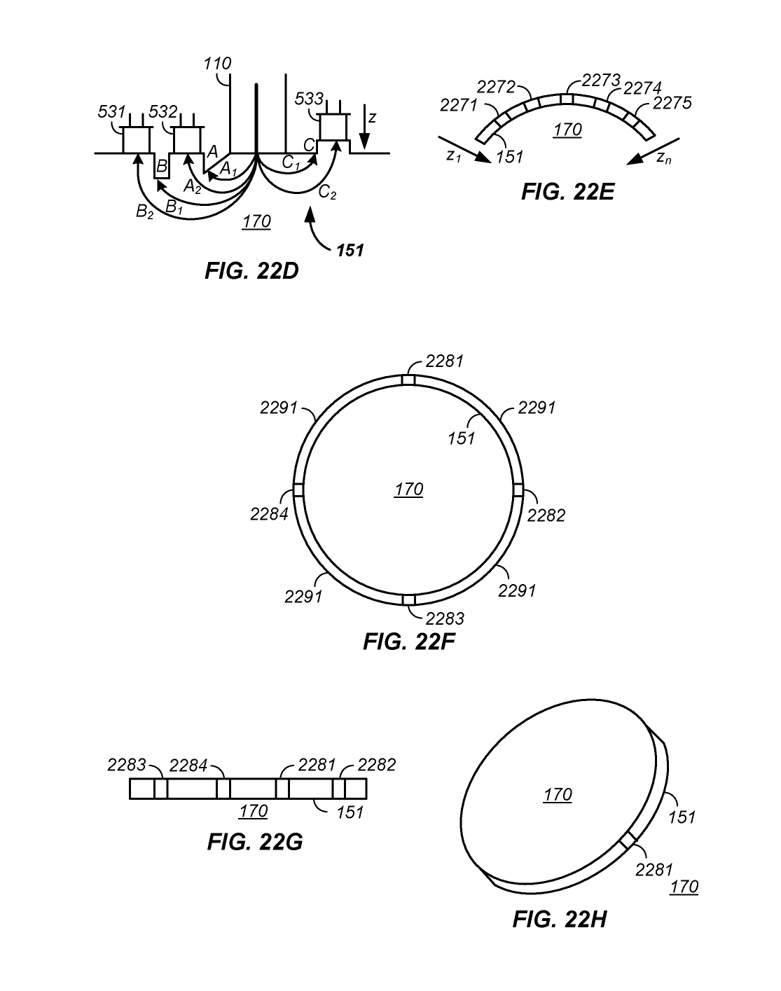

Referring now to FIG. 22A and FIG. 22B, a pathlength control system 2200 is illustrated. Generally, the pathlength control system 2200 uses: (1) a detector array, such as the two-dimensional detector array 134, which is an example of the detector 132, and/or (2) an illumination array, such as a two-dimensional LED array 2210. Optionally the detector array comprises n rings or arcs of detectors, such as an inner arc of detectors 2225 or detection zones, a middle arc of detectors 2226 or detection zones, and/or an outer arc of detectors 2227 or detection zones, where n is a positive integer of 2, 3, 4, 5, or more. Similarly, the two-dimensional LED array 2210 optionally comprises n rings or arcs of illumination zones, such as a central illumination zones 2215, a middle arc of illumination zones 2216, and/or an outer arc of illumination zones 2217, where n is a positive integer of 2, 3, 4, 5, or more. Optionally and preferably, the two-dimensional detector array 134 and the two-dimensional LED array 2210 are proximate the skin during a data collection period, such as within less than 7, 6, 5, 4, 3, 2, 1, or 0.5 millimeters from the surface of the skin.

Referring again to FIG. 22A, a first example of the pathlength control system 2200 is provided where the two-dimensional LED array 2210 is a set of at least six LEDs in an m×n array, where m and n are positive integers of at least 2, 3, 4, 5, 6, 7, 8, 9, or 10. In the illustrated example, the two-dimensional LED array 2210 and the two-dimensional detector array are: (1) each orientated substantially perpendicular to a common plane of the skin surface at the analyzer-sample interface and/or (2) are optionally separated by the spacer gap 121, described supra.

Still referring to FIG. 22A, a second example of the pathlength control system 2200 is illustrated where the two-dimensional LED array 2210 comprises a plurality of columns of LEDs, such as a first column of LEDs 2211, a second column of LEDs 2212, and a third column of LEDs 2213. As illustrated, the two-dimensional detector array 134 comprises a plurality of columns of detectors, such as a first column of detectors 2221, a second column of detectors 2222, and a third column of detectors 2223. As illustrated, individual detector elements of the two-dimensional detector array 134 comprise several surface areas, as described supra. Generally, one or more LEDs provide one or more groups of wavelengths, one group per LED type, and the detectors detect the one or more groups of wavelengths in series and/or in parallel. If detected in parallel, wavelength selection is performed by: (1) use of a time-domain spectrometer, such as a Fourier transform spectrometer; (2) use of a grating based spectrometer; (3) use of optical filters; and/or (4) using the sample and/or optical paths to selectively detect a given group or groups of wavelengths from the two-dimensional LED array 2210 with a given detector element of the two-dimensional detector array 134.

Still referring to FIG. 22A, a third example of the pathlength control system 2200 is provided where an LED of the two-dimensional LED array 2210 is optically matched with a detector element of the two-dimensional detector array 134. For instance, a first LED emits wavelengths in a first narrow spectral region. A first detector element that has a first optical filter isolating the first narrow spectral region will detect light from the first LED without sensing light emitted from other LEDs emitting light outside of the wavelength range isolated by the first optical filter. Since signal from the first detector element is linked to light from the first LED and a first distance between the first LED and the first detector element is known, the pathlength may be deduced along with the depth of penetration of the detected photons. Similarly, a second detector element with a second optical filter isolating a second wavelength range only emitted by a second LED yields a second known/narrow pathlength and/or a second known mean depth of penetration into the skin of the detected photons. Similarly, a third detector element or a third group of detector elements optically linked to a third optical filter or a third combination of optical filters isolating a third wavelength range emitted by a third LED or third group of LEDs, to the extent not overlapped with other LED output, yields a third pathlength or third group of pathlengths, where the third pathlength or third group of pathlengths comprise a standard deviation less than a standard deviation from all of a light illumination area to a single detector covering all of the detector elements. As further described, infra, a reduction in the error in the pathlength yields a corresponding reduction in error in a calculated concentration via Beer's Law. Generally, dividing an illumination area into sections, such as via the use of the two-dimensional LED array 2210, leads to a reduction in deviation of the pathlength. Similarly, dividing a detection area into sections, such as via the use of the two-dimensional detector array 134 leads to a reduction in deviation of the pathlength. When a distance between the illumination area and the detection area decreases, the reduction in deviation of the pathlength is more pronounced. Similarly, when an area of the illuminated area increases and/or an area of the detection area increases, the reduction in deviation of the pathlength is again more pronounced. In the particular case of noninvasive glucose concentration determination using near-infrared light in the range of 900 to 2600 nanometers where the distance between the middle of an illumination area and the middle of a detection area ranges from zero to four millimeters, the reduction in pathlength error with a 0.1 to 3 square millimeter illumination area and a detection area of 0.1 to 5 square millimeters is a reduction in pathlength error of at least 10, 20, 30, 40, 50, 60, 70, 80, or 90 percent. For example, for a two millimeter diameter illumination area and contacting a two millimeter diameter detection area, the radial pathlength between illuminator and detector ranges from 0.0 to 4.0 millimeters, whereas a ¼ millimeter diameter LED and a ¼ millimeter detector element at the center of the same areas yields a radial pathlength between the ¼ millimeter illuminator and the ¼ millimeter detector with ranges between 1.75 to 2.25 mm, or a range of 0.5 millimeters versus 4.0 millimeters, which is one-eighth the error. Thus, the small illumination area and smaller detection area leads to a reduction in pathlength uncertainty and a corresponding decrease in error of a determined analyte concentration.

Still referring to FIG. 22A and referring again to FIG. 22B, a fourth example of the pathlength control system 2200 is provided where properties of LEDs and position of the LEDs in the two-dimensional LED array 2210 are selected based upon at least one tissue property, such as: (1) absorbance of the sample in a wavelength range of the LED and/or (2) light scattering of the sample in the wavelength range of the LED. For clarity of presentation and without loss of generality, the use of tissue light absorbance is described for selection of a LED/LED position, though the principles apply to other tissue properties. Generally, in the near-infrared water, from 1000 to 2600 nm, is the dominant absorber of tissue. Thus, the absorbance of water or tissue as a whole is optionally used to select an LED position for a given LED. If the absorbance is high, then the pathlength is necessarily short due to inherent detectivity, D*, and bit depth of detector systems. Thus, in a spectral range of high absorbance, the LED is preferably placed at a first distance, d₁, near the detection zone, such as in a first column 2211 or outer arc 2227 of the two-dimensional LED array 2210 relative to the two-dimensional detector array 134. Similarly, if the absorbance is low, then the mean pathlength is longer. Thus, in a spectral range of low absorbance, the LED is preferably positioned away or at a third distance, d₃, from the detection zone, such as in a third column 2213 or outer arc 2227 of the two-dimensional LED array 2210 relative to the two-dimensional detector array 134. Also, in a spectral range of medium absorbance, the LED should be placed centrally or at a middle distance, d₂, such as in a second column 2212 or middle arc 2226 or ring of the two-dimensional LED array 2210 relative to the two-dimensional detector array 134. Generally, in the two-dimensional LED array 2210, LEDs emitting light in regions of high sample absorbance are preferably placed nearer a detector and vise-versa. Preferably, absorbance by the sample of light emitted by the LED correlates with distance from the detection zone with a correlation factor having an absolute value of at least 0.5, 0.6, 0.7, 0.8, or 0.9.

Still referring to FIG. 22A and FIG. 22B, a fifth example of the pathlength control system 2200 is provided where properties of the optical filters, such as an optical element of: (1) a two-dimensional filter array, (2) the two-dimensional longpass filter array 1512, and/or (3) the two-dimensional shortpass filter array 1514, coupled to the two-dimensional detector array 134 are selected based upon at least one tissue property, such as: (1) absorbance of the sample in a wavelength range targeted by a given detector element, (2) light scattering of the sample in the wavelength range targeted by a detector element, (3) anisotropy of the sample in the wavelength range targeted by the detector element and/or (4) location of a preferably coupled LED element in the two-dimensional LED array 2210. Generally, an absorbance of tissue in a wavelength range passed by an optical filter element and distance from an illumination zone has a correlation with an absolute value of at least 0.5, 0.6, 0.7, 0.8, or 0.9. For instance, referring again to FIG. 9B, at 2272 nm the absorbance is low, so a first optical filter element-detector element combination associated with this region is preferably further away from the illumination zone than a second optical filter element-detector element combination associated with a region about 2350 nm that has a relatively higher sample absorbance, which in turn is preferably further away from the illumination zone than a third optical filter element-detector element combination associated with a region about 2425 nm, that has a still higher relative sample absorbance. The same absorbance pattern repeats at both higher and lower relative wavelengths in each of the first overtone spectral region 960 and second overtone spectral regions 970.

Still referring to FIG. 22A and FIG. 22B, a sixth example of the pathlength control system 2200 is essentially a combination of the fourth and fifth examples provided in the previous two paragraphs. In this sixth example a distance between a given LED element of the two-dimensional LED array 2210 and a given optical filter characteristic, such as a bandpass, and associated detector element of the two-dimensional detector array 134 is correlated with an absorbance of the sample and/or a scattering coefficient of the tissue with a correlation coefficient having an absolute value of at least 0.5, 0.6, 0.7, 0.8, or 0.9.

The inventor notes that the LED element-optical filter parameter-detector element combination allows for the use of a multiplexed spectral analyzer that can readout in parallel signals associated with multiple wavelength regions of light passing through tissue without use of: (1) a time-domain spectrometer, such as a Fourier transform/interferogram based spectrometer; (2) use of a grating based spectrometer; and/or (3) using the sample and/or optical paths to selectively eliminate via absorbance a given set of wavelengths.

### Wavelength Weighted Analyzer

Traditionally, a spectrometer resolves broadband light into narrow bands of light where: (1) the intensity of each of narrow bands when summed yields the intensity of the broadband light, (2) the intensity of a given wavelength range of a narrow band is limited to the intensity of the broadband light source in the given wavelength range, and (3) the ratio of intensity of two wavelengths of light from the source is defined by a blackbody radiator curve. Hence, the percentage of light in a wavelength range provided by a broadband source is a fixed percentage of the broadband source light output. In stark contrast, herein in another embodiment, the percentage of light in a first wavelength range is not limited to the percentage of photons in the first wavelength range of the blackbody radiator. More particularly, two or more light sources are used to provide additional light in a preferred wavelength range and/or two or more detector elements are used to receive light in the preferred wavelength range to yield a wavelength weighted analyzer. Several examples are provided in the subsequent paragraphs to further clarify the wavelength weighted analyzer.

Referring now to Table 4, benefits of a wavelength weighted analyzer are provided. Table 4 provides approximate concentrations of four constituents of a blood/skin tissue sample. Notably, water has a very high concentration; the total protein concentration, such as from albumin and globulin, is an order of magnitude smaller; the triglyceride concentration is another order of magnitude smaller; and the glucose concentration is still smaller. In terms of Beer's law, with common assumptions such as molar absorptivities and molecular weights, this makes water very easy to analyze, protein harder, triglycerides still harder, and glucose the hardest to quantitatively determine.

As noted above, traditionally a broadband light source provides a first intensity at a first wavelength and a second intensity at a second wavelength where the ratio of the first intensity to the second intensity is fixed and is derived directly from a blackbody radiator curve. Hence, in the traditional spectrometer, the blackbody radiating light provides a fixed ratio of light at a first wavelength where water absorbs to a second wavelength where glucose absorbs. Since, some sample constituents are readily measured, such as water at a first wavelength, and some constituents are more difficult to measure, such as glucose at a second wavelength, it is beneficial to alter the fixed ratio of light provided by the single blackbody source at the first and second wavelengths. Herein, a system is provided where more light is optionally provided at wavelength ranges strongly correlated with sample constituents at lower concentration, such as glucose, and relatively less light is provided at wavelength ranges strongly correlated with second sample constituents at higher concentration, such as water. Similarly, the system optionally provides more detectors for detecting wavelengths strongly correlated with sample constituents at lower concentration and relatively fewer detectors for detecting wavelengths associated with sample constituents at higher concentration. Thus, referring again to Table 4, for the four representative sample constituents of blood/tissue, it is observed that the number of sources increases as the sample constituent concentration decreases and/or the number of detector elements increases as the sample constituent concentration decreases. Additional algorithmic, chemical, and/or physical parameters are optionally included in determining the relative number of sources and/or detectors associated with two or more wavelength regions; however, the important factor is that the ratio of provided light is not limited by the standard blackbody radiator curve nor is the number of detectors necessarily equal for different wavelength regions. Multiple examples of this are provided throughout and especially in the next set of paragraphs.

TABLE 4

| Sample | Concentration | Relative Number of | Relative Number of |
|---|---|---|---|
| Constituent | (mg/dL) | Source Elements | Detector Elements |
| Water | 50,000 | 1-2 | 2-4 |
| Total Protein | 2000-8000 | 2-5 | 4-10 |
| Triglycerides | 100-600 | 5-10 | 10-20 |
| Glucose | 80-120 | 10+ | 20+ |

Referring now to FIG. 22C, a sample probe tip 151 comprising multiple illumination zones and multiple detection zones is illustrated. The sample probe tip is described in terms of: (1) groups of illuminators and groups of detectors and (2) sub-groups of illuminators and sub-groups of detectors.

Still referring to FIG. 22C, a first group 2230 of linked sources and detectors is described. The first group 2230 comprises three spatially separated illuminators, where the number of spatially separated illuminators is optionally more than 5, 10, or 20. As illustrated, the three illuminators, denoted as Glu_{λ1}, Glu_{λ2}, and Glu_{λ3}, provide light in three wavelength regions, such as from LEDs covering bands of light. Optionally, the three LEDs each provide light in a region covered by one of the three glucose absorbance bands in the first overtone region 960.

As illustrated, six detector elements circumferentially surround each of the three illumination zones associated with the three illuminators, Glu_{λ1}, Glu_{λ2}, and Glu_{λ3} to form three sub-groups 2231, 2232, 2233. Optionally, any number of detector elements circumferentially surround each illumination zone in each of the sub-groups. However, optionally and preferably, each detector covers a narrow range of radial distances, in this case a first radius, r₁, where the first radius is optionally and preferably a radius yielding a long relative pathlength in the glucose rich dermal layer, as described above. Thus, each detector element receives light with a small standard deviation of pathlength and a resultant enhancement of confidence in the accuracy of a measured analyte, as described above. Further, the six detectors in each sub-groups each yield six times the signal compared to a traditional spectrometer with one detector element for each wavelength. Thus, in the illustrated embodiment, the analyzer 100 weights the wavelength ranges strongly correlated with glucose absorbance. Notably, the three spatially separated illuminators are far enough apart that light from a first illuminator, such as Glu_{λ1}, is not appreciably detected by detectors associated, as illustrated circumferentially surrounding, the second or third illuminator, Glu_{λ2}, Glu_{λ3}, which maintains the narrow standard deviation of pathlengths/pathways observed by each detection element.

Still referring to FIG. 22C, a second group 2240 of linked sources and detectors is described comprising a fourth, fifth, and sixth sub-groups 2241, 2242, 2243. As illustrated, the second group 2230 uses replicates of the three illuminators, Glu_{λ1}, Glu_{λ2}, and Glu_{λ3}, used in the first group 2230. Each of the three sub-groups 2241, 2242, 2243 in the second group are associated with six detection zones and/or six detectors though the number of detectors at a given radius is optionally increased with greater radii and/or is any positive integer, such as more than 1, 2, 5, 10, or 20. As illustrated, combined with the first group 2230, the percentage of light for the three glucose rich wavelength ranges, also referred to herein as spectral regions, is further increased versus that of a blackbody illuminator of a traditional spectrometer as long as the relative number of illuminators at wavelengths associated with the higher concentration sample constituents is kept lower, as is described below. More particularly, since the second sub-group 2240 also uses multiple detector elements for each illuminator, the relative number of detectors associated with wavelength regions corresponding to the low glucose concentration sample constituent is similarly enhanced compared to that of a traditional spectrometer. Notably, the second group 2240 uses a second, longer, radial distance, r₂, between each illumination zone and detector element compared to the first radial distance, r₁, used with the first group. The slightly longer second radial distance, r₂, compared to the first radial distance, r₁, has many benefits, including a new set of mean pathlengths with a corresponding greater chance of having detection signals to select from catching a dermis layer for a given subject with a dermis at a given depth, and maintained narrow standard deviations of pathlength/pathway associated with each individual detector element. As illustrated, radial distances from more than one illumination area have a common radial distance to some detection elements, such as the detection element directly between the illuminator for the first sub-group 2241, Glu_{λ1}, and the illuminator for the second sub-group 2242, Glu_{λ1}. However, the second radial distance is long enough to greatly decrease illumination from the first illuminator to detection elements not at the second radius, r₂, about the first illuminator, which again maintains a small standard deviation of pathways/pathlengths observed by each detection element.

Still referring to FIG. 22C, a third group 2250 of linked sources and detectors is described comprising multiple sub-groups. The sub-groups of the third group 2250 illustrate how different sub-groups optionally overlap, optionally weight illumination wavelengths, and/or optionally weight detection areas for a given wavelength region. As illustrated, the third group 2250 uses three of the first sub-group 2231 and/or analyte, which results in three times the light in wavelengths associated with the first glucose wavelengths, Glu_{λ1}, and eighteen (6 detectors×3 sub-groups) times the detection area for wavelengths associated with the first glucose wavelengths, Glu_{λ1}. In contrast, a seventh sub-group 2251 with an illumination area denoted, H₂O_{λ1}, uses only a single LED to provide light over a wavelength band strongly associated with water and only uses detectors, illustrated with a rising and falling cross-hatch, positioned over a small arc of an associated radius and/or a fewer detection areas, which combine to weight light and/or detection efficiency of the analyzer less to wavelengths of the more concentrated sample constituent and more to the less concentrated sample constituent. Further, an eighth sub-group 2252 provides light associated with strongly protein absorbing wavelengths at the position denoted Prot₁ and has a larger number of associated detector areas, illustrated with a rising fill. Generally, since protein has a lower concentration than water and a higher concentration than glucose, the total number of mean optical pathways between an illumination area of protein weighted wavelengths and associated detector elements is: (1) greater than a total number of mean optical pathways between an illumination area of water weighted wavelengths and associated detector elements and (2) less than a total number of mean optical pathways between an illumination area of glucose weighted wavelengths and associated detector elements. As illustrated, detectors associated with the protein weighted wavelengths cover an arc along a third of a circumference about the associated illuminator. Still further, an eighth sub-group 2252 with an illumination area denoted Trig₁ provides light with wavelengths of relatively high triglyceride absorbance and has detectors, denoted with a cross-hatch, covering a complete circumference about the triglyceride wavelength illumination zone. Generally, the third group 2250 illustrates: (1) a fewest number of illuminator-detector mean optical pathways at wavelengths dominated by the most concentrated water sample constituent; (2) a larger number of illuminator-detector mean optical pathways at wavelengths associated with the less concentrated protein sample constituent; (3) a still larger number of illuminator-detector mean optical pathways at wavelengths of strong absorbance and/or total relative signal of the less concentrated triglyceride sample constituent; and (4) a largest number of illuminator-detector mean optical pathways at peak glucose absorbance wavelengths as the glucose concentration is the lowest of the four illustrated sample constituents. More generally, any function is used to weight, relatively, more heavily analyzer performance in terms of number of source-detector combinations as the concentration of the sample constituent decreases. Further, the specific layout of the third group 2250 is only illustrative in nature. Optionally, the illuminators are placed together and/or apart in any pattern and relative distances to associated detection zones are preferably configured in terms of signal strength, sample absorbance, sample scattering, and/or sample anisotropy.

Still referring to FIG. 22C and referring again to the first group 2230 and the second group 2240, the detector elements circumferentially positioned around a given illuminator are optionally positioned at different radial distances, which yields a beneficial larger number of mean total pathlengths and/or mean pathlength in a sample layer of interest, as described in detail above. Referring now to the third group 2250, a ninth sub-group 2254 uses the wavelength of illumination used in the third sub-group 2253 while detectors in the ninth sub-group, denoted with falling lines, are eccentrically positioned about the ninth sub-group illuminator yielding a desirable range of separated mean optical pathlengths.

Still referring to FIG. 22C, region A illustrates optional multiple grouped illumination zones, where more than 2, 3, 4, or 5 sub-illumination zones provide differing wavelength bands of light with mean wavelength of illumination separated by at least 10 nm.

Still referring to FIG. 22C, region B illustrates a group of detector elements, in this case detection elements for triglyceride weighted wavelengths denoted by a cross-hatch, covering more than one radial distance from an associated illuminator, in this case the illumination area denoted, Trig₁. Generally, groups of detectors are useful for signal integration, outlier detection, and/or selection of best detector areas for a determined skin tissue type, as further discussed in the algorithm section below.

### Manufacturability

Still referring to FIG. 22C, several approaches are optionally used to enhance manufacturability and/or to reduce costs that still give the benefits described throughout. First, repetitive sub-units are optionally used as is illustrated in the first group 2230 where only the central illuminator LED is changed between groups. Second, entire groups are optionally repeated. For example, the first group 2230 is optionally used more than 1, 2, 3, 4, 5, or 10 times in the analyzer 100. Third, optical filters are optionally overlaid allowing a small number of filters to create a large number of filter zones over a range of radial distances, such as described above in relation to FIG. 20A and FIG. 20B. Fourth, the arced, circumferential, and intersecting loops of detectors described in relation to the third group 2250 are optionally laid out in a grid as described in terms of a fourth group 2260, described below.

Still referring to FIG. 22C, the fourth group 2260 of linked sources and detectors is described. The fourth group 2260 is illustrated with multiple sub-groups. A first sub-group, denoted by region C uses multiple common first illuminators, λ₁ to weight a first wavelength range and pairs of detectors at three radial distances from the first illuminators. Each pair of detectors is optionally used for signal averaging and/or for outlier detection. Variance in radial distance yields separated information on sample depth as described above. The sample itself provides some wavelength separation as a function of radial distance, as described above. A first filter 2261 and a second filter are optionally used to provide further wavelength separation, as described above. Notably, the first sub-group denoted by region C is optionally repeated, as illustrated in region F. A second sub-group, denoted by region D, uses an illuminator with a second wavelength range and multiple detectors as a function of radial distance, where more detectors are used at a larger radial distance to enhance signal-to-noise ratios as described above. The second sub-group, denoted by region D, is illustrated as coupling with a no optical filter region; an optical filter region using the second filter, which reduces costs; and a stacked filter region, where a fifth filter 2265 overlaps the second filter 2262 to create an isolated wavelength zone for resolution, as described above. A third sub-group, denoted by region E, uses an illuminator to provide light in a third wavelength region, λ₃, and three detector elements at radial distances with corresponding wavelength ranges created by a third filter 2263, a fourth filter 2264, and a combination of the fourth filter 2264 and the fifth filter 2265. As illustrated in the fourth subgroup denoted by region G, the m sub-groups provide light in illumination zones over m wavelength ranges to detection zones at detectors at n radial distances, where each illumination zone to detection zone combination is optionally filtered with the simple geometry cut optical filters, where m and n are, preferably unequal, positive integers. The inventor notes that any of the detector elements in the fourth group 2260 are optionally repeated on the opposite side of their respective illuminator with the same or different optical filters, as described above in the description in terms of FIG. 5I, FIG. 8A, FIG. 8B, FIG. 17, FIG. 18A, and/or FIG. 18B.

Still referring to FIG. 22C, in stark contrast with tradition fiber optic sample probes, such as a bifurcated fiber optic probe, that are specifically configured to interface to a small sample area to allow close spacing of all of the illumination and detection fibers, the sample probes/probe tips described herein are not so limited. Particularly, the groups of illuminators and detectors described herein optionally cover large areas of the sample, such as along the arm, around the arm, and/or over an area greater than 1, 2, 3, 4, 5, 6, 8, 10, 15, 25, or 50 cm². More particularly, the sub-groups in the groups allow the benefits of tightly and/or optimally spaced illumination zone to detection zone distances for each wavelength, while use of multiple sub-groups and/or multiple groups additionally allows sampling a large sample volume without cross-talk. Further, sampling a large surface area of the subject increases the probability of obtaining a representative sample compared to traditional fiber optic based probes that sample less than 0.5 or 1 cm² of sample surface area.

### Non-Planar Sample Probe Tip

Referring now to FIGS. 22(D-H), in an optional embodiment the sample probe tip 151 is non-planar. Generally, a protrusion from the sample probe tip is optionally used for blocking light to a detector element, referred to herein as shadowing the detector element, for providing a resistance point to sliding movement of the sample probe tip 151 relative to the subject, for forming a desired shape of a surface of the skin of the subject 170, for applying a pressure to a point of the skin of the subject 170, and/or for restricting movement of an optical coupling agent, compound, fluid, or gel. Several examples are provided to illustrate the optional non-planar nature of the sample probe tip 151 and to elucidate some of the benefits thereof.

### Example I

In a first example, referring now to FIG. 22D, the illustrated sample probe tip 151 comprises a plurality of protrusions and indentations. The sample probe tip 151 is illustrated with a first protrusion at position A, which blocks light from the source system 110 along optical path A₁ having a shallow depth of penetration into the tissue of the subject while passing light along optical path A₂ to a first light detection element 531, optical path A₂ having a deeper depth of penetration into a representative glucose concentration tissue layer of the subject 170. Similarly, the sample probe tip 151 is illustrated with a second protrusion at position B that blocks light along optical path B₁ while passing light along optical path B₂, to a second light detection element 532, which aids depth of penetration resolution, radial resolution, and/or pathlength resolution with a corresponding increase in certainty of the detected mean pathlength and the benefits thereof described above. The first protrusion optionally has a sharp edge to the sample probe tip 151 of the analyzer 100 to resist the sample probe sliding along the surface of the skin of the subject 170. Generally, a protrusion is optionally used for each detector element or group and/or any number of protrusions on the sample probe tip are optionally used. Protrusions are optionally manufactured using additive manufacturing technology, such as 3-D printing. The sample probe is also illustrated with an optional sample probe tip 151 indentation at position C. Optionally, a detector element is mounted at the base of the indentation and/or an optic is inserted into the indentation. Generally, the indentation forms physical absorbing light and/or a refractive index block change block along the z-axis that prevents light along optical path C₁ from reaching the third light detector element 523. Optionally, protrusion elements are added and/or channels or gaps are formed on a tip of the sample probe using additive technology. The protrusions and/or indentations are optionally and preferable more than 10, 20, 50, or 100 microns in depth along the z-axis and/or are optionally and preferably less than 1, ½, ¼, ⅛, or 1/16 of a millimeter in depth along the z-axis.

### Example II

In a second example, referring now to FIG. 22E, the sample probe tip 151 of the analyzer 100 forms a concave surface that couples to a convex skin tissue surface area of the subject 170, such as with a radius of curvature of more than 0.3, 0.5, 1, 2, 3, 4, 5, or 6 centimeters to couple with a finger, wrist, arm, leg, or torso. Referring again to FIG. 22C and still referring to FIG. 22E, the sample probe tip is illustrated with a fifth group 2271, a sixth group 2272, a seventh group 2273, an eighth group 2274, and a ninth group 2275 of linked sources and detectors each optionally comprising multiple sub-groups as described above in terms of the first group 2230, second group 2240, third group 2250, and fourth group 2250. Generally, any number of sources, optics, and/or detectors are coupled to the convex skin surface, such as via use of a group, to form a band of sub-sensor elements, each functioning as a sensor, to illuminate the sample along a plurality of z-axes where each z-axis is local to a respective illumination zone. Optionally, incident light from one group is detected by a detector of another group.

### Example III

Referring now to FIG. 22F, FIG. 22G, and FIG. 22H, a third example of an analyzer having a curved sample probe tip 151 is provided, where FIG. 22F, FIG. 22G, and FIG. 22H are an end view, an unrolled side view, and a perspective view, respectively, of an analyzer circumferentially wrapped about a body part during use, such as around a wrist, forearm, arm, leg, or torso. Now additionally referring to FIG. 22C, the sample probe tip is illustrated with a tenth group 2281, an eleventh group 2282, a twelfth group 2283, and a thirteenth group 2284 of linked sources and detectors, referred to herein as a circumferential group, each optionally comprising multiple sub-groups as described above in terms of the first group 2230, second group 2240, third group 2250, and fourth group 2250. Here, the circumferential group has illuminators and/or detectors positioned around the sample, such as two groups forming an angle with the center of the subject of more than 10, 20, 45, 90, or 170 degrees. Optionally and preferably, each of the tenth through thirteenth optic groups 2281, 2282, 2283, 2284 are rigid to maintain distances between respective illumination zones and detection zones. However, the substrate or connection 2291 connecting the optic groups and/or positioned between the optic groups is optionally flexible and/or elastic allowing the sample probe tip to form to the circumferential contour of the subject 170.

### Analyzer and Subject Variation

As described, supra, Beer's Law states that absorbance, A, is proportional to pathlength, b, times concentration, C. More precisely, Beer's Law includes a molar absorbance, ε, term, as shown in equation 1, repeated here for clarity:

A=εbC (eq. 1)

Typically, spectroscopists consider the molar absorbance as a constant due to the difficulties in determination of the molar absorbance for a complex sample, such as skin of the subject 170. However, information related to the combined molar absorbance and pathlength product for skin tissue of individuals is optionally determined using one or both of the spatially resolved method and time resolved method, described supra. In the field of noninvasive glucose concentration determination, the product of molar absorbance and pathlength relates at least to the dermal thickness of the particular individual or subject 170 being analyzed. Examples of spatially resolved analyzer methods used to provide information on the molar absorbance and/or pathlength usable in reduction of analyte property estimation and/or uncertainty determination are provided infra.

### Spatially Resolved Analyzer

Herein, an analyzer 100 using fiber optics is used to describe obtaining spatially resolved information, such as pathlength and/or molar absorbance, of skin of an individual, which is subsequently used by the data processing system 140. The use of fiber optics in the examples is used without limitation, without loss of generality, and for clarity of presentation. More generally, photons are delivered in quantities of one or more through free space, through optics, via LEDs, and/or off of reflectors to the skin of the subject 170 as a function of distance from a detection zone.

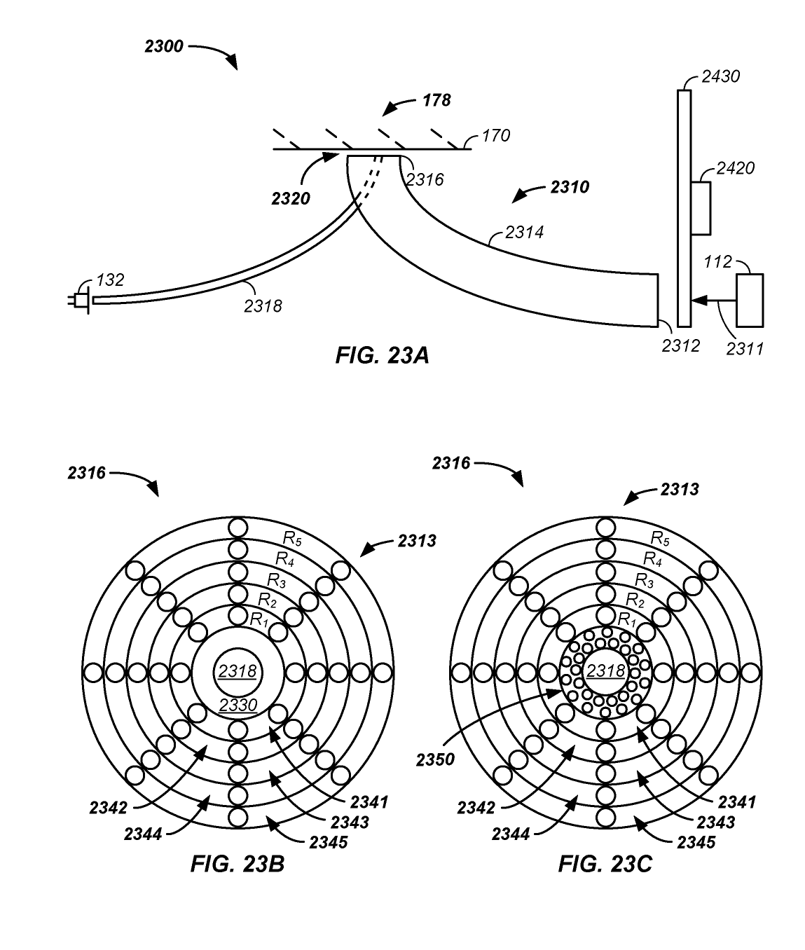

Referring again to FIG. 1 and referring now to FIG. 23A, an example of a fiber optic interface system 2300 of the analyzer 100 to the subject 170 is provided, which is an example of the sample interface 150 or of a sample interface system. Light from the source system 110 of the analyzer 100 is coupled into a fiber optic illumination bundle 2314 of a fiber optic bundle 2310. The fiber optic illumination bundle 2314 guides light to a sample site 178 of the subject 170. The sample site 178 has a surface area and a sample volume. In a first case, a sample interface tip 2316 of the fiber optic bundle 2310 contacts the subject 170 at the sample site 178. In a second case, the sample interface tip 2316 of the fiber optic bundle 2310 proximately contacts the subject 170 at the sample site 178, but leaves a sample interface gap 2320 between the sample interface tip 2316 of the fiber optic bundle 2310 and the subject 170. In one instance, the sample interface gap 2320 is filled with a contact fluid, a sample interface tape, such as a flexible fluorocarbon membrane, a Teflon® (DuPont, Wilmington, Del.) membrane, and/or an optical contact fluid. In a second instance, the sample interface gap 2320 is filled with air, such as atmospheric air. Light transported by the fiber optic bundle 2310 to the subject 170 interacts with tissue of the subject 170 at the sample site 178. A portion of the light interacting with the sample site is collected with one or more fiber optic collection fibers 2318, which is optionally and preferably integrated into the fiber optic bundle 2310. Optionally detectors, filter-detector combinations, and/or optic-filter-detector combinations are used in place of the fiber optics, as described supra. As illustrated, a single collection fiber 2318 is used. The collection fiber 2318 transports collected light to the detector 132 of the detection system 130.

Referring now to FIG. 23B, a first example of a sample side light collection end 2316 of the fiber optic bundle 2310 is illustrated. In this example, the single collection fiber 2318 is circumferentially surrounded by an optional spacer 2330, where the spacer has an average radial width of less than about 200, 150, 100, 50, or 25 micrometers. The optional spacer 2330 is circumferentially surrounded by a set of fiber optic elements 2313. As illustrated, the set of fiber optic elements 2313 are arranged into a set of radially dispersed fiber optic rings, such as a first ring 2341, a second ring 2342, a third ring 2343, a fourth ring 2344, and an n^{th} ring 2345, where n comprises a positive integer of at least 2, 3, 4, 5, 6, 7, 8, 9, or 10. Optionally, the fiber optic elements 2313 are in any configuration, such as in a close-packed configuration about the collection fiber 2318 or in an about close-packed configuration about the collection fiber 2318. The distance of each individual fiber optic of the set of fiber optic elements 2313, or light collection element, from the center of the collection fiber 2318 is preferably known.

In another embodiment, the sample interface tip 2316 of the fiber optic bundle 2310 includes optics that change the mean incident light angle of individual fibers of the fiber optic bundle 2316 as they first hit the subject 170. For example, a first optic at the end of a fiber in the first ring 1041 aims light away from the collection fiber optic 2318; a second optic at the end of a fiber in the second ring 2342 aims light nominally straight into the sample; and a third optic at the end of a fiber in the third ring 2342 aims light toward the collection fiber 2318. Generally, the mean direction of the incident light varies by greater than 5, 10, 15, 20, or 25 degrees.

Referring now to FIG. 23C, a second example of the sample side light collection end 2316 of the fiber optic bundle 2310 is provided. In this example, the centrally positioned collection fiber 2318 is circumferentially surrounded by a set of spacer fibers 2350. The spacer fibers combine to cover a radial distance from the outside of the collection fiber of less than about 300, 200, 150, 100, 75, 60, 50, or 40 micrometers. The spacer fibers 2350 are circumferentially surrounded by the radially dispersed fiber optic rings, such as the first ring 2341, the second ring 2342, the third ring 2343, the fourth ring 2344, and the n^{th} ring 2345. Optionally, fiber diameters of the spacer fibers 2350 are at least ten, twenty, or thirty percent larger or smaller than fiber diameters of the set of fiber optic elements 2313. Further, optionally the fiber optic elements 2313 are arranged in any spatial configuration radially outward from the spacer fibers 2350. More generally, the set of fiber optic elements 2313 and/or spacer fibers 2350 optionally contain two, three, four, or more fiber optic diameters, such as any of about 40, 50, 60, 80, 100, 150, 200, or more micrometers. Optionally, smaller diameter fiber optics, or light collection optics, are positioned closer to any detection fiber and progressively larger diameter fiber optics are positioned, relative to the smaller diameter fiber optics, further from the detection fiber.

### Radial Distribution System

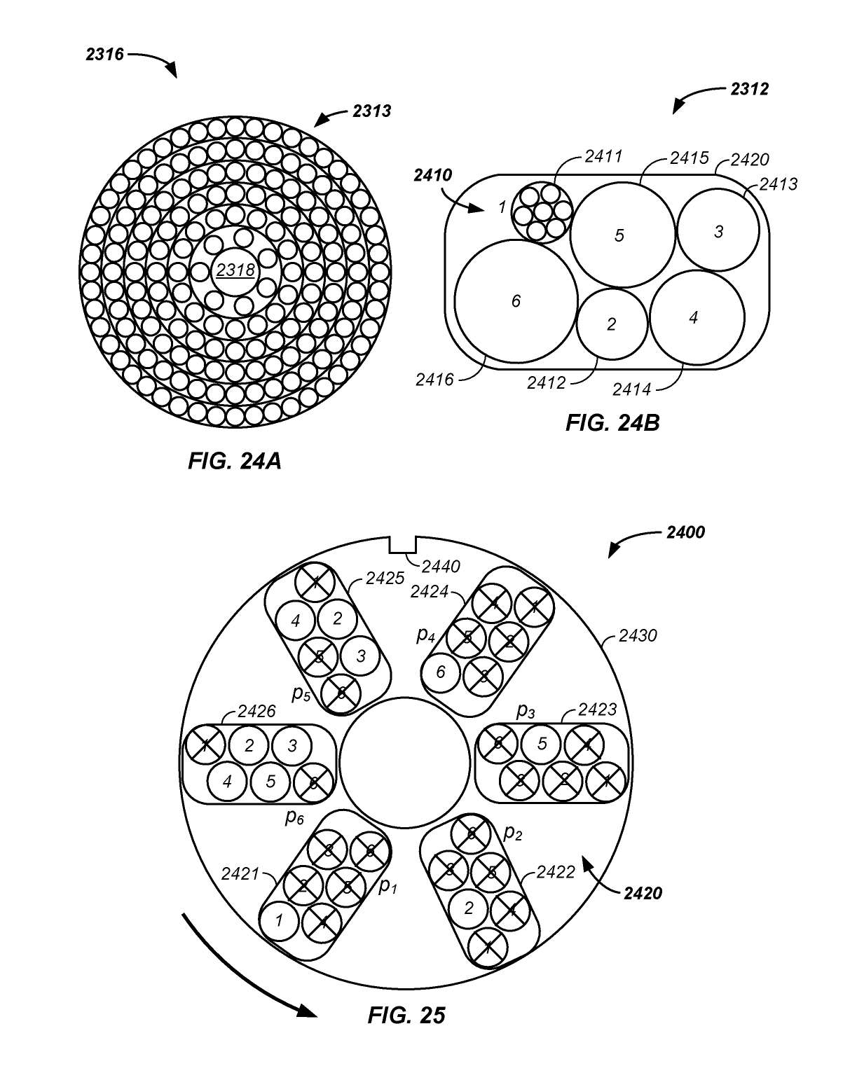

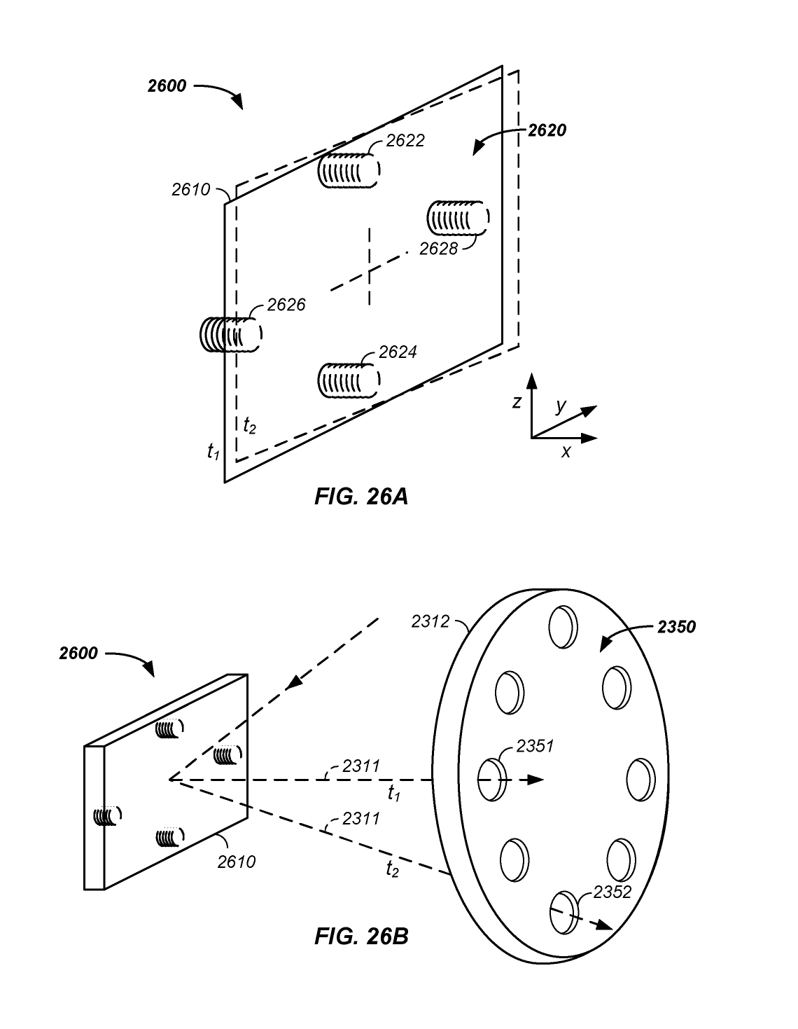

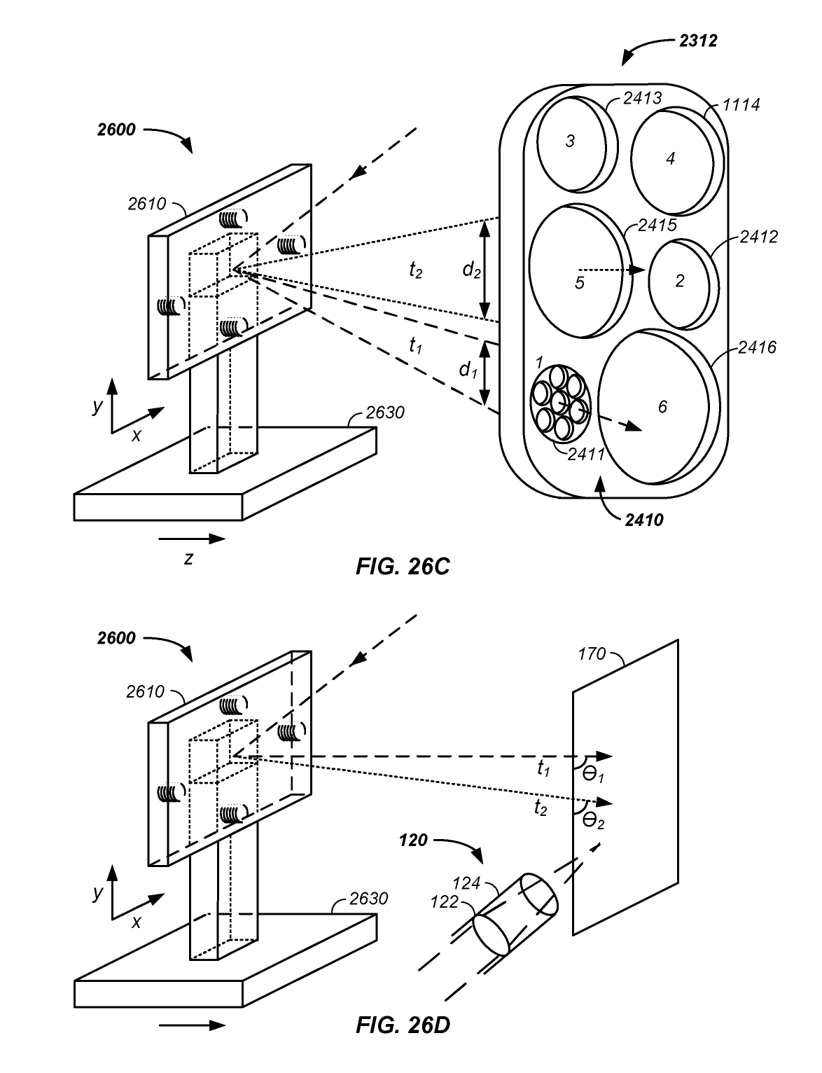

Referring now to FIG. 24A, FIG. 24B, FIG. 25, and FIGS. 26(A-D) a system for spatial illumination 2400 of the sample site 178 of the subject 170 is provided. The spatial illumination system 2400 is used to control distances between illumination zones and detection zones as a function of time. In a first case, light is distributed radially relative to a detection zone using a fiber optic bundle. In a second case, light is distributed radially relative to a detection zone using a reflective optic system and/or a lens system. Generally, the first case and second case are non-limiting examples of radial distribution of light about one or more detection zones as a function of time.

### Radial Position Using Fiber Optics

Referring now to FIG. 24A, a third example of the sample side light collection end 2316 of the fiber optic bundle 2310 is provided. In this example, the collection fiber 2318 or collection optic is circumferentially surrounded by the set of fiber optic elements 2313 or irradiation points on the skin of the subject 170. For clarity of presentation and without loss of generality, the fiber optic elements 2313 are depicted in a set of rings radially distributed from the collection fiber 2318. However, it is understood that the set of fiber optics 2313 are optionally close packed, arranged in a random configuration, or arranged according to any criterion. Notably, the distance of each fiber optic element of the set of fiber optic elements 2313 from the collection fiber 2318 is optionally determined using standard measurement techniques through use of an algorithm and/or through use of a dynamically adjustable optic used to deliver light to the sample, such as through air. Hence, the radial distribution approach, described infra, is optionally used for individual fiber optic elements and/or groups of fiber optic elements arranged in any configuration. More generally, the radial distribution approach, described infra, is optionally used for any set of illumination zone to detection zone distances using any form of illuminator and any form of detection system, such as through use of the spatially resolved system and/or the time resolved system.

Referring now to FIG. 24B, an example of a light input end 2312 of the fiber optic bundle 2310 is provided. In this example, individual fibers of the set of fiber optics 2313 having the same or closely spaced radial distances from the collection fiber 2318 are grouped into a set of fiber optic bundles or a set of fiber optic bundlets 2410. As illustrated, the seven fibers in the first ring circumferentially surrounding the collection fiber 2318 are grouped into a first bundlet 2411. Similarly, the sixteen fibers in the second ring circumferentially surrounding the collection fiber 2318 are grouped into a second bundlet 2412. Similarly, the fibers from the third, fourth, fifth, and sixth rings about the collection fiber 2318 at the sample side illumination end 2316 of the fiber bundle 2310 are grouped into a third bundlet 2413, a fourth bundlet 2414, a fifth bundlet 2415, and a sixth bundlet 2416, respectively. For clarity of presentation, the individual fibers are not illustrated in the second, third, fourth, fifth, and sixth bundlets 2412, 2413, 2414, 2415, 2416. Individual bundles and/or individual fibers of the set of fiber optic bundlets 2410 are optionally selectively illuminated using a mask 2420, described infra.

Referring now to FIG. 25 and FIG. 23A, a mask wheel 2430 is illustrated. Generally, the mask wheel 2430 rotates, such as through use of a wheel motor 2420. As a function of mask wheel rotation position, holes or apertures through the mask wheel 2430 selectively pass light from the source system 110 to the fiber optic input end 2312 of the fiber optic bundle 2310. In practice, the apertures through the mask wheel are precisely located to align with (1) individual fiber optic elements of the set of fiber optics at the input end 2312 of the fiber optic bundle or (2) individual bundlets of the set of fiber optic bundlets 2410. Optionally an encoder or marker section 2440 of the mask wheel 2430 is used for tracking, determining, and/or validating wheel position in use.

Still referring to FIG. 25, an example of use of the mask wheel 2430 to selectively illuminate individual bundlets of the set of fiber optic bundlets 2410 is provided. Herein, for clarity of presentation the individual bundlets are each presented as uniform size, are exaggerated in size, and are repositioned on the wheel. For example, as illustrated a first mask position, p₁, 2421 is illustrated at about the seven o'clock position. The first mask position 2421 figuratively illustrates an aperture passing light from the source system 110 to the first bundlet 2411 while blocking light to the second through sixth bundlets 2412-2416. At a second point in time, the mask wheel 2430 is rotated such that a second mask position, p₂, 2422 is aligned with the input end 2312 of the fiber optic bundle 2310. As illustrated, at the second point in time, the mask wheel 2430 passes light from the source system 110 to the second bundlet 2412, while blocking light to the first bundlet 2411 and blocking light to the third through six bundlets 2413-2416. Similarly, at a third point in time the mask wheel uses a third mask position, p₃, 2423 to selectively pass light into only the fifth bundlet 2415. Similarly, at a fourth point in time the mask wheel uses a fourth mask position, p₄, 2424 to selectively pass light into only the sixth bundlet 2416.

Still referring to FIG. 25, thus far the immediately prior example has only shown individual illuminated bundlets as a function of time. However, combinations of bundlets are optionally illuminated as a function of time. In this continuing example, at a fifth point in time, the mask wheel 2430 is rotated such that a fifth mask position, p₅, 2425 is aligned with the input end 2312 of the fiber optic bundle 2310. As illustrated, at the fifth point in time, the mask wheel 1130 passes light from the source system 110 to all of (1) the second bundlet 2412, (2) the third bundlet 2413, and (3) the fourth bundlet 2414, while blocking light to all of (1) the first bundlet 2411, (2) the fifth bundlet 2415, and (3) the sixth bundlet 2416. Similarly, at a sixth point in time a sixth mask position, p₆, 2426 of the mask wheel 2430 passes light to the second through fifth bundlets 2412-2415 while blocking light to both the first bundlet 2411 and sixth bundlet 2416.

In practice, the mask wheel 2430 contains an integral number of n positions, where the n positions selectively illuminate and/or block any combination of: (1) the individual fibers of the set of fiber optics 2313 and/or (2) bundlets 2410 of the set of fiber optic optics 2313. Further, the filter wheel is optionally of any shape and uses any number of motors to position mask position openings relative to selected fiber optics. For example, the mask wheel is optionally rectangular and uses two motors to move the rectangle along two perpendicular axes, where apertures in the rectangle mask control light throughput to associated fibers of the fiber bundle. Still further, in practice the filter wheel is optionally any electromechanical and/or electro-optical system used to selectively illuminate the individual fibers of the set of fiber optics 2313. Yet still further, in practice the filter wheel is optionally any illumination system that selectively passes light to any illumination optic or illumination zone, where various illumination zones illuminate various regions of the subject 170 as a function of time. The various illumination zones alter the effectively probed sample site 178 or region of the subject 170 as well as pathway and pathlength as described, supra.

Radial Position Using a Mirror and/or Lens System

Referring now to FIGS. 26(A-D), a dynamically positioned optic system 2600 for directing incident light to a radially changing position about a collection zone is provided.

Referring now to FIG. 26A, a mirror 2610 is illustrative of any mirror, lens, mirror system, and/or lens system used to dynamically and positionally direct incident light to one or more illumination zones of the subject 170 relative to one or more detection zones and/or volumes monitored by the photon transport system 120 and/or the detector system 130, such as the optic-filter-detector system described above or a detector linked to a grating based spectrometer or to a time domain to frequency domain based spectrometer, such as a Fourier transform based spectrometer.

Still more generally, the data processing system 140 and/or the system controller 180 optionally control one or more optics, figuratively illustrated as the mirror 2610, to dynamically control incident light 2311 on the subject 170 relative to a detection zone on the subject 170 that combine to form the sample site 178 through control of one or more of:
x-axis position of the incident light on the subject 170;
y-axis position of the incident light on the subject 170;
solid angle of the incident light on a single fiber of the fiber bundle 2410;
solid angle of incident light on a set of fibers of the fiber bundle 2410;
a cross-sectional diameter or width of the incident light;
an incident angle of the incident light on the subject 170 relative to an axis perpendicular to skin of the subject 170, where the incident light interfaces to the subject 170;
focusing of the incident light; and/or
depth of focus of the incident light on the subject 170.

Several examples are provided, infra, to further illustrate the use of the system controller 180 to control shape, position, and/or angle of the incident light 2311 reaching a fiber optic bundle, skin of the subject 170, and/or an element of the photon transport system 120.

Referring again to FIG. 26A, an example is provided of light directed by the photon transport system 120 from the source system 110 to the subject directly, through one or more fiber optics of the fiber optic bundle 2410, and/or through the photon transport system 120. However, orientation of the mirror 2610 is varied as a function of time relative to an incident set of photons pathway. For example, the mirror 2610 is translated along the x-axis of the mean optical path, is rotated about the y-axis of the mean optical path, and/or is rotated about the z-axis of the mean optical path of the analyzer 100. For example, a first mirror movement element 2622, such as a first spring or piezoelectric device, and a second mirror movement element 2624, such as a second spring, combine to rotate the mirror about a first axis, such as the y-axis as illustrated. Similarly, a third mirror movement element 2626, such as a third spring, and a fourth mirror movement element 2628, such as a fourth spring, combine to rotate the mirror about a second axis, such as the z-axis as illustrated, in the second time position, t₂, relative to a first time position, t₁.

Referring now to FIG. 26B, an example of the dynamically positioned optic system 2600 directing the incident light 2311 to a plurality of positions as a function of time is provided. As illustrated, the mirror 2610 directs light to the light input end 2312 of the fiber bundle 2310. Particularly, the incident light 2311 is directed at a first time, t₁, to a first fiber optic 2351 and the incident light 2311 is directed at a second time, t₂, to a second fiber optic 2352 of a set of fiber optics 2350. However, more generally, the dynamically positioned optic system 2600 directs the incident light using the mirror 2610 to any y-, z-axis position along the x-axis of the incident light as a function of time, such as to any optic and/or to a controlled position of skin of the subject 170.

Referring now to FIG. 26C, an example of the dynamically positioned optic system 2300 directing the incident light to a plurality of positions with a controllable and varying as a function of time solid angle is provided. Optionally, the solid angle is fixed as a function of time and the position of the incident light 2311 onto the light input end 2312 of the fiber bundle 2310 is varied as a function of time. As illustrated, the mirror 2610 directs light to the light input end 2312 of the fiber bundle 2310 where the fiber bundle 2310 includes one or more bundlets, such as the set of fiber optic bundlets 2410. In this example, the incident light is directed at a first time, t₁, with a first solid angle to a first fiber optic bunch or group, such as the first bundlet 2411, described supra, and at a second time, t₂, with a second solid angle to a second fiber optic bunch, such as the second bundlet 2412, described supra. In one case, the first solid angle and second solid angle do not overlap, such as at the fiber optic interface. In another case, the first solid angle and the second solid angle overlap by less than 20, 40, 60, or 80 percent. However, more generally, the dynamically positioned optic system 2600 directs the incident light to any y-, z-axis position along the x-axis of the incident light as a function of time at any solid angle or with any focusing angle, such as to any optic, any group of optics, and/or to a controlled position and/or size of skin of the subject 170 relative to a detection zone.

Referring now to FIG. 26D, an example is provided of the dynamically positioned optic system 2600 directing the incident light to a plurality of positions with a varying incident angle onto skin of the subject 170. As illustrated, the mirror 2610 directs light directly to the subject 170 without an optic touching the subject 170 or without touching a coupling fluid on the subject 170. However, alternatively the light is redirected after the mirror 2610, such as with a grins lens on a fiber optic element of the fiber optic bundle 2310. In this example, the incident light is directed at a first time, t₁, with a first incident angle, θ₁, and at a second time, t₂, with a second incident angle, θ₂. However, more generally, the dynamically positioned optic system 2600 directs the incident light to any y-, z-axis position along the x-axis of the incident light as a function of time at any solid angle, with any focusing depth, and/or at any incident angle, such as to any optic and/or to a controlled position and/or size of skin of the subject 170 relative to a detection zone. In this example, the detection zone is a volume of the subject monitored by the photon transport system 120 and/or a lens or mirror of the photon transport system 120 coupled with the detector system 130 and a detector therein.

### Adaptive Subject Measurement

Delivery of the incident light 2311 to the subject 170 is optionally varied in time in terms of position, radial position relative to a point of the skin of the subject 170, solid angle, incident angle, depth of focus, energy, and/or intensity. Herein, without limitation a spatial illumination system is used to illustrate the controlled and variable use of incident light.

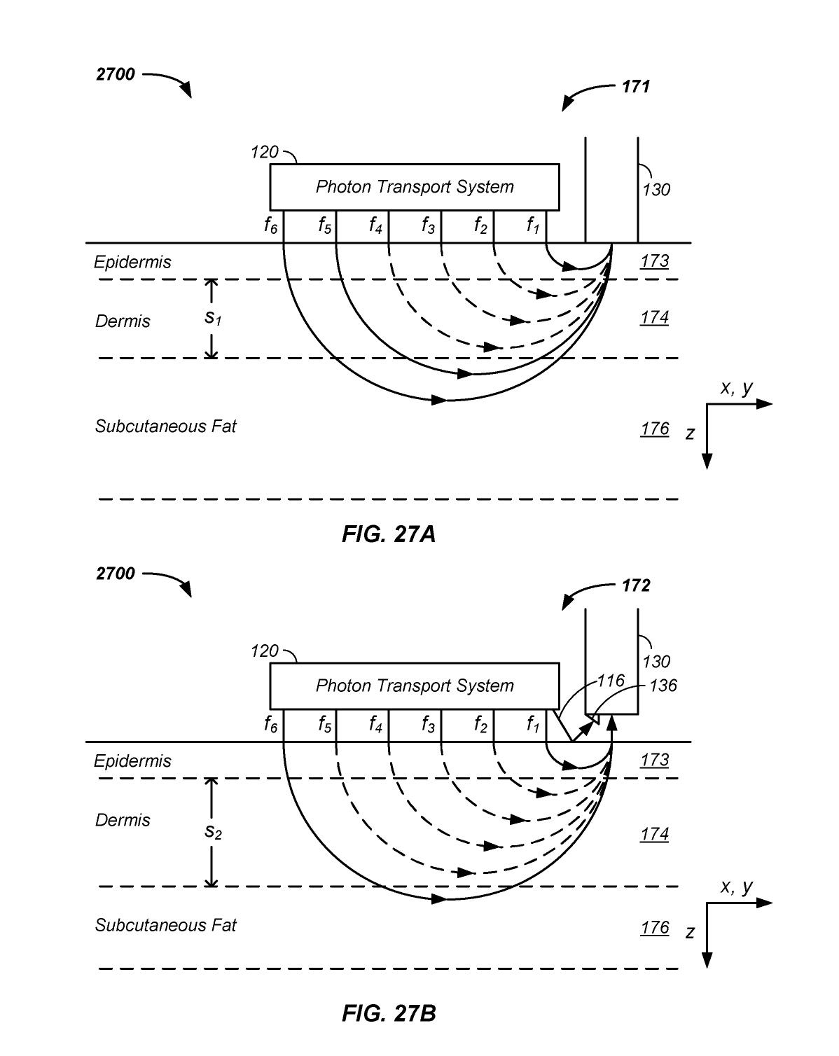

Referring now to FIG. 27A and FIG. 27B, examples of use of a spatial illumination system 2700 are illustrated for a first subject 171 and a second subject 172. However, while the examples provided in this section use a fiber optic bundle to illustrate radially controlled irradiation or illumination of the sample, the examples are also illustrative of use of the dynamically positioned optic system 2600 for directing incident light to a radially changing position about a collection zone. Still more generally the photon transport system 120 in FIGS. 27A and 27B is used in any spatially resolved system and/or in any time resolved system to deliver photons as a function of radial distance to a detector and/or to a detection zone.

Referring now to FIG. 27A and FIG. 25, an example of application of the spatial illumination system 2400 to the first subject 171 is provided. At a first point in time, the first position, p₁, 2421 of the filter wheel 2430 is aligned with the light input end 2312 of the fiber bundle 2310, which results in the light from the first bundlet 2411, which corresponds to the first ring 2341, irradiating the sample site 178 at a first radial distance, r₁, and a first depth, d₁, which as illustrated in FIG. 24A has a mean optical path through the epidermis. Similarly, at a second point in time, the filter wheel 2430 at the second position 2422 passes light to the second bundlet 2412, which corresponds to the second ring, irradiating the sample site 178 at a second increased distance and a second increased depth, which as illustrated in FIG. 27A has a mean optical path through the epidermis and dermis. The dynamically positioned optic system 2600 is optionally used to direct light as a function of time to the first position 2421 and subsequently to the second position 2422. Similarly, results of interrogation of the subject 170 with light passed through the six illustrative fiber illumination rings in FIG. 24A is provided in Table 5. The results of Table 5 demonstrate that for the first individual, the prime illumination rings for a blood analyte concentration determination are rings two through four as the first ring, sampling the epidermis, does not sample the blood filled dermis layer; rings two through four probe the blood filled dermis layer; and rings five and six penetrate through the dermis into the subcutaneous fat where photons are lost and the resultant signal-to-noise ratio for the blood analyte decreases.

TABLE 5
Subject 1

| Illumination | Deepest Tissue Layer |
|---|---|
| Ring | Probed |
| 1 | Epidermis |
| 2 | Dermis |
| 3 | Dermis |
| 4 | Dermis |
| 5 | Subcutaneous Fat |
| 6 | Subcutaneous Fat |

Referring now to FIG. 27B and FIG. 24A, an example of application of the spatial illumination system 2400 to the second subject 172 is provided. Again, the dynamically positioned optic system 2600 is optionally used to deliver light to the spatial illumination system 2400. Results of interrogation of the subject 170 with light passed through the six illustrative fiber illumination rings in FIG. 24A is provided in Table 6. For the second subject, it is noted that interrogation of the sample with the fifth radial fiber ring, f₅, results in a mean optical path through the epidermis and dermis, but not through the subcutaneous fat. In stark contrast to the first subject 171, the mean optical path using the fifth radial fiber ring, f₅, for the second subject 172 has a deepest penetration depth into the dermis 174. Hence, the fifth radial fiber ring, f₅, yields photons probing the subcutaneous fat 176 for the first subject 171 and yields photons probing the dermis 174 of the second subject 172. Hence, for a water soluble analyte and/or a blood borne analyte, such as glucose, the analyzer 100 is more optimally configured to not use both the fifth fiber ring, f₅, and the sixth fiber ring, f₆, for the first subject 171. However, analyzer 100 is more optimally configured to not use only the sixth fiber ring, f₆, for the second subject 172, as described infra.

TABLE 6
Subject 2

| Illumination | Deepest Tissue Layer |
|---|---|
| Ring | Probed |
| 1 | Epidermis |
| 2 | Dermis |
| 3 | Dermis |
| 4 | Dermis |
| 5 | Dermis |
| 6 | Subcutaneous Fat |

In yet another example, light is delivered with known radial distance to the detection zone, such as with optics of the analyzer, without use of a fiber optic bundle and/or without the use of a filter wheel. Just as the illumination ring determines the deepest tissue layer probed, control of the irradiation zone/detection zone distance determines the deepest tissue layer probed, as described in detail above.

### Incident Light Control

Referring again to FIG. 27A and FIG. 27B, specular light 116 and/or light penetrating only into the outer layers of the epidermis 173 is optionally and preferably blocked, as the specular light 116 has not transmitted through sufficient pathlength of the skin of the subject 170 to yield a usable signal-to-noise ratio in the near-infrared spectral region. As illustrated, the sample probe head is optionally non-planar. In a first optional case, as illustrated in FIG. 27A, the detection system 130 extends closer to the subject 170 than the photon transport system 120, such as an light-emitting diode element, an illumination optic, and/or an illumination fiber optic. In a second case, as illustrated in FIG. 27B, a surface area of the detector system 130 closest to the subject 170 is non-planar. As illustrated, a detection element light blocker 136 protrudes from the surface of the sample probe and/or protrudes from a surface of the detector system 130 to block at least 10, 20, 40, 60, or 80 percent of the specular light 116, such as by casting a shadow over one or more detection elements. In a third case, a portion of the sample interface 150 proximate an illumination area is non-planar to block incident photons having an angle that does not readily penetrate into the epidermis. Permutations and/or combinations of the three cases are optionally used. More generally, the sample interface 150 contains one or more bumps, extension elements, rings, and/or protrusions that extend from a base plane of the sample interface 150. Generally, the extensions shadow one or more detector elements.

Referring again to FIGS. 26A-D, the dynamically positioned optic system 2600 is optionally used as a function of time to control one or more of:
delivery of the incident light 2311 to a single selected fiber optic of the fiber optic bundle 2310;
delivery of the incident light 2311 to a selected bundlet of the set of fiber optic bundlets 2410, such as to the first bundlet 2411 at a first point in time and to the second bundlet 2412 at a second point in time;
variation of solid angle of the incident light 2311 to an optic and/or to the subject 170;
variation of radial position of delivery of the incident light 2311 relative to a fixed location, such as a center of an optic, a target point on skin of the subject 170, or a center of the sample site 178;
range or width of simultaneously illuminated radial positions for pathlength control;
incident angle of the incident light 2311 relative to a plane tangential to the skin of the subject 170 and/or an axis normal to the skin of the subject 170 at the sample site 178;
apparent focus depth of the incident light 2311 into the skin of the subject 170;
energy; and
intensity, such as number of photon per second varying from one point in time to another by greater than 1, 10, 50, 100, 500, 1000, or 5000 percent.

Optionally, any of the Michelson interferometer/time-based interferometer/fiber optic bundle systems described above use 2, 3, 4, 5, 6, or more detector elements, each detector element associated with a group of fiber optics to allow a range of bundlet patterns, fiber optic layouts, and/or parallel detection of multiple mean tissue pathways.

Optionally, the illumination system of the LED based pathmeter/analyzer described above is used in place of the illumination system of the fiber optic/grating/Michelson interferometer based system described in this section.

Optionally, detector array system described above is used in place of the detection system of the fiber optic/grating/Michelson interferometer based system described in this section.

### Data Processing System

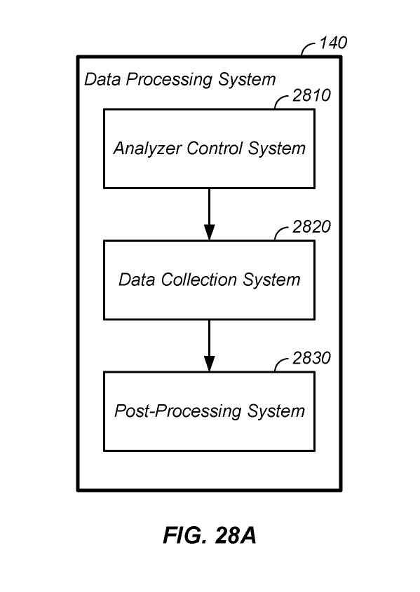

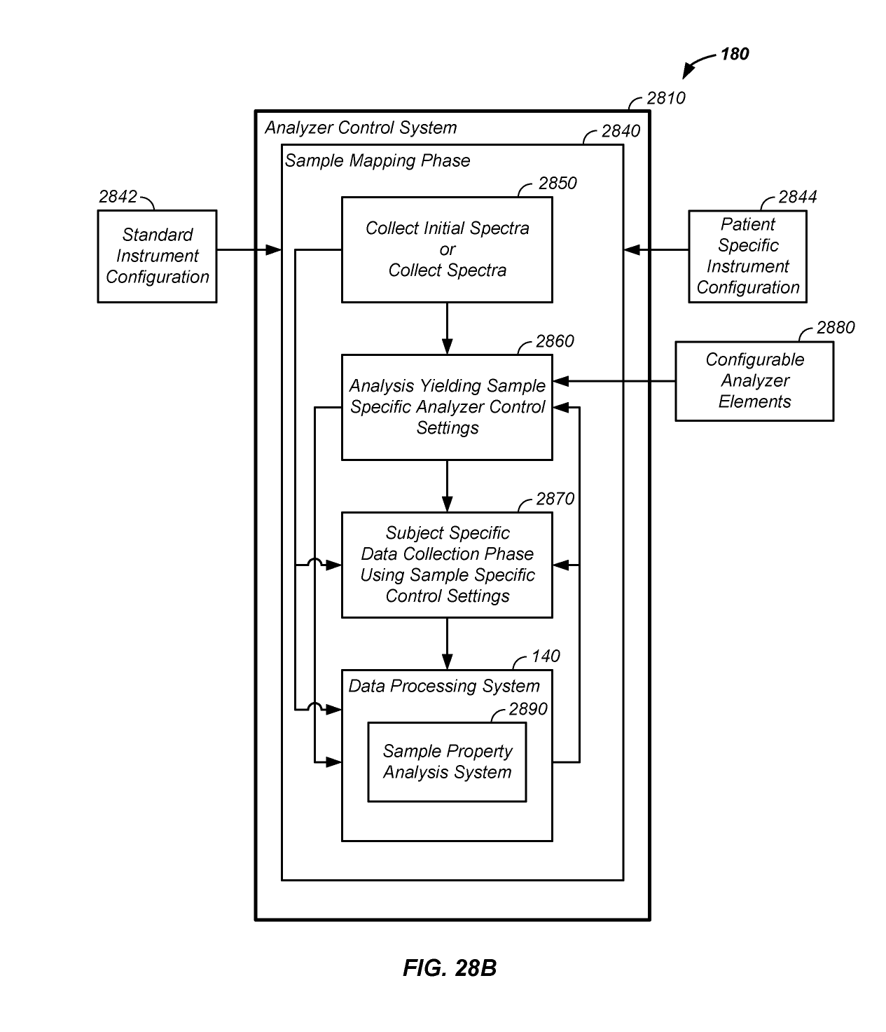

Referring now to FIG. 28A to FIG. 36C, the data processing system 140 is further described. Referring now to FIG. 28A, generally the data processing system 140 comprises: (1) an optional analyzer control system 2810, (2) a data collection system 2820, and (3) a post-processing system 2830. The analyzer control system 2810 is optionally supplemented with a sample mapping phase to yield sample specific analyzer control values. Herein, initially the optional analyzer control system 2810 is described, while the data collection system 2820 and post-processing system 2830 are described infra.

### Analyzer Control System

Referring now to FIG. 28B, the system controller 180 and the analyzer control system 2810 are further described. The analyzer control system 2810 optionally uses a sample mapping phase 2840 to define and/or fine-tune the analyzer configuration. Several configurations are provided to further describe the initial steps of the sample mapping phase 2840.

In a first configuration, initial spectra and/or all spectra are collected 2850 with a standard instrument configuration 2842. For a new patient or subject 170, the standard instrument configuration 2842 is used to collect initial spectra, where the initial spectra are optionally used to characterize the subject 170. For example, an analysis is performed on the initial spectra to yield sample and/or subject 170 analyzer control settings 2860, which are subsequently used by the analyzer 100 in a subject specific data collection phase 2870 to collect a data set for analysis of an analyte property, such as a noninvasive glucose concentration, using the data processing system 140 and/or a sample property analysis system 2890 thereof. The process of configuring the configurable analyzer elements 2880 of the analyzer 100 to the subject 170 is described infra.

In a second configuration, a patient specific instrument configuration 2844 is known, such as from a prior analysis of the subject 170 and/or through an algorithmic analysis of skin tissue type, such as using age, height, gender, and/or weight. With the known patient specific instrument configuration 2844, the analyzer 100 is configured with a correlated instrument configuration and subject specific data are optionally collected 2870 without an initial analysis. The data processing system 140 processes the resulting subject specific data, such as with the sample property analysis system 2890, to yield an analyte property, such as a glucose concentration. Again, the process of configuring configurable analyzer elements 2880 of the analyzer 100 to the subject 170 is described infra.

### Analyzer Control

The optional process of configuring the analyzer to the subject 170 is described herein in terms of a two-phase measurement system. Generally, the two-phase measurement system uses: (1) a sample mapping phase, such as a subject or group mapping phase and (2) a subject specific data collection phase/data analysis phase as described above. Multiple non-limiting examples of the sample mapping phase are provided to clarify the invention.

### Example I

In one example, in a first sample mapping phase, skin of the subject 170 is analyzed with the analyzer 100 using a first optical configuration. Subsequently, resultant sample mapping phase spectra are analyzed 2860. In the second phase, the subject specific data collection phase 2870, the analyzer 100 is setup in a second optical configuration based upon data collected in the sample mapping phase. The second optical configuration is preferably configured to enhance performance of the analyzer 100 in terms of accuracy and/or precision of estimation and/or determination of an analyte property, such as a noninvasive glucose concentration.

The examples provided herein use a single subject 170. However, more generally the sample mapping phase is optionally used to classify the subject into a group or cluster and the analyzer 100 is subsequently setup in a second optical configuration for the group or cluster, which represents a subset of the human population, such as by gender, age, skin thickness, water absorbance, fat absorbance, protein absorbance, epidermal thickness, dermal thickness, depth of a subcutaneous fat layer, and/or a model fit parameter. Grouping into clusters provides an advantage in terms of a fewer number of parameters to pass through regulatory agencies, such as the Food and Drug Administration. For clarity of presentation, several additional examples are provided, infra, describing use of a sample mapping phase and a subsequent subject specific data collection phase.

### Example II

In a second example, referring again to FIG. 28A and FIG. 28B, a first optional two-phase measurement approach is herein described. Optionally, during the first sample mapping phase, the photon transport system 120 provides interrogation photons to a particular test subject at controlled, but varying, radial distances from the detector system 130. One or more spectral markers, or an algorithmic/mathematical representation thereof, are used to determine the radial illumination distances best used for the particular test subject. An output of the first phase is the data processing system 140 selecting how to illuminate/irradiate the subject 170. Subsequently, during the second subject specific data collection phase, the system controller 180 controls the photon transport system 120 to deliver photons over selected conditions and/or optical configuration to the subject 170.

In a third, fourth, and fifth example, the dynamically positioned optic system 2300 described above in relation to FIGS. 24A to 27B is used to described control of incident photon positions; however, examples are also relevant to the controlled illumination zone to detection zone distances described above in relation to FIGS. 2A to 22C.

### Example III

Referring again to FIGS. 24A to 27B, in a third example, a first spectral marker is optionally related to the absorbance of the layer of subcutaneous fat 176 for the first subject 171. During the first sample mapping phase, the fifth and sixth radial positions of the fiber probe illustrated in FIG. 24A, yield collected signals for the first subject 171 that contain larger than average fat absorbance features, which indicates that the fifth and sixth fiber rings of the example fiber bundle are not beneficially used in the subsequent second data collection phase, which more generally establishes an outer radial distance for subsequent illumination. Still in the first sample mapping phase, probing the tissue of the subject with photons from the fourth fiber ring yields a reduced signal for the first spectral marker and/or a larger relative signal for a second spectral marker related to the dermis 174, such as a protein absorbance band or an algorithmic/mathematical representation thereof. Hence, the data processing system 140 yields a result that the fifth and sixth radial fiber optic rings or distance of the fiber bundle 170 are preferably not be used in the second subject specific data collection phase and that the fourth radial fiber optic ring or distance should be used in the second subject specific data collection phase. Subsequently, in the second subject specific data collection phase, data collection for analyte determination ensues using the first through fourth radial positions of the fiber bundle, which yields a larger signal-to-noise ratio for dermis constituents, such as glucose, compared to the use of all six radial positions of the fiber bundle. Optionally, data already collected in the mapping phase is subsequently re-used in the data analysis phase.

### Example IV

Still referring to FIGS. 24A to 27B, in a fourth example, the first sample mapping phase of the previous example is repeated for the second subject 172. The first sample mapping phase indicates that for the second subject, the sixth radial illumination ring of the fiber bundle illustrated in FIG. 24A should not be used, but that the fourth and fifth radial illumination ring should be used.

### Example V

Still referring to FIGS. 24A to 27B, in a fifth example, the first mapping phase determines positions on the skin where papillary dermis ridges are closest to the skin surface and positions on the skin where the papillary dermis valleys are furthest from the skin surface. In the subsequent subject specific data collection phase, the incident light is optionally targeted at the papillary dermis valleys, such as greater than 50, 60, or 70 percent of the incident light is targeted at the papillary dermis valley and less than 30, 40, or 50 percent of the incident light is targeted at the papillary dermis ridge. The increased percentage of the incident light striking the papillary dermis valley increases the number of photons sampling the underlying dermis layer, where blood borne analytes reside, which increases the signal-to-noise ratio of collected data and lowers resultant errors in blood borne analyte property determination. Similarly, the first mapping phase is optionally used to target incident light around any interference, such as a hair follicle or localized skin damage.

### Example VI

Referring again to FIGS. 2A to 23C, in a sixth example, the test parameters and results of the previous three examples are optionally applicable to selection of the illumination zone to detection zone distances described above in terms of the LED/two-dimensional illumination array and/or the two-dimensional detector array systems.

Generally, a particular subject is optionally probed in a sample mapping phase or initial spectra collection phase 2850, the resulting spectra are analyzed to yield sample specific analyzer control settings 2860, and the analyzer 100 is configured with the analyzer control settings in a subsequent subject specific data collection phase 2870. While for clarity of presentation, and without loss of generality, radial distance was varied in the provided examples, any optical parameter of the analyzer is optionally varied in the sample mapping phase, such as sample probe position, incident light solid angle, incident light angle, focal length of an optic, position of an optic, energy of incident light, and/or intensity of incident light. Optionally, the sample mapping phase and sample specific data collection phase occur within less than 1, 5, 10, 20, or 30 seconds of each other. Optionally, the subject 170 does not move away from the sample interface 150 between the sample mapping phase and the subject specific data collection phase. Generally, the spatial methods yield information on pathlength, b, and/or a product of the molar absorptivity and pathlength, εb, which is not achieved using a standard spectrometer.

The inventor recognizes that post-processing allows selection of optical pathways that have penetrated to appropriate depths in the tissue. However, multiple benefits exist from altering the hardware configuration prior to data collection. In a first example, time is not spent collecting unneeded data, such on the fifth and/or sixth collection rings in the third and fourth examples described above. The inventor has determined that a reduction in data collection time results more accurate noninvasive glucose concentration determination as the dynamic skin tissue has less time to change into confounding states. In a second example, some analyzer parameters may only be optimized prior to data collection, such as source-detector integration time, detector gain setting, and/or position of a dynamic optic. To still further clarify the analyzer control system 2810 or analyzer configuration system, additional non-limiting examples provided with an emphasis on integration time, gain setting, and dynamic optics.

### Integration Time Controlled Analyzer

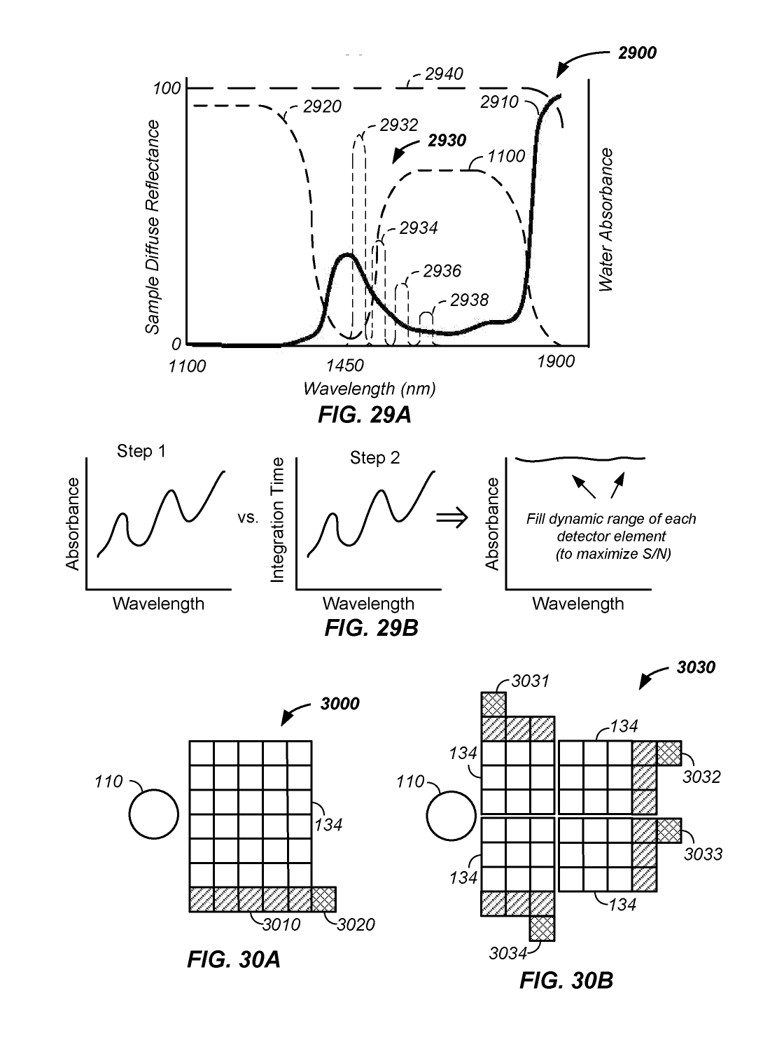

Referring now to FIG. 29A, an optional integration control system 2900 for the analyzer 100 is described. A typical non-integrated noninvasive spectrum 2920 is illustrated where the response signal, illustrated using diffuse reflectance, is dominantly an inversion of the water absorbance 2910 of the sample. As a result, the intensity to noise ratio varies across the spectrum. For example, the low sample/water absorbance in the second overtone region 970, results in: (1) a single detector element, such as in a Fourier transform based analyzer, being dominated by light in the second overtone region 970 while much less light is detected in the first overtone region 960 or (2) for an array detector, the integration time of a column or row of detectors representing different wavelengths, the readout time to prevent detector saturation is controlled by the amount of light in the second overtone region 970 or region of highest observed intensity. As a result, spectral regions that have lower intensity, such as the first overtone region 960, collect less light and for a uniform signal absorbance and have a lower signal-to-noise ratio. However, using an illumination array and/or a detector array, as detailed above in relation to FIGS. 2A to 22C, the collected signal optionally fills all detector wells to a uniform extent or optionally to within less than 30, 20, or 10 percent of each other as described in the next paragraphs.

Still referring to FIG. 29A, the integration control system 2900 for the analyzer 100 is further described. As illustrated, at a first wavelength region 2932 with high sample absorbance and/or low detected light intensity, multiple approaches are used to increase observed intensity, such as one or more of: (1) providing at least a 10, 20, 50, 100, or 200 percent longer integration time of illumination from a first light emitting diode at the first wavelength region 2932 of high sample absorbance relative to a second light emitting diode at a second wavelength region 2934 of lower sample absorbance; (2) providing multiple filter-detector elements for the first wavelength region, such as a ring of detector elements about a corresponding source; (3) using multiple bundlets/row of source-detector combinations, such as the three sub-groups 2231, 2232, 2233 described above; and/or (4) using varying integration/detector area size, such as described above in relation to FIG. 18A and FIG. 18B. Similarly, the second wavelength region 2934 with medium-high sample absorbance and/or medium low detected light intensity optionally uses any of the four approaches relative to a third wavelength region 2936 with medium sample absorbance and/or medium detected light intensity and/or a fourth wavelength region 2938 with low sample absorbance and/or high detected light intensity. More generally, any two or more wavelength regions are optionally weighted in the spectrometer by: (1) providing at least a 10, 20, 50, 100, 200, or 500 percent longer integration time of illumination at one wavelength relative to the other; (2) providing 2, 3, 4, 5, 10, or more source-detector element combinations at one wavelength relative to the other; (3) using 2, 3, 4, 5, or more bundlets/row of source-detector combinations at one wavelength relative to the other, and/or (4) using at least a 10, 20, 50, 100, or 200 percent larger integration/detector area size at one wavelength relative to the other. By taking a set of sources providing light at different wavelengths, such as a set of more than 5, 10, or 15 LEDS, and varying the relative use of each type of LED as described throughout, such as in this paragraph and in the description of FIG. 2A to FIG. 22C, a spectrum is optionally obtained that fills detector wells evenly, such as in an integrated noninvasive spectrum 2940. By comparing the integrated noninvasive spectrum 2940 to the traditional non-integrated noninvasive spectrum 2920, in view of knowledge of signal-to-noise ratio optimization, it is observed that relatively higher signal-to-noise ratios are observed for the integrated noninvasive spectrum 2940 in regions of higher water absorbance, regions of higher sample absorbance, and/or regions of less collected light per unit time compared to the traditional non-integrated noninvasive spectrum 2920.

Still referring to FIG. 29A and referring now to FIG. 29B, a generalized integration time approach to filling the dynamic range of a set of detector elements is illustrated. As illustrated, an integration time as a function of wavelength is optionally: (1) directly related to absorbance as a function of wavelength and/or (2) inversely related to observed non-integrated intensity as a function of wavelength, which yields a uniformly filled dynamic range of detector elements as a function of wavelength. Generally, compared to a reference detector element, such as responsive to a given wavelength range, other detector elements are preferably filled to at least 60, 70, 80, 90, or 95 percent of the dynamic range of the reference detector element to yield the highest signal-to-noise ratios as each detector element has a constant background noise level and increased intensity yields a higher intensity/signal-to-noise ratio.

Referring now to FIG. 30A, a traditional detector array, such as on a single-lens reflex (SLR) camera, contains an array of detector elements 134, a serial register 3010 that shifts one row of the detector element response at a time into a readout vector of elements, and a readout element 3020 and/or amplifier that sequentially reads the readout vector of elements.

Referring now to FIG. 30B, a multi-two-dimensional detector array readout system 3030 is illustrated having multiple benefits. First, since in a noninvasive glucose concentration analyzer the majority of the signals are close to the illumination zone of the source system 110, the serial register 3010 and the readout element for a given detector array are optionally and preferably positioned radially away from the illumination zone relative to detector elements of associated rows and/or columns of the detector array. Second, since the readout element 3020 operates in series, optionally and preferable multiple detector arrays are used to speed the data transfer process, which allows more sample integration time and a resultant higher signal-to-noise ratio in a limited data collection time period. Third, the position of the multiple detector arrays are optionally orientated to place corresponding serial arrays and readout elements about a perimeter of the combined detector arrays. Fourth, as illustrated a single detector array is optionally used multiple times and/or in multiple orientations to reduce manufacturing costs, to ease production, and/or to reuse software control/readout code.

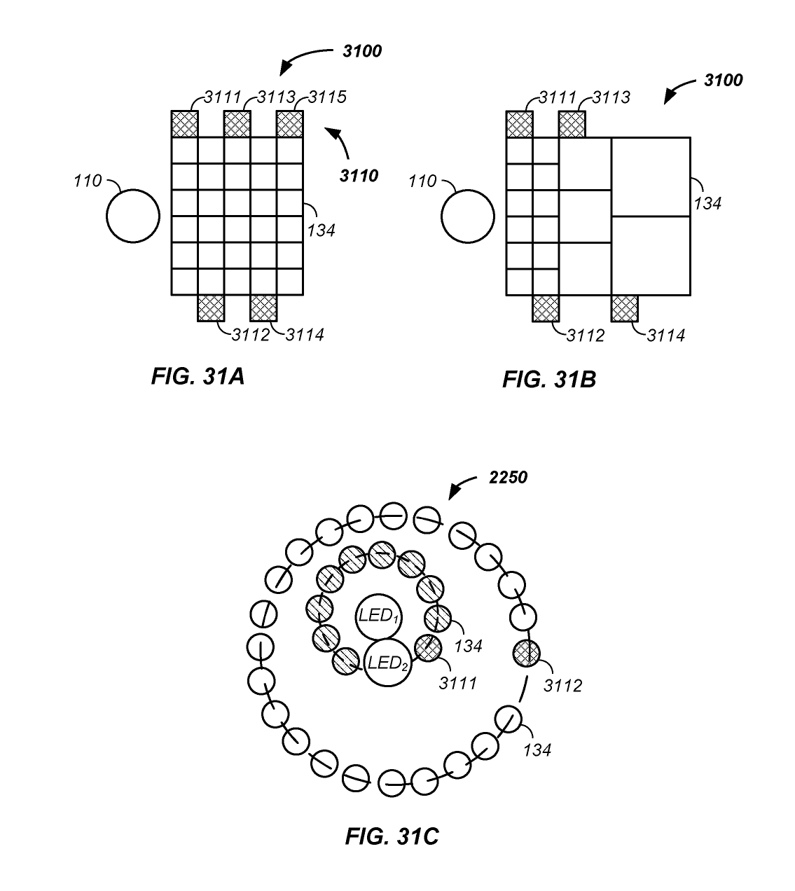

Referring again to FIGS. 29A and B and referring now to FIG. 31A, a multiple/parallel two-dimensional detector array readout system 3100 is described. As described above, wells of detector elements closest to an illumination zone of a source system 110 typically fill more rapidly than wells of detector elements positioned at larger radial distances from the illumination zone. Thus, if all detector elements must be read out together, to avoid saturating radially inward detector elements, the radially outward detector elements are read before optimally filling the dynamic range of the detector wells. However, as illustrated using multiple readout elements, different detectors at different radial distances are optionally read out after different integration time and/or at different read time frequencies. For example, a first column of detector elements of the detector array 134 is read out using a first readout element, while detector elements of progressively larger average radial distances of the detector array 134 are optionally read out with a second, third, fourth, or fifth readout element, 3112, 3113, 3114, 3115, of n readout elements in a readout array 3310, which allows each column of the detector array to be read out independently, with progressively longer integration times as a function of mean radial distance from the illumination zone, and/or with different frequencies, such as a progressively longer frequency as a function of mean radial distance of associated detector elements from the illumination zone. Thus, each mean radial distance of detector elements, optionally and preferably associated with different wavelength ranges, such as through coupling with optical filters, is read out with integration times properly filling the dynamic range of the associated detector elements, which is used to enhance signal-to-noise ratios for each wavelength, radial distance, and/or path in the pathmeter.

Still referring to FIG. 31A and referring now to FIG. 31B, the multiple and/or parallel readout of the multiple/parallel two-dimensional detector array readout system 3100 is optionally used in combination with a larger sample detection zone area as a function of radial distance, as illustrated in FIG. 31B. The larger detection zone area is optionally achieved by one or more of: (1) binning more detector elements at larger radial distances from a corresponding illumination zone; (2) using a larger detector element size as a function of increasing distance from the illumination zone; and (3) using a larger surface area of a surface of a focusing optic facing the subject 170 as a function of increasing radial distance from a corresponding incident light irradiation zone. Each of the larger sample detection zone area approaches increase the number of photons collected per unit time and lead to enhanced collected signal-to-noise ratios.

Still referring to FIG. 31A and referring now to FIG. 31C, the multiple and/or parallel readout of the multiple/parallel two-dimensional detector array readout system 3100 is illustrated in an alternative layout where individual readout elements are electronically coupled with detector elements in an arc and/or ring, such as about a corresponding illumination element. As illustrated, the first readout element 3111 is used to read out detector responses from an inner ring of detectors about a first light emitting diode and the second readout element 3112 is used to read out detector responses from an outer ring of detectors about a second light emitting diode. Further, 3, 4, 5, 10, 15, or more readout elements are optionally electronically coupled to detector elements in corresponding arcs, rings, concentrically oriented rings, eccentrically orientated rings, lines, or groups of detector elements, such as in the detector layouts illustrated in FIG. 22C and FIG. 24A described above. Generally any number of readout elements are independently coupled with any number of detector elements in any layout configuration to more effectively use dynamic well size of the coupled detector elements by allowing independent, optionally parallel, readout of detector elements when integration detector wells are determined, calculated, and/or estimated to be sufficiently full. Optionally, the time resolved/non-uniform readout process allows serial data transfer while data collection continues, as described infra.

### Data Processing System

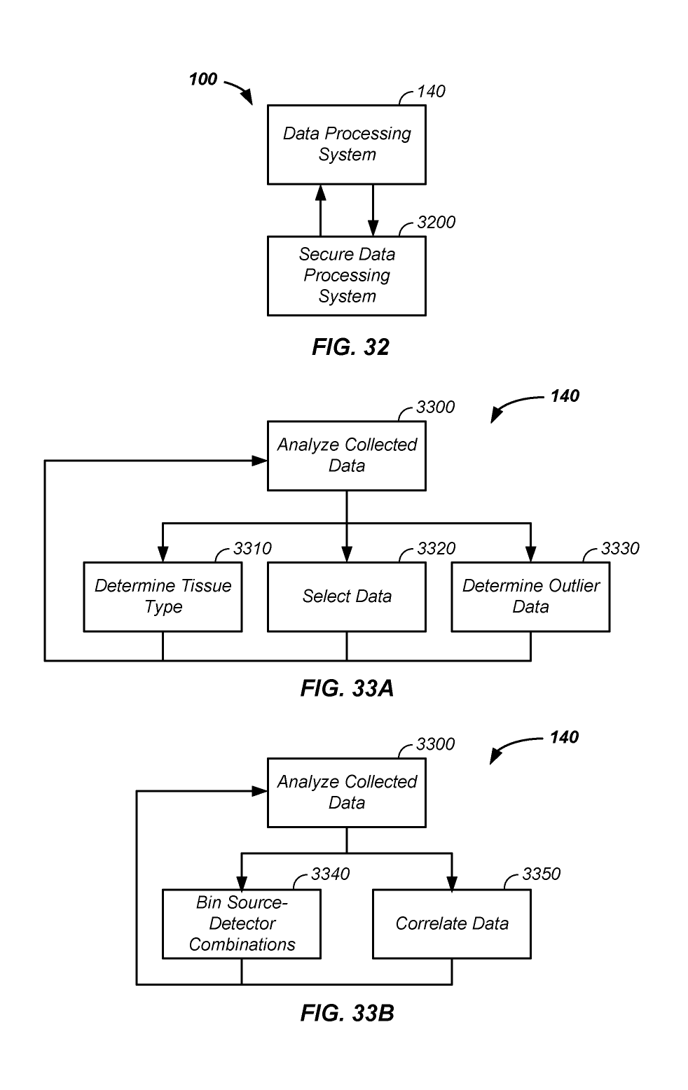

Referring again to FIG. 1 and referring now to FIG. 32, the data processing system 140 of the analyzer 100 is further described. Optionally, the data processing system 140 and/or the system controller 180 transfers data to a secure data processing system 3200, which is an example of the remote system 194. Optionally, the data transfer uses the personal communication device 192 and/or the wireless communication system 196.

### Parallel Data Collection/Processing

Still referring to FIG. 32 and referring again to FIGS. 31(A-C), in an optional embodiment, data transfer, such as to the secure data processing system 3200, initiates using data from one of the readout elements 3110 while data collection is simultaneously performed using two or more other readout elements. Similarly, data transfer from one or more of the readout elements 3110 occurs in parallel with: (1) continued data collection with additional members of the readout elements 3110; (2) in parallel with data processing, such as at the secure data processing system 3200; and/or (3) while the analyzer 100 and/or personal communication device 192 receives processed results from the remote system 194, which allows: (a) more data collection per unit time, (b) semi-continuous use of the analyzer 100, and/or (c) continuous use of the analyzer 100.

### Data Analysis/Algorithm

Referring now to FIG. 33A, the data processing system 140 is further described. When the data processing system 140 analyzes the collected data 3300, a determination of the subject tissue type 3310 is optionally performed. For example, any of a thickness of the stratum corneum at the sample interface, depth of the epidermis 173, thickness of the epidermis 173, depth of the dermis 174, thickness of the dermis 174, depth of the subcutaneous fat 176 or a subcutaneous fat layer are determined for the subject 170.

Still referring to FIG. 33A, the data processing system 140 performs a step of data selection 3320, which optionally uses information for the step of determining the subject tissue type 3310. Further, an outlier determination step 3310 is optionally used to further narrow time periods of data to be used from a given detector element or if any data is to be used from a given detection element of the two-dimensional detector array 134. The outlier determination step 3310 is also optionally used to determine which optical paths are to be included in data analysis, such as in the LED-filter-detector system described above in terms of FIG. 2A to FIG. 22C or in the Fourier transform-fiber optic-detector system described above in terms of FIG. 23A to FIG. 27B.

Referring now to FIG. 33B, the data processing system 140 is further described. The step of analyzing collected data 3300 optionally bins source-detector combinations 3340; bins source-filter-detector combinations; and/or bins signals as a function of time. Generally, the binning step trades spatial resolution and/or time resolution for an increase in the signal-to-noise ratio, which has sample specific benefits. For example, if the tissue type determination step 3310, described above, determines a thickness of the dermis 174 that covers two source-detector combinations with similar mean pathlengths in the dermis layer, then signals from the two source-detector combinations are preferably binned.

Still referring to FIG. 33B, the data processing system 140 is further described. The step of analyzing collected data optionally correlates data 3350 to yield additional information on outlier determination and/or sample property information.

### Sample Mapping

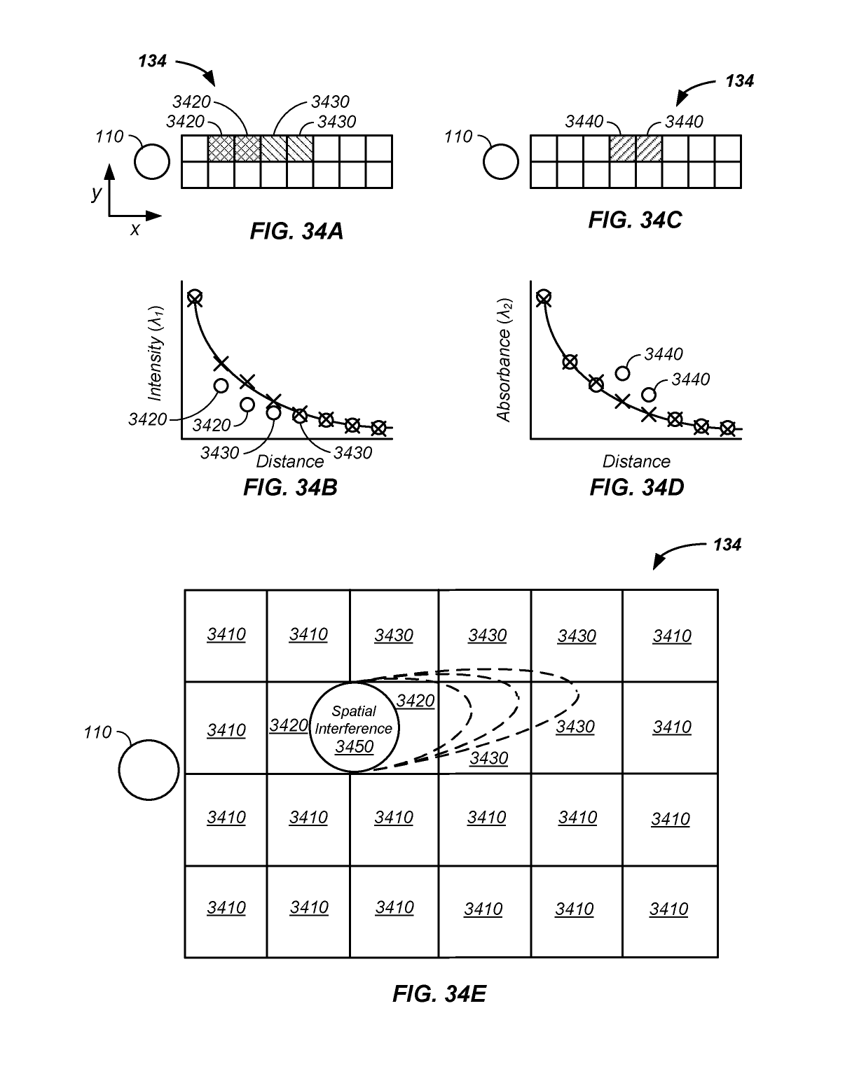

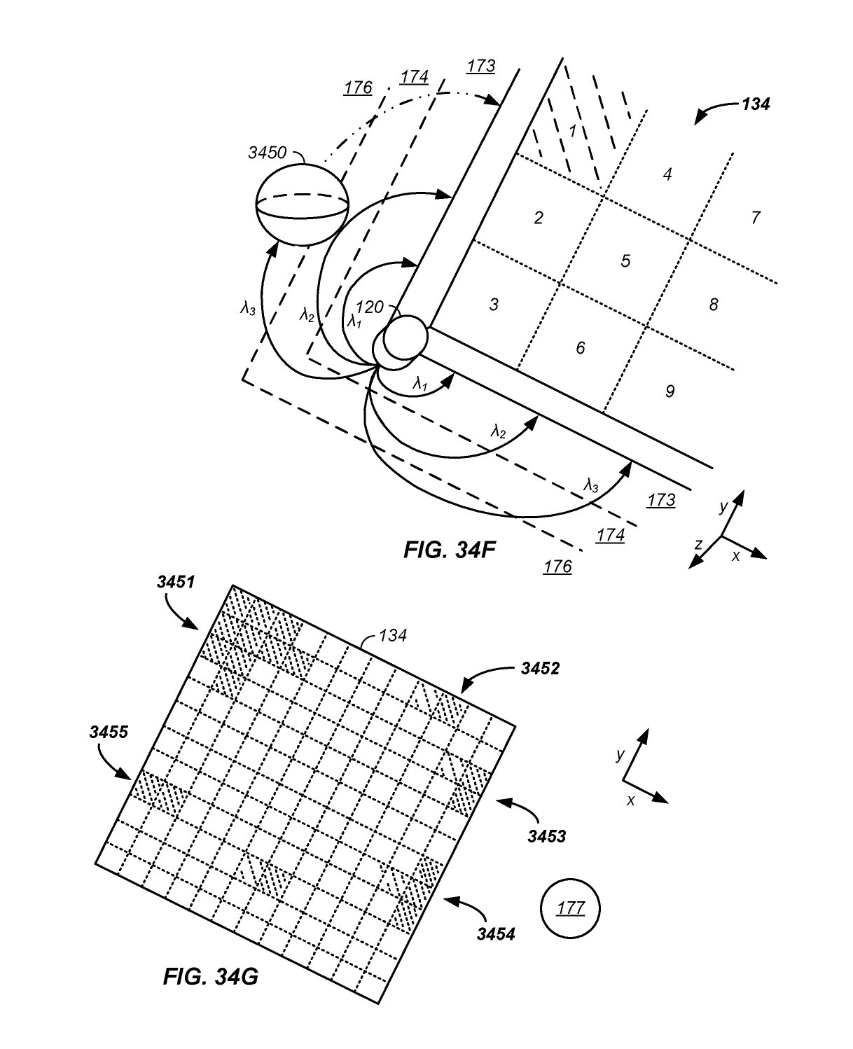

Referring now to FIGS. 34(A-G), generally, changes in spectra as a function of time, position, and/or radial distance are a continuum with offsets observed at sample interfaces, such as in the presence of a new tissue layer and/or in the presence of a interfering element. Thus, changes in intensity and absorbance are predicted to generally follow basic model parameters. For instance, observed intensity at a fixed wavelength is generally expected to decrease with radial distance between an illumination zone and a detection zone. Similarly, observed absorbance and/or an observed diffuse reflectance response is expected to increase with radial distance between the illumination zone and the detection zone. Further, the observed changes for intensity or absorbance are predicted to behave in a similar manner with small lateral changes in detection distance, especially with common radial distance from an illumination zone. Differences from expectations are indicators of a tissue change, such as a layer interface; a tissue inhomogeneity, such as from a blood vessel or non-uniform layer interface as with the spatially oscillating papillary dermis interface; and/or represent a physical interference, such as a hair follicle. Observed commonalities of response and/or observed differences in response are optionally used in the above described steps of binning detector-source combinations 3340, determination of outlier data 3330, correlation of data 3350; and/or selection of data 3320. Multiple examples of sample mapping.

### Example I

Referring now to FIG. 34A and FIG. 34B, a first non-limiting example provided for clarity of presentation is presented using a representative sample illumination zone from the source system 110 adjacent to a two-row detector array, representative of the two-dimensional detector array 134. As illustrated in FIG. 34B by the solid line, the observed intensity at a first wavelength, λ₁, is expected to fall off logarithmically as a function of distance along the x-axis. Further, due to the symmetry between the illumination zone and the two illustrated rows of detector elements, the observed signal from each paired set of detector elements in each column are expected to match, as they are observed to do for the first, sixth, seventh, eighth, and ninth columns in FIG. 34B. However, a first response difference 3420 is observed for the responses in the second and third detector columns, representative of a tissue difference observed between the first and second detector row. Further, using the smooth expected intensity response, it can be determined that an observed first interference or interfering sample constituent is observed by the first detector row and not the second detector row. Combined, the first interference is inferred to be at a radial position observed by the second and third detector element of the first row of detectors and at a lateral position observed by the first row of detectors. Still further, since the differences were observed in the detector elements at a second and third radial distance for the first wavelength, λ₁, a tissue model is optionally used to determine the depth of the first interference. Generally, an x,y-position and a z-depth of the first interference is roughly determined from the method. The position is optionally and preferably more precisely determined using responses from more than one wavelength and/or by providing photons to a separate, optionally overlapped, illumination zone relative to the detector array 134. Optionally, the entire sample interface 150 is optionally moved along the tissue of the subject to provide new set(s) of illumination zone-detection zone data to further determine the spatial position of the first interference.

### Example II

Still referring to FIG. 34A and FIG. 34B and additionally referring to the previous example, a second example is provided to illustrate interference shadowing on elements of the representative two-row detector array. Particularly, a second response difference 3430 is presented, where the observed comparison of detector response signals from the fourth and fifth columns are ambiguous, due to a small observed difference. However, the previous example determined an interference directly between the questioned detection area of the first row-fourth and fifth columns and the illumination zone. As the detected pathways are not absolute and, rather, statistical pathways are represented herein as of generally a banana shape as described in reference to FIG. 2A, a shadow effect from the interference is inferred to cause the slightly smaller response of the fourth and fifth detector element of the first row of detectors compared to the fourth and fifth detector element of the second row of detectors. Thus, in this case the response from the second row of detection elements is preferred for each of the second through fifth column of detectors. Generally, an x,y-position and a z-depth of a shadow of an interference is determined from the method.

### Example III

Referring now to FIG. 34C and FIG. 34D, a third example is provided to illustrate interference shadowing on elements of the representative two-row detector array, where an absorbance axis is used as a response signal. Generally, the example illustrates that any response function as a function of radial and lateral distance is used. In the illustrative example, the larger than expected absorbance at a second wavelength, λ₂, indicates a second interference or interfering sample constituent at a radial distance of the fourth and fifth column and in a lateral position of the first row of the two-dimensional detector array.

### Example IV

Referring again to FIG. 34A and FIG. 34B, a fourth example is presented using a representative sample illumination zone from the source system 110 adjacent to a multiple row-multiple column detector array, representative of the two-dimensional detector array 134. As illustrated, the first sample interference 3450 is observed in detection responses from detectors positioned in the second row and third/fourth columns of the detector array as the first response difference 3420 and the second, shadowing, response difference 3430 is observed as described above, while additionally being observed in another lateral row, illustrated as a first row, of detection elements. Generally, it is observed that outlier responses extend outward from the spatial interference 3450 in an approximate teardrop pattern along an axis extending outward from the illumination zone through the spatial interference 3450.

### Example V

Referring now to FIG. 34F, a fifth example is provided to further illustrate the process of determination of depth of a first sample interference 3450. As illustrated, the second wavelength, λ₂, arriving at the second and sixth detector elements does not pass through the first sample interference 3450 and approximately equal signals are detected. However, photons at the third wavelength, λ₃, interact with the first sample interference 3450 before detection at the first detector element, while no sample interference is interacted with by photons at the third wavelength, λ₃, on pathways to the ninth detector element. Again, the differences allow detection and placement of interferences in the skin of the subject 170.

### Example VI

Referring now to FIG. 34G, a sixth example is provided that illustrates combinations of the previous five examples to determine outlier positions and depths for a second through sixth sample interference, 3451-3455.

Each of the previous six examples provide means for detecting sub-surface anomalies, sample interferences, and/or for discarding data so that subsequent correlations established between observed signals and an analyte property, such as a glucose concentration, are more robust, accurate, and/or precise. The methods described separately in the example for clarity of presentation are synergistic and are optionally and preferably combined.

### Chemical Correlation

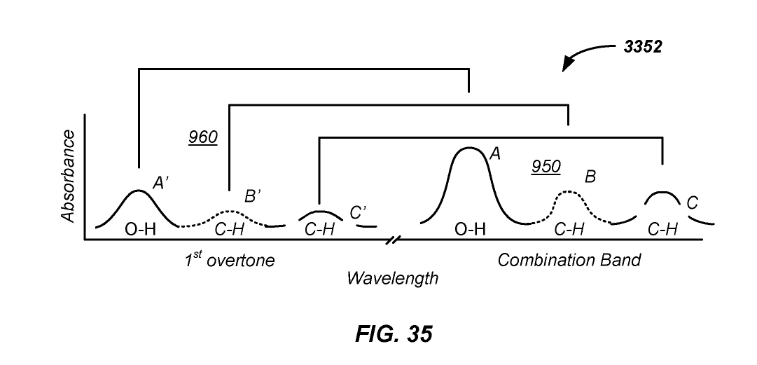

Referring now to FIG. 35, chemical correlation is described. Traditional noninvasive glucose concentration analyzers look at a response at each wavelength, but fail to use chemical correlations between wavelengths. FIG. 35 illustrates a first absorbance band, A, that absorbs in the combination band 950 where the chemical bond, such as an oxygen-hydrogen bond, has a higher energy state or correlated second absorbance band, A′, such as illustrated in the first overtone region 960. Thus an absorbance observed in the combination band 950 has a correlated absorbance in the first overtone region 960. Further, if the absorbance band is observed in the combination band region, then it is inferred that the molecule is at a shallow depth in the tissue due to the large absorbance of water limiting optical penetration depth, with subsequent photon detection. Additional chemical correlation bands are identified for a first carbon-hydrogen absorbance band, B, and a second carbon-hydrogen bond, C, of the glucose molecule. Similar relations are observed for the oxygen-hydrogen bond of glucose and the carbon-hydrogen bond of glucose between the combination band region 950 and/or the first overtone region 960 with absorbance bands observed in the second overtone region 970. Depth analysis of the absorbance bands is optionally performed in conjunction with water absorbance, scattering, and/or anisotropy as a function of wavelength, as described supra.

### Data Processing System

The data processing system 140 is further described herein. Generally, the data processing system uses an instrument configuration analysis system to determine an optical configuration of the analyzer 100 and/or a software configuration of the analyzer 100 while a sample property analysis system is used to determine a chemical, a physical, and/or a medical property, such as an analyte concentration, measured or represented by collected spectra. Further, the data processing system 140 optionally uses a preprocessing step and/or a processing step to determine an instrument configuration and/or to determine an analyte property.

In one embodiment, the data processing system 140 uses a preprocessing step to achieve any of: lower noise and/or higher signal. Representative and non-limiting forms of preprocessing include any of: use of a digital filter, use of a convolution function, use of a derivative, use of a smoothing function, use of a resampling algorithm, and/or a form of assigning one or more spectra to a cluster of a whole. The data processing system subsequently uses any multivariate technique, such as a form of principal components regression, a form of partial least squares, and/or a form of a neural network to further process the pre-processed data.

In another embodiment, the data processing system 140 and/or the sample property analysis system operates on spectra collected by the analyzer 100, such as in the subject specific data collection phase 2930, using a first step of defining finite width channels and a second step of feature extraction, which are each further described, infra.

### Finite Width Channels

In one example, the sample property analysis system defines a plurality of finite width channels, where the channels relate to changes in an optical parameter, software setting of the analyzer 100, a chemical condition, a physical property, a distance, and/or time. Still further, the channels optionally relate to radial distance between the incident light from the analyzer 100 entering skin of the subject 170 and detected light exiting the skin of the subject 170 and detected by the detector system 130, a focal length of an optic, a solid-angle of a photon beam from the source system 110, an incident angle of light onto skin of the subject, a pathlength, a pathway, and/or a software setting, such as control over spectral resolution. For clarity of presentation, the channels are described herein in terms of wavelength channels. For example, a spectrum is collected over a range of wavelengths and the finite width channels represent finite width wavelength channels within the spectrum. Generally, the channels are processed to enhance localized signal, to decrease localized noise, and/or are processed using a cross-wavelet transform.

In one case, the sample property analysis system 2950 defines a plurality of finite width wavelength channels, such as more than 3, 5, 10, 15, 20, 30, 40, or 50 wavelength channels, which are al referred to as a pathway, contained in a broader spectral region, such as within a spectrum from 900 to 2500 nanometers or within a sub-range therein, such as within 1100 to 1800 nanometers. The plurality of multiple finite width wavelength channels enhance accessibility to content related to: (1) a target analyte, such as a glucose concentration, and (2) a measurement context, such as the state of skin of the subject 170, which is used as information in a self-correcting background.

### Feature Extraction

In one case, feature extraction determines and/or calculates coherence between channels, which is referred to herein as cross-coherence, to identify and/or enhance information common to the analytical signal, such as frequency, wavelength, shift, and/or phase information. Subsequently, cross-coherence terms are selected using a metric, such as to provide maximum contrast between: (1) the target analyte or signal and (2) the measurement context or background. Examples of background include, but are not limited to: spectral interference, instrument drift impacting the acquired signal, spectral variation resultant from physiology and/or tissue variation, temperature impact on the analyzer, mechanical variations in the analyzer as a function of time, and the like. Generally, the cross-coherence terms function to reduce monotonicity detected variation as a function of analyte concentration. In a particular instance, an N×N grid is generated per spectrum, which is symmetric about the diagonal of the N×N grid, with each grid element representing an M term coherence estimate versus frequency, where N is a positive integer of at least three.

### Model

Typically, a model, such as a nonlinear model or transform, is constructed to map the extracted features to the analyte property, such as a glucose concentration. For example, the total differential power of the cross-coherence estimate is determined between features related to the analyte versus the background and a separate nonlinear function is calculated for multiple analyte ranges.

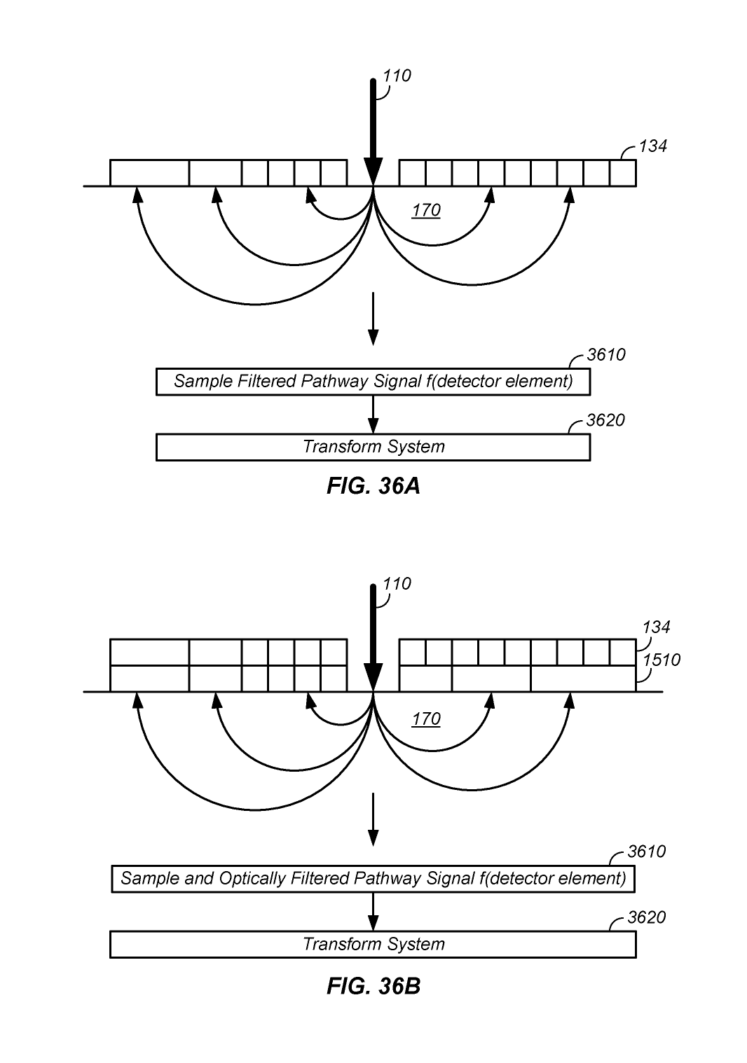

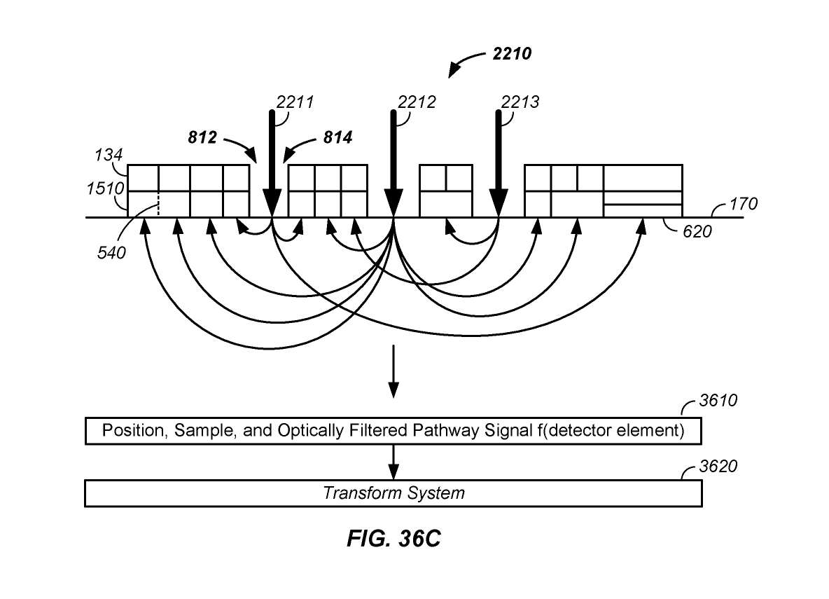

Referring now to FIGS. 36(A-C), a method is illustratively presented that links sample filtered pathways 3610 between the source system 110 and the two-dimensional detector array 134 to the transform system 3620 of the analyzer 100, which transforms the detected signals to an analyte property estimation and/or determination.

### Example I

Referring now to FIG. 36A, a first non-limiting example is provided to clarify the invention. In this example, the sample itself performs as the optical filter separating signals as a function of pathway. As illustrated, photons enter the subject 170 over a single illumination region and are separated by the sample as a function of radial distance before detection by elements of the two-dimensional detector array 134 as described in the description of FIGS. 2A to 23C. The two-dimensional detector array 134 is illustrated with detector elements that optionally increase in radial cross-section length as a function of radial distance.

### Example II

Referring now to FIG. 36B, a second non-limiting example is provided to clarify the invention. In this example, an optical filter array, such as the first optical filter array 1510, is used to filter sample filtered pathways 3610 between the source system 110 and the two-dimensional detector array 134. Optionally, the optical filter array is any mechanism that separates wavelengths of light, such as a time domain to frequency domain spectrometer. The optical filter array 1510 is illustrated with optional filter elements that increase in radial cross-section length as a function of radial distance.

### Example III

Referring now to FIG. 36C, a third non-limiting example is provided to clarify the invention. In this example, multiple illumination zones are illustrated where the multiple illumination zones initiate a set of overlapping, yet distinguishable, pathways, as described throughout and emphasized in the description of FIGS. 2A to 22C. The example illustrates many optional elements and/or systems described throughout, such as: (1) use of multiple separated illumination zones and/or use of multiple illumination wavelengths, such as via use of multiple sets of LED types 2210, such as a first LED set type 2211, a second LED set type 2212, and a third LED set type 2213; (2) use of multiple detector elements, such as the two-dimensional detector array 134; (3) use of multiple illumination zone to detection zone gap distances, such as the first illuminator/detector gap 812 and the second illuminator/detector gap 814; (4) use of a focusing optic and/or focusing optic array, such as the optical detector filter 620; and/or (5) use of one or more segmented spacers 540. Further, as described throughout, the illumination wavelength-filter combinations are coordinated with sample properties, such as absorbance, scattering, and anisotropy, to aid the skin of the subject 170 separating mean wavelength bands, mean depths of penetration, mean pathlength, and/or mean pathlength in a tissue layer. Still further, the apparatus elements and methods described herein are optionally and preferably used in combination to yield synergistic channels of information for the algorithm, transform system, and/or model of the analyzer.

Referring again to FIGS. 1-22C, a noninvasive near-infrared analyzer is configured to limit variation of detected optical pathlength of the sample and/or spatially separate collected light having probed different sample volumes using a combination of limits including:
discrete illumination zones as illustrated in FIG. 4B;
discrete detection zones as illustrated in FIG. 5B;
segmented spacers as illustrated in FIG. 5D;
rings and/or arcs of detector elements as illustrated in FIG. 5G;
intersecting groups of detector elements as illustrated in FIG. 5N;
distributed illumination areas and/or interconnected detection zones as illustrated in FIG. 5N;
illuminator to detector distance control as illustrated in FIG. 5O;
more than one detection zone size as illustrated in FIG. 18A;
a two-dimensional optical filter array as illustrated in FIG. 20B;
a two-dimensional micro-optic array as illustrated in FIG. 20B;
controlled or varying detector types having different materials, detector element thickness, chemical doping, and/or response shapes;
a sub-set of detector elements optically coupled to a smaller detection zone versus combined signals from a larger number of detector elements detecting photons from a larger range of illumination zone to detection zone distances;
control of numerical aperture of coupling optics as a function of distance from an illumination zone; and/or
detecting and removing outlier signals resultant from probing photons interacting with tissue inhomogeneities, such as a hair follicle, a localized refractive index change, a localized scattering change, and/or a geometric variation at an interface of two or more tissue layers.

Generally, control of optical depth of penetration, total pathlength in a desired tissue zone, and/or total tissue pathlength is achieved with any one of the described limits. However, the inventor has determined that combinations of three or more, such as 4, 5, 6, 7, 8 or more, of the described limits interact to still further control the optical depth of penetration, total pathlength in a desired tissue zone, and/or total tissue pathlength with a resulting improvement in accuracy and/or prediction of an analyte concentration in tissue using near-infrared spectroscopy in the range of 1100 to 2500 nm.

### Personal Communication Device

Herein, a personal communication device comprises any of a wireless phone, a cell phone, a smart phone, a tablet, a phablet, a wearable internet connectable accessory, a wearable internet connectable garment, and/or a smart wearable accessory, such as a watch with internet and/or phone communication ability. Optionally, the analyzer 100 has no display screen and results are transmitted to a personal communication device of the user, which allows a smaller analyzer 100 and/or the analyzer to be semi-continuously worn in a non-conspicuous location, such as under a shirt or around the torso of the individual.

Optionally, the personal communication device and/or the analyzer communicate with a data processing center. For example, the data processing center received data from the analyzer 100 through use of at least one wireless step, processes the data, and sends a result and/or a model parameter to the personal communication device of the user, resulting in one or more of: a displayed analyte concentration, a description of detection of an analyzer error, and/or an alert, such as a rapidly falling glucose concentration, an abnormally high glucose concentration, and/or a request for use of an alternative glucose determination method.

Herein, numbers illustrating distance and/or comparative numbers, such as 1, 2, 5, 10, optionally refer to: (1) at least the provided number or (2) less than the provided number.

Still yet another embodiment includes any combination and/or permutation of any of the analyzer and/or sensor elements described herein.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

In the foregoing description, the invention has been described with reference to specific exemplary embodiments; however, it will be appreciated that various modifications and changes may be made without departing from the scope of the present invention as set forth herein. The description and figures are to be regarded in an illustrative manner, rather than a restrictive one and all such modifications are intended to be included within the scope of the present invention. Accordingly, the scope of the invention should be determined by the generic embodiments described herein and their legal equivalents rather than by merely the specific examples described above. For example, the steps recited in any method or process embodiment may be executed in any order and are not limited to the explicit order presented in the specific examples. Additionally, the components and/or elements recited in any apparatus embodiment may be assembled or otherwise operationally configured in a variety of permutations to produce substantially the same result as the present invention and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

Herein, a set of fixed numbers, such as 1, 2, 3, 4, 5, 10, or 20 optionally means at least any number in the set of fixed number and/or less than any number in the set of fixed numbers.

Herein, specific wavelengths are used to facilitate communication of key spectroscopic points. However, the specific wavelengths presented are optionally plus and/or minus 1, 2, 5, 10, 20, 30, 40, 50, 75, or 100 nm.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. An apparatus for detecting distributed probing light emerging from a subject, comprising:
a noninvasive analyzer, comprising:
a near-infrared light source configured to provide photons during use;
an optical interface to the subject, comprising:
an illumination area; and
a detection area comprising, during use, a surface area of the subject,
wherein, during use, the photons sequentially pass through the illumination area into a portion of the subject and out of the subject through the detection area;
a set of detectors within ten centimeters of the illumination area, said set of detectors comprising:
a first detector optically coupled to a first detection zone of the detection area; and
a second detector optically coupled to a second detection zone of said detection area,
the second detection zone: (1) at least twenty percent larger than the first detection zone and (2) positioned radially outward from the illumination area relative to the first detection zone.

2. The apparatus of claim 1, said set of detectors further comprising:
a first group of detectors, electrically connected for readout in series, said first group of detectors optically linked to a first set of detection zones at least partially circumferentially encircling the illumination area at a first radial distance;
a second group of detectors, electrically connected for readout in series, said second group of detectors optically linked to a second set of detection zones at least partially circumferentially encircling the illumination area at a second radial distance.

3. The apparatus of claim 2, further comprising:
a segmenting spacer extending perpendicularly from the detection area into said optical interface between: (1) said first group of detectors and (2) said second group of detectors, said segmenting spacer comprising at least one of:
an air gap;
a mirrored surface; and
a change in refractive index, as measured by Snell's Law, sufficient to redirect photons striking the segmented spacer back toward an axis running both perpendicular to the detection area and through a point of exit of the photons from the subject during use.

4. The apparatus of claim 2, the illumination area further comprising:
a first ring illumination area coupled to a first light source; and
a second ring illumination area coupled to a second light source.

5. The apparatus of claim 2, further comprising:
a two-dimensional optical filter array; and
a two-dimensional micro-optic array comprising a first face substantially in proximate contact with said two-dimensional optical filter array, at least one of said two-dimensional optical filter array and said two-dimensional micro-optic array comprising a second face substantially in proximate contact with said set of detectors.

6. The apparatus of claim 2, said set of detectors further comprising:
a first hardware gain linked to said first group of detectors; and
a second hardware gain linked to said second group of detectors, said second hardware gain at least ten percent larger than said first hardware gain.

7. The apparatus of claim 1, said set of detectors further comprising:
at least three detectors optically linked to a corresponding at least three detection zones of the detection area, the at least three detection zones of increasing area as a function of radial distance from the illumination zone.

8. The apparatus of claim 1, further comprising:
a first geometric unit of detectors positioned a first direction from the illumination zone; and
a second geometric unit of detectors positioned a second direction from the illumination zone, said second geometric unit of detectors comprising a common geometry with said first geometric unit of detectors, said second geometric unit of detectors rotated relative to an orientation of said first geometric unit of detectors.

9. The apparatus of claim 8, further comprising:
a set of detector readout elements placed proximate an outer perimeter of a space occupied by a union of the illumination zone, the first geometric unit of detectors, and the second geometric unit of detectors.

10. The apparatus of claim 1, further comprising:
a set of micro-optics aligned with detection elements of said set of detectors, said set of micro-optics comprising a first micro-optic and a second micro-optic,
said first micro-optic, coupled to the first detection zone, comprising a first numerical aperture,
said second micro-optic, coupled to the second detection zone, comprising a second numerical aperture,
said first numerical aperture coupled to the first detection zone, positioned radially inward from the second detection zone, at least ten percent greater than said second numerical aperture.

11. The apparatus of claim 10, said set of micro-optics further comprising:
at least three optics positioned at increasing radial distance from the illumination zone, said at least three optics comprising three numerical apertures that decrease as a function of distance from the illumination zone.

12. The apparatus of claim 1, the illumination area further comprising:
a set of illumination zones, comprising:
a first illumination zone at least partially circumferentially encircled by a first group of detection zones, the first group of detection zones comprising at least two rings of detection zones linked to at least two groups of detectors of said set of detectors; and
a second illumination zone at least partially circumferentially encircled by a second group of detection zones linked to a third group of detectors, the second illumination zone positioned outside of the first group of detection zones.

13. The apparatus of claim 12, wherein a member of said at least two groups of detectors is a member of said third group of detectors.

14. The apparatus of claim 2, wherein said first detector further comprises a first indium gallium arsenide detection element of a first thickness,
wherein said second detector further comprises a second indium gallium arsenide detection element of a second thickness, said second thickness at least ten percent thicker than said first thickness,
wherein a difference between said first thickness and said second thickness alters a wavelength response profile of said first detector relative to said second detector.

15. A method for detecting distributed probing light emerging from a subject, comprising the steps of:
providing a noninvasive analyzer, comprising:
a near-infrared light source;
an optical interface to the subject comprising: an illumination area and a detection area comprising a surface area of the subject; and
a set of detectors within ten centimeters of the illumination area, said set of detectors comprising:
a first detector optically coupled to a first detection zone of the detection area; and
a second detector optically coupled to a second detection zone of the detection area,
the second detection zone: (1) at least twenty percent larger than the first detection zone and (2) positioned radially outward from the illumination area relative to the first detection zone;
providing photons with said near-infrared light source; and
sequentially passing the photons through the illumination area into a portion of the subject and out of the subject through the detection area to said set of detectors.

16. The method of claim 15, further comprising the steps of:
collecting light using the first detection zone, the first detection zone at least partially circumferentially encircling the illumination area, the first detection zone optically linked to a first set of detectors; and
collecting light using the second detection zone, the second detection zone at least partially circumferentially encircling the illumination area, the second detection zone optically linked to a second set of detectors.

17. The method of claim 16, further comprising the step of:
using signals from said first set of detectors to determine an outlier signal corresponding to a member of said signals, said outlier signal comprising a statistically significant difference from a mean signal generated from said signals, said first set of detectors comprising at least six detectors.

18. The method of claim 17, further comprising the step of:
using an algorithm to determine an analyte property of the sample, said algorithm discarding at least one response from a radially outward detector of said set of detectors, wherein said radially outward detector falls on a line passing through the illumination zone and an outlier detector generating said outlier signal.

19. The method of claim 17, further comprising the step of:
mathematically combining said signals from said first set of detectors after removal of the outlier signal, wherein said first set of detectors overlap a single radial distance from the illumination zone.

20. The method of claim 10, further comprising the steps of:
illuminating a first sub-area of the illumination area at a first time; and
illuminating a second sub-area of the illumination area at a second time, wherein a difference in distance between: (1) the first-sub area and the first detection zone and (2) the second-sub area and the first detection zone yields detection of differing mean total pathlengths of the photons in the subject using said set of detectors.
